SEQUENCE LISTING (40 sequences)

SEQ ID NO: 1 - protein, 4 aa, synthetic construct
DRDC

SEQ ID NO: 2 - protein, 4 aa, synthetic construct
DKDC

SEQ ID NO: 3 - skipped

SEQ ID NO: 4 - protein, 4 aa, synthetic construct
  MOD_RES 3: note = homoArginine
DRDG

SEQ ID NO: 5 - protein, 4 aa, synthetic construct
DKDK

SEQ ID NO: 6 - protein, 4 aa, synthetic construct
DKDV

SEQ ID NO: 7 - protein, 4 aa, synthetic construct
  MOD_RES 2: note = HomoArginine
DRDC

SEQ ID NO: 8-11 - skipped (4 entries)

SEQ ID NO: 12 - protein, 386 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKEFSSSSGSSSSGSSSSGAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 5 aa, synthetic construct
SFSMS

SEQ ID NO: 14 - protein, 17 aa, synthetic construct
SISGSSGTTYYADSVKG

SEQ ID NO: 15 - protein, 7 aa, synthetic construct
PFPYFDY

SEQ ID NO: 16 - protein, 12 aa, synthetic construct
RASQSVSSSFLA

SEQ ID NO: 17 - protein, 7 aa, synthetic construct
YASSRAT

SEQ ID NO: 18 - protein, 9 aa, synthetic construct
QQTGRIPPT

SEQ ID NO: 19 - protein, 116 aa, synthetic construct [sequence omitted]

SEQ ID NO: 20 - protein, 108 aa, synthetic construct [sequence omitted]

SEQ ID NO: 21 - protein, 133 aa, synthetic construct [sequence omitted]

SEQ ID NO: 22 - protein, 12 aa, synthetic construct
GDGSSGGSGGAS

SEQ ID NO: 23 - protein, 17 aa, synthetic construct
EFSSSSGSSSSGSSSSG

SEQ ID NO: 24 - protein, 15 aa, synthetic construct
GGGGSGGGGSGGGGS

SEQ ID NO: 25 - protein, 15 aa, synthetic construct
GSLDGAGGSAGADGG

SEQ ID NO: 26 - protein, 5 aa, synthetic construct
GSSGG

SEQ ID NO: 27 - protein, 5 aa, synthetic construct
GGSGG

SEQ ID NO: 28 - protein, 229 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGSSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 29 - protein, 473 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGSSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGSLDGAGGSAGADGGEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGSSGGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 30 - protein, 30 aa, synthetic construct
EVQLLESGGGLVQPGGSLRLSCAASGFTFS

SEQ ID NO: 31 - protein, 14 aa, synthetic construct
WVRQAPGKGLEWVS

SEQ ID NO: 32 - protein, 32 aa, synthetic construct
RFTISRDNSKNTLYLQMNSLRAEDTAVYYCAK

SEQ ID NO: 33 - protein, 11 aa, synthetic construct
WGQGTLVTVSS

SEQ ID NO: 34 - protein, 23 aa, synthetic construct
EIVLTQSPGTLSLSPGERATLSC

SEQ ID NO: 35 - protein, 15 aa, synthetic construct
WYQQKPGQAPRLLIY

SEQ ID NO: 36 - protein, 32 aa, synthetic construct
GIPDRFSGSGSGTDFTLTISRLEPEDFAVYYC

SEQ ID NO: 37 - protein, 10 aa, synthetic construct
FGQGTKVEIK

SEQ ID NO: 38 - protein, 236 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 39 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 40 - protein, 10 aa, synthetic construct
SSVGYYTVTG

(19) United States
(12) Patent Application Publication
(10) Pub. No.: US 2026/0364214 A1
(43) Pub. Date: Nov. 19, 2026

(54) MULTIVALENT FIBROBLAST ACTIVATION PROTEIN LIGANDS FOR TARGETED DELIVERY APPLICATIONS

(71) Applicant: Philochem AG, Otelfingen (CH)

(72) Inventors: Matilde BOCCI, Otelfingen (CH); Samuele CAZZAMALLI, Otelfingen (CH); Andrea GALBIATI, Otelfingen (CH); Dario NERI, Otelfingen (CH); Aureliano ZANA, Otelfingen (CH); Ettore GILARDONI, Otelfingen (CH); Lucrezia PRINCIPI, Otelfingen (CH)

(21) Appl. No.: 19/109,087

(22) PCT Filed: Sep. 5, 2023
(86) PCT No.: PCT/EP2023/074310

§ 371 Date: Mar. 5, 2025

(30) Foreign Application Priority Data
Sep. 6, 2022 (EP) 22194233.7
Oct. 17, 2022 (EP) 22201953.1
Mar. 12, 2023 (EP) 23161360.5

(51) Int. Cl. A61K 51/08; A61K 47/64; A61K 47/68; A61K 51/04; A61P 35/00

(52) U.S. Cl. CPC A61K 51/088; A61K 47/64; A61K 47/6813; A61K 47/6843; A61K 51/0482; A61P 35/00

(57) ABSTRACT

The present invention relates to ligands of Fibroblast Activation Protein (FAP) for the active delivery of various therapeutic payloads (e.g., cytotoxic drugs, therapeutic radionuclides, proteins and immunomodulators) at the site of disease. In particular, the present invention relates to the development of multivalent FAP ligands for therapeutic applications, in relation to a disease or disorder, such as cancer, inflammation or another disease characterized by overexpression of FAP.

### INTRODUCTION

### Field

The present invention relates to ligands of Fibroblast Activation Protein (FAP) for the active delivery of various therapeutic payloads (e.g., cytotoxic drugs, therapeutic radionuclides, proteins and immunomodulators) at the site of disease. In particular, the present invention relates to the development of multivalent FAP ligands for therapeutic applications, in relation to a disease or disorder, such as cancer, inflammation or another disease characterized by overexpression of FAP.

### Background of the Invention

Chemotherapy is still widely applied for the treatment of cancer patients and of other diseases. Conventional anti-cancer chemotherapeutic agents act on basic mechanisms of cell survival and cannot distinguish between healthy cells and malignant cells. Moreover, those drugs do not accumulate efficiently to the site of the disease upon systemic administration. Unspecific mechanism of actions and inefficient localization at the tumor site account for unsustainable side-effects and poor therapeutic efficacy of conventional chemotherapy.

The development of therapeutic drugs, able to selectively localize at the site of the disease after systemic administration, is highly desirable. A strategy to generate such therapeutic drugs is represented by the chemical conjugation of a therapeutic payload, like cytotoxic drugs or therapeutic radionuclides, to a ligand specific to a marker of a disease. Disease-specific monoclonal antibodies, peptides and small ligands have been considered as ligands of choice for the development of targeted therapeutic drug products. The use of small ligands for therapeutic applications has several advantages compared to bigger molecules like peptides and antibodies: more rapid and efficient tumor penetration, lower immunogenicity and lower manufacturing costs.

Small organic ligands specific to prostate-specific membrane antigen, folate receptor and carbonic anhydrase IX have shown excellent biodistribution profiles in preclinical models of cancer and in patients. These ligands have been conjugated to cytotoxic drugs and to radionuclides to generate small molecule-drug conjugate and small molecule-radio conjugate products (SMDCs and SMRCs) for the treatment of cancer. 177-Lutetium-PSMA-617 represents an example of a late stage SMRC which is now being investigated in a phase III trial for the treatment of metastatic castrate-resistant prostate cancer (mCRPC) patients (VISION trial).

Fibroblast activation protein (FAP) is a membrane-bound gelatinase which promotes tumor growth and progression and is overexpressed in cancer-associated fibroblasts. FAP represents an ideal target for the development of therapeutic SMDCs and SMRCs due to its low expression in normal organs.

WO2019154886 and WO2019154859 describe heterocyclic compounds as fibroblast activation protein-alpha inhibitors used to treat different cancer types. WO2019118932 describes substituted N-containing cyclic compounds as fibroblast activation protein alpha inhibitors used to treat different pathological conditions. WO2019083990 describes imaging and radiotherapeutic targeting fibroblast-activation protein-alpha (FAP-alpha) compounds as FAP-alpha inhibitors used for imaging disease associated with FAP-alpha and to treat proliferative diseases, and notes that the 4-isoquinolinoyl and 8-quinolinoyl derivatives described therein are characterized by very low FAP-affinity. WO2013107820 describes substituted pyrrolidine derivatives used in the treatment of proliferative disorders such as cancers and diseases indicated by tissue remodeling or chronic inflammation such as osteoarthritis. WO2005087235 describes pyrrolidine derivatives as dipeptidyl peptidase IV inhibitors to treat Type II diabetes. WO2018111989 describes conjugates comprising fibroblast activation protein (FAP) inhibitor, bivalent linker and e.g. near infrared (NIR) dye, useful for removing cancer-associated fibroblasts, imaging population of cells in vitro, and treating cancer. WO2021/160825 A1, WO2022/171811 A1 and WO2021/016392 A1 describe ligands of FAP for the active delivery of various payloads. Zboralski, D., Hoehne, A., Bredenbeck, A. et al. Eur J Nucl Med Mol Imaging 49, 3651-3667 (2022) describe FAP-2286 (a FAP-binding peptide coupled to a radionuclide chelator), and report that radiolabeled FAP-2286 demonstrated high tumor uptake and retention, as well as potent efficacy in FAP-positive tumors.

Nevertheless, there remains a demand for further improved FAP binders for the above and further applications.

The present invention thus aims at the problem of providing improved therapeutic binders (ligands) of fibroblast activation protein (FAP) suitable for therapeutic applications. The binders should be suitable for inhibition of FAP and/or targeted delivery of a therapeutic payload, to a site afflicted by or at risk of disease or disorder characterized by overexpression of FAP, such as FAP-positive tumors. Preferably, the binders should exhibit improved binding parameter(s) (e.g., FAP inhibitory activity, FAP binding affinity and/or prolonged binding to FAP-positive cells), while showing advantageous tumor uptake or tumor-to-organ ratio (e.g., tumor-to-kidney ratio).

### SUMMARY OF THE INVENTION

The present inventors have found novel trivalent organic ligands of fibroblast activation protein (FAP) (“Tri-ESV6”) suitable for therapeutic applications. The compounds according to the present invention (also referred to as ligands or binders) comprise more than two (e.g., three) small binding moieties A having the following structure:

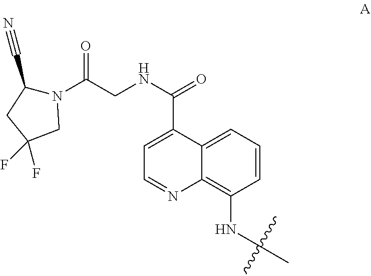 [SMILES: *Nc1cccc2c(C(=O)NCC(=O)N3CC(F)(F)C[C@H]3C#N)ccnc12]

A compound according to the present invention may be represented by following general Formula I,

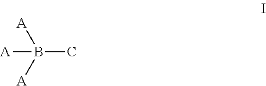

its individual diastereoisomers, its hydrates, its solvates, its crystal forms, its individual tautomers or a pharmaceutically acceptable salt thereof, wherein A is a binding moiety; B is a multifunctional moiety comprising a ramification point, and covalently connects the moieties A to C; and C is an atom, a molecule or a particle, and is a therapeutic agent. An exemplary structure is represented by general Formula II as defined hereinbelow.

Alternatively, moiety C comprises a ramification point, such that three moieties A are attached to C, and B is absent, i.e., moiety C can also serve as a ramification point therefore substituting B, preferably when C is a chelator. An exemplary structure is represented by general Formula III as defined hereinbelow.

The present invention further provides a pharmaceutical composition comprising said compound and a pharmaceutically acceptable excipient.

The present invention further provides said compound or pharmaceutical composition for use in a method for treatment of the human or animal body therapy; as well as a method for treatment of the human or animal body by therapy comprising administering a therapeutically effective amount of said compound or pharmaceutical composition to a subject in need thereof.

The present invention further provides said compound or pharmaceutical composition for use in a method for therapy of a subject suffering from or having risk for a disease or disorder; as well as a method for treatment therapy of a disease or disorder comprising administering a therapeutically effective amount of said compound or pharmaceutical composition to a subject suffering from or having risk for said disease or disorder.

The present invention further provides said compound or pharmaceutical composition for use in a method for targeted delivery of a therapeutic agent to a subject suffering from or having risk for a disease or disorder; as well as a method for targeted delivery of a therapeutically effective amount of said compound or pharmaceutical composition to a subject suffering from or having risk for a disease or disorder.

Preferably, the aforementioned disease or disorder is characterized by overexpression of FAP and is independently selected from cancer, inflammation, atherosclerosis, fibrosis, tissue remodeling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, esophageal cancer, hypopharynx cancer, nasopharynx cancer, larynx cancer, myeloma cells, bladder cancer, cholangiocarcinoma, clear cell renal carcinoma, neuroendocrine tumor, oncogenic osteomalacia, sarcoma, CUP (carcinoma of unknown primary), thymus cancer, desmoid tumors, glioma, astrocytoma, cervix cancer, skin cancer, kidney cancer and prostate cancer.

### BRIEF DESCRIPTION OF THE DRAWINGS

FIG. 1: Inhibition assay with recombinant hFAP at a concentration of 66 μM. Tri-ESV6-DOTAGA exhibits a remarkably lower IC₅₀ compared to ESV6-DOTAGA, Bi-ESV6-DOTAGA and FAP-2286.

FIG. 2: Fluorescence Polarization assay with recombinant hFAP, with fluorescent ligands at a concentration of 1 nM. Both ESV6-PEG₂-FITC and Tri-ESV6-PEG₂-FITC exhibit a remarkably lower K_{D} compared to FAP-2286-PEG₂-FITC.

FIG. 3: Efflux assay on SK-RC-52.hFAP cells with ¹⁷⁷Lu-labeled ligands. ¹⁷⁷Lu-Tri-ESV6 exhibits the longer residence time in FAP-positive tumor cells, followed by ¹⁷⁷Lu-Bi-ESV6, ¹⁷⁷Lu-ESV6 and ¹⁷⁷Lu-FAP2286.

FIG. 4: Quantitative biodistribution analysis with ¹⁷⁷Lu-ESV6, ¹⁷⁷Lu-Bi-ESV6, ¹⁷⁷Lu-Tri-ESV6, ¹⁷⁷Lu-Tetra-ESV6, ¹⁷⁷Lu-Hexa-ESV6, ¹⁷⁷Lu-Octa-ESV6 and ¹⁷⁷Lu-FAP-2286 (each 250 nmol/kg; 50 MBq/kg) on SK-RC-52.hFAP tumor bearing mice. Tri-ESV6-DOTAGA exhibits a remarkably high tumor uptake even after 48, 72 and 96 hours post injection. At the same dosage, the Tri-ESV6 binder exhibits the best biodistribution profile, when considering both tumor uptake over time and uptake in healthy organs.

FIG. 5: Therapeutic activity of ¹⁷⁷Lu-ESV6-DOTAGA, ¹⁷⁷Lu-Bi-ESV6-DOTAGA and ¹⁷⁷Lu-Tri-ESV6-DOTAGA or saline as single agents (single administration on day 8, 250 nmol/kg, 250 MBq/kg) in BALB/c nu/nu mice bearing SK-RC-52.hFAP tumors. The efficacy of the different treatments is assessed by daily measurement of tumor volume (mm³) during and after administration of the drugs. Data points represent mean tumor volume±SEM (n=4 per group). ****P<0.0001; *P<0.1 (two-way ANOVA test, followed by Bonferroni posttest). Tri-ESV6-DOTAGA exhibits a remarkable therapeutic activity.

FIG. 6: Therapeutic activity of ¹⁷⁷Lu-ESV6-DOTAGA, ¹⁷⁷Lu-Bi-ESV6-DOTAGA and ¹⁷⁷Lu-Tri-ESV6-DOTAGA or saline as single agents (single administration on day 8, 250 nmol/kg, 250 MBq/kg) in BALB/c nu/nu mice bearing SK-RC-52.hFAP tumors. The efficacy of the different treatments is assessed by daily measurement of tumor volume (mm³) during and after administration of the drugs. Curves represents single mouse tumor growth values. CR=Complete Remission. Tri-ESV6-DOTAGA exhibits a remarkable therapeutic activity.

FIG. 7: The tolerability of the different treatments with ¹⁷⁷Lu-ESV6-DOTAGA, ¹⁷⁷Lu-Bi-ESV6-DOTAGA and ¹⁷⁷Lu-Tri-ESV6-DOTAGA as assessed by the evaluation of changes (%) in body weight during the experiment. All conjugates were very well tolerated in a comparable manner.

FIG. 8: Inhibition assay with recombinant hFAP at a concentration of 66 μM. Surprisingly, Tri-ESV6-DOTAGA exhibits a ~1000-fold lower IC₅₀ compared to its derivative (Tri-ESV6-PEG12-DOTAGA) with longer spacers between the ESV6 moiety and the ramification.

FIG. 9: Therapeutic activity in BALB/c nu/nu mice bearing SK-RC-52.hFAP tumors following intravenous administration of (i) L19-IL2 as single agent at a dose of 0.05 mg/mouse on day 8, 10 and 12, (ii) ¹⁷⁷Lu-Tri-ESV6 as single agent at different doses of 5 MBq/mouse, 15 MBq/mouse or 30 MBq/mouse on day 7, (iii) ¹⁷⁷Lu-Tri-ESV6+L19-IL2 combination at a dose of 5 MBq/mouse of ¹⁷⁷Lu-Tri-ESV6 followed by three injections of L19-IL2 at a dose of 0.05 mg/mouse on day 8, 10 and 12 or (iv) saline. The efficacy of the different treatments is assessed by daily measurement of tumor volume (mm3) during and after administration of the drugs. Data points represent mean tumor volume±SEM (n=4 per group). ¹⁷⁷Lu-Tri-ESV6 exhibits a dose-dependent therapeutic activity.

FIG. 10: Inhibition assay with recombinant hFAP at a concentration of 66 μM. The compound with the shortest distance between the targeting moiety and the ramification point (PEG Unit=0) was surprisingly found to have the lowest IC50.

FIG. 11: Inhibition assay with recombinant hFAP at a concentration of 66 μM. The inhibitory activity is directly proportional to the valency until Tetra-ESV6-DOTAGA (tetravalent compound). Surprisingly, despite their superior valency, Hexa-ESV6-DOTAGA (hexavalent compound) and Octa-ESV6-DOTAGA (octavalent compound) lose over 10'000-fold activity compared to the tetravalent derivative. Compounds with alternative linker (ESV6-L-DOTAGA and Bi-ESV6-DOTAGA) present IC50 comparable to the ones of original derivatives (ESV6-DOTAGA and Bi-ESV6-DOTAGA in FIG. 1).

FIG. 12: Preferred immunocytokine for use in the present invention, namely an L19-IL2 conjugate immunocytokine, in which, in each polypeptide chain, an IL2 polypeptide sequence is fused, at its N-terminus via a linker, to the C-terminus of the VL domain of a single-chain variable fragment (scFv) sequence comprising the VH and VL domains of L19, and the scFv unit of one L19-IL2 polypeptide chain forms a homodimer with an scFv unit of another L19-IL2 polypeptide chain (“scFv2-format immunocytokine”).

FIG. 13: Therapeutic activity in BALB/c nu/nu mice bearing SK-RC-52.hFAP tumors following intravenous administration of: (i) L19-IL2 as single agent at a dose of 0.05 mg/mouse on day 8, 10, 12, (ii) ¹⁷⁷Lu-Tri-ESV6 as single agent at a dose of 5 MBq/mouse on day 7, (iii) ¹⁷⁷Lu-Tri-ESV6+L19-IL2 combination (¹⁷⁷Lu-Tri-ESV6 on day 7 at a dose of 5 MBq/mouse followed by three administrations of L19-IL2 at a dose 0.05 mg/mouse on day 8, 10 and 12 (SCHEDULE 1), or by three administrations of L19-IL2 at a dose 0.05 mg/mouse on day 12, 17 and 22 (SCHEDULE 2), or by a single administration of L19-IL2 at a dose 0.05 mg/mouse on day 8 (SCHEDULE 3), or by a single administration of L19-IL2 at a dose 0.05 mg/mouse on day 12 (SCHEDULE 4), or (iv) saline. The efficacy of the different treatments is assessed by daily measurement of tumor volume (mm³) during and after administration of the drugs. Data points represent mean tumor volume±SEM (n=4 per group). ¹⁷⁷Lu-Tri-ESV6 exhibits a dose-dependent therapeutic activity.

FIG. 14: Quantitative in vivo MMAE released in tumor and healthy organs by ESV6-GlyPro-MMAE (Conjugate 58a of EP3891138B1), Bi-ESV6-GlyPro-MMAE (Conjugate 11 of WO2022/171811) and Tri-ESV6-GlyPro-MMAE (Conjugate 9 of the present invention). Each conjugate was injected at a dose of 250 nmol/Kg and the MMAE release recorded at different timepoints after administration. Values are expressed as percentage of injected dose per gram of tissue (% ID/g). The quantification of MMAE released after administration of Tri-ESV6-GlyPro-MMAE revealed a cleaner biodistribution profile, with high and prolonged accumulation at the tumor site, and lower release in the healthy organs as compared to ESV6-GlyPro-MMAE and Bi-ESV6-GlyPro-MMAE.

FIG. 15: To identify the best dose for radiotherapeutic applications, ¹⁷⁷Lu-Tri-ESV6-DOTAGA was injected in tumour bearing mice at eight different doses ranging from 3 nmol/Kg to 2250/Kg. The results show that at the highest doses (>90 nmol/kg) the % ID/g in tumor drops (A), while the lowest doses (<10 nmol/kg) are partially uptaken in healthy organs (B). The optimal mouse dose range lies between 90 and 250 nmol/kg, which is equivalent to 1 to 3 mg per human patient.

FIG. 16: Therapeutic anti-cancer efficacy evaluation of ESV6-GlyPro-MMAE and Tri-ESV6-GlyPro-MMAE in HT-1080.hFAP tumor bearing mice, in terms of tumor volume over time (A) and percent survival (B). The tolerability was assessed by the evaluation of changes (%) in body weight during the experiment, indicating that all conjugates were well tolerated (C).

FIG. 17: Data sets (single plots; one curve for each animal) underlying the results shown in FIG. 16: ESV6-GlyPro-MMAE at 50 nmol/kg (A) or 125 nmol/kg (B); Tri-ESV6-GlyPro-MMAE at 50 nmol/kg (D) or 125 nmol/kg (E); and saline (C).

FIG. 18: Comparative hFAP inhibition assays with trivalent binders 11, 12, 13 and 14 having, inter alia, different linker groups B and/or different payload groups C.

FIG. 19: ¹⁷⁷Lu biodistribution results at different radioisotope molar activities (MBq of ¹⁷⁷Lu per nmol of ligand): low (0.2 MBq/nmol) and high (4.8 MBq/nmol, preferably used in clinical practice) with binder compound ¹⁷⁷Lu-Tri-ESV6-DOTAGA at 24 h post injection, suggesting that varying the molar activity does not significantly interfere with the advantageous biodistribution of the binder.

FIG. 20: Comparative hFAP inhibition assays with tetravalent binders 10 and 29.

### DETAILED DESCRIPTION OF THE INVENTION

The present inventors have identified small molecule binders of fibroblast activation protein (FAP) which are suitable for therapeutic applications.

As shown by the in vitro data provided herein, therapeutic binders according to the invention can provide high FAP inhibitory activity, high FAP binding affinity and prolonged binding to FAP-positive cells, and are therefore suitable candidates for targeted delivery of a therapeutic payload, to a site afflicted by or at risk of disease or disorder characterized by overexpression of FAP.

As shown by the in vivo data provided herein, therapeutic binders according to the invention can provide high and prolonged tumor uptake, and potent antitumor effect. At the same time, remarkably high tumor-to-organ ratios (in particular: tumor-to-kidney) can be achieved.

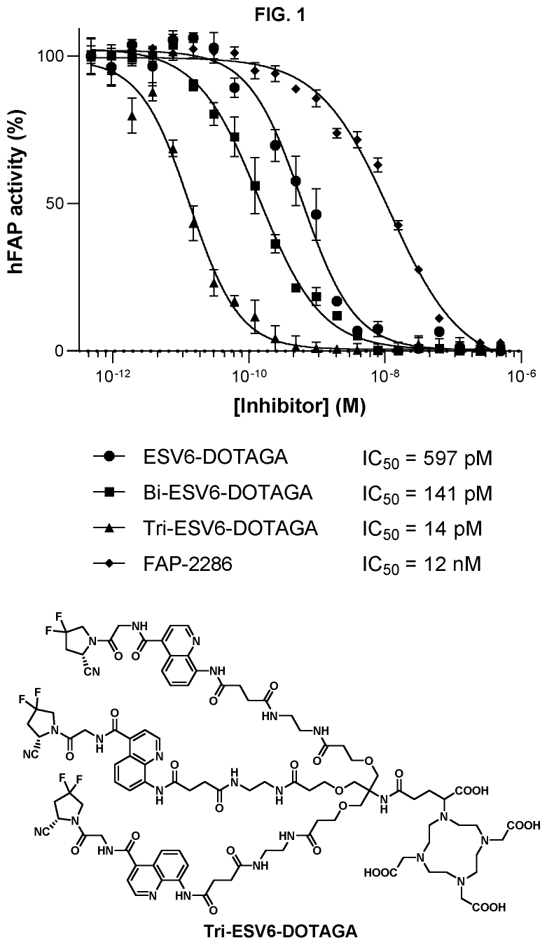

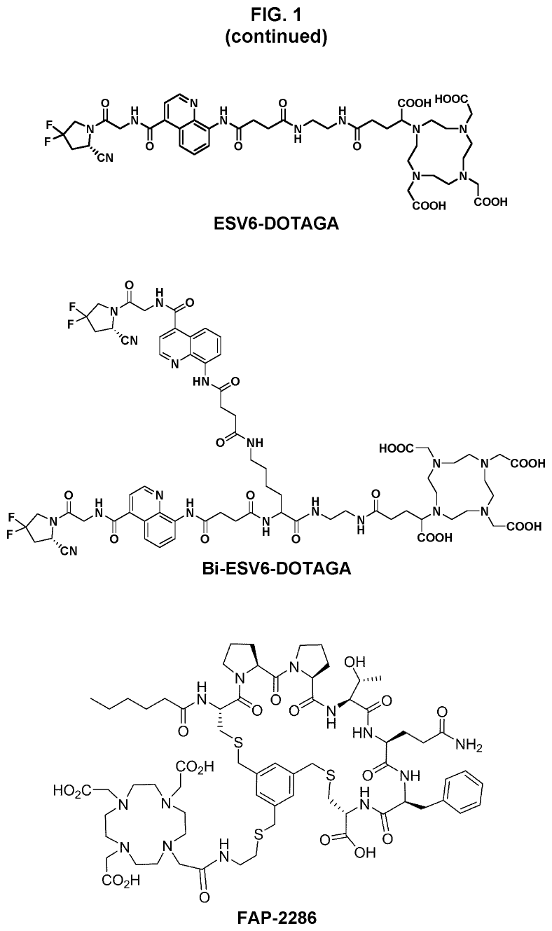

The therapeutic binders according to the invention are surprisingly advantageous in terms of one or more of the above-mentioned and further effects as compared to related prior art FAP-binders, such as FAP-2286. As evident from the results in FIGS. 1 to 5, binders according to the invention exhibit improved FAP inhibitory activity, improved FAP-binding affinity, prolonged binding to FAP-positive cells, higher tumor uptake and better tumor-to-organ ratios than FAP-2286.

The therapeutic binders according to the invention are surprisingly advantageous in terms of one or more of the above-mentioned and further effects as compared to monovalent FAP-binders, e.g., ESV6-DOTAGA having only one FAP-binding moiety A. As evident from the results in FIG. 1, the trivalent binder according to the invention having three FAP-binding moieties A. (Tri-ESV6-DOTAGA) shows a 43-fold improvement in IC50 as compared to the monovalent binder ESV6-DOTAGA, which is surprising and exceeds by far a 3-fold improvement which would normally be expected when assuming additive behavior. The trivalent binder according to the invention (Tri-ESV6-DOTAGA) also shows a 10-fold improvement in IC50 as compared to the bivalent binder Bi-ESV6-DOTAGA, which is surprising and exceeds by far a 1.5-fold improvement which would normally be expected when assuming additive behavior. Hence, binders according to the present invention can provide unexpected synergistic improvement in FAP inhibitory activity.

As evident from the results in FIG. 3, the trivalent binder according to the invention shows surprisingly improved efflux duration in FAP-positive tumor cells when compared to the bivalent binder, which is even further improved when compared to monovalent binders. Hence, binders according to the present invention can thus provide unexpected improvement in terms of prolonged tumor uptake duration.

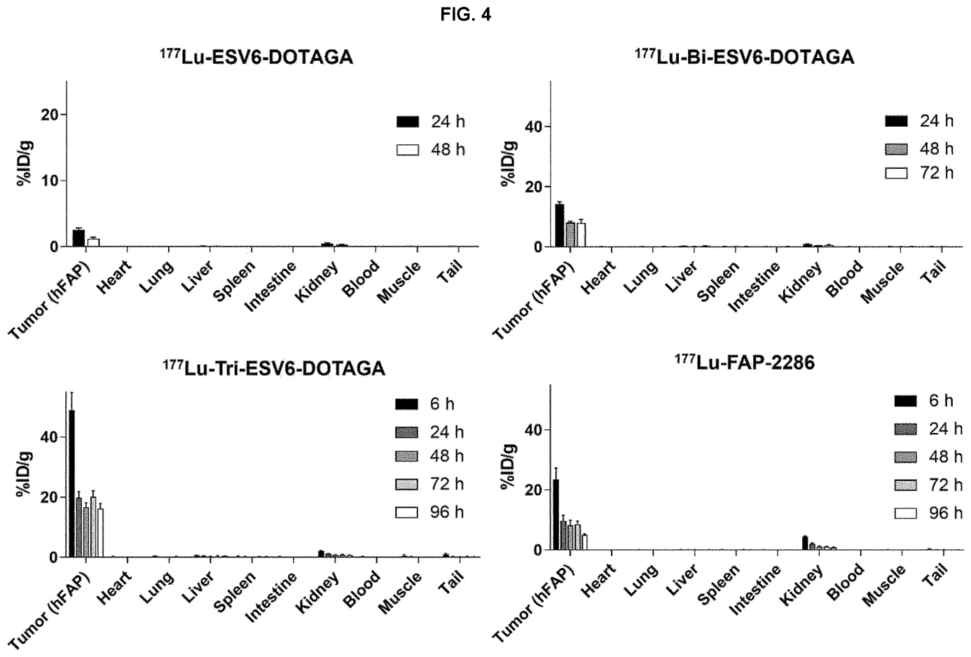

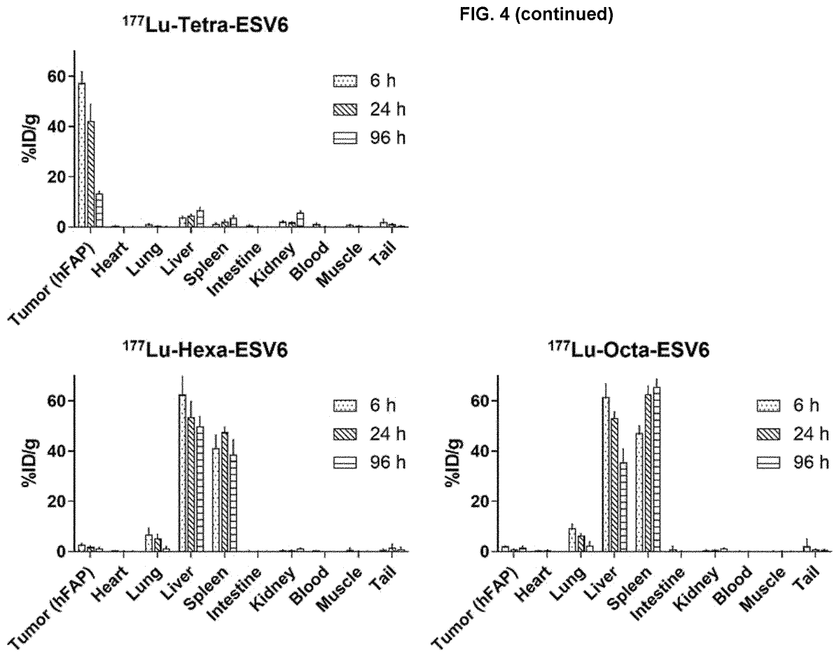

As evident from the results in FIG. 4, the trivalent binder according to the invention shows surprisingly improved in vivo tumor uptake in FAP-positive tumors when compared to monovalent, bivalent, hexavalent and octavalent binders (see also Tables 1 to 3 and Tables 11 to 12), while maintaining a surprisingly high tumor-to-organ ratios, in particular tumor-to-kidney (see also Tables 5 to 7 and Tables 13 to 15). This is surprising, since increasing the binder valency would normally be expected to lead to higher organ accumulation and deterioration of the tumor-to-organ ratios. For instance, at the same dosage, the Tri-ESV6 binder exhibits much better (cleaner) biodistribution, i.e., more advantageous tumor-to-organ ratio, as compared to other tested binders (e.g., hexavalent and octavalent).

Overall, ¹⁷⁷Lu-TriESV6-DOTAGA presents the best biodistribution profile, considering both tumor uptake over time (i.e., ~49% ID/g, 6 h post injection) and uptake in healthy organs (e.g., spleen uptake of ~0.3% ID/g and liver uptake of ~0.7% ID/g, 6 h post injection), thereby highlighting the trivalent TriESV6-based binders as best-in-class tumor-targeting ligands. Moreover, the trivalent compounds are smaller (lower molecular weight), present a higher atom economy, and are easier and cheaper to manufacture compared to their, e.g., tetravalent counterparts.

Hence, binders according to the present invention can provide unexpected improvement in tumor uptake while at the same time providing advantageous biodistribution, thus having an improved therapeutic potential.

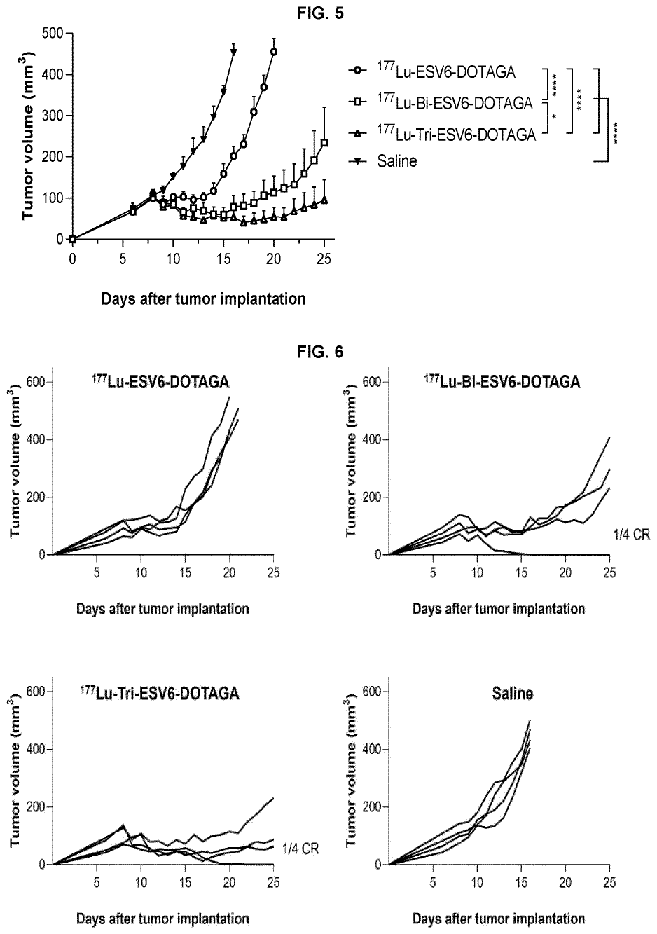

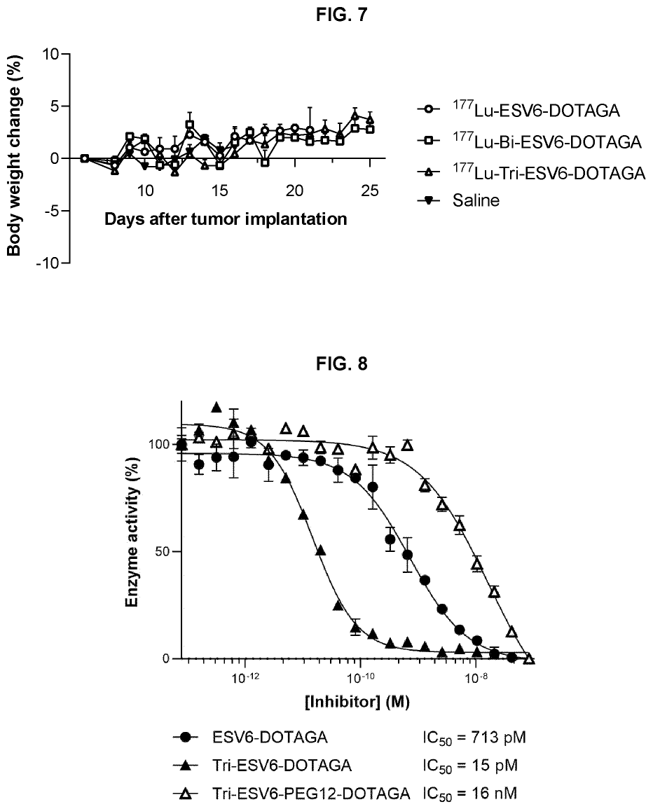

As evident from the results in FIG. 5 and FIG. 6, the trivalent binder according to the invention shows surprisingly improved in vivo therapeutic activity on FAP-positive tumors when compared to mono- and bivalent binders, while at the same time maintaining the tolerability, as evident from FIG. 7. Hence, binders according to the present invention can provide unexpected improvement in therapeutic efficacy and/or targeting specificity.

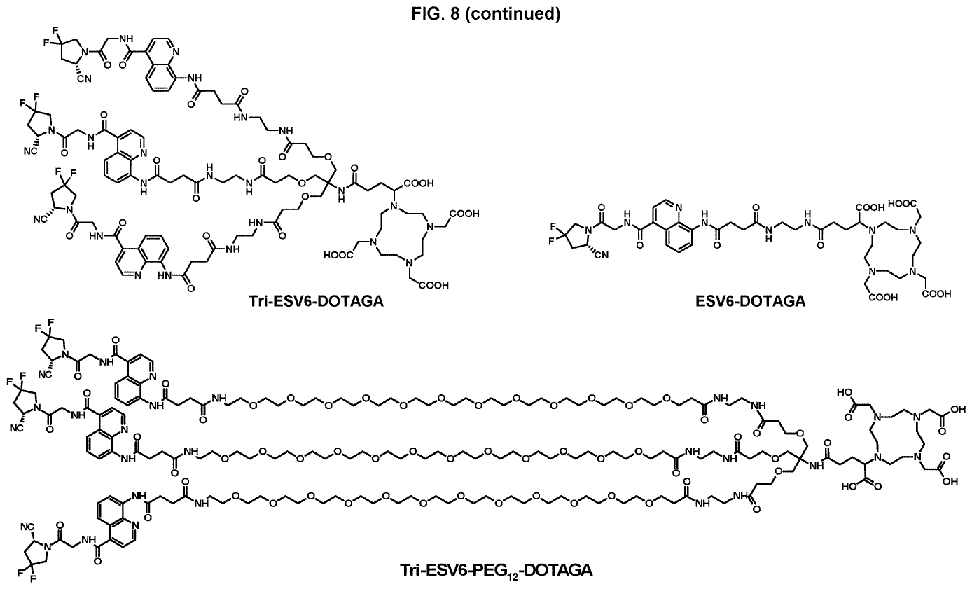

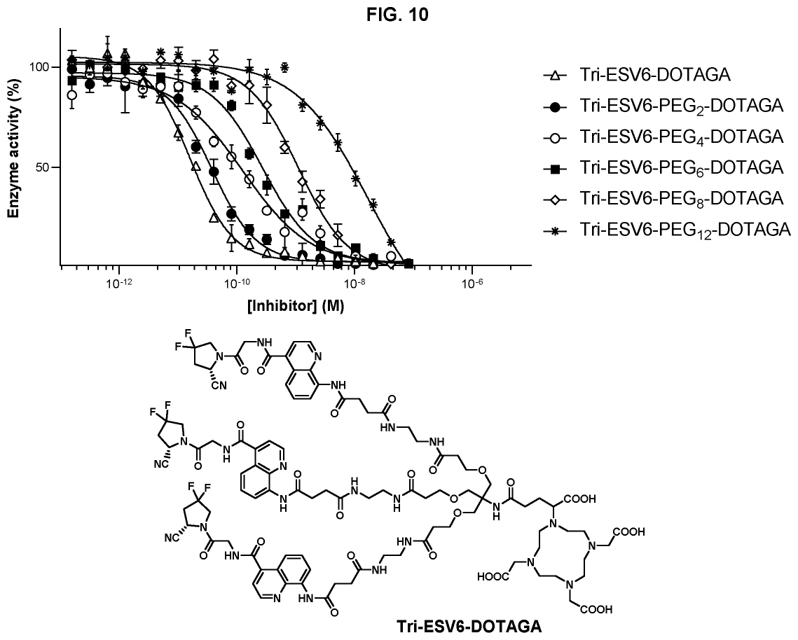

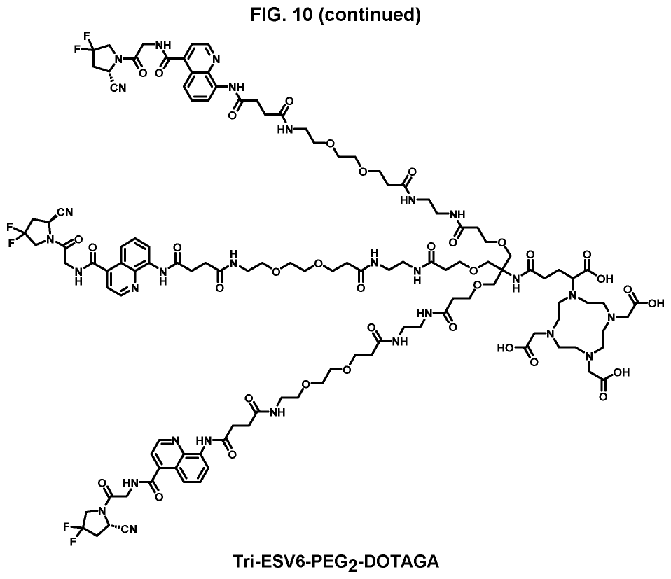

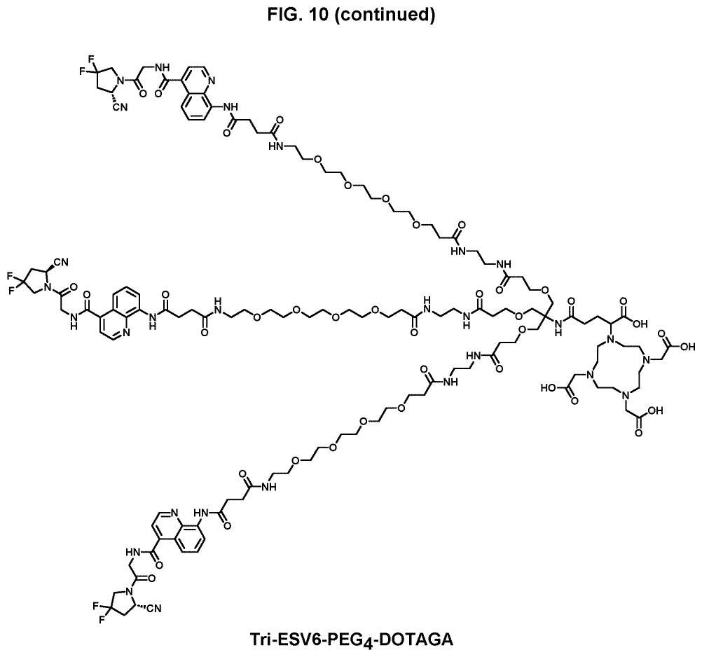

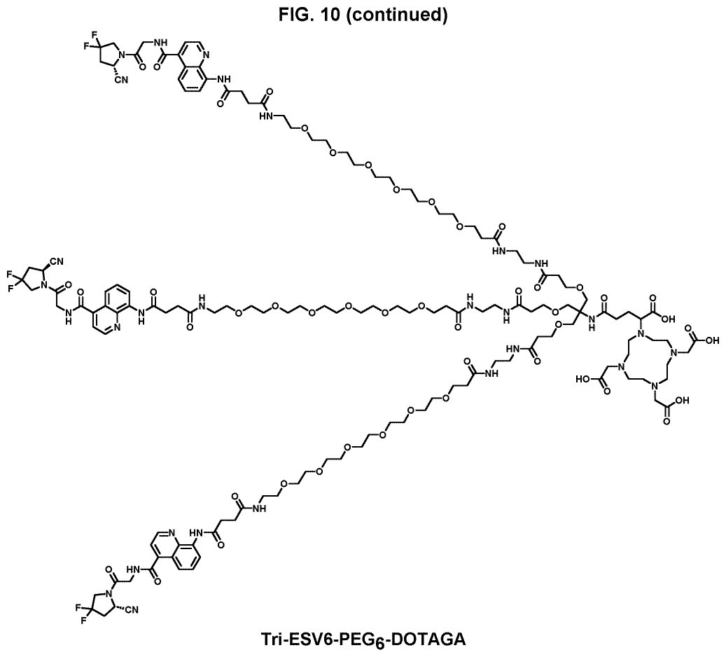

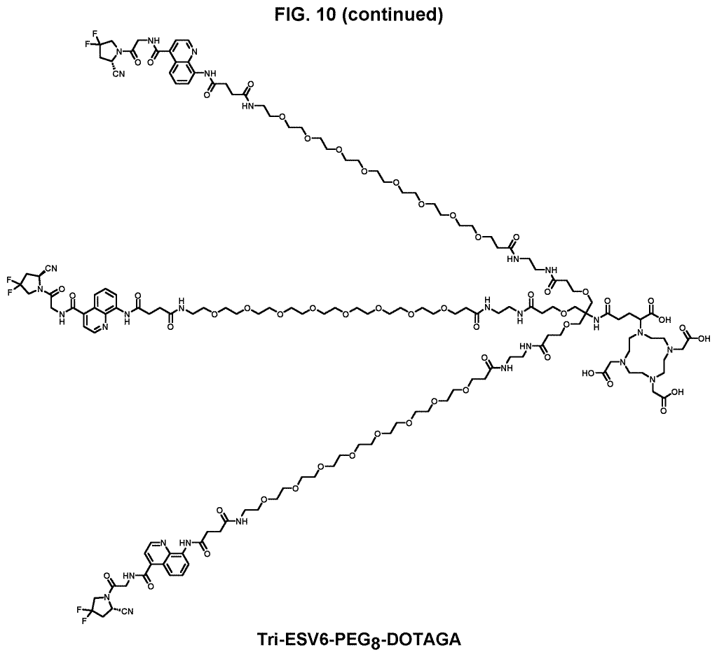

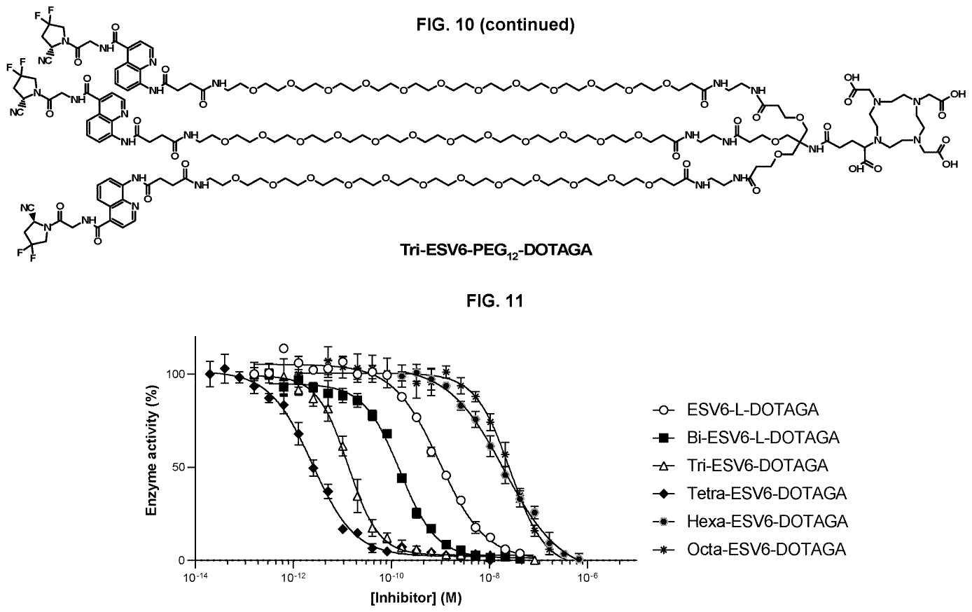

As evident from the results in FIG. 8 and FIG. 10, a short spacer (L)ₐ between the trivalent ESV6 moiety and the ramification point confers a superior inhibitory activity as compared to longer spacers (see also Table 16). This is surprising, considering that spacers in multivalent compounds with enough length to reach multiple adjacent FAP's on the target cell would have rather been expected to be advantageous.

In view of the above, the present invention provides particularly advantageous therapeutic compounds which can be administered at lower dosages (due to their improved binding properties), and are expected provide advantageous side effect profile (due to the lower required dosage, on the one hand, and their improved biodistribution, particularly high tumor: kidney ratio and tolerability profile, on the other hand).

In a preferred embodiment, the compound of the present invention is represented by the following Formula II:

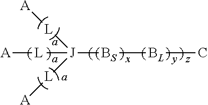

wherein each occurrence of L, B_{S} and B_{L} is independently a moiety comprising or consisting of a structural unit selected from alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, oxoalkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, and tetrapeptide, each of which is substituted or unsubstituted;
J is a moiety comprising a ramification point, comprising or consisting of a structural unit independently selected from the group consisting of alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, (oxo)alkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, tetrapeptide, each of which is substituted or unsubstituted;
each x is an integer independently selected from 0, 1, 2, 3, 4, 5, 6, 7, 8, 9, 10, 11, 12, 13, 14, 15, 16, 17, 18, 19 and 20;
each y is an integer independently selected from 0, 1, 2, 3, 4, 5, 6, 7, 8, 9, 10, 11, 12, 13, 14, 15, 16, 17, 18, 19 and 20;
each z is an integer independently selected from 0, 1, 2, 3, 4 and 5; and
each a is an integer independently selected from 0, 1, 2, 3, 4 and 5.

In a further preferred embodiment, moiety C comprises a ramification point, such that three moieties A are attached to C, and B is absent, i.e., moiety C serves as a ramification point therefore substituting B, preferably when C is a chelator (e.g., a chelating agent group suitable for radiolabeling with therapeutic nuclides. An exemplary structure is represented by general Formula III below, wherein all groups and variables are as defined in the claims, and wherein preferably each occurrence of a is 0:

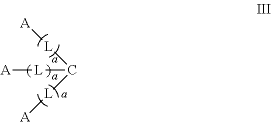

In this embodiment, when C is a chelator bearing multiple —COOH groups, each A-(L)ₐ- arm is attached to a —C(O)— groups in the chelator structure derived from the respective —COOH group. For instance, if the terminal atom of A-(L)ₐ- is oxygen (i.e., the arm has the general structure A-R—O—), this arm may be attached to a respective —COOH group of the chelator, thereby forming an ester (—C(O)O—R-A); if the terminal atom of A-(L)ₐ- is an amino nitrogen (i.e., the arm has the general structure A-R—NH—), this arm may be attached to a respective-COOH group of the chelator, thereby forming an amide (—CONH—R-A), wherein R is the particular structure of (L)ₐ in the respective arm excluding the terminal atom.

As used herein, the term “ramification point”, “R.P.” or ◯ denotes, unless specified otherwise, a central atom bound to at least three non-hydrogen atoms, wherein at least two of said non-hydrogen atoms are located on a covalent chain which represents the shortest path between a moiety A and said central atom. If the path comprising the smallest number of covalent bonds between two or more moieties A does not go through the same atom but through the same ring system, then the geometrical center of such ring system shall be considered a ramification point. A molecule comprising three moieties A can comprise, e.g., one or two ramification points, and preferably comprises only one ramification point. If the molecule comprises more than one ramification point, the number of covalent bonds between each ramification point and the closest ramification point is preferably 7 or less, more preferably 5 or less, most preferably 3 or less.

As used herein, the terms “distance” or “length” between moieties or groups denote, unless specified otherwise, the longest interatomic (through space) distance in the most expanded conformation between the two non-hydrogen atoms belonging to the respective groups which atoms are connected through the path comprising the smallest number of covalent bonds. For instance, in the distance between groups G¹ (=¹³CH₃) and G² (=OH) in the compound G¹-CH₂—CH₂-G² would be the longest interatomic (through space) distance between atom ¹³C and atom O in the most expanded (stretched) conformation.

Without wishing to be bound by theory, it is considered that the multiple FAP-binding moieties A present in the molecule of a binder according to the present invention, preferably up to 4, more preferably 3, engage in binding and rebinding to the very same FAP target molecule and/or establishing additional synergistic interactions that a single moiety cannot. This is considered to result from an interplay between the relatively voluminous active pocket of the FAP protein, on the one hand, and the particular structural characteristics of the present binders, on the other hand. It is therefore contemplated that one or more of the advantageous effects described herein are particularly pronounced when the intramolecular distances (lengths) between each moiety A and the closest ramification point are short, and preferably such that the binder engages in binding to one FAP target molecule, rather than multiple adjacent FAP molecules (e.g., on a target cell). That is, the moieties (L)ₐ and J are preferably such that they do not provide length for the arms of the multivalent compound to reach multiple adjacent FAPs on the target cell.

This is confirmed by the results shown in FIG. 8, showing that Tri-ESV6-DOTAGA (having a distance between each moiety A and the closest ramification point of approx. 17 Å) exhibits a much stronger FAP inhibition (~1000-fold lower IC₅₀) as compared to Tri-ESV6-PEG12-DOTAGA with a longer distance (approx. 60 Å) between each moiety A and the point of ramification. In this regard, particularly suitable distances (d₁+d₂) between each moiety A and the closest ramification point are considered to be 30 Å or less, preferably 24 Å or less, more preferably 17 Å or less.

Without wishing to be bound by theory, lowering the distance d₁+d₂ is considered to contribute to improving the binding properties.

Preferably, the distance between each moiety A and J is 24 Å or less, preferably 18 Å or less, more preferably 11 Å or less. This distance represents the length of the spacer that connects a ligand A (that binds to fibroblast activation protein (FAP) on a target cell) with a multipoint template J to which the multiple arms of the compound connect, and may also be denoted as d₁, as exemplified in the below schematic representation. In other words, d₁ is the distance between the two atoms directly bound to each end of moiety (L)ₐ.

Without wishing to be bound by theory, lowering distance d₁ is considered to contribute to improving the binding properties.

Preferably, the distance between each moiety L and the closest ramification point is 19 Å or less, preferably 12 Å or less, more preferably 6 Å or less. This distance represents the length from the ramification point to the “first” atom belonging to moiety L along the chain which represents the shortest path between the ramification point and the respective moiety A, and circumscribes the part of the distance between each moiety A and the closest ramification point which belongs to the multipoint template J. This distance may also be denoted as d₂, as exemplified in the below schematic representation. In other words, d₂ is the distance between the atom belonging to group moiety (L)ₐ (or A, if a is 0) directly bound to moiety J, and the ramification point.

Without wishing to be bound by theory, lowering distance d₂ is considered to contribute to improving the binding properties.

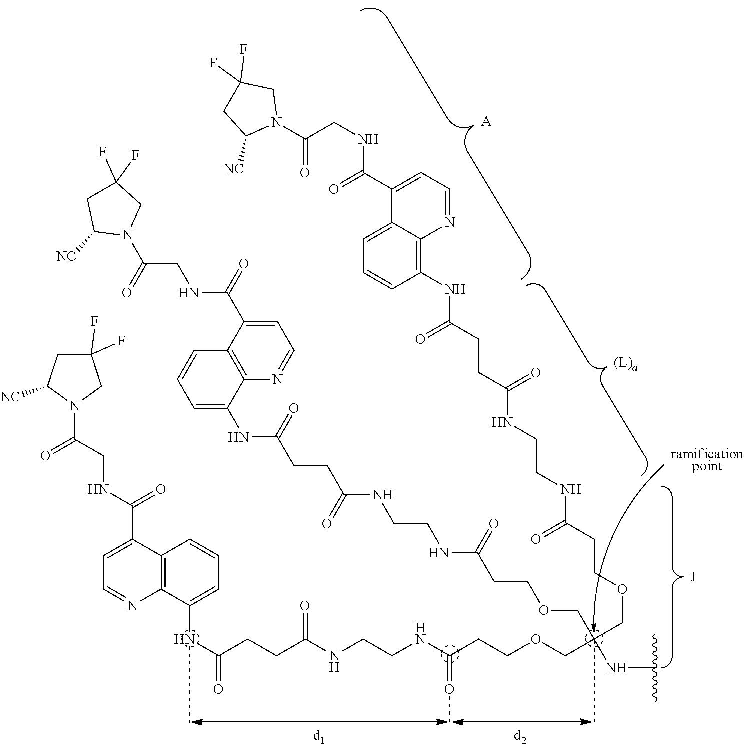

### Moiety

Without wishing to be bound by any theory, it is contemplated that some of the surprising technical effects are associated with the particular structure of the small binding moieties A wherein the quinoline ring is substituted at the 8-position by a nitrogen-containing group, such as an amino or amido group:

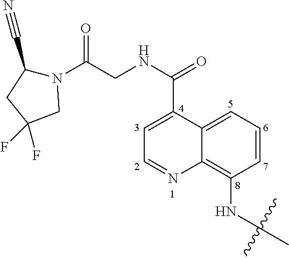 [SMILES: *Nc1cccc2c(C(=O)NCC(=O)N3CC(F)(F)C[C@H]3C#N)ccnc12]

It has been previously shown that the higher target protein affinity of a compound may result in longer tumor residence in vivo (Wichert et al., Nature Chemistry 7, 241-249 (2015)). The therapeutic compounds of the present invention have an increased affinity, slower dissociation rate with respect to FAP as compared to prior art compounds, and have prolonged residence at the disease site at a therapeutically relevant level, preferably beyond 1 h, more preferably beyond 6 h post injection. Preferably, the highest enrichment is achieved after 5 min, 10 min, 20 min, 30 min, 45 min, 1 h, 2 h, 3 h, 4 h, 5 h or 6 h; and/or enrichment in the disease site is maintained at a therapeutically relevant level, over a period of or at least for 5 min, 10 min, 20 min, 30 min, 45 min, 1 h, 2 h, 3 h, 4 h, 5 h or 6 h, 24 h, 48 h, 72 h, 96 h, more preferably beyond 6 h, even more preferably beyond 24 h post injection.

### Moiety B

Moiety B is a covalent bond or a moiety comprising a chain of atoms that covalently connects the moieties A to the payload C. The moiety B may be cleavable or non-cleavable, multifunctional moiety which can be used to link one or more payload and/or binder moieties to form the targeted conjugate of the invention.

Specifically, moiety B is a multifunctional moiety linking one or more moieties C and/or moieties A. The structure of the compound can comprise 3 moieties A per molecule. The structure of the compound may comprise more than one moiety C, preferably 2, 3, 4, 5, 6, 7, 8, 9 or 10 moieties C per molecule. Preferably, the structure of the compound comprises 3 moieties A and 1 moiety C per molecule.

When cleavable linker units are present within moiety B, release mechanisms can be identical to those specific to antibodies linked to cytotoxic payloads. Indeed, the nature of the binding moieties is independent in that respect. Therefore, there is envisaged pH-dependent [Leamon, C. P. et al (2006) Bioconjugate Chem., 17, 1226; Casi, G. et al (2012) J. Am. Chem. Soc., 134, 5887], reductive [Bernardes, G. J. et al (2012) Angew. Chem. Int. Ed. Engl., 51. 941; Yang, J. et al (2006) Proc. Natl. Acad. Sci. USA, 103, 13872] and enzymatic release [Doronina S. O. et al (2008) Bioconjugate Chem, 19, 1960; Sutherland, M. S. K. (2006) J. Biol. Chem, 281, 10540]. In a specific setting, when functional groups are present on either the binding moiety or payloads (e.g. thiols, alcohols) a linkerless connection can be established thus releasing intact payloads, which simplifies substantially pharmacokinetic analysis.

Moiety B can comprise or consist of a unit shown in Table A below wherein the substituents R and Rⁿ shown in the formulae may suitably be independently selected from H, halogen, substituted or unsubstituted (hetero)alkyl, (hetero)alkenyl, (hetero)alkynyl, (hetero) aryl, (hetero) arylalkyl, (hetero)cycloalkyl, (hetero)cycloalkylaryl, heterocyclylalkyl, a peptide, an oligosaccharide or a steroid group. Preferably, each of R, R₁, R₂ and R₃ is independently selected from H, OH, SH, NH₂, halogen, cyano, carboxy, alkyl, cycloalkyl, aryl and heteroaryl, each of which is substituted or unsubstituted. Suitably R and Rⁿ are independently selected from H, or C1-C7 alkyl or heteroalkyl. More suitably, R and Rⁿ are independently selected from H, methyl or ethyl.

TABLE A

| Linker | Structure | Release mechanism |
|---|---|---|
| Amide | 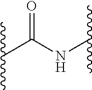 [SMILES: *NC(*)=O] | Proteolysis |
| | 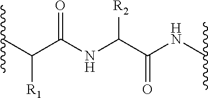 | |
| Ester | 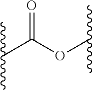 [SMILES: *OC(*)=O] | Hydrolysis |
| Carbamate | 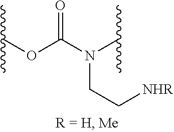 | Hydrolysis |
| Hydrazone | 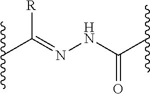 | Hydrolysis |
| Thiazolidine | 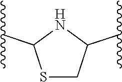 [SMILES: *C1CSC(*)N1] | Hydrolysis |
| Methylene alkoxy carbamate | 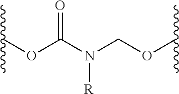 | Hydrolysis |
| Disulfide | 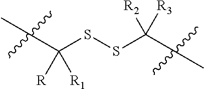 | Reduction |

Moiety B, unit(s) B_{L} and/or unit(s) B_{S} may suitably comprise as a cleavable bond a disulfide linkage since these linkages are stable to hydrolysis, while giving suitable drug release kinetics at the target in vivo, and can provide traceless cleavage of drug moieties including a thiol group.

Moiety B, unit(s) B_{L} and/or unit(s) B_{S} may be polar or charged in order to improve water solubility of the conjugate. For example, the linker may comprise from about 1 to about 20, suitably from about 2 to about 10, residues of one or more known water-soluble oligomers such as peptides, oligosaccharides, glycosaminoglycans, polyacrylic acid or salts thereof, polyethylene glycol, polyhydroxyethyl (meth) acrylates, polysulfonates, etc. Suitably, the linker may comprise a polar or charged peptide moiety comprising e.g. from 2 to 10 amino acid residues. Amino acids may refer to any natural or non-natural amino acid. The peptide linker suitably includes a free thiol group, preferably a N-terminal cysteine, for forming the said cleavable disulfide linkage with a thiol group on the drug moiety. Any peptide containing L- or D-aminoacids can be suitable; particularly suitable peptide linkers of this type are Asp-Arg-Asp-Cys and/or Asp-Lys-Asp-Cys.

In these and other embodiments, moiety B, unit(s) B_{L} and/or unit(s) B_{S} may comprise a cleavable or non-cleavable peptide unit that is specifically tailored so that it will be selectively enzymatically cleaved from the drug moiety by one or more proteases on the cell surface or the extracellular regions of the target tissue. The amino acid residue chain length of the peptide unit suitably ranges from that of a single amino acid to about eight amino acid residues. Numerous specific cleavable peptide sequences suitable for use in the present invention can be designed and optimized in their selectivity for enzymatic cleavage by a particular tumor-associated enzyme e.g. a protease. Cleavable peptides for use in the present invention include those which are optimized toward the proteases MMP-1, 2 or 3, or cathepsin B, C or D. Especially suitable are peptides cleavable by Cathepsin B. Cathepsin B is a ubiquitous cysteine protease. It is an intracellular enzyme, except in pathological conditions, such as metastatic tumors or rheumatoid arthritis. An example for a peptide cleavable by Cathepsin B is containing the sequence Val-Cit.

In any of the above embodiments, the moiety B and in particular, unit(s) B_{L} suitably further comprise(s) self-immolative moiety can or cannot be present after the linker. The self-immolative linkers are also known as electronic cascade linkers. These linkers undergo elimination and fragmentation upon enzymatic cleavage of the peptide to release the drug in active, preferably free form. The conjugate is stable extracellularly in the absence of an enzyme capable of cleaving the linker. However, upon exposure to a suitable enzyme, the linker is cleaved initiating a spontaneous self-immolative reaction resulting in the cleavage of the bond covalently linking the self-immolative moiety to the drug, to thereby effect release of the drug in its underivatized or pharmacologically active form. In these embodiments, the self-immolative linker is coupled to the binding moiety through an enzymatically cleavable peptide sequence that provides a substrate for an enzyme to cleave the amide bond to initiate the self-immolative reaction. Suitably, the drug moiety is connected to the self-immolative moiety of the linker via a chemically reactive functional group pending from the drug such as a primary or secondary amine, hydroxyl, sulfhydryl or carboxyl group.

Examples of self-immolative linkers are PABC or PAB (para-aminobenzyloxycarbonyl), attaching the drug moiety to the binding moiety in the conjugate (Carl et al (1981) J. Med. Chem. 24:479-480; Chakravarty et al (1983) J. Med. Chem. 26:638-644). The amide bond linking the carboxy terminus of a peptide unit and the para-aminobenzyl of PAB may be a substrate and cleavable by certain proteases. The aromatic amine becomes electron-donating and initiates an electronic cascade that leads to the expulsion of the leaving group, which releases the free drug after elimination of carbon dioxide (de Groot, et al (2001) Journal of Organic Chemistry 66 (26): 8815-8830). Further self-immolating linkers are described in WO2005/082023.

In yet other embodiments, the linker comprises a glucuronyl group that is cleavable by glucoronidase present on the cell surface or the extracellular region of the target tissue. It has been shown that lysosomal beta-glucuronidase is liberated extracellularly in high local concentrations in necrotic areas in human cancers, and that this provides a route to targeted chemotherapy (Bosslet, K. et al. Cancer Res. 58, 1195-1201 (1998)).

In any of the above embodiments, the moiety B suitably further comprises a spacer unit. A spacer unit can be the unit B_{S}, which may be linked to the binding moiety A, for example via an amide, amine or thioether bond. The spacer unit is of a length that enables e.g. the cleavable peptide sequence to be contacted by the cleaving enzyme (e. g. cathepsin B) and suitably also the hydrolysis of the amide bond coupling the cleavable peptide to the self-immolative moiety X. Spacer units may for example comprise a divalent radical such as alkylene, arylene, a heteroarylene, repeating units of alkyloxy (e.g. polyethylenoxy, PEG, polymethyleneoxy) and alkylamino (e.g. polyethyleneamino), or diacid ester and amides including succinate, succinamide, diglycolate, malonate, and caproamide.

In any of the embodiments described therein, * represents a point of attachment to moiety A or a point of attachment for which the shortest path to moiety A comprises less atoms than that for., as the case may be; and · represents a point of attachment a point of attachment to therapeutic moiety C or a point of attachment to therapeutic moiety C for which the shortest path to therapeutic moiety C comprises less atoms than that for *, as the case may be. The same applies also for cases where a reactive moiety L is present rather than the therapeutic payload moiety C. The following notations and all have the meaning of a point of attachment of a certain group or atom (e.g., R) to a further moiety:

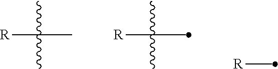

If the structure of relevance is a peptide mono- or oligomer, each * represents a point of attachment for which the shortest path to moiety A comprises less atoms than that for ·; and each · represents a point of attachment for which the shortest path to therapeutic moiety C comprises less atoms than that for *, with the proviso that when n is >1 and a respective point of attachment is indicated on any one of R^{a}, R^{b} and R^{c}, then it can be independently present in one or more of the peptide monomeric units, preferably in one peptide monomeric unit most distant from the other point of attachment indicated in the respective structure.

In any of the embodiments described herein, the terms “peptide”, “dipeptide”, “tripeptide”, “tetrapeptide” etc. refer to peptide mono- or oligomers having a backbone formed by proteinogenic and/or a non-proteinogenic amino acids. As used herein, the terms “aminoacyl” or “aminoacid” generally refer to any proteinogenic or a non-proteinogenic amino acid. Preferably, in any of the embodiments disclosed therein, the side-chain residues of a proteinogenic or a non-proteinogenic amino acid are represented by any of R^{a}, R^{b} and R^{c}, each of which is selected from the following list:

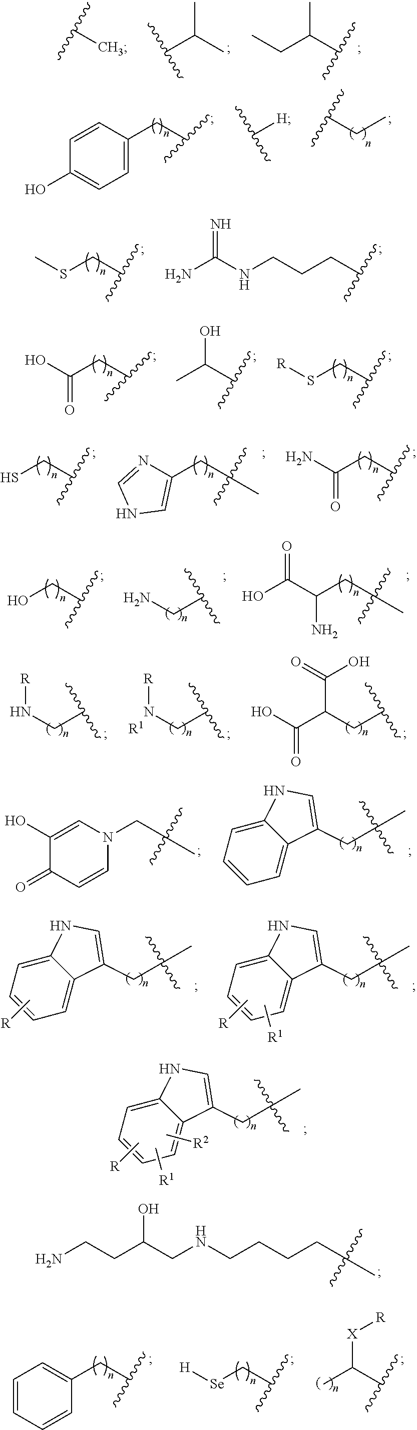

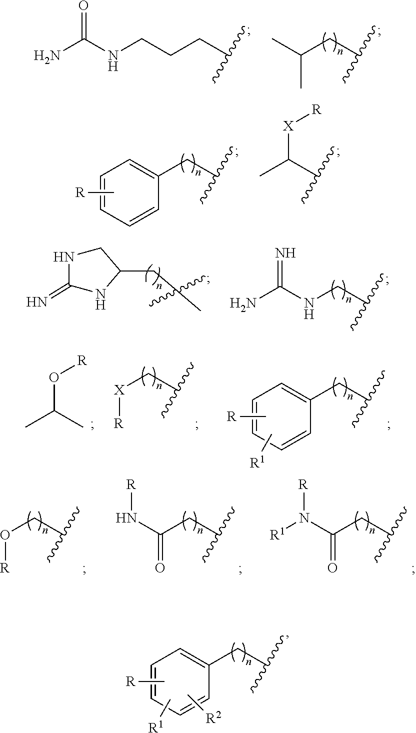

wherein each of R, R¹, R² and R³ is independently selected from H, OH, SH, NH₂, halogen, cyano, carboxy, alkyl, cycloalkyl, aryl and heteroaryl, each of which is substituted or unsubstituted;
each X is independently selected from NH, NR, S, O and CH₂, preferably NH; and
each n and m is independently an integer preferably selected from 0, 1, 2, 3, 4, 5, 6, 7, 8, 9, 10, 11, 12, 13, 14, 15, 16, 17, 18, 19 and 20.

Preferably, in any of the embodiments disclosed therein, side-chain residues of a proteinogenic or a non-proteinogenic amino acid are represented by any of R^{a}, R^{b} and R^{c},
each of which may be part of a 3-, 4-, 5-, 6- or 7-membered ring. For instance, the side chain alpha, beta and/or gamma position of said proteinogenic or non-proteinogenic amino acid can be part of a cyclic structure selected from an azetidine ring, pyrrolidine ring and a piperidine ring, such as in the following aminoacids (proline and hydroxyproline):

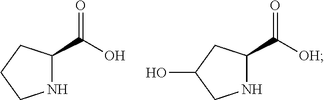 [SMILES: O=C(O)[C@@H]1CCCN1 ; O=C(O)[C@@H]1CC(O)CN1]

or
each of which may independently be part of an unsaturated structure (i.e, wherein the H atom geminal to the respective group R^{a}, R^{b} and R^{c} is absent), e.g.:

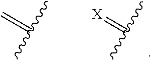

As used herein, the following notation of peptide sequences refers to a sequence from N to C terminus, and attachment of group through a horizontal bond (here: moiety C) means covalent attachment to the peptide backbone via amide bond to the respective terminal amino acid (here: AA₃):

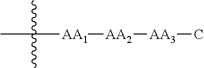

As used herein, the following notation of peptide sequences refers to a sequence from N to C terminus, and attachment of group through a vertical bond (here: moiety C) means covalent attachment via the sidechain of the respective amino acid (here: AA₃):

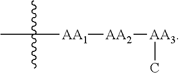

Further preferable non-proteinogenic amino acids can be selected from the following list:

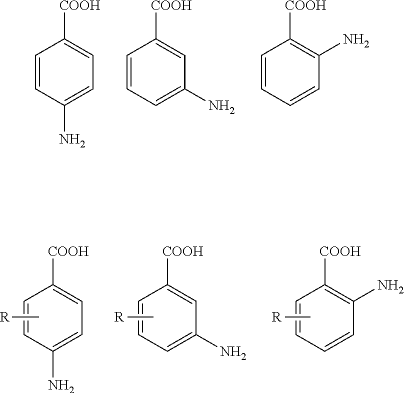

Particularly preferred embodiments for the moiety B as well as the compound according to the present invention are shown in the items further below and in the appended claims.

Preferably, B comprises the following structure

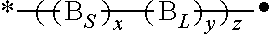

wherein each x is an integer independently selected from the range of 0 to 100, preferably 0 to 50, more preferably 0 to 30, yet more preferably selected from 0, 1, 2, 3, 4, 5, 6, 7, 8, 9, 10, 11, 12, 13, 14, 15, 16, 17, 18, 19 and 20;
each y is an integer independently selected from the range of 0 to 30, preferably selected from 0, 1, 2, 3, 4, 5, 6, 7, 8, 9, 10, 11, 12, 13, 14, 15, 16, 17, 18, 19 and 20;
each z is an integer independently selected from the range of 0 to 5, preferably selected from selected from 0, 1, 2, 3 and 4
* represents a point of attachment to a moiety A; and
· represents a point of attachment to therapeutic moiety C.

Each of L, B_{S} and B_{L} can be independently a moiety comprising or consisting of a structural unit selected from alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, oxoalkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, and tetrapeptide, each of which is substituted or unsubstituted;

Each of L, B_{S} and B_{L} can be independently selected from:

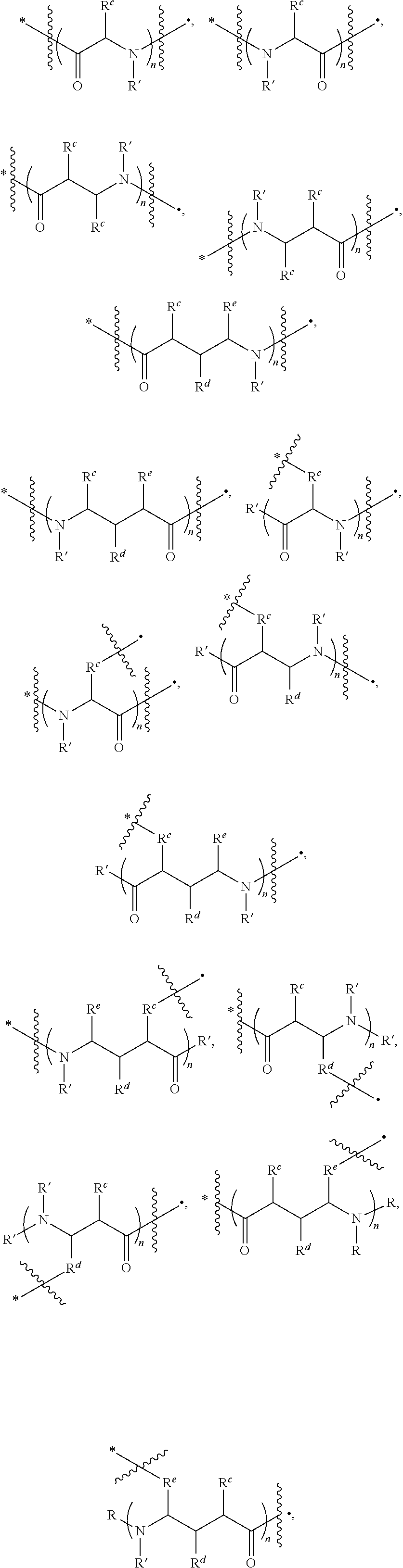

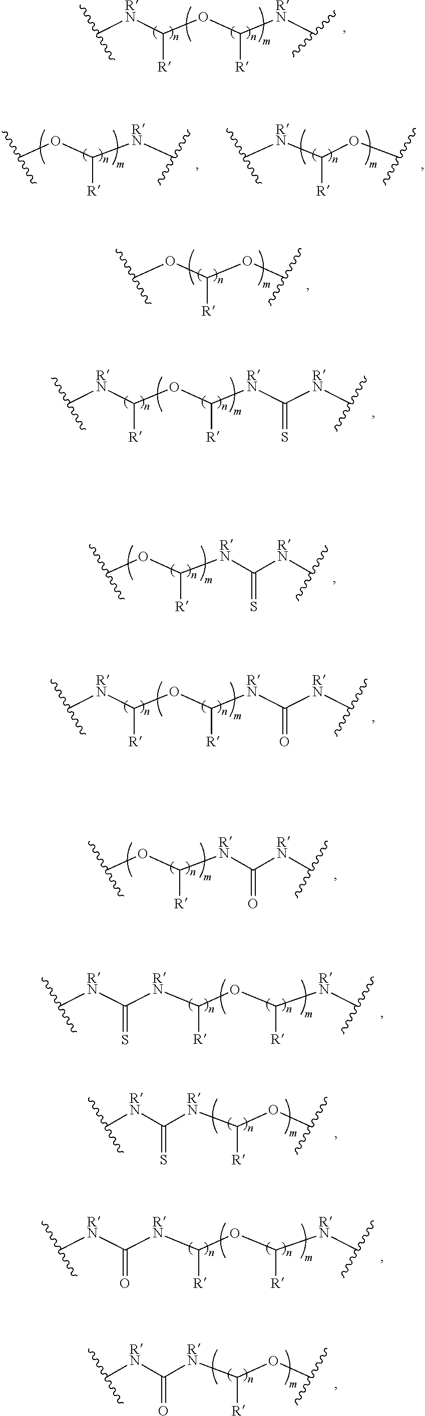

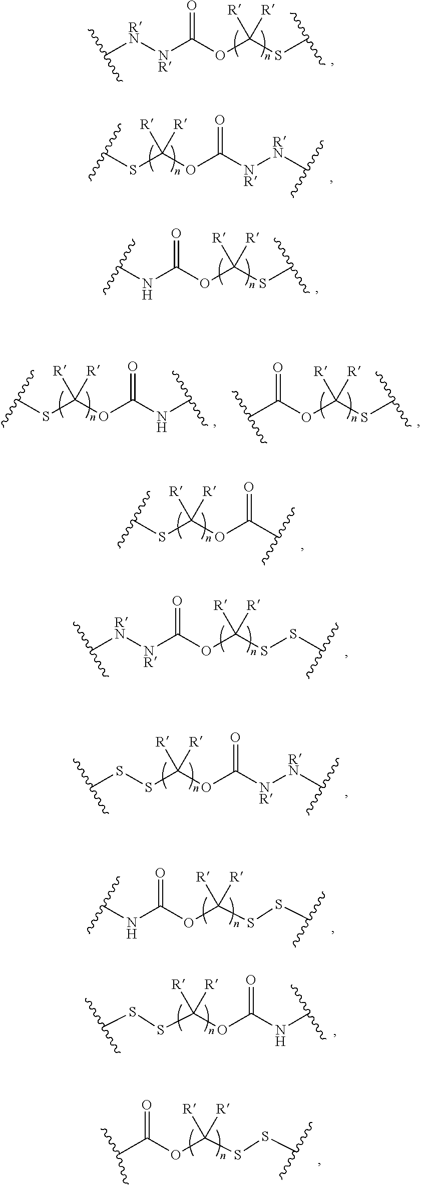

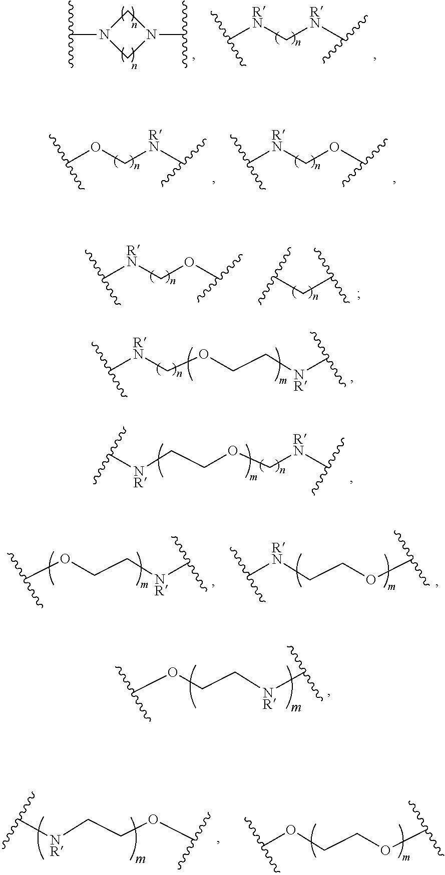

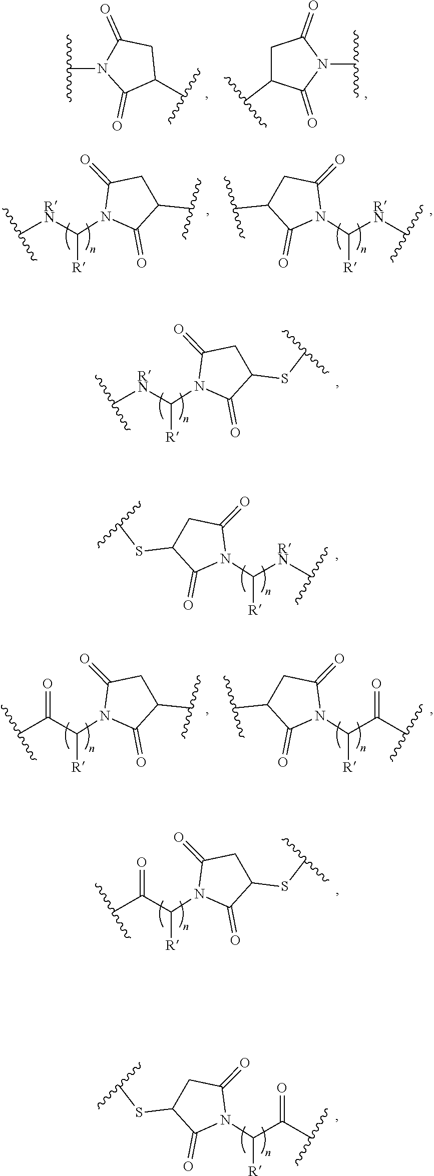

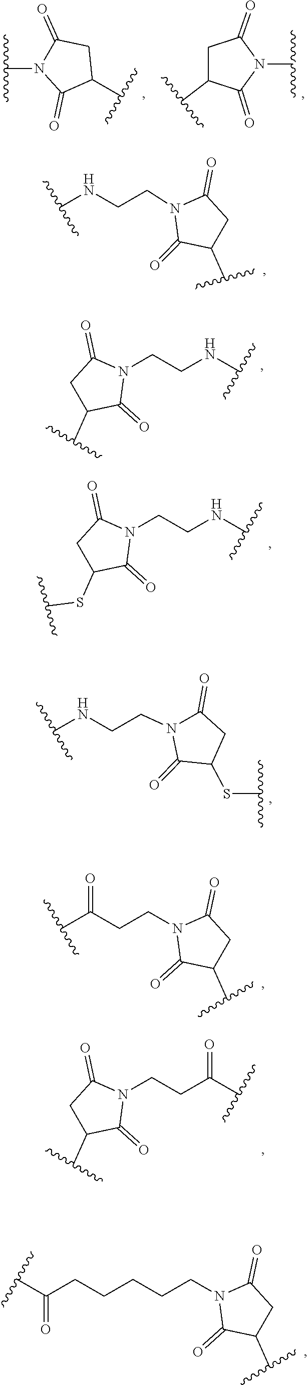

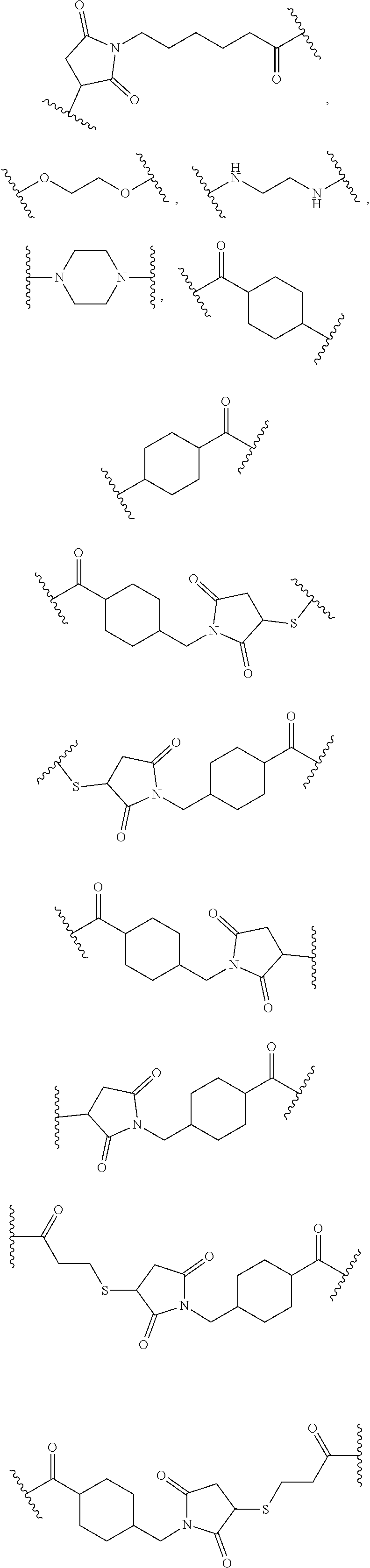

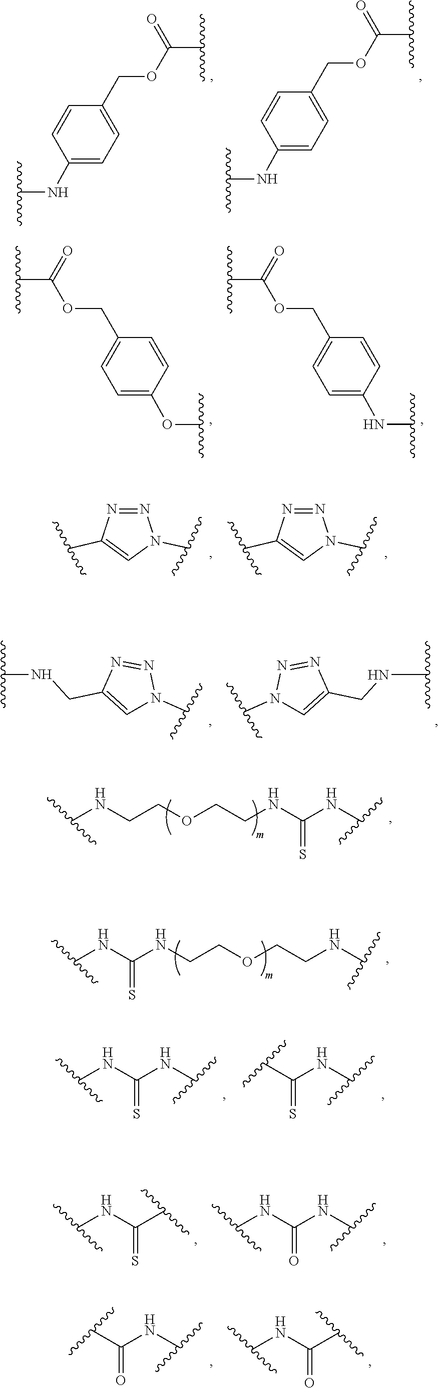

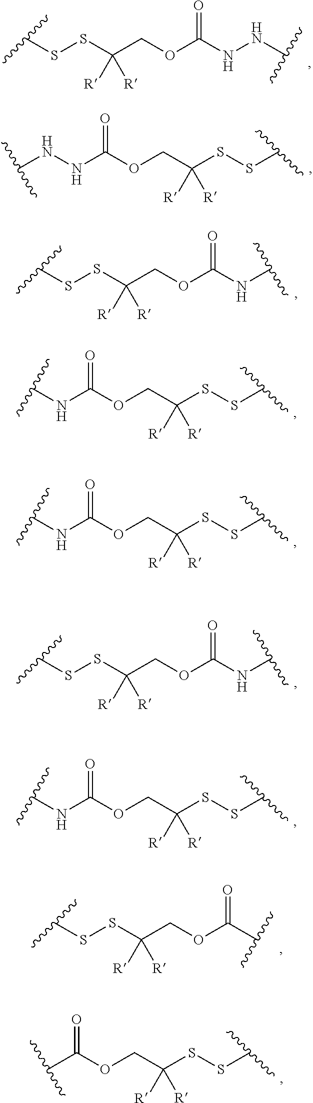

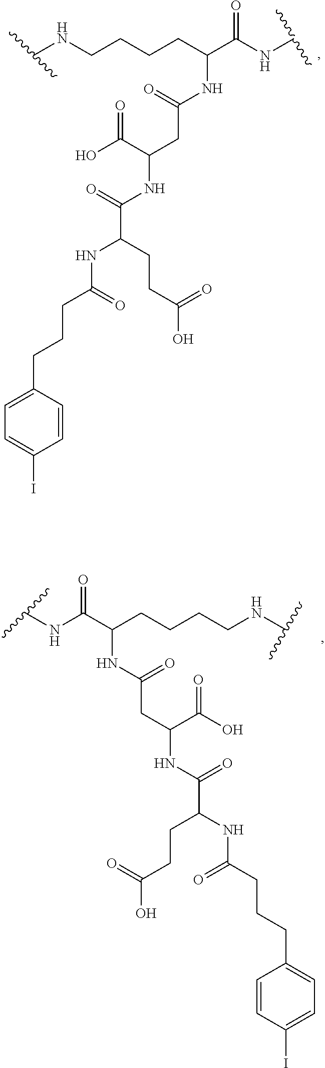 [SMILES: *NCCCCC(NC(=O)CC(NC(=O)C(CCC(=O)O)NC(=O)CCCc1ccc(I)cc1)C(=O)O)C(=O)N* ; *NCCCCC(NC(=O)CC(NC(=O)C(CCC(=O)O)NC(=O)CCCc1ccc(I)cc1)C(=O)O)C(=O)N*]

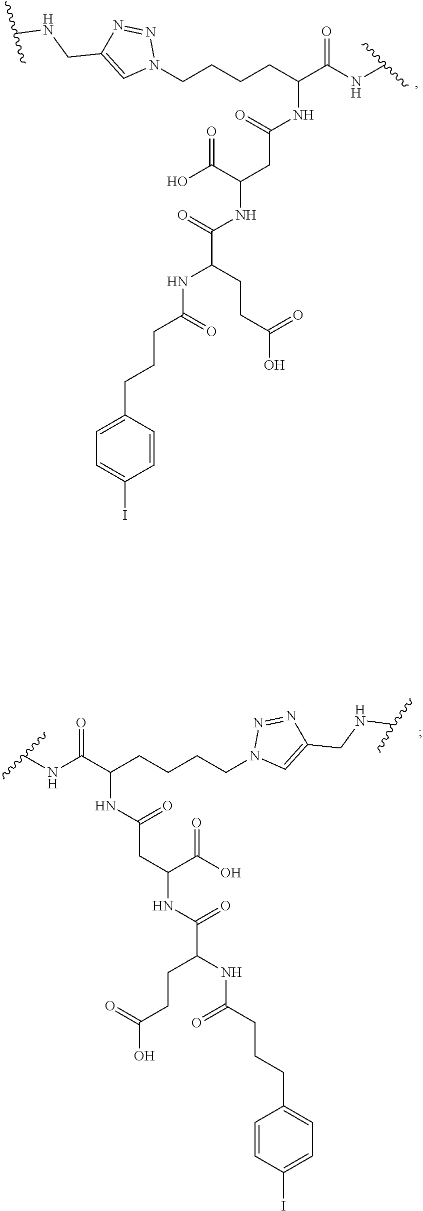 [SMILES: *NCc1cn(CCCCC(NC(=O)CC(NC(=O)C(CCC(=O)O)NC(=O)CCCc2ccc(I)cc2)C(=O)O)C(=O)N*)nn1 ; *NCc1cn(CCCCC(NC(=O)CC(NC(=O)C(CCC(=O)O)NC(=O)CCCc2ccc(I)cc2)C(=O)O)C(=O)N*)nn1]

wherein in each of the above structures:
each n is independently 0, 1, 2, 3, 4, 5, 6, 7, or 8;
each m is independently 0, 1, 2, 3, or 4;
each R′ is independently H or selected from H, SH, NH₂, halogen, cyano, carboxy, C₁₋₆-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C₁₋₁₀ cycloheteroalkenyl, C₆₋₁₀ aryl, and (C₆₋₁₀ aryl)C₁₋₆ alkyl, each of which being optionally substituted with from 1 to 3 substituents selected from —OH, oxo and halo,
each R^{c}, R^{d}, and R^{e} is independently is selected from H, optionally substituted C₁₋₆ alkyl, (C₃-C₁₀ carbocyclyl)C₁₋₆ alkyl, (C₆-C₁₀ aryl)C₁₋₆ alkyl, (C₁-C₁₀ heterocyclyl)C₁₋₆ alkyl, C₂₋₆ alkenyl, C₂₋₆ alkynyl, and C₆-C₁₀ aryl, in each of which optionally one or more of the carbon atoms can be replaced by heteroatoms; preferably selected from side-chain residues of proteinogenic or a non-proteinogenic amino acids;
each * represents a point of attachment for which the shortest path to a moiety A comprises less atoms than that for ·; and each · represents a point of attachment for which the shortest path to a therapeutic moiety C comprises less atoms than that for *, with the proviso that when n is >1 and a respective point of attachment is indicated on any one of R^{c}, R^{d} and R^{e}, then it can be independently present in one or more of the peptide monomeric units, preferably in one peptide monomeric unit most distant from the other point of attachment indicated in the respective structure, wherein each of the above structures optionally comprises a further attachment point to a moiety A or C.

### Moiety J

Moiety J forms part of the linking moiety B, and is characterized in that it comprises a ramification point, i.e., a central atom bound to at least three non-hydrogen atoms, wherein at least two of said non-hydrogen atoms are located on a covalent chain which represents the shortest path between a moiety A and said central atom. It can thus be regarded as a multipoint template to which the multiple arms bearing moieties A are joined together.

The moiety J together with the groups (L)ₐ are herein collectively denoted as moiety K.

J can be independently a moiety comprising a ramification point, comprising or consisting of a structural unit independently selected from the group consisting of alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, (oxo)alkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, tetrapeptide, each of which is substituted or unsubstituted.

### Moiety C

Moiety C in the present invention represents a therapeutic payload, which can be generally any atom (including H), molecule or particle. Preferably, moiety C is not a hydrogen atom.

The payload may be a chelator for radiolabeling a therapeutic conjugate with a therapeutic nuclide. Suitably, the radionuclide is not released. Chelators are well known to those skilled in the art, and for example, include chelators such as sulfur colloid, diethylenetriaminepentaacetic acid (DTPA), ethylenediaminetetraacetic acid (EDTA), 1,4,7,10-tetraazacyclododecane-N,N′,N″,N′-tetraacetic acid (DOTA), 1,4,7,10-tetraazacyclododececane, N-(glutaric acid)-N′,N″,N′″-triacetic acid (DOTAGA), 1,4,7-triazacyclononane-N,N′,N″-triacetic acid (NOTA), 1,4,8,11-tetraazacyclotetradecane-N,N′,N″,N′-tetraacetic acid (TETA), or any of the preferred chelator structures recited in the in the items further below or in the appended claims.

The therapeutic payload may be a radioactive group comprising or consisting of a therapeutic radioisotope, including isotopes such as ²²³Ra, ⁸⁹Sr, ⁹⁰Y, ¹²¹Sn, ¹⁷⁷Lu, ¹³¹I, ²¹¹At, ²²⁵Ac, ¹⁸⁸Re, ¹⁴⁹Tb, ¹⁶¹Tb and ²²⁷Th, preferably ⁹⁰Y, ²²⁵Ac or ¹⁷⁷Lu, more preferably ¹⁷⁷Lu; which may not be used for diagnostic applications.

The therapeutic payload may be a chelate of a therapeutic radioactive isotope, preferably of an isotope listed under above, with a chelating agent, preferably a chelating agent listed above or any of the preferred chelator structures recited further below; or a group selected from the structures listed further below.

The payload may be a cytotoxic and/or cytostatic agent. Such agents can inhibit or prevent the function of cells and/or cause destruction of cells. Examples of cytotoxic agents include radioactive therapeutic isotopes, chemotherapeutic agents, and toxins such as small molecule toxins or enzymatically active toxins of bacterial, fungal, plant or animal origin, including synthetic analogues and derivatives thereof. The cytotoxic agent may be selected from the group consisting of an auristatin, a DNA minor groove binding agent, a DNA minor groove alkylating agent, an enediyne, a lexitropsin, a duocarmycin, a taxane, a puromycin, a dolastatin, a maytansinoid and a vinca alkaloid or a combination of two or more thereof. Preferred cytotoxic and/or cytostatic payload moieties are listed in item 8 (e) further below.

In one embodiment the payload is a therapeutic chemotherapeutic agent selected from the group consisting of a topoisomerase inhibitor, an alkylating agent (e.g., nitrogen mustards; ethylenimes; alkylsulfonates; triazenes; piperazines; and nitrosureas), an antimetabolite (e.g., mercaptopurine, thioguanine, 5-fluorouracil), an antibiotics (e.g., anthracyclines, dactinomycin, bleomycin, adriamycin, mithramycin. dactinomycin) a mitotic disrupter (e.g., plant alkaloids—such as vincristine and/or microtubule antagonists—such as paclitaxel), a DNA methylating agent, a DNA intercalating agent (e.g., carboplatin and/or cisplatin, daunomycin and/or doxorubicin and/or bleomycin and/or thalidomide), a DNA synthesis inhibitor, a DNA-RNA transcription regulator, an enzyme inhibitor, a gene regulator, a hormone response modifier, a hypoxia-selective cytotoxin (e.g., tirapazamine), an epidermal growth factor inhibitor, an anti-vascular agent (e.g., xanthenone 5,6-dimethylxanthenone-4-acetic acid), a radiation-activated prodrug (e.g., nitroarylmethyl quaternary (NMQ) salts) or a bioreductive drug or a combination of two or more thereof. In some embodiments, the payload (i.e., moiety C) is not derived from an anthracycline, preferably not derived from PNU 159682.

The therapeutic chemotherapeutic agent may selected from the group consisting of Erlotinib (TARCEVA®), Bortezomib (VELCADE®), Fulvestrant (FASLODEX®), Sutent (SU11248), Letrozole (FEMARA®), Imatinib mesylate (GLEEVEC®), PTK787/ZK 222584, Oxaliplatin (Eloxatin®), 5-FU (5-fluorouracil), Leucovorin, Rapamycin (Sirolimus, RAPAMUNE®), Lapatinib (GSK572016), Lonafarnib (SCH 66336), Sorafenib (BAY43-9006), and Gefitinib (IRESSA®), AG1478, AG1571 (SU 5271; Sugen) or a combination of two or more thereof.

The therapeutic chemotherapeutic agent may be an alkylating agent—such as thiotepa, CYTOXAN® and/or cyclosphosphamide; an alkyl sulfonate—such as busulfan, improsulfan and/or piposulfan; an aziridine—such as benzodopa, carboquone, meturedopa and/or uredopa; ethylenimines and/or methylamelamines—such as altretamine, triethylenemelamine, triethylenepbosphoramide, triethylenethiophosphoramide and/or trimethylomelamine; acetogenin—such as bullatacin and/or bullatacinone; camptothecin; bryostatin; callystatin; cryptophycins; dolastatin; duocarmycin; eleutherobin; pancratistatin; sarcodictyin; spongistatin; nitrogen mustards—such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide and/or uracil mustard; nitrosureas—such as carmustine, chlorozotocin, fotemustine, lomustine, nimustine, and/or ranimnustine; dynemicin; bisphosphonates—such as clodronate; an esperamicin; a neocarzinostatin chromophore; aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN®. doxorubicin—such as morpholino-doxorubicin, cyanomorpholino-doxorubicin, 2-pyrrolino-doxorubicin and/or deoxydoxorubicin, epirubicin, esorubicin, idarubicin, marcellomycin, mitomycins—such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; antimetabolites—such as methotrexate and 5-fluorouracil (5-FU); folic acid analogues—such as denopterin, methotrexate, pteropterin, trimetrexate; purine analogues—such as fludarabine, 6-mercaptopurine, thiamiprine, thioguanine; pyrimidine analogues—such as ancitabine, azacitidine, 6-azauridine, carmofur, cytarabine, dideoxyuridine, doxifluridine, enocitabine, floxuridine; androgens—such as calusterone, dromostanolone propionate, epitiostanol, mepitiostane, testolactone; anti-adrenals—such as aminoglutethimide, mitotane, trilostane; folic acid replenisher—such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; macrocyclic depsipeptides such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; razoxane; rhizoxin; sizofiran; spirogermanium; tenuazonic acid; triaziquone; 2,2′,2″-trichlorotriethylamine; trichothecenes—such as verracurin A, roridin A and/or anguidine; urethan; vindesine; dacarbazine; mannomustine; mitobronitol; mitolactol; pipobroman; gacytosine; arabinoside; cyclophosphamide; thiotepa; taxoids—such as TAXOL®. paclitaxel, abraxane, and/or TAXOTERE®, doxetaxel; chloranbucil; GEMZAR®. gemcitabine; 6-thioguanine; mercaptopurine; methotrexate; platinum analogues—such as cisplatin and carboplatin; vinblastine; platinum; etoposide; ifosfamide; mitoxantrone; vincristine; NAVELBINE®, vinorelbine; novantrone; teniposide; edatrexate; daunomycin; aminopterin; xeloda; ibandronate; topoisomerase inhibitor RFS 2000; difluoromethylomithine (DMFO); retinoids—such as retinoic acid; capecitabine; and pharmaceutically acceptable salts, acids, derivatives or combinations of two or more of any of the above.

The therapeutic payload may be a tubulin disruptor including but are not limited to: taxanes—such as paclitaxel and docetaxel, vinca alkaloids, discodermolide, epothilones A and B, desoxyepothilone, cryptophycins, curacin A, combretastatin A-4-phosphate, BMS 247550, BMS 184476, BMS 188791; LEP, RPR 109881A, EPO 906, TXD 258, ZD 6126, vinflunine, LU 103793, dolastatin 10, E7010, T138067 and T900607, colchicine, phenstatin, chalcones, indanocine, T138067, oncocidin, vincristine, vinblastine, vinorelbine, vinflunine, halichondrin B, isohomohalichondrin B, ER-86526, pironetin, spongistatin 1, spiket P, cryptophycin 1, LU103793 (cematodin or cemadotin), rhizoxin, sarcodictyin, eleutherobin, laulilamide, VP-16 and D-24851 and pharmaceutically acceptable salts, acids, derivatives or combinations of two or more of any of the above.

The therapeutic payload may be a DNA intercalator including but are not limited to: acridines, actinomycins, anthracyclines, benzothiopyranoindazoles, pixantrone, crisnatol, brostallicin, CI-958, doxorubicin (adriamycin), actinomycin D, daunorubicin (daunomycin), bleomycin, idarubicin, mitoxantrone, cyclophosphamide, melphalan, mitomycin C, bizelesin, etoposide, mitoxantrone, SN-38, carboplatin, cis-platin, actinomycin D, amsacrine, DACA, pyrazoloacridine, irinotecan and topotecan and pharmaceutically acceptable salts, acids, derivatives or combinations of two or more of any of the above.

The therapeutic payload may be an anti-hormonal agent that acts to regulate or inhibit hormone action on tumors—such as anti-estrogens and selective estrogen receptor modulators, including, but not limited to, tamoxifen, raloxifene, droloxifene, 4-hydroxytamoxifen, trioxifene, keoxifene, LY117018, onapristone, and/or fareston toremifene and pharmaceutically acceptable salts, acids, derivatives or combinations of two or more of any of the above. The therapeutic payload may be an aromatase inhibitor that inhibits the enzyme aromatase, which regulates estrogen production in the adrenal glands—such as, for example, 4 (5)-imidazoles, aminoglutethimide, megestrol acetate, AROMASIN®. exemestane, formestanie, fadrozole, RIVISOR®. vorozole, FEMARA®. letrozole, and ARIMIDEX® and/or anastrozole and pharmaceutically acceptable salts, acids, derivatives or combinations of two or more of any of the above.

The therapeutic payload may be an anti-androgen such as flutamide, nilutamide, bicalutamide, leuprolide, goserelin and/or troxacitabine and pharmaceutically acceptable salts, acids, derivatives or combinations of two or more of any of the above.

The therapeutic payload may be a protein or an antibody. Preferably, the payload is a cytokine (e.g., an interleukin such as IL2, IL10, IL12, IL15; a member of the TNF superfamily; or an interferon such as interferon gamma).

Any therapeutic payload may be used in unmodified or modified form. Combinations of therapeutic payloads in which some are unmodified and some are modified may be used. For example, the therapeutic payload may be chemically modified. One form of chemical modification is the derivatisation of a carbonyl group—such as an aldehyde.

In a preferred embodiment, the payload moiety C is a topoisomerase inhibitor; preferably camptothecin (CPT) or a derivative thereof; more preferably derived (e.g., by replacing a hydrogen atom) from topotecan, irinotecan, silatecan, cositecan, exatecan, lurtotecan, gimatecan, belotecan, rubitecan, deruxtecan, DXd; even more preferably exatecan; even more preferably

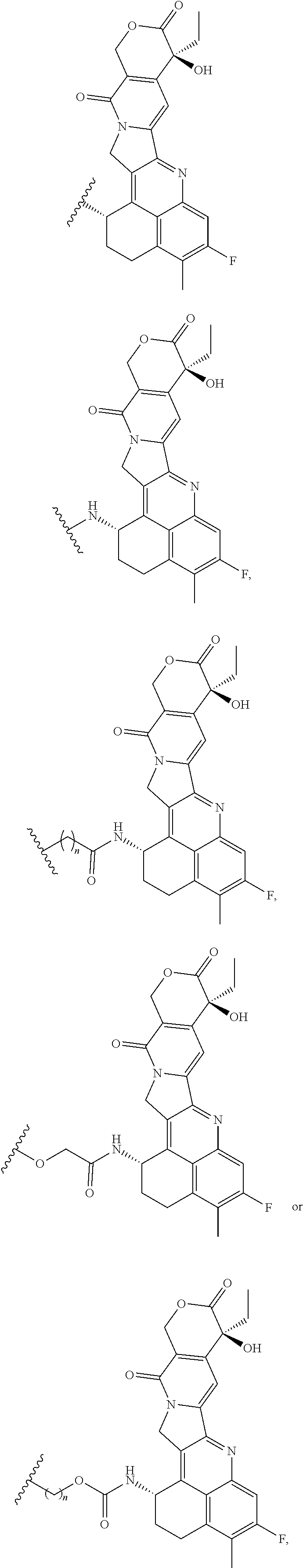

wherein each n is 0, 1, 2, 3, 4, 5 or 6; and most preferably

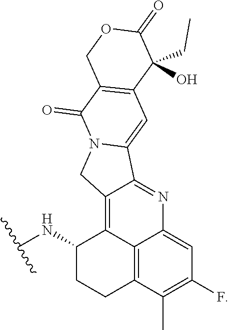 [SMILES: *N[C@H]1CCc2c(C)c(F)cc3nc4c(c1c23)Cn1c-4cc2c(c1=O)COC(=O)[C@]2(O)CC]

In a preferred embodiment, therapeutic moiety C is an auristatin (i.e., having a structure derived from an auristatin compound family member) or an auristatin derivative. More preferably, therapeutic moiety C has a structure according to the following formula:

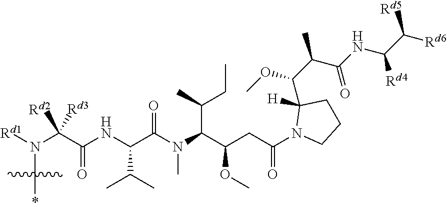

wherein:
R^{1d} is independently H or C₁-C₆ alkyl; preferably H or CH₃;
R^{2d} is independently C₁-C₆ alkyl; preferably CH₃ or iPr;
R^{3d} is independently H or C₁-C₆ alkyl; preferably H or CH₃;
R^{4d} is independently H, C₁-C₆ alkyl, COO(C₁-C₆ alkyl), CON(H or C₁-C₆ alkyl), C₃-C₁₀ aryl or C₃-C₁₀ heteroaryl; preferably H, CH₃, COOH, COOCH₃ or thiazolyl;
R^{5d} is independently H, OH, C₁-C₆ alkyl; preferably H or OH; and
R^{6d} is independently C₃-C₁₀ aryl or C₃-C₁₀ heteroaryl; preferably optionally substituted phenyl or pyridyl.

More preferably, therapeutic moiety C is derived from MMAE or MMAF.

In a preferred embodiment, therapeutic moiety C has a structure according to the following formula:

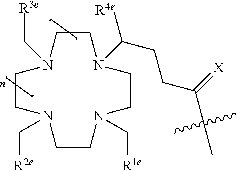

wherein:
n is 0, 1, 2, 3, 4 or 5; preferably 1;
R^{1e} is independently H, COOH, aryl-COOH or heteroaryl-COOH; preferably COOH;
R^{2e} is independently H, COOH, aryl-COOH or heteroaryl-COOH; preferably COOH;
each R^{3e} is independently H, COOH, aryl-COOH or heteroaryl-COOH; preferably COOH;
R^{4e} is independently H, COOH, aryl-COOH or heteroaryl-COOH; preferably COOH; and
X is O, NH or S; preferably O.

In a preferred embodiment, therapeutic moiety C has a structure according to the following formula:

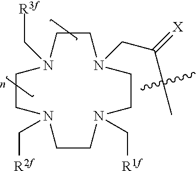

wherein:
n is 0, 1, 2, 3, 4 or 5; preferably 1
R^{1f} is independently H, COOH, aryl-COOH or heteroaryl-COOH; preferably COOH;
R^{2f} is independently H, COOH, aryl-COOH or heteroaryl-COOH; preferably COOH;
R^{3f} is independently H, COOH, aryl-COOH or heteroaryl-COOH; preferably COOH; and
X is O, NH or S; preferably O.

Particularly preferred embodiments for the therapeutic moiety C as well as the compound according to the present invention are shown in the items further below and the appended claims.

As noted above, a preferred embodiment when C is a chelator is represented by Formula III. An exemplary, particularly preferred structure of this type is represented by Formula IIIa, wherein all variables and groups are as defined in the claims, and which may be present in the form of a complex with any of the therapeutic radioactive isotopes disclosed herein, preferably 90-Yttrium, 225-Actinium or 177-Lutetium, more preferably 177-Lutetium:

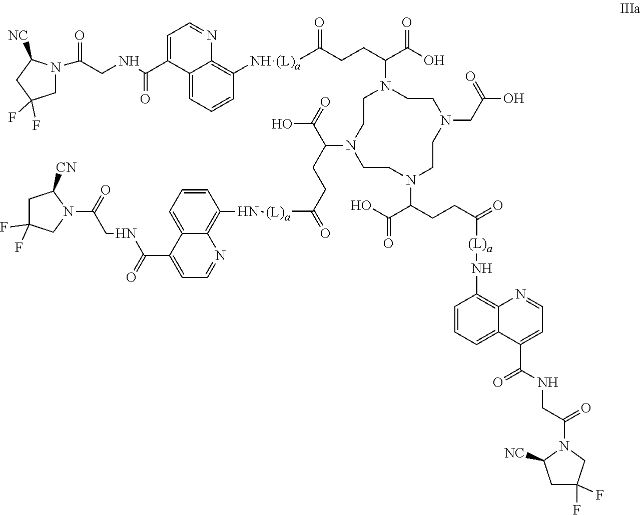

### Further Aspects

The fragment ((B_{S})ₓ(B_{L})_{y})_{z} can be represented by one of the following structures:
single bond, (B_{S})ₓ,

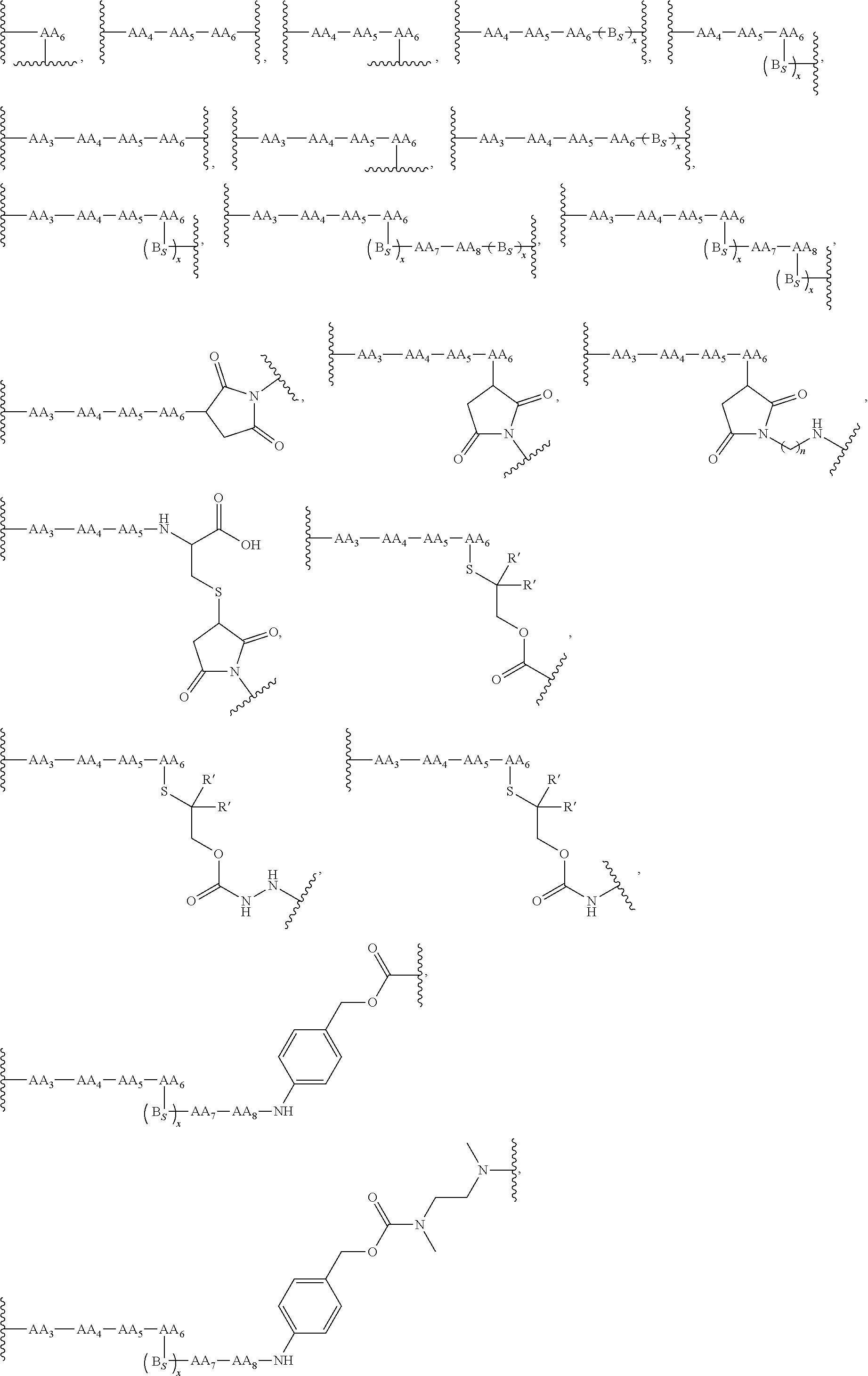

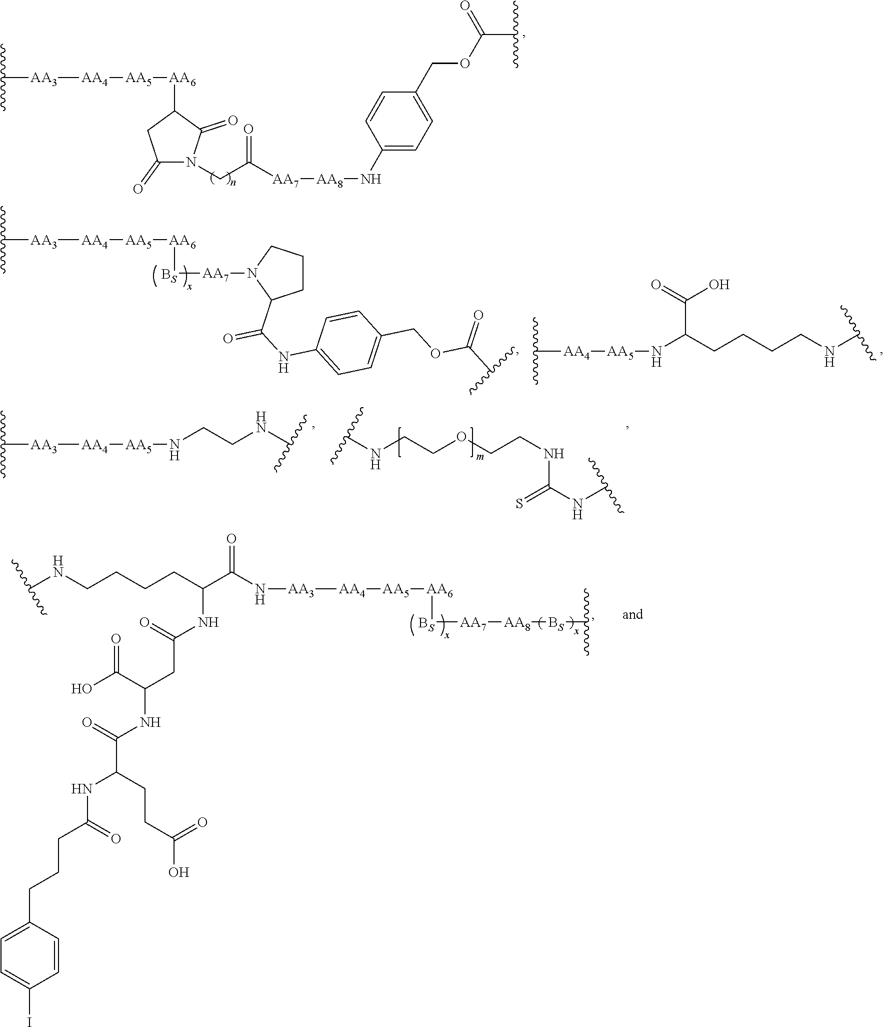

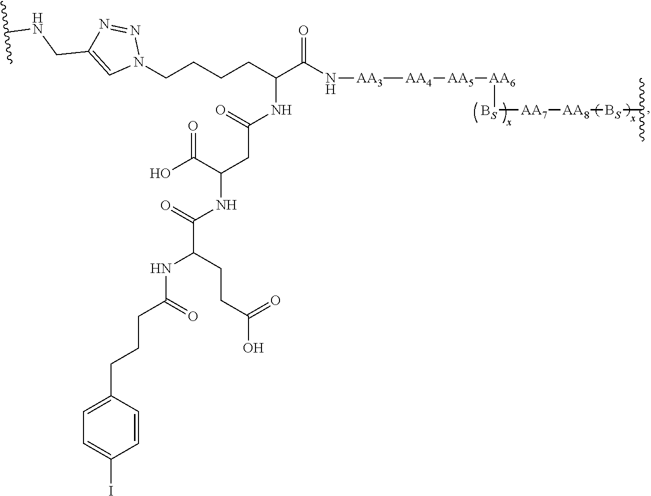

wherein each of AA₃, AA₄, AA₅, AA₆, AA₇, and AA₈ represents a proteinogenic or non-proteinogenic amino acid, or is absent;
wherein preferably:
   each proteinogenic or non-proteinogenic amino acid is preferably independently represented by one of the following structures:

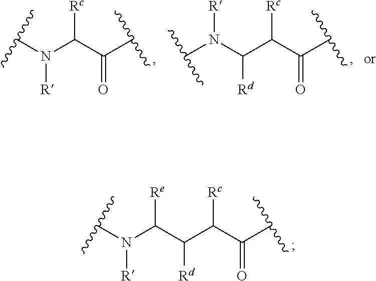

and/or AA₄ is an amino acid with a charged sidechain, and AA₇ is an amino acid with an aliphatic sidechain;
wherein more preferably:
AA₃ is selected from Asp, Glu, and Lys, or is absent; preferably Asp;
AA₄ is selected from Arg, HomoArg, Lys, Asp, and Glu, or is absent; preferably Lys or Arg;
AA₅ is selected from Asp, Glu, and Lys; preferably Asp;
AA₆ is selected from Cys, Lys, Gly and Val; preferably Cys or Lys;
AA₇ is selected from Gly, Ala, Val, Arg, Ile, Pro; and
AA₈ is selected from Pro and citrulline (Cit);
even more preferably according to one of the sequences shown in the below table:

| AA₃ | AA₄ | AA₅ | AA₆ | AA₇ | AA₈ |
|---|---|---|---|---|---|
| — | — | — | Lys | — | — |
| Asp | Arg | Asp | Cys | — | — |
| Asp | Arg | Asp | Gly | — | — |
| Asp | Lys | Asp | Lys | — | — |
| Asp | Lys | Asp | Val | — | — |
| Asp | Arg | Asp | Cys | Val | Ala |
| Asp | Arg | Asp | Cys | Val | Cit |
| Asp | HomoArg | Asp | Cys | Val | Cit |
| Asp | Lys | Asp | Cys | any AA | Pro |
| Asp | Lys | Asp | Cys | Ala | Pro |
| Asp | Lys | Asp | Cys | Ala | Val |
| Asp | Lys | Asp | Cys | Arg | Pro |
| Asp | Lys | Asp | Cys | Gly | Pro |
| Asp | Lys | Asp | Cys | Ile | Pro |
| Asp | Lys | Asp | Cys | Pro | Pro |
| Asp | Lys | Asp | Cys | Val | Cit |
| Asp | Lys | Asp | Cys | Val | Pro |

or
the fragment ((B_{S})ₓ(B_{L})_{y})_{z} can be represented by one of the following structures:
single bond,

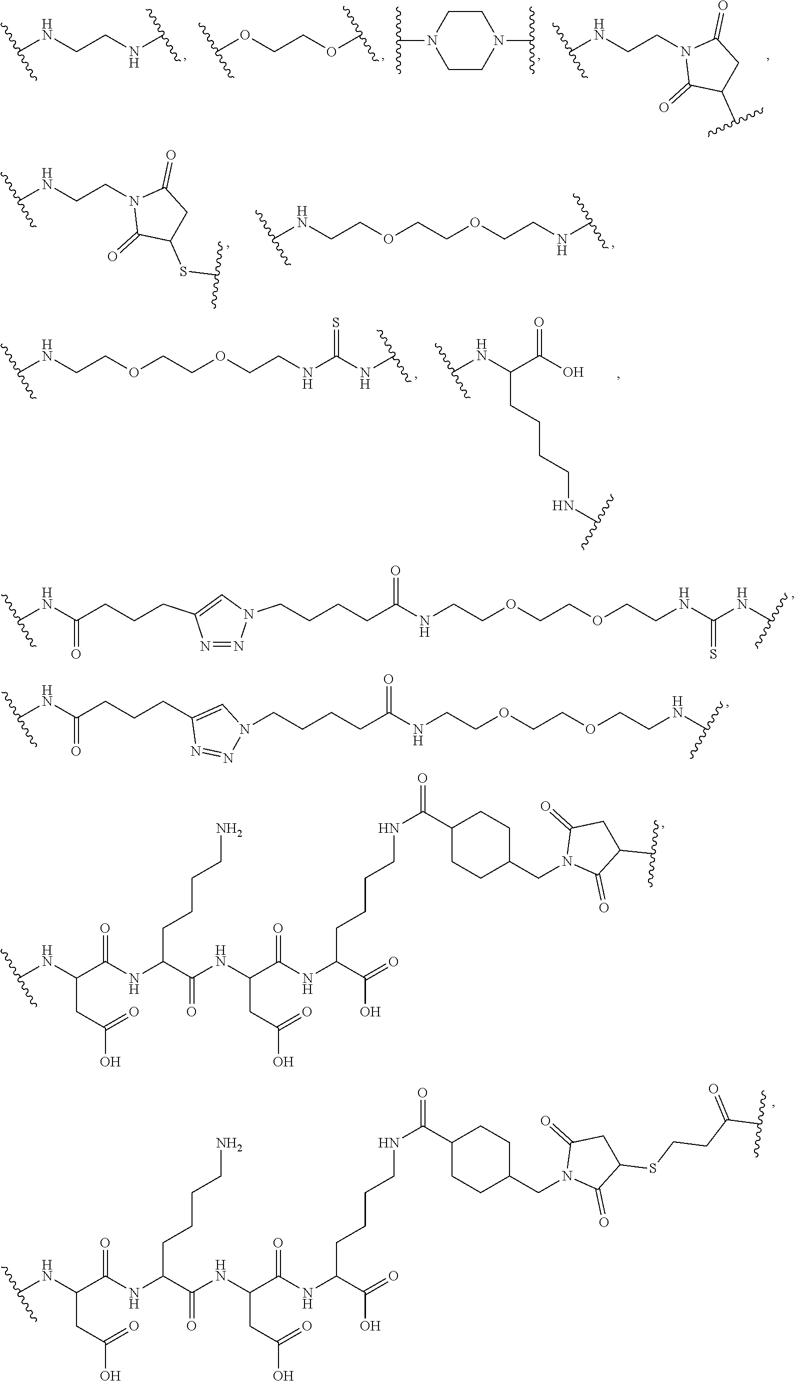

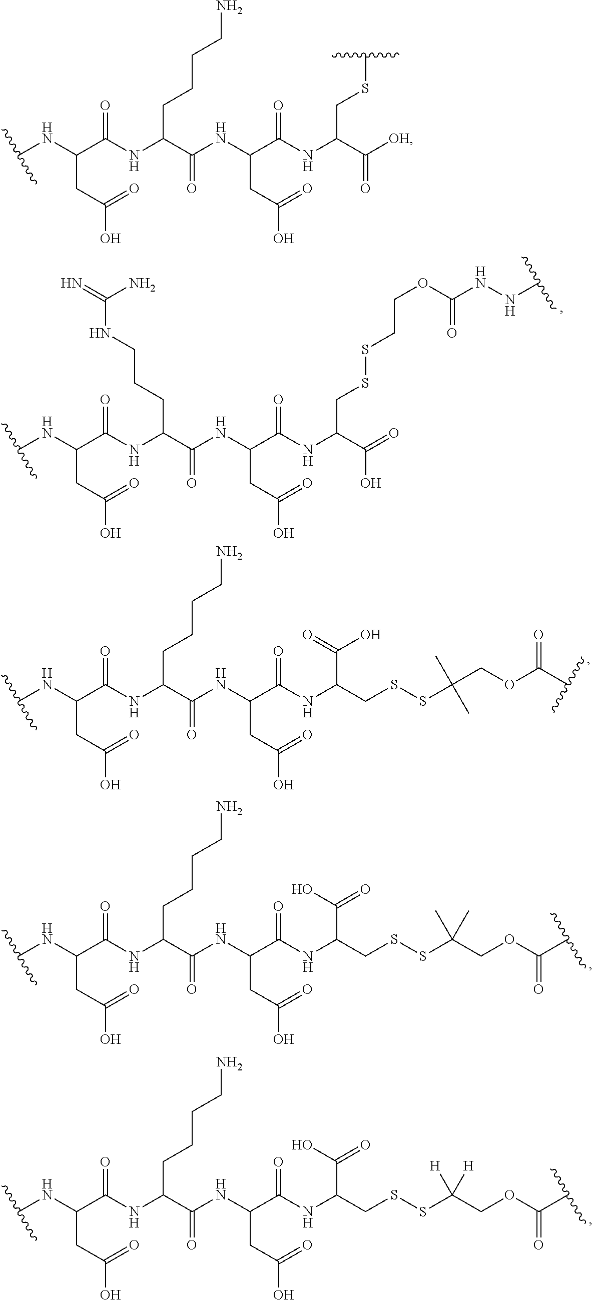 [SMILES: *NC(CC(=O)O)C(=O)NC(CCCCN)C(=O)NC(CC(=O)O)C(=O)NC(CS*)C(=O)O ; *NNC(=O)OCCSSCC(NC(=O)C(CC(=O)O)NC(=O)C(CCCNC(=N)N)NC(=O)C(CC(=O)O)N*)C(=O)O ; *NC(CC(=O)O)C(=O)NC(CCCCN)C(=O)NC(CC(=O)O)C(=O)NC(CSSC(C)(C)COC(*)=O)C(=O)O ; *NC(CC(=O)O)C(=O)NC(CCCCN)C(=O)NC(CC(=O)O)C(=O)NC(CSSC(C)(C)COC(*)=O)C(=O)O ; *NC(CC(=O)O)C(=O)NC(CCCCN)C(=O)NC(CC(=O)O)C(=O)NC(CSSCCOC(*)=O)C(=O)O]

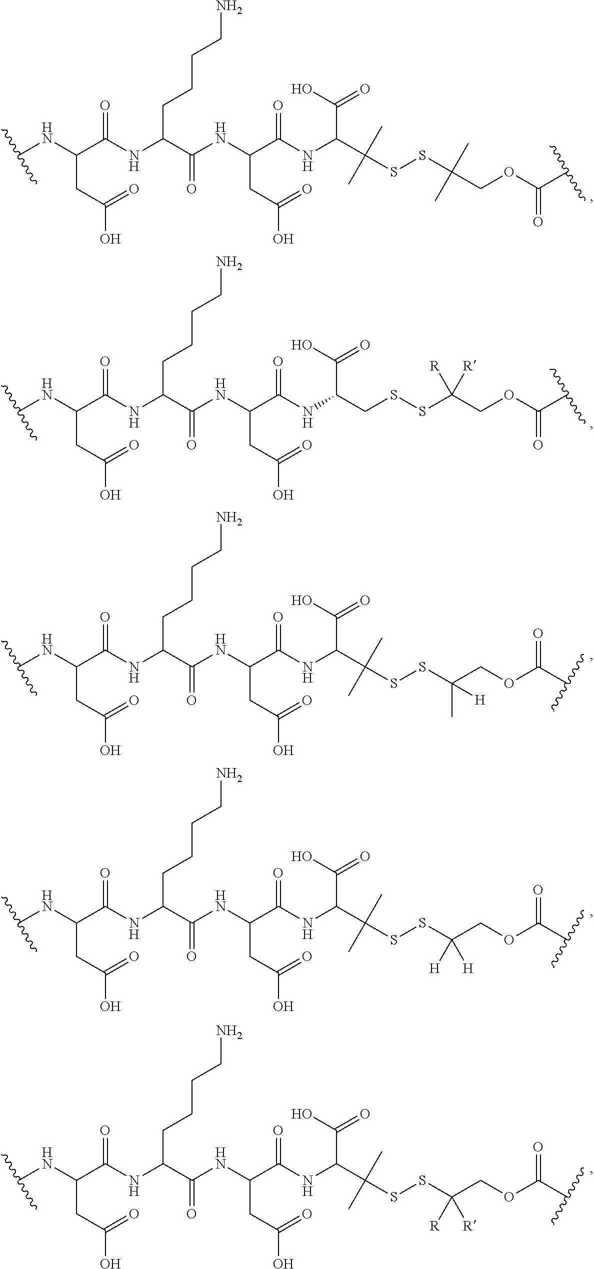

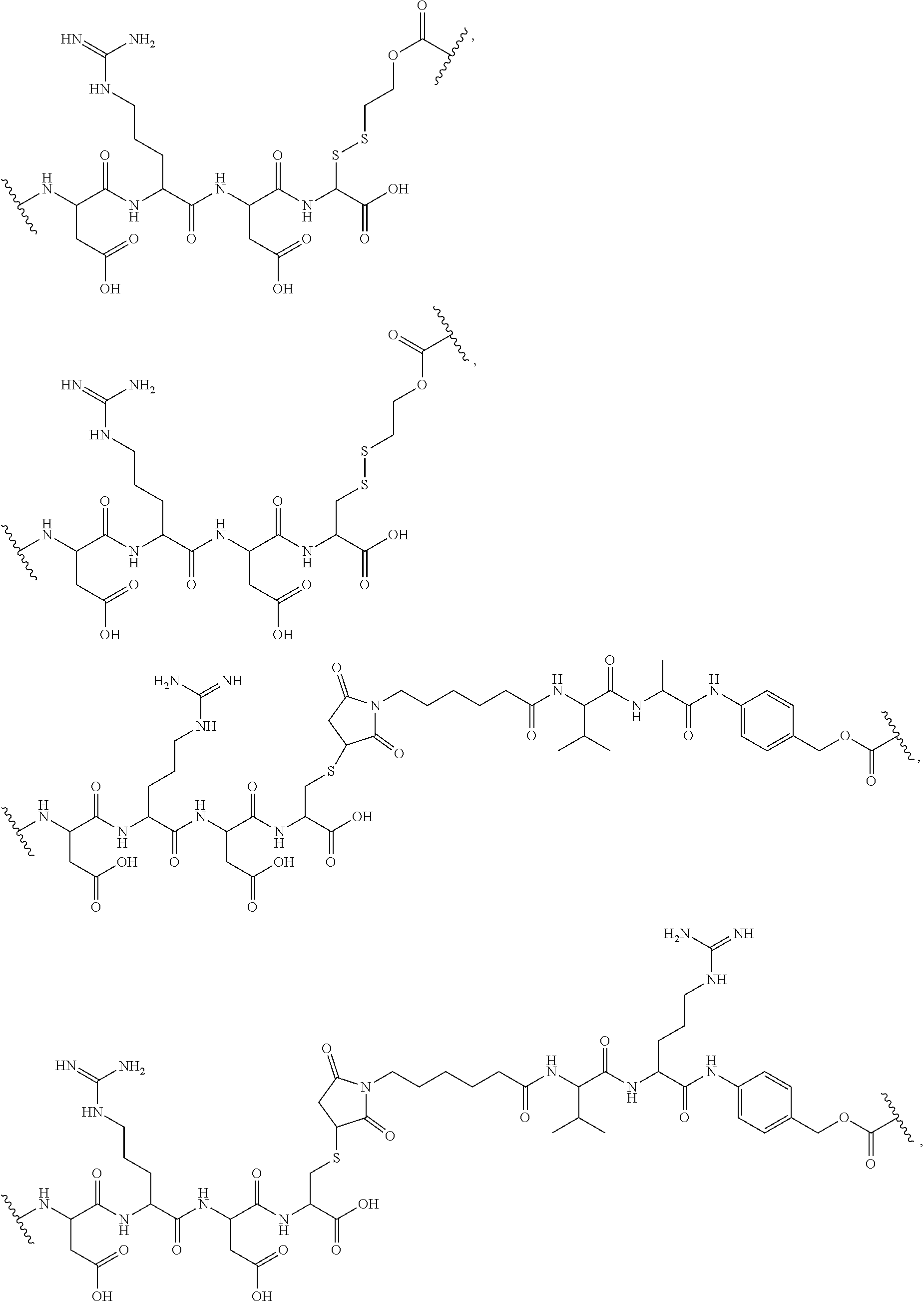 [SMILES: *NC(CC(=O)O)C(=O)NC(CCCNC(=N)N)C(=O)NC(CC(=O)O)C(=O)NC(SSCCOC(*)=O)C(=O)O ; *NC(CC(=O)O)C(=O)NC(CCCNC(=N)N)C(=O)NC(CC(=O)O)C(=O)NC(CSSCCOC(*)=O)C(=O)O ; *NC(CC(=O)O)C(=O)NC(CCCNC(=N)N)C(=O)NC(CC(=O)O)C(=O)NC(CSC1CC(=O)N(CCCCCC(=O)NC(C(=O)NC(C)C(=O)Nc2ccc(COC(*)=O)cc2)C(C)C)C1=O)C(=O)O ; *NC(CC(=O)O)C(=O)NC(CCCNC(=N)N)C(=O)NC(CC(=O)O)C(=O)NC(CSC1CC(=O)N(CCCCCC(=O)NC(C(=O)NC(CCCNC(=N)N)C(=O)Nc2ccc(COC(*)=O)cc2)C(C)C)C1=O)C(=O)O]

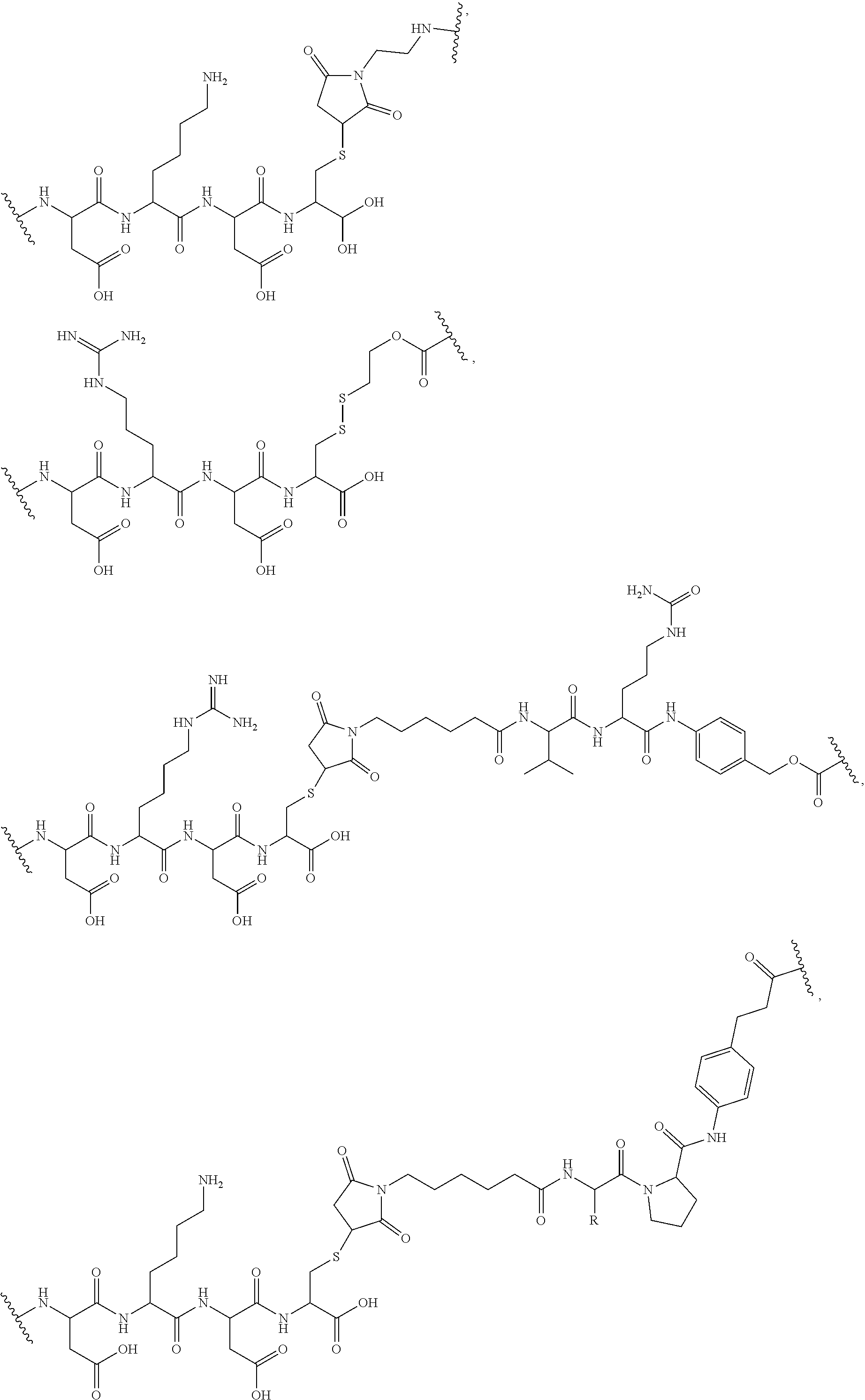

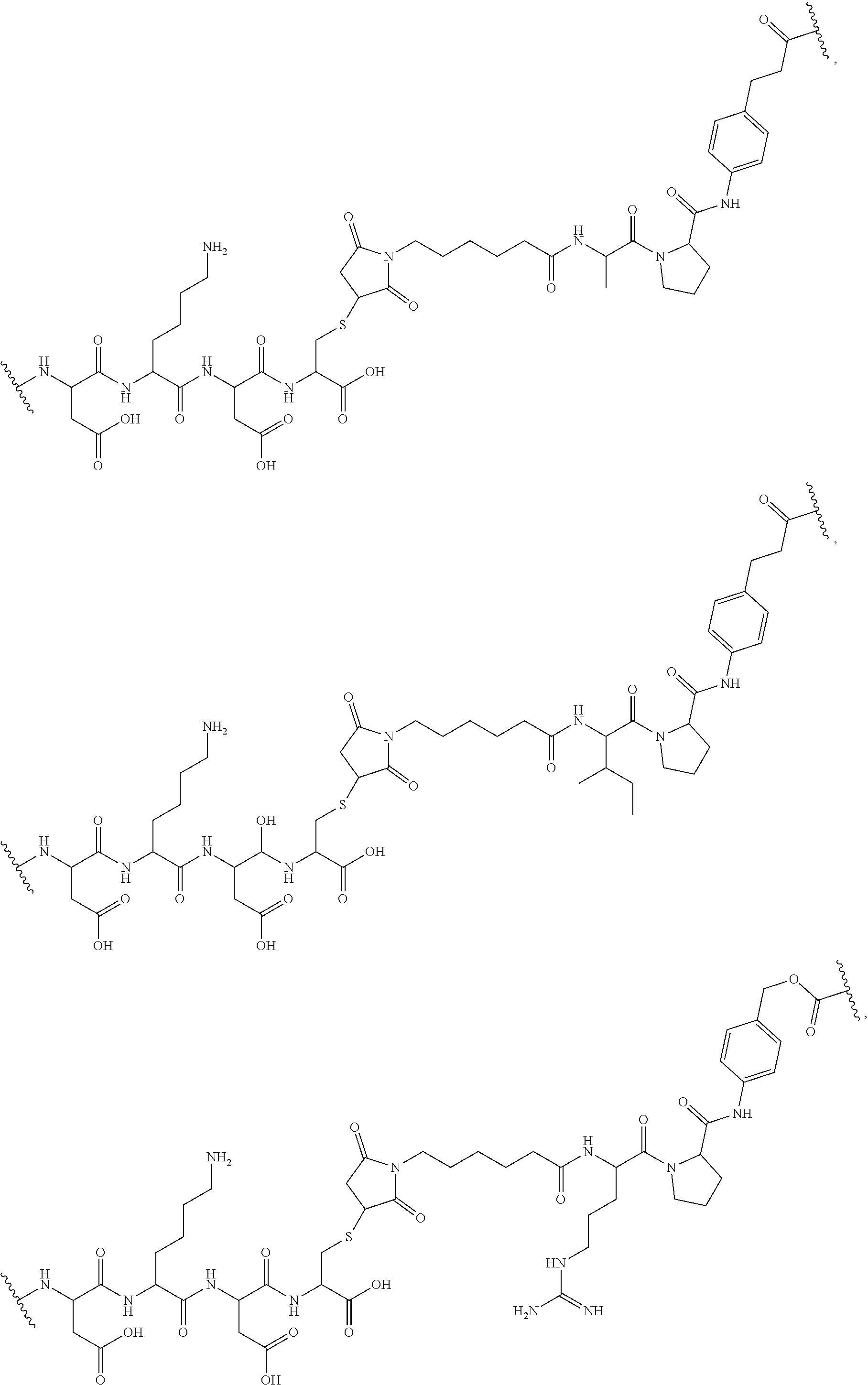 [SMILES: *NC(CC(=O)O)C(=O)NC(CCCCN)C(=O)NC(CC(=O)O)C(=O)NC(CSC1CC(=O)N(CCCCCC(=O)NC(C)C(=O)N2CCCC2C(=O)Nc2ccc(CCC(*)=O)cc2)C1=O)C(=O)O ; *NC(CC(=O)O)C(=O)NC(CCCCN)C(=O)NC(CC(=O)O)C(O)NC(CSC1CC(=O)N(CCCCCC(=O)NC(C(=O)N2CCCC2C(=O)Nc2ccc(CCC(*)=O)cc2)C(C)CC)C1=O)C(=O)O ; *NC(CC(=O)O)C(=O)NC(CCCCN)C(=O)NC(CC(=O)O)C(=O)NC(CSC1CC(=O)N(CCCCCC(=O)NC(CCCNC(=N)N)C(=O)N2CCCC2C(=O)Nc2ccc(COC(*)=O)cc2)C1=O)C(=O)O]

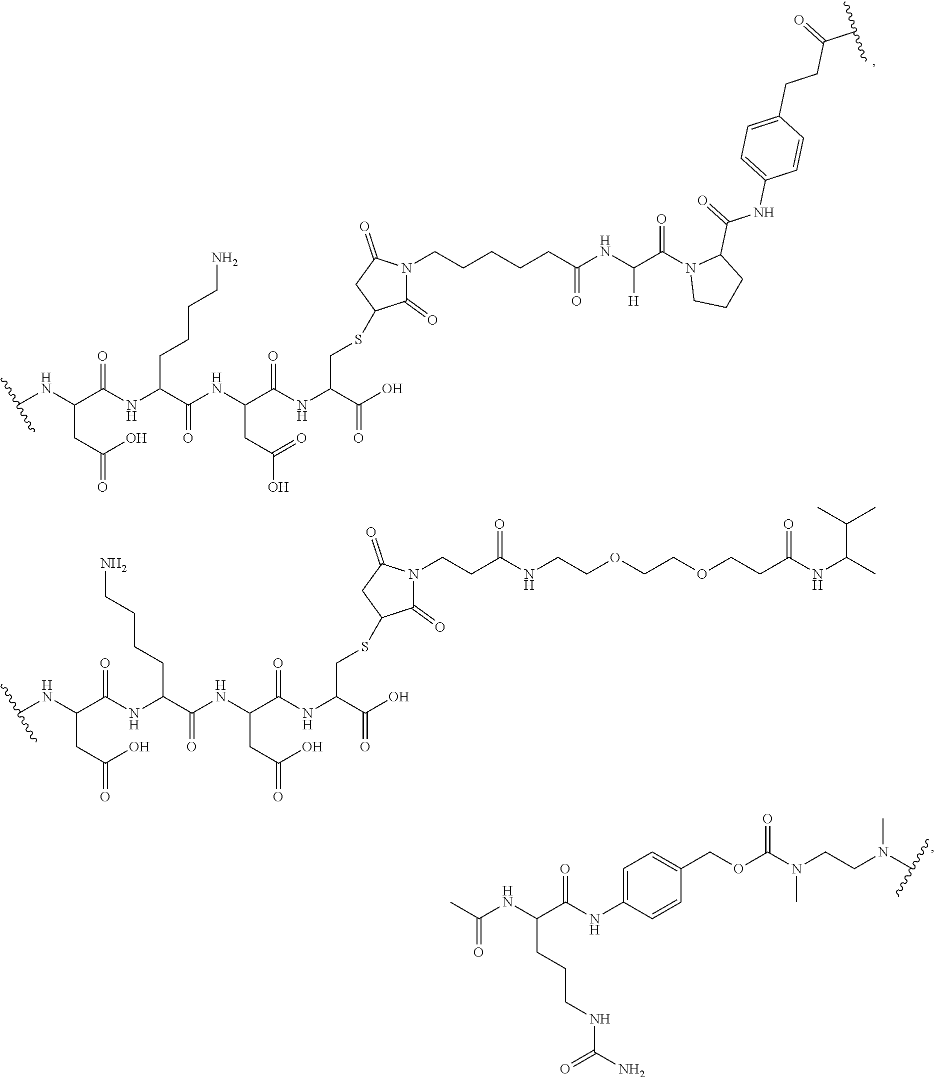

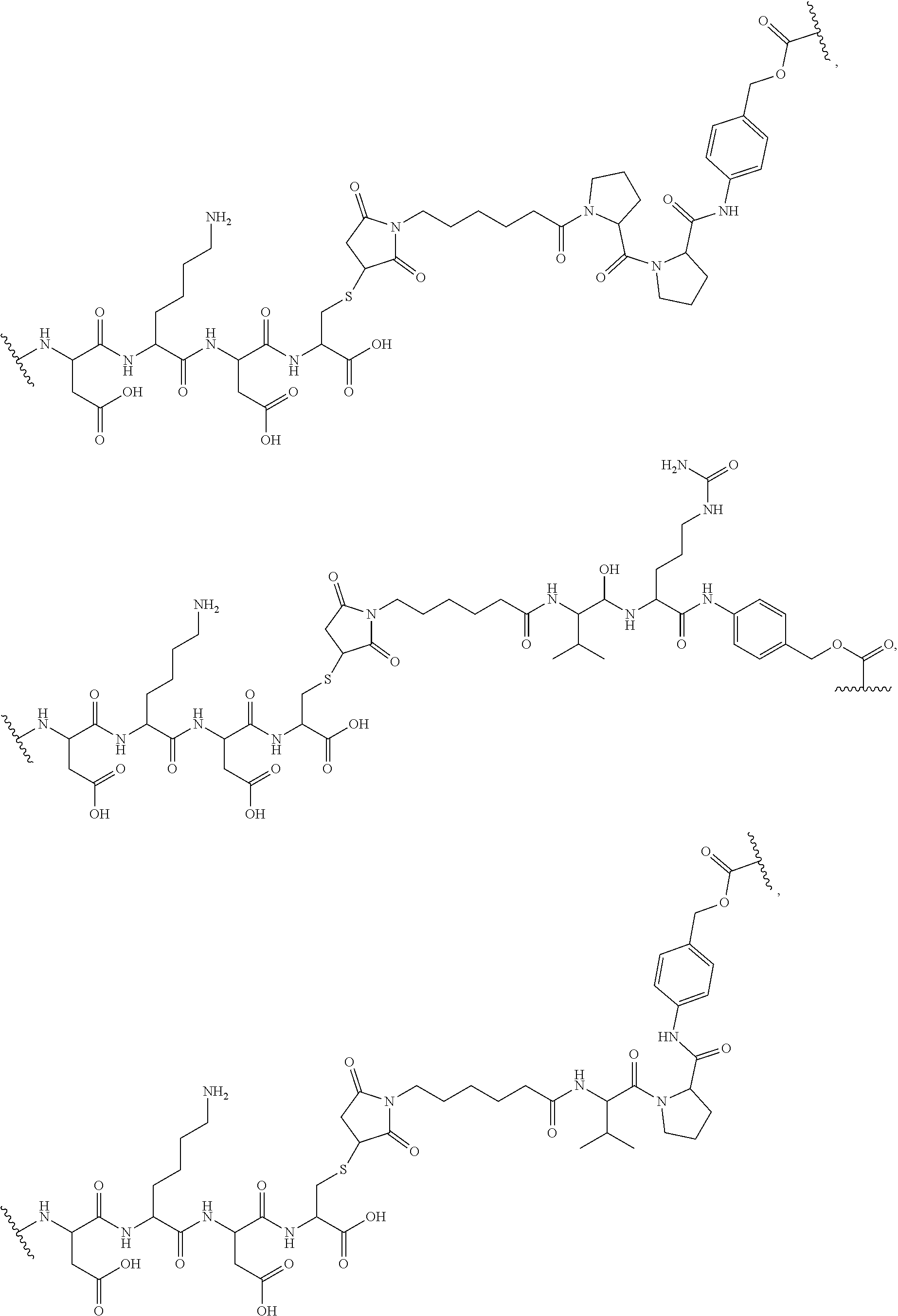 [SMILES: *NC(CC(=O)O)C(=O)NC(CCCCN)C(=O)NC(CC(=O)O)C(=O)NC(CSC1CC(=O)N(CCCCCC(=O)N2CCCC2C(=O)N2CCCC2C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCCCCC(=O)NC(C(C)C)C(O)NC(CCCNC(N)=O)C(=O)Nc2ccc(COC(*)=O)cc2)C1=O)C(=O)O ; *NC(CC(=O)O)C(=O)NC(CCCCN)C(=O)NC(CC(=O)O)C(=O)NC(CSC1CC(=O)N(CCCCCC(=O)NC(C(=O)N2CCCC2C(=O)Nc2ccc(COC(*)=O)cc2)C(C)C)C1=O)C(=O)O]

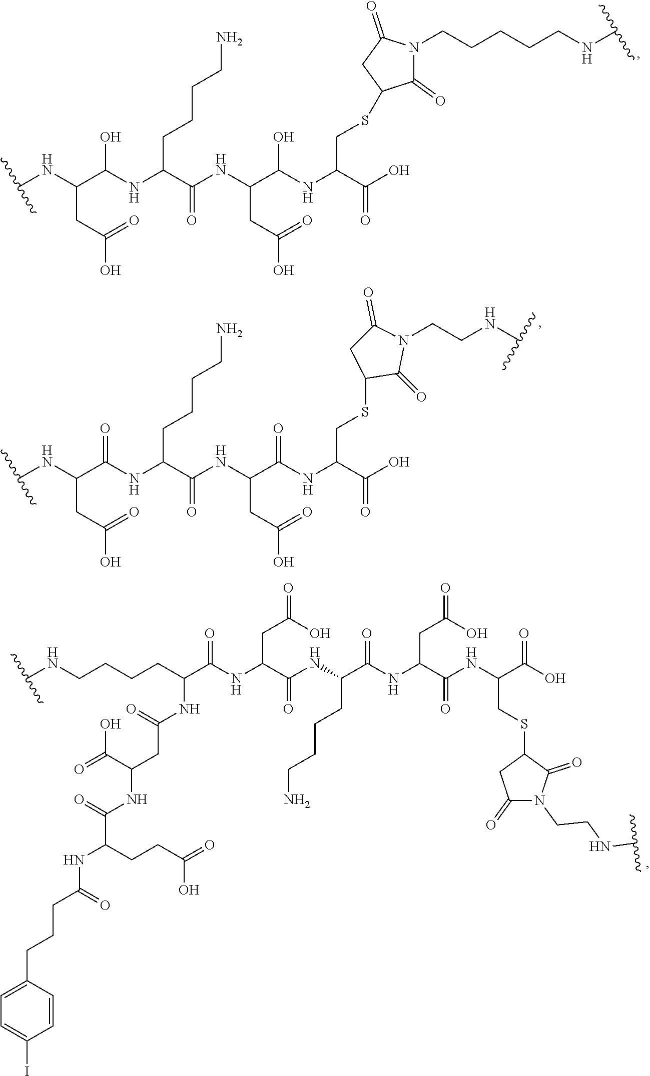

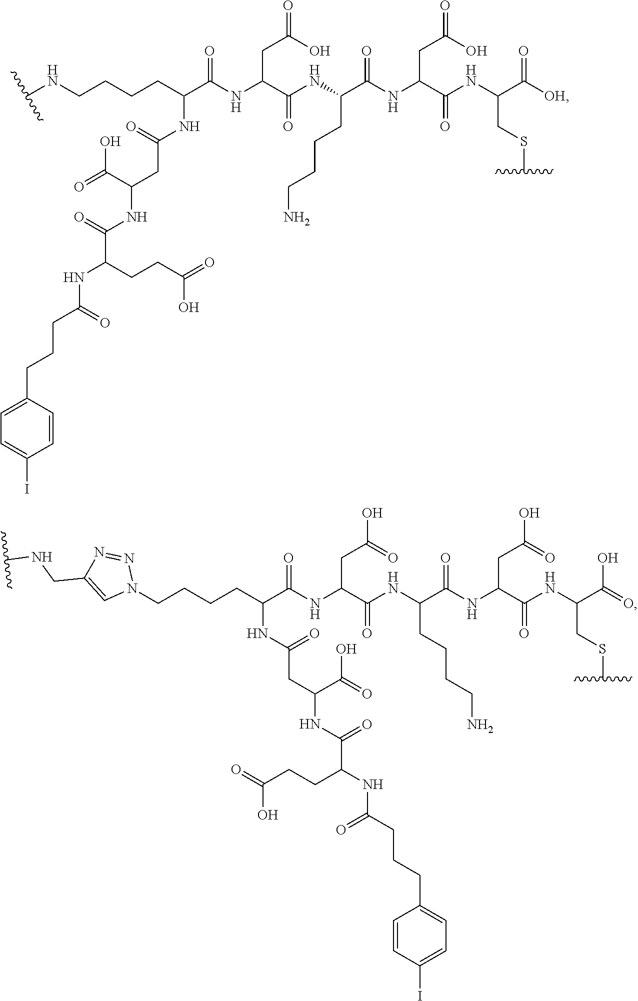 [SMILES: *NCCCCC(NC(=O)CC(NC(=O)C(CCC(=O)O)NC(=O)CCCc1ccc(I)cc1)C(=O)O)C(=O)NC(CC(=O)O)C(=O)N[C@@H](CCCCN)C(=O)NC(CC(=O)O)C(=O)NC(CS*)C(=O)O ; *NCc1cn(CCCCC(NC(=O)CC(NC(=O)C(CCC(=O)O)NC(=O)CCCc2ccc(I)cc2)C(=O)O)C(=O)NC(CC(=O)O)C(=O)NC(CCCCN)C(=O)NC(CC(=O)O)C(=O)NC(CS*)C(=O)O)nn1]

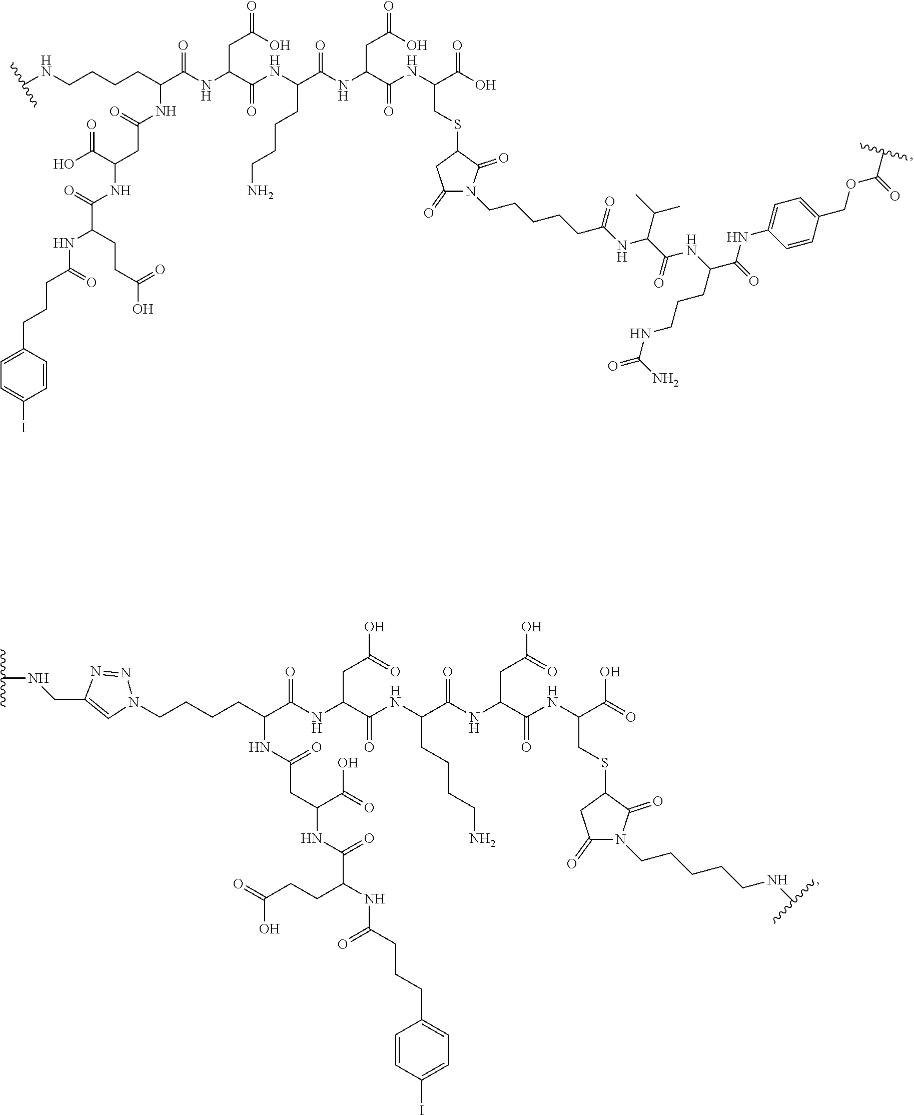 [SMILES: *NCCCCC(NC(=O)CC(NC(=O)C(CCC(=O)O)NC(=O)CCCc1ccc(I)cc1)C(=O)O)C(=O)NC(CC(=O)O)C(=O)NC(CCCCN)C(=O)NC(CC(=O)O)C(=O)NC(CSC1CC(=O)N(CCCCCC(=O)NC(C(=O)NC(CCCNC(N)=O)C(=O)Nc2ccc(COC(*)=O)cc2)C(C)C)C1=O)C(=O)O ; *NCCCCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O]

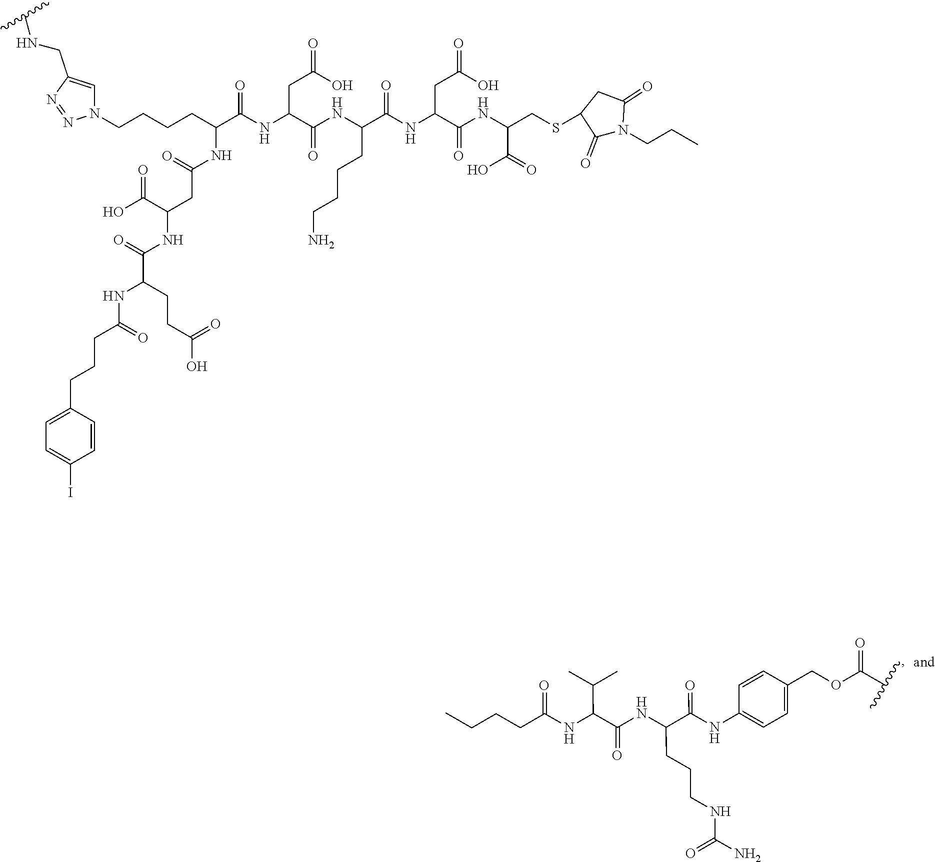

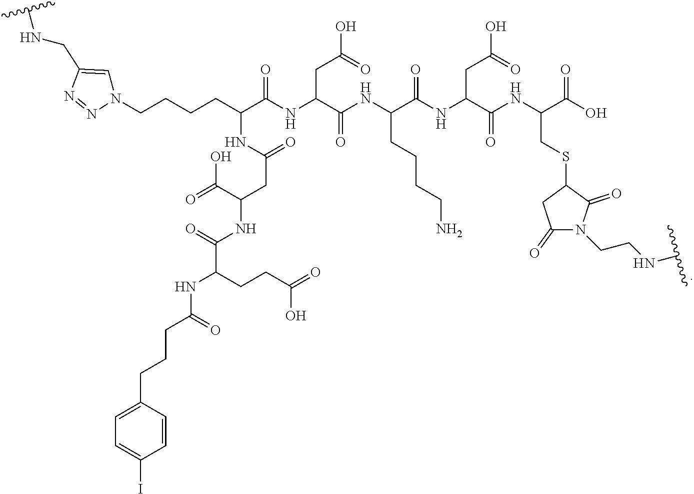 [SMILES: *NCCN1C(=O)CC(SCC(NC(=O)C(CC(=O)O)NC(=O)C(CCCCN)NC(=O)C(CC(=O)O)NC(=O)C(CCCCn2cc(CN*)nn2)NC(=O)CC(NC(=O)C(CCC(=O)O)NC(=O)CCCc2ccc(I)cc2)C(=O)O)C(=O)O)C1=O]

In some embodiments, the compound may be represented by one of the following structures:

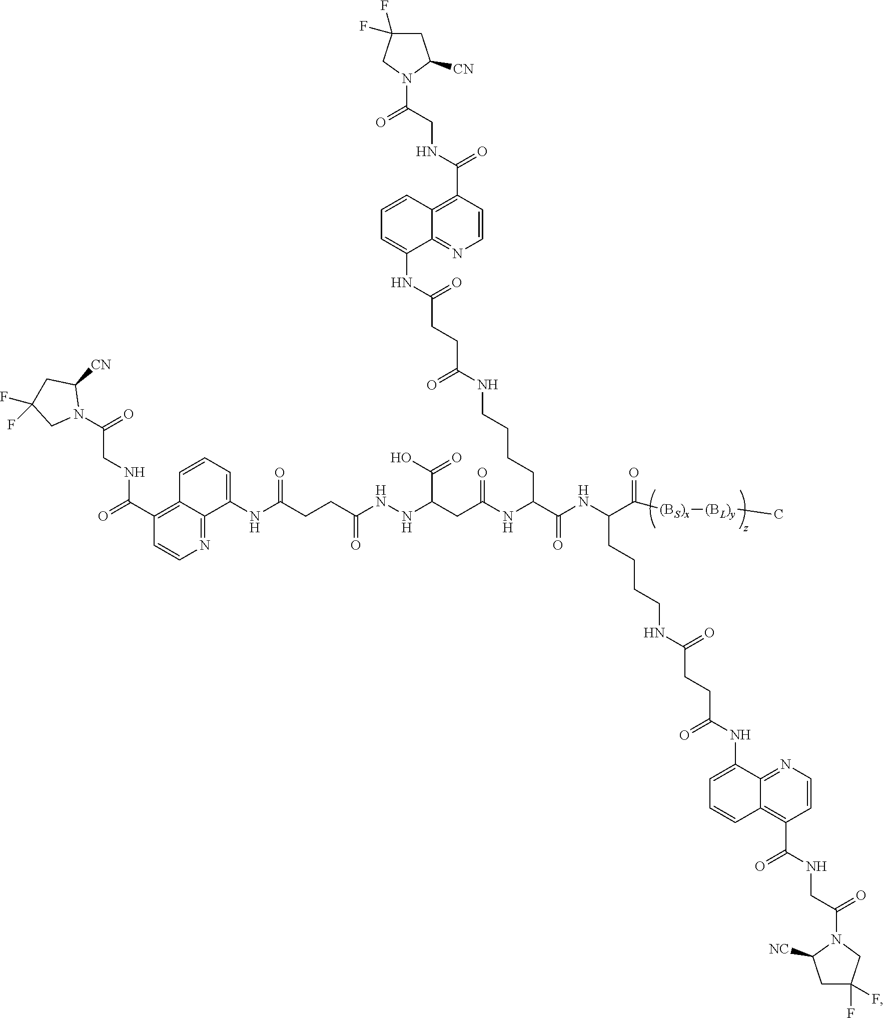

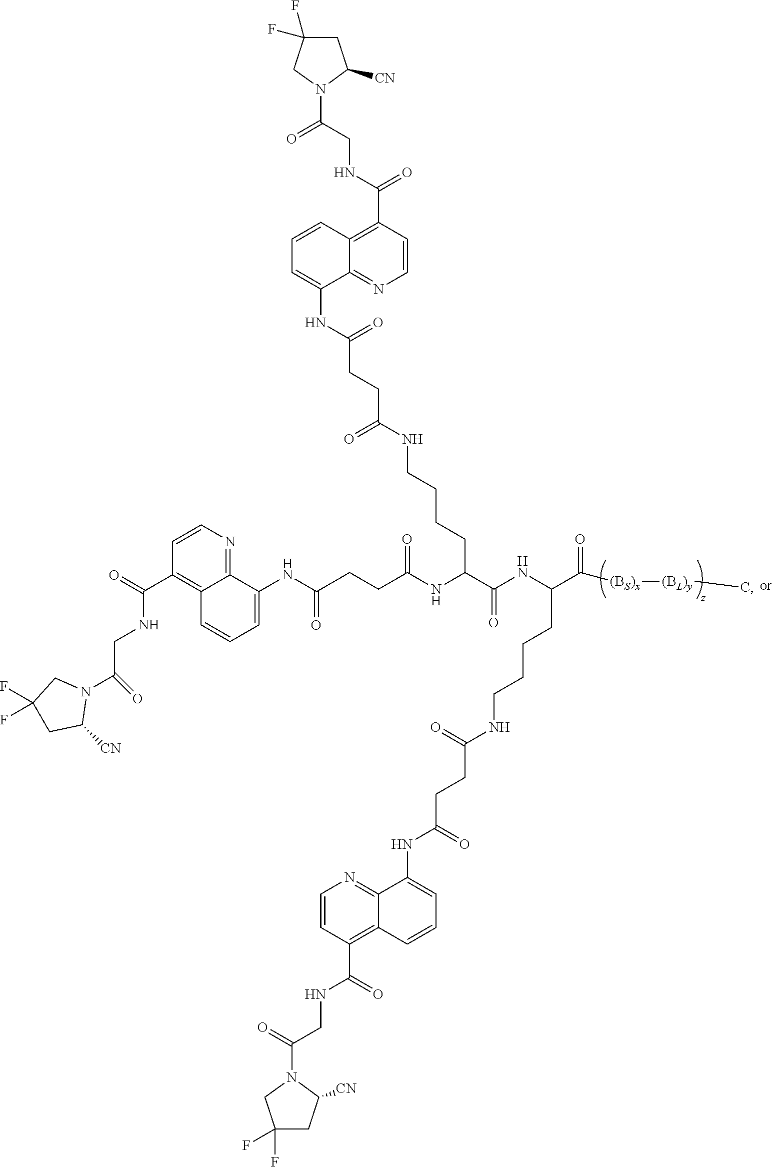

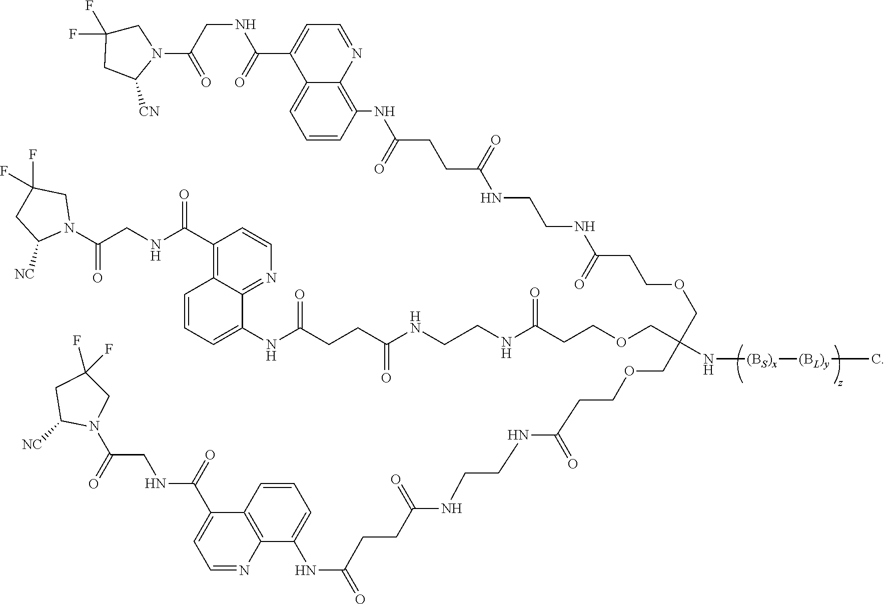

Preferred compounds according to the present invention may be represented by:

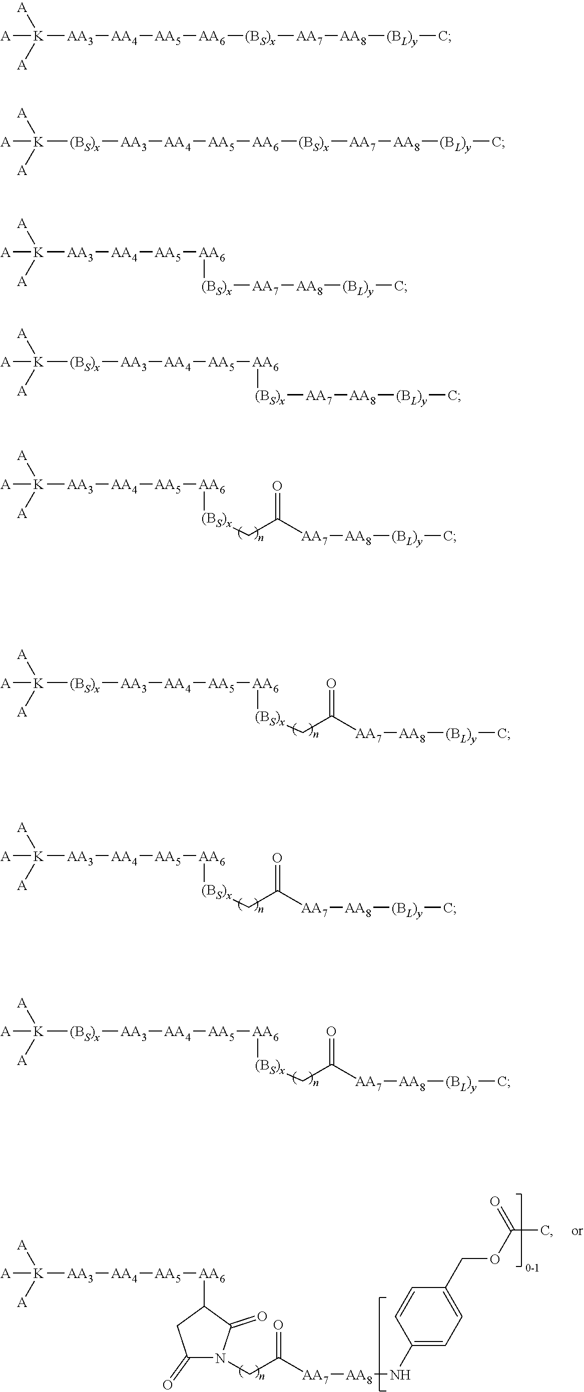

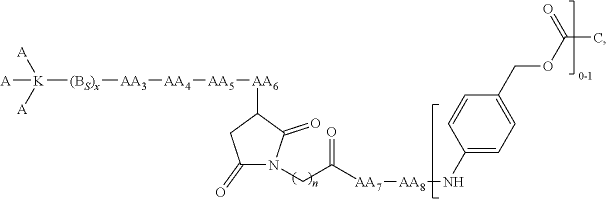

wherein B_{S}, B_{L}, x, y and n, and the remaining groups are as defined elsewhere herein,
wherein each of AA₃, AA₄, AA₅, AA₆, AA₇ and AA₈ represents a proteinogenic or non-proteinogenic amino acid, or is absent;
preferably wherein each (B_{S})ₓ and (B_{L})_{y} is independently represented by bond, —NHC(O)(CH₂)ₙC(O)—, —NH(CH₂)ₙC(O)—, —NHC(O)(CH₂CH₂O)ₘ(CH₂)ₙ—, —C(O)(CH₂CH₂O)ₘ(CH₂)ₙ—, —C(O)(CH₂CH₂O)ₘ(CH₂)ₙNH—, —(CH₂CH₂O)ₘ(CH₂)ₙ—, —(CH₂CH₂O)ₘ(CH₂)ₙNH—, —(CH₂CH₂O)ₘ(CH₂)ₙNHC(O)—, —(CH₂)ₙO(CH(CH₂CH₂O)ₘ(CH₂)ₙ—, —(CH₂)ₙO(CH₂CH₂O)ₘ(CH₂)ₙNH—, —(CH₂CH₂O)ₘ(CH₂)ₙNHC(O)—, —C(O)(CH₂)ₙO(CH₂CH₂O)ₘ(CH₂)ₙ—, —C(O)(CH₂)ₙO(CH₂CH₂O)ₘ(CH₂)ₙNH—, —(CH₂)ₙO(CH₂CH₂O)ₘ(CH₂)ₙNH—, —C(O)(CH₂)ₙC(O)—, —C(O)(CH₂)ₙ—, —C(O)(CH₂)ₙNH— or —(CH₂)ₙC(O)—, more preferably —C(O)(CH₂)ₙC(O)— or —(CH₂)ₙC(O)—, and wherein each n and m is independently an integer, preferably selected from 0, 1, 2, 3, 4, 5 and 6;
more preferably

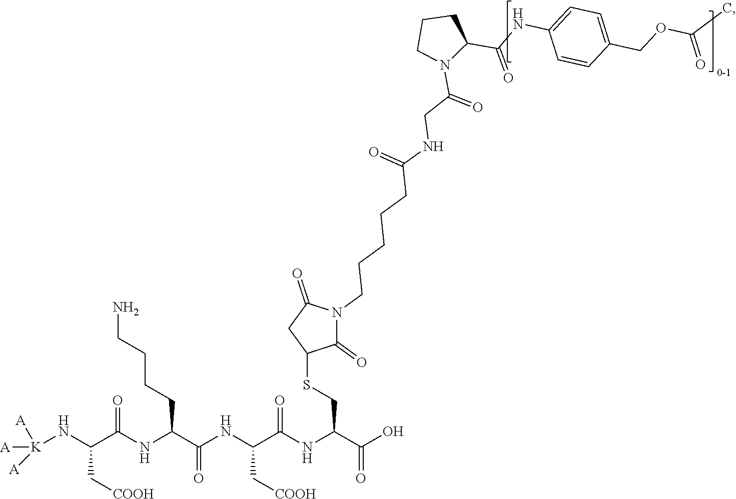

most preferably

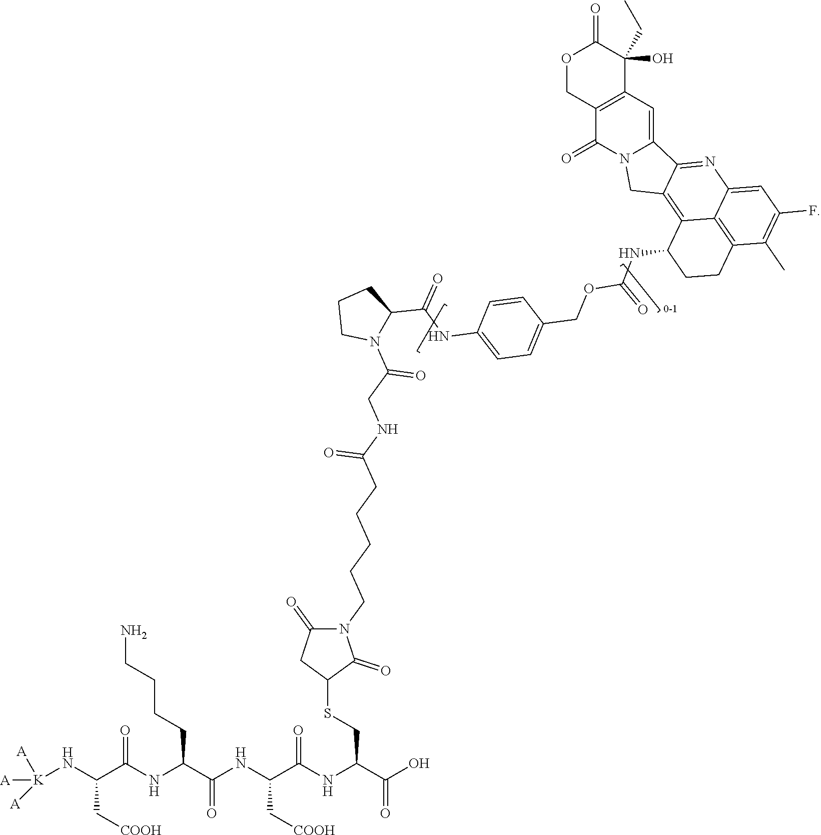

Preferred compounds according to the present invention may also be represented by:

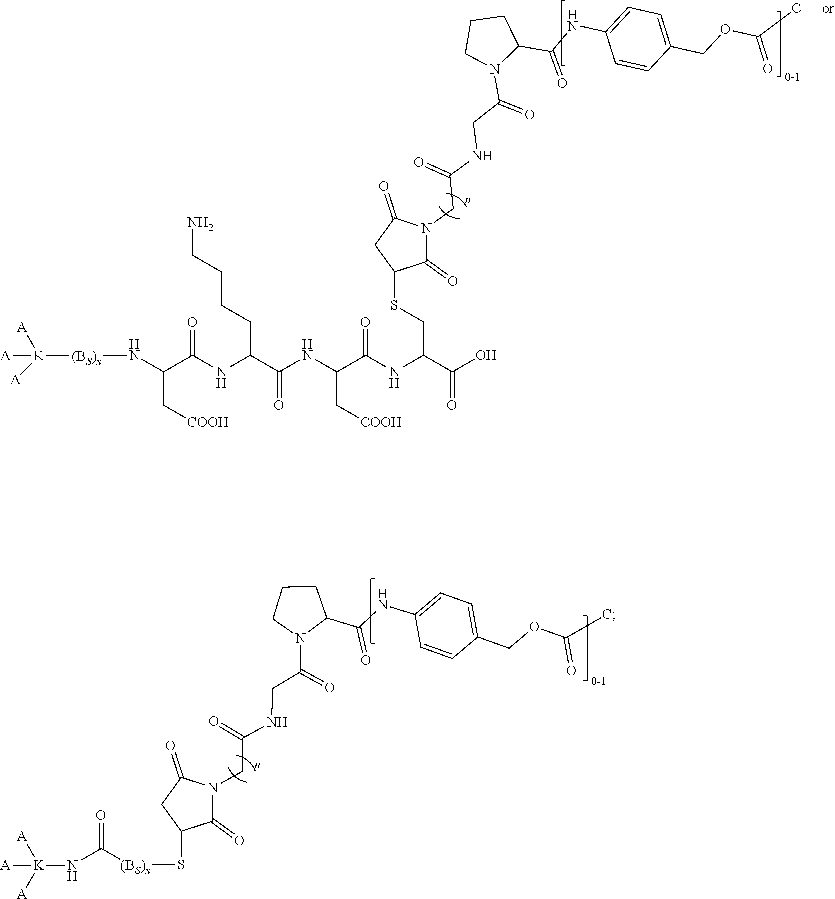

more preferably:

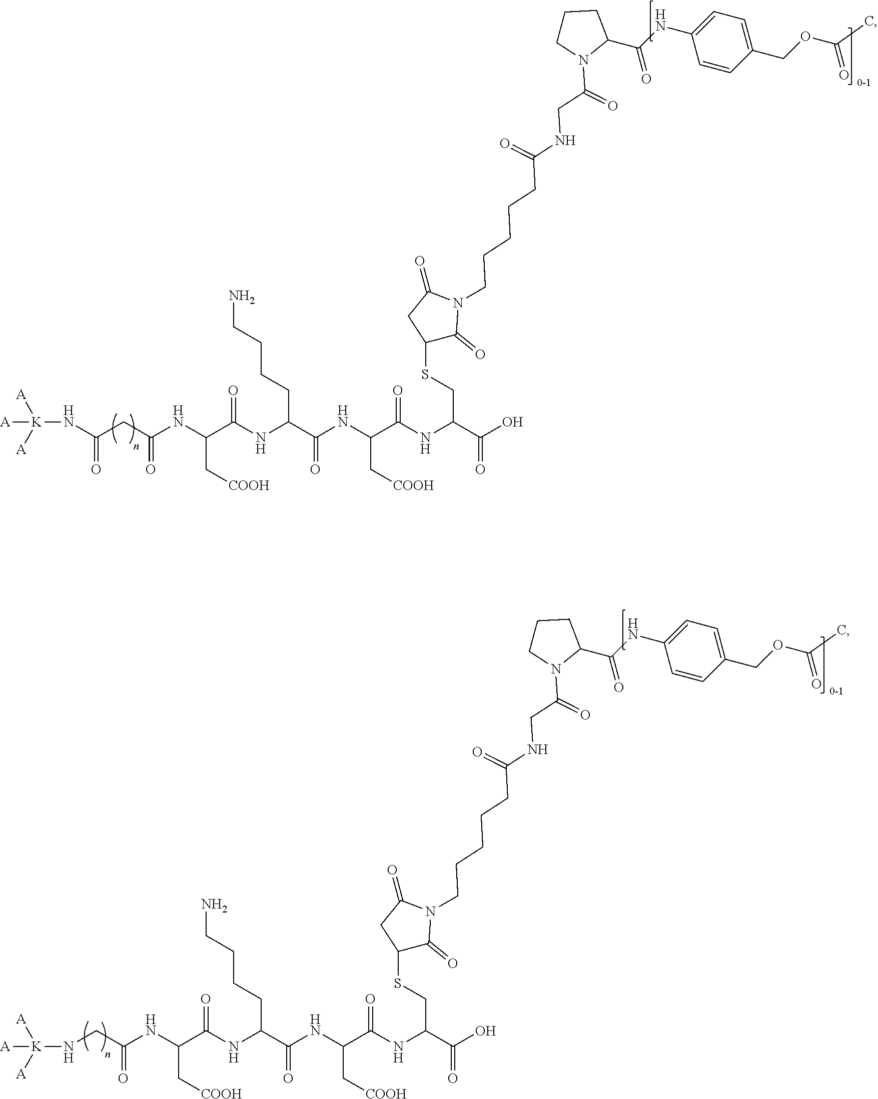

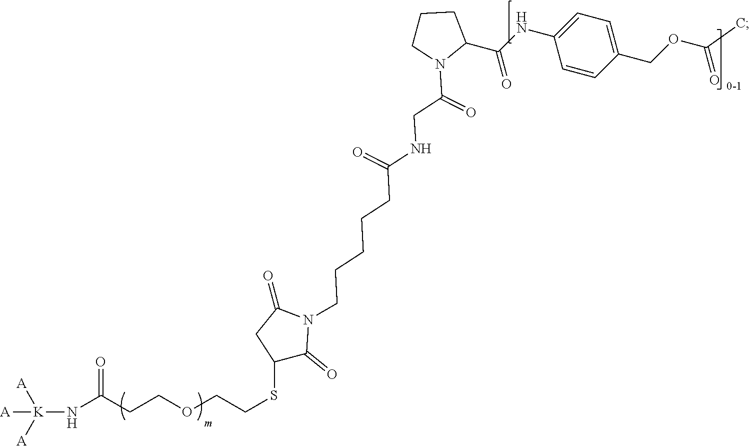

most preferably:

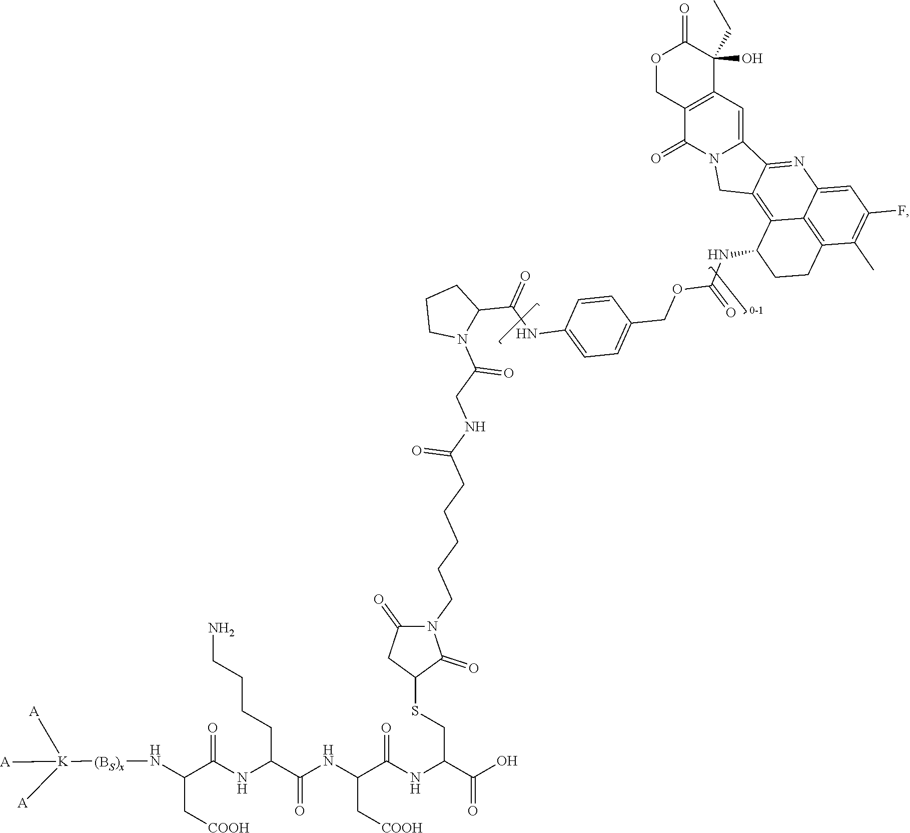

wherein B_{S}, B_{L}, x, y and n, and the remaining groups are as defined elsewhere herein,
preferably wherein each (B_{S})ₓ and (B_{L})_{y} is independently represented by bond, —NHC(O)(CH₂)ₙC(O)—, —NH(CH₂)ₙC(O)—, —NHC(O)(CH₂CH₂O)ₘ(CH₂)ₙ—, —C(O)(CH₂CH₂O)ₘ(CH₂)ₙ—, —C(O)(CH₂CH₂O)ₘ(CH₂)ₙNH—, —(CH₂CH₂O)ₘ(CH₂)ₙ—, —(CH₂CH₂O)ₘ(CH₂)ₙNH—, —(CH₂CH₂O)ₘ(CH₂)ₙNHC(O)—, —(CH₂)ₙO(CH(CH₂CH₂O)ₘ(CH₂)ₙ—, —(CH₂)ₙO(CH₂CH₂O)ₘ(CH₂)ₙNH—, —(CH₂CH₂O)ₘ(CH₂)ₙNHC(O)—, —C(O)(CH₂)ₙO(CH₂CH₂O)ₘ(CH₂)ₙ—, —C(O)(CH₂)ₙO(CH₂CH₂O)ₘ(CH₂)ₙNH—, —(CH₂)ₙO(CH₂CH₂O)ₘ(CH₂)ₙNH—, —C(O)(CH₂)ₙC(O)—, —C(O)(CH₂)ₙ—, —C(O)(CH₂)ₙNH— or —(CH₂)ₙC(O)—, more preferably —C(O)(CH₂)ₙC(O)— or —(CH₂)ₙC(O)—, and wherein each n and m is independently an integer, preferably selected from 0, 1, 2, 3, 4, 5 and 6.

The moiety C is a therapeutic agent which may be: (a) a chelating agent group suitable for radiolabelling with therapeutic nuclides; (b) a therapeutic radioactive group comprising a therapeutic radioisotope; (c) a chelate of a therapeutic radioactive isotope with a chelating agent; (d) a cytotoxic and/or cytostatic therapeutic agent; (f) immunomodulator agent; or (g) a protein, preferably:
(a) the chelating agent group suitable for radiolabeling with therapeutic nuclides is selected from sulfur colloid, diethylenetriaminepentaacetic acid (DTPA), ethylenediaminetetraacetic acid (EDTA), 1,4,7,10-tetraazacyclododecane-N,N′,N″,N″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA), iminodiacetic acid, bis(carboxymethylimidazole)glycine, 6-Hydrazinopyridine-3-carboxylic acid (HYNIC),

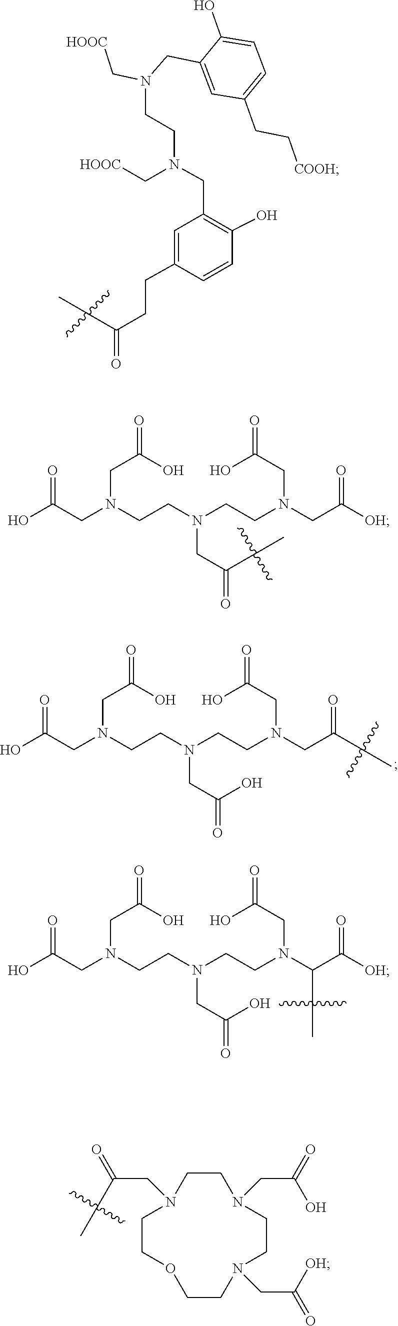 [SMILES: *C(=O)CCc1ccc(O)c(CN(CCN(CC(=O)O)Cc2cc(CCC(=O)O)ccc2O)CC(=O)O)c1 ; *C(=O)CN(CCN(CC(=O)O)CC(=O)O)CCN(CC(=O)O)CC(=O)O ; *C(=O)CN(CCN(CCN(CC(=O)O)CC(=O)O)CC(=O)O)CC(=O)O ; *C(C(=O)O)N(CCN(CCN(CC(=O)O)CC(=O)O)CC(=O)O)CC(=O)O ; *C(=O)CN1CCOCCN(CC(=O)O)CCN(CC(=O)O)CC1]

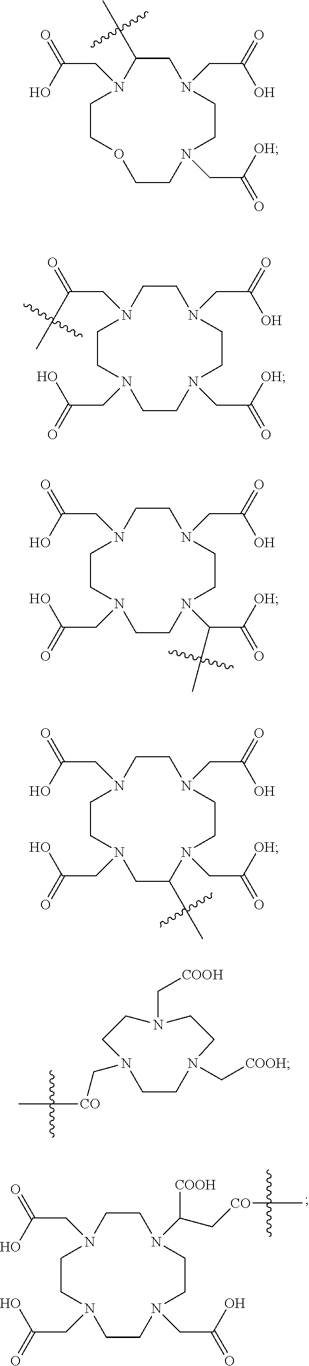 [SMILES: *C1CN(CC(=O)O)CCN(CC(=O)O)CCOCCN1CC(=O)O ; *C(=O)CN1CCN(CC(=O)O)CCN(CC(=O)O)CCN(CC(=O)O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O ; *C(=O)CN1CCN(CC(=O)O)CCN(CC(=O)O)CC1 ; *C(=O)CC(C(=O)O)N1CCN(CC(=O)O)CCN(CC(=O)O)CCN(CC(=O)O)CC1]

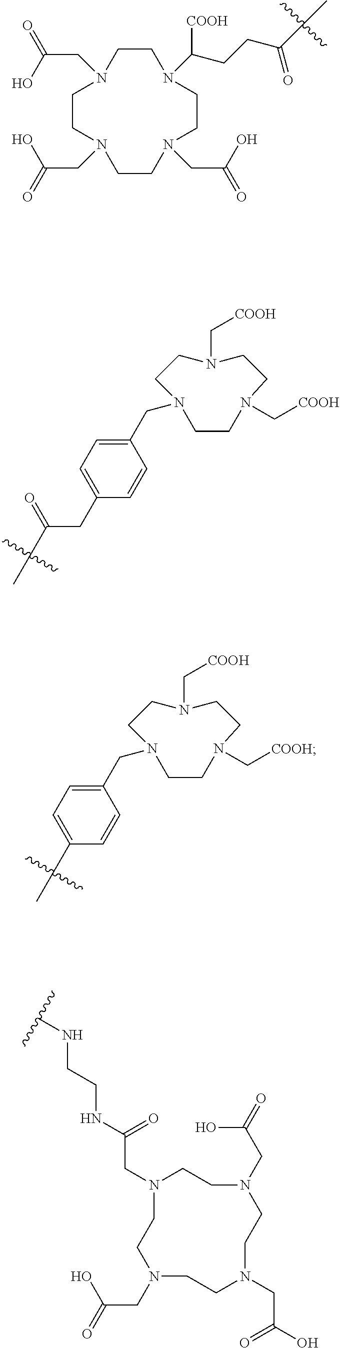 [SMILES: *C(=O)CCC(C(=O)O)N1CCN(CC(=O)O)CCN(CC(=O)O)CCN(CC(=O)O)CC1 ; *C(=O)Cc1ccc(CN2CCN(CC(=O)O)CCN(CC(=O)O)CC2)cc1 ; *c1ccc(CN2CCN(CC(=O)O)CCN(CC(=O)O)CC2)cc1 ; *NCCNC(=O)CN1CCN(CC(=O)O)CCN(CC(=O)O)CCN(CC(=O)O)CC1]

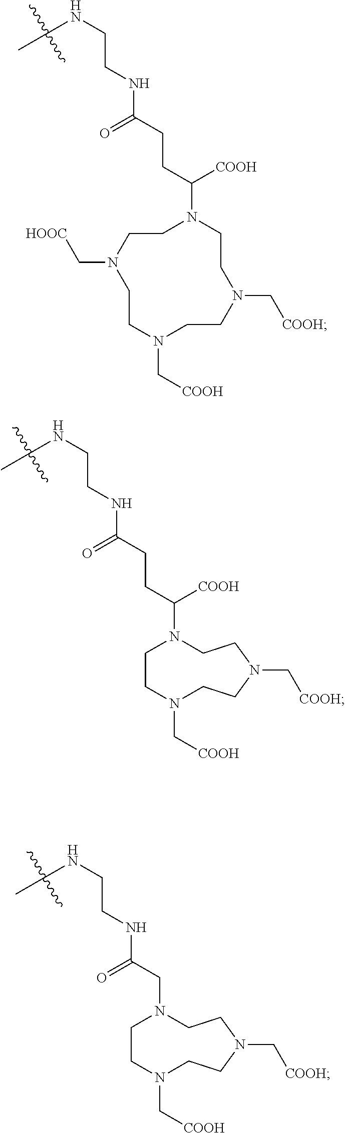 [SMILES: *NCCNC(=O)CCC(C(=O)O)N1CCN(CC(=O)O)CCN(CC(=O)O)CCN(CC(=O)O)CC1 ; *NCCNC(=O)CCC(C(=O)O)N1CCN(CC(=O)O)CCN(CC(=O)O)CC1 ; *NCCNC(=O)CN1CCN(CC(=O)O)CCN(CC(=O)O)CC1]

or
has a structure according to the following formula:

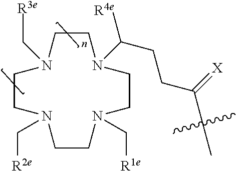

wherein:
n is 0, 1, 2, 3, 4 or 5; preferably 1;
R^{1e} is independently H, COOH, aryl-COOH or heteroaryl-COOH; preferably COOH;
R^{2e} is independently H, COOH, aryl-COOH or heteroaryl-COOH; preferably COOH;
each R^{3e} is independently H, COOH, aryl-COOH or heteroaryl-COOH; preferably COOH;
R^{4e} is independently H, COOH, aryl-COOH or heteroaryl-COOH; preferably COOH; and
X is O, NH or S; preferably O; or
has a structure according to the following formula:

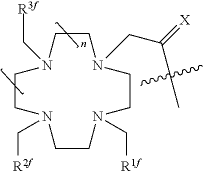

wherein:
n is 0, 1, 2, 3, 4 or 5; preferably 1
R^{1f} is independently H, COOH, aryl-COOH or heteroaryl-COOH; preferably COOH;
R^{2f} is independently H, COOH, aryl-COOH or heteroaryl-COOH; preferably COOH;
R^{3f} is independently H, COOH, aryl-COOH or heteroaryl-COOH; preferably COOH; and
X is O, NH or S; preferably O;
(b) the therapeutic radioactive group comprises a radioisotope selected from ²²³Ra, ⁸⁹Sr, ⁹⁰Y, ¹²¹Sn, ¹⁷⁷Lu, ¹³¹I, ²¹¹At, ²²⁵Ac, ¹⁸⁸Re, ¹⁴⁹Tb, ¹⁶¹Tb and ²²⁷Th, preferably ⁹⁰Y, 225Ac or ¹⁷⁷Lu, more preferably ¹⁷⁷Lu; which may not be used for diagnostic applications;
(c) the chelate of a therapeutic radioactive isotope is a chelate of an isotope listed under (b) above and/or with a chelating agent listed under (a) above; or moiety C is a group selected from any of the following structures:

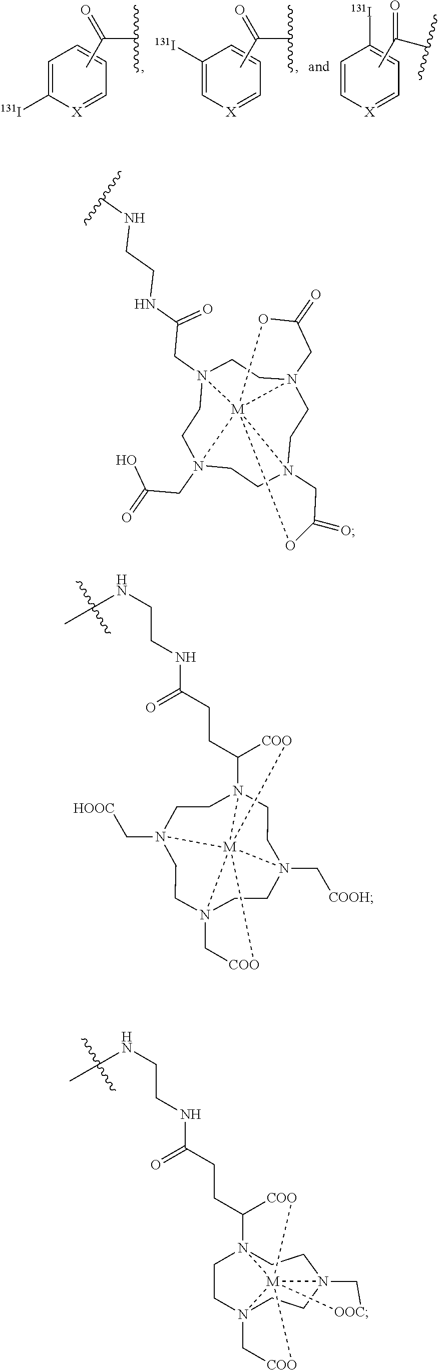

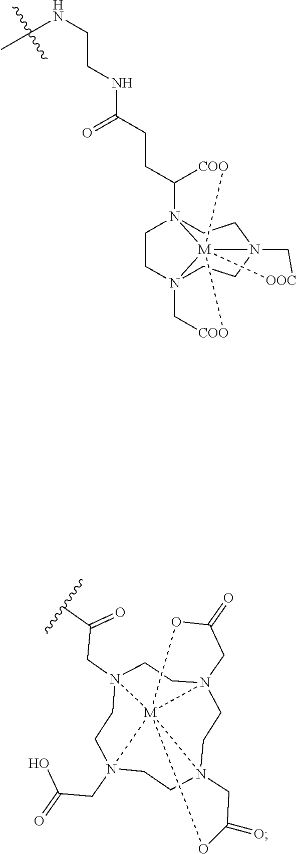

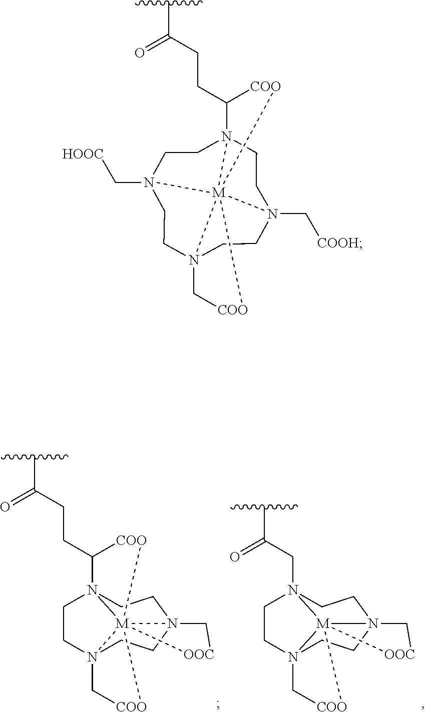

wherein M is a therapeutic radioactive isotope, preferably selected among the list under (b) above;
more preferably:

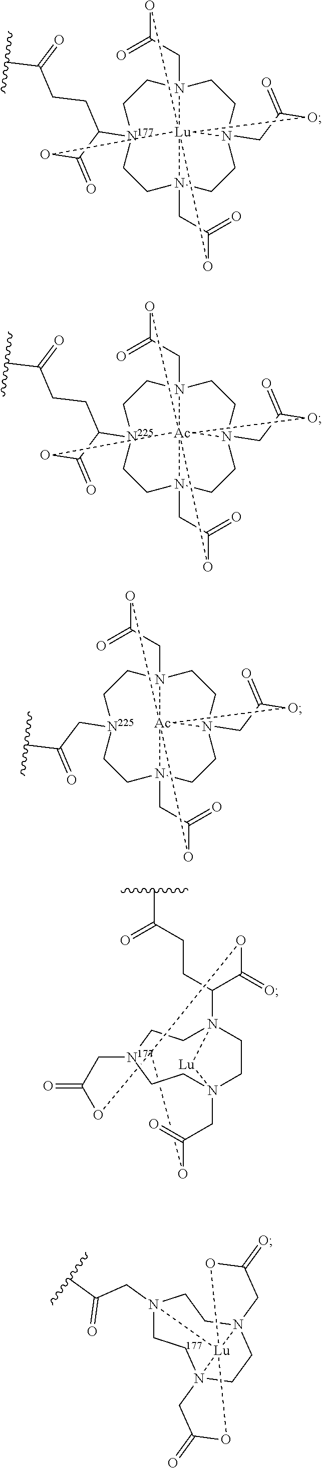

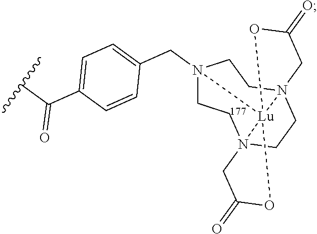

or
(d) the cytotoxic and/or cytostatic therapeutic agent is selected from chemotherapeutic agent selected from the group consisting of topoisomerase inhibitors, alkylating agents, antimetabolites, antibiotics, mitotic disrupters, DNA intercalating agents, DNA synthesis inhibitors, DNA-RNA transcription regulator, enzyme inhibitors, gene regulators, hormone response modifiers, hypoxia-selective cytotoxins, epidermal growth factor inhibitors, anti-vascular agents and a combination of two or more thereof, preferably selected from the following structures:

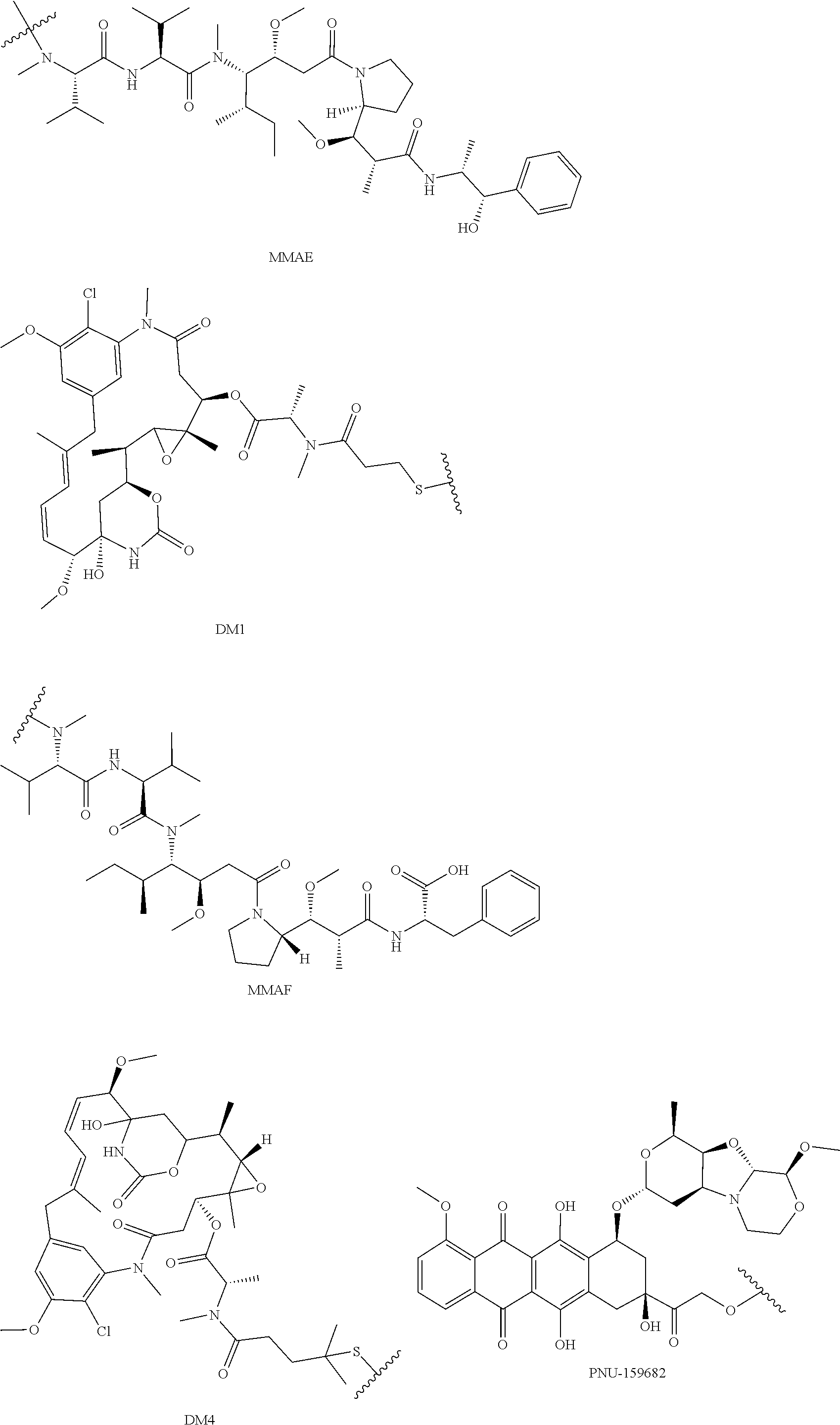

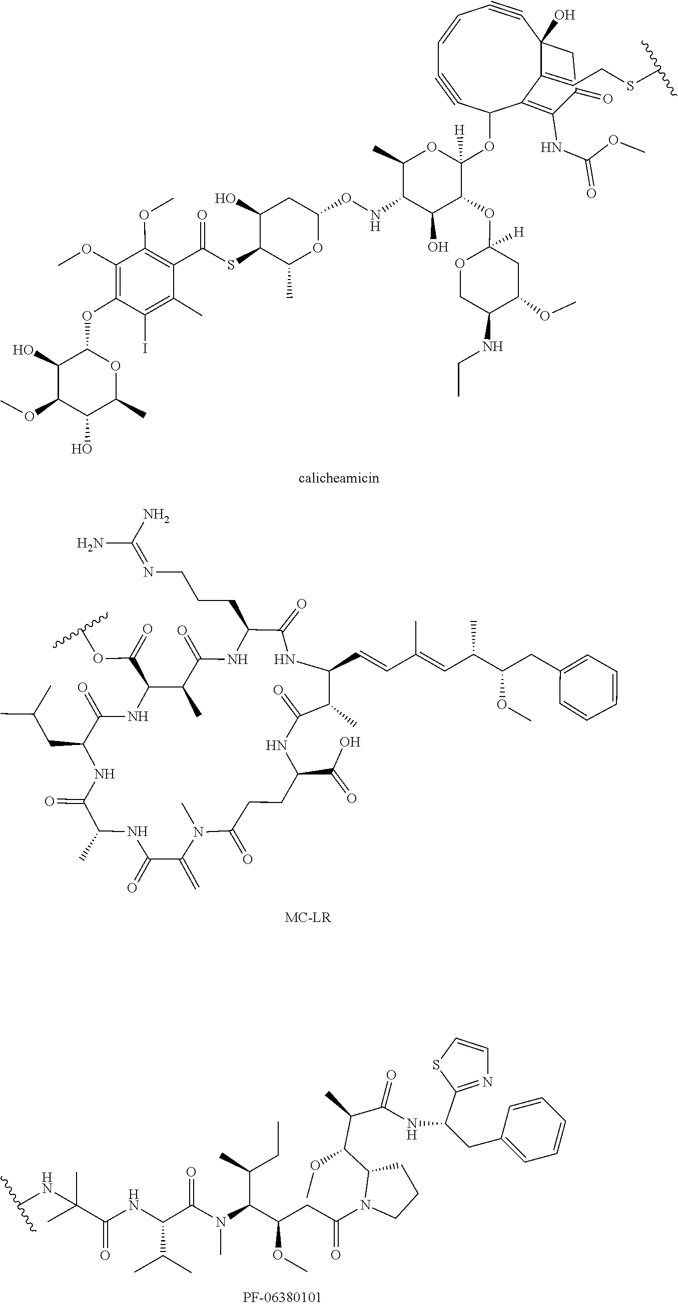

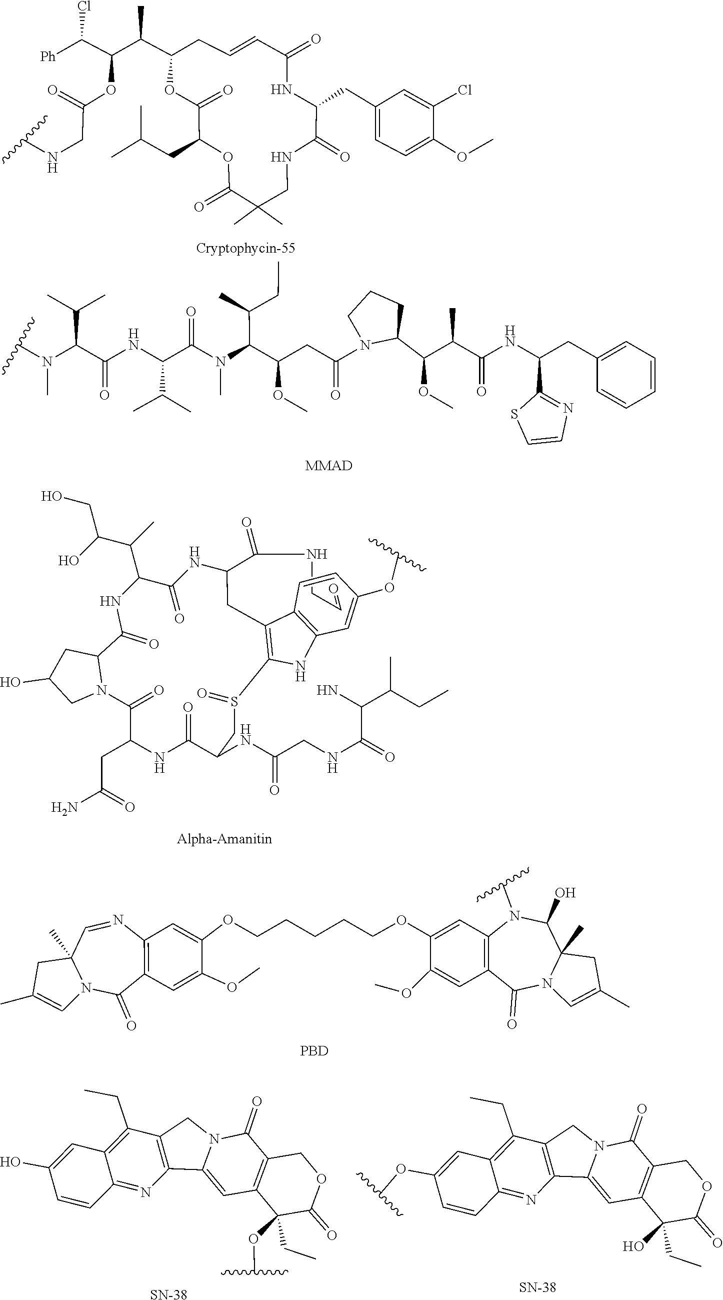

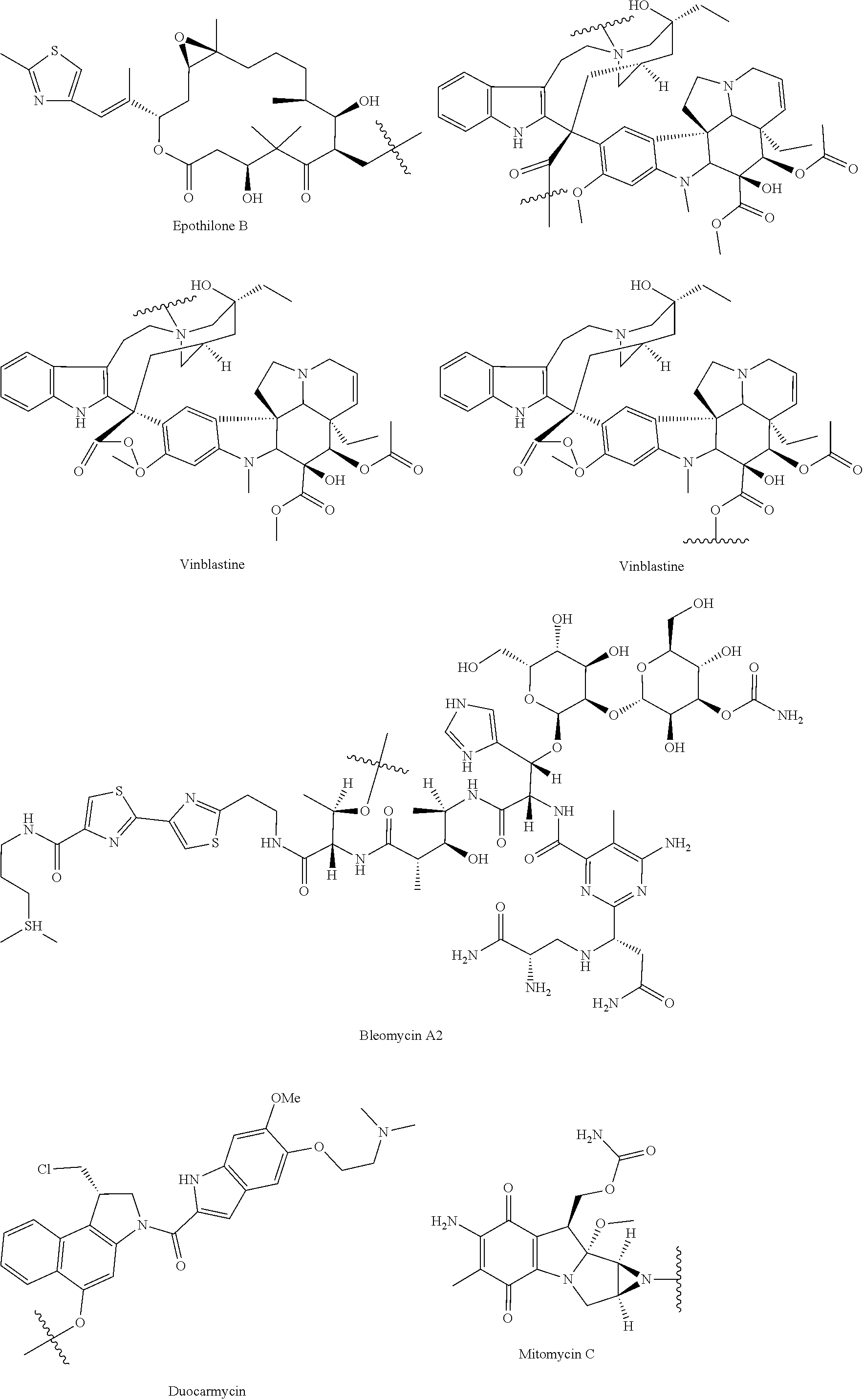

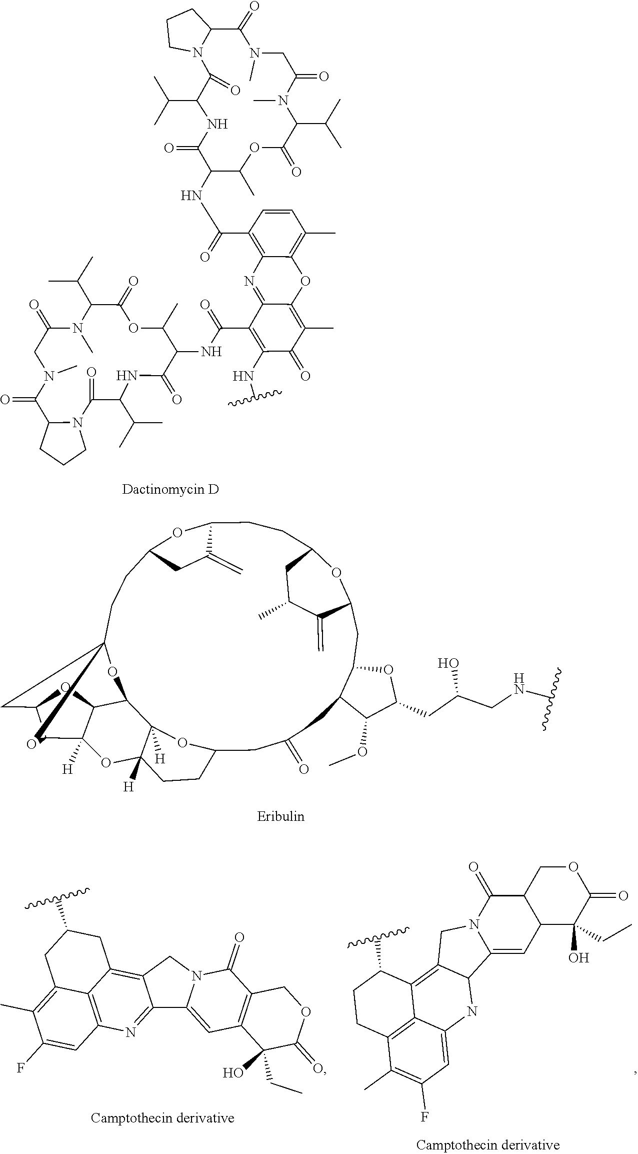

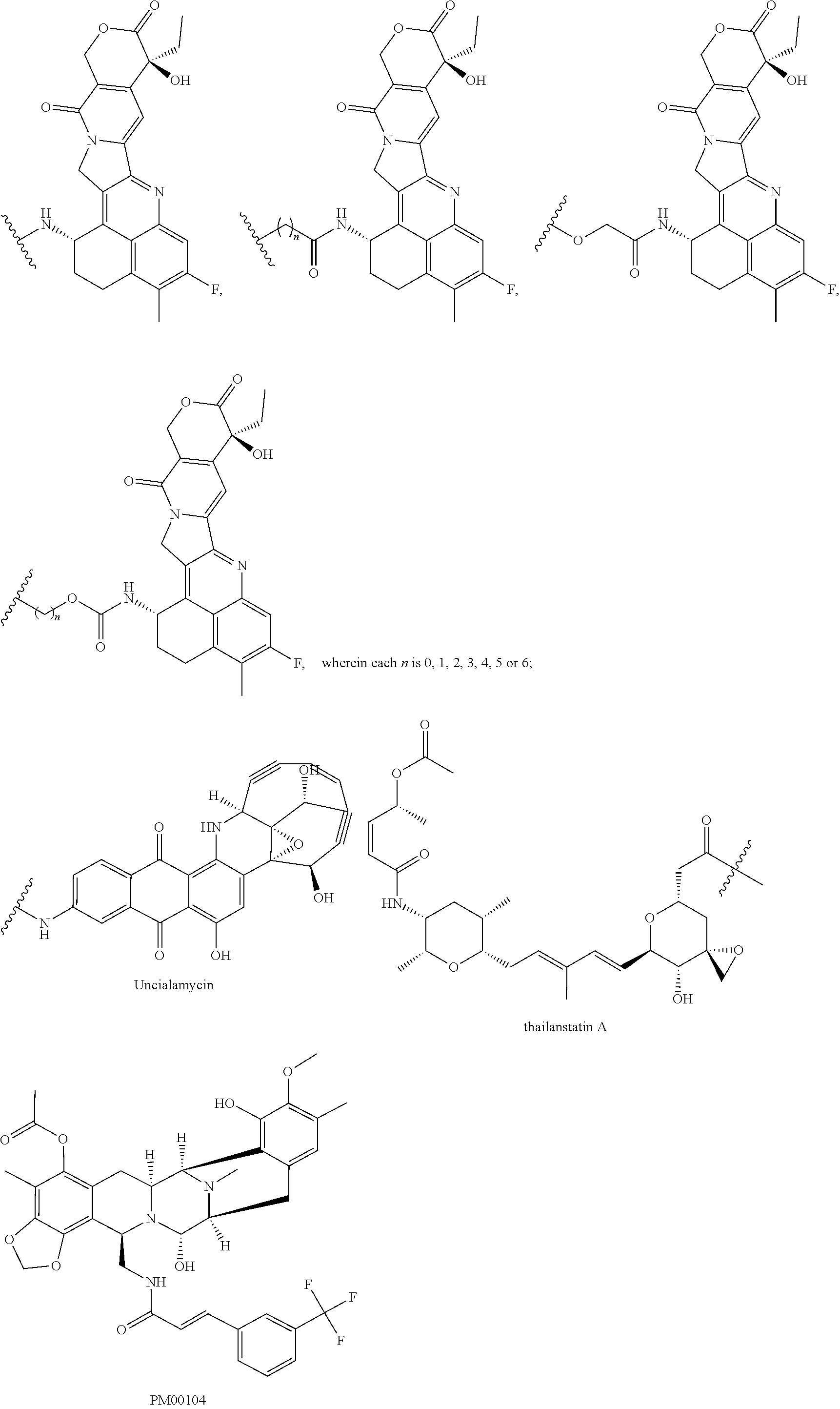

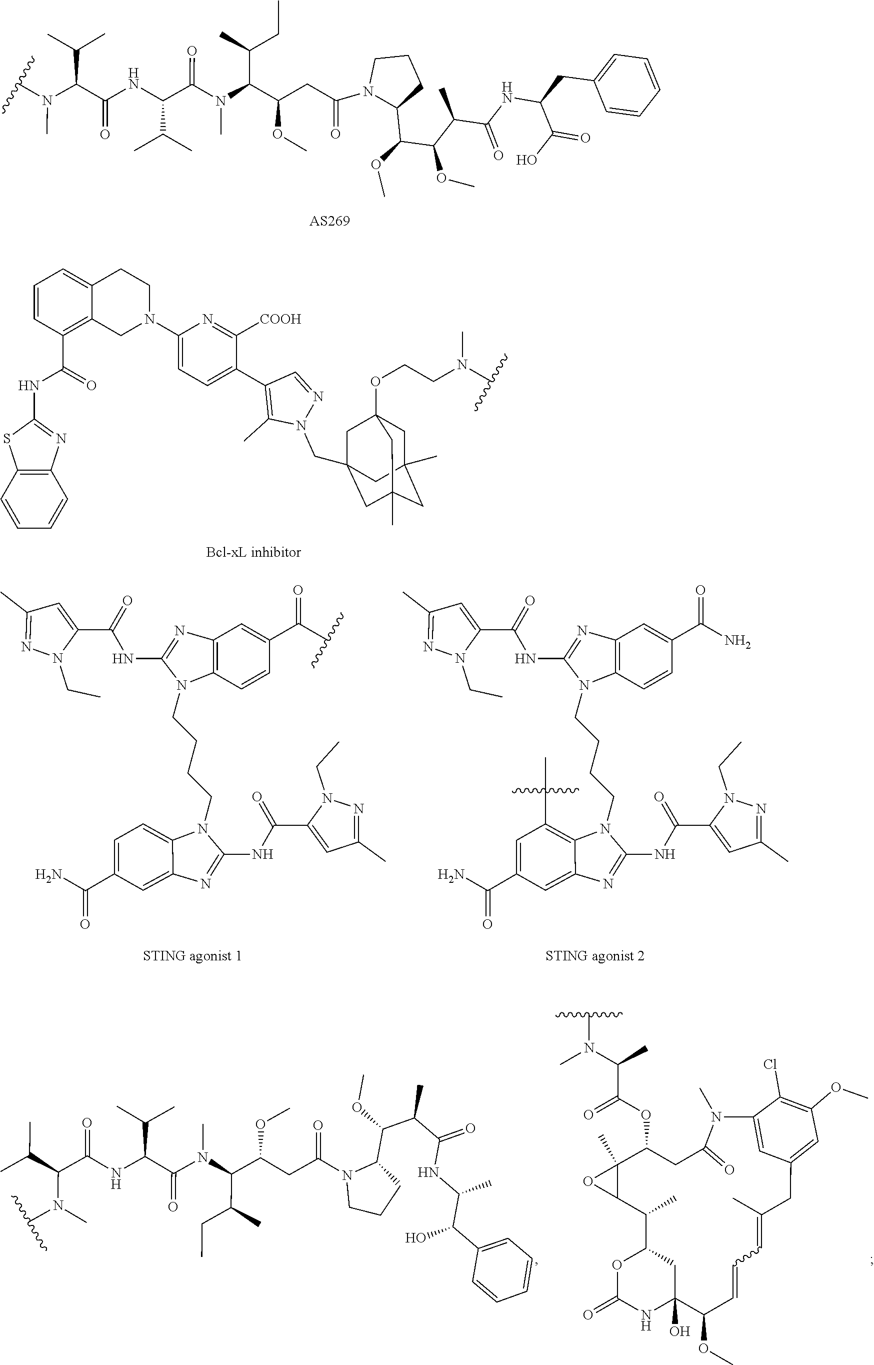

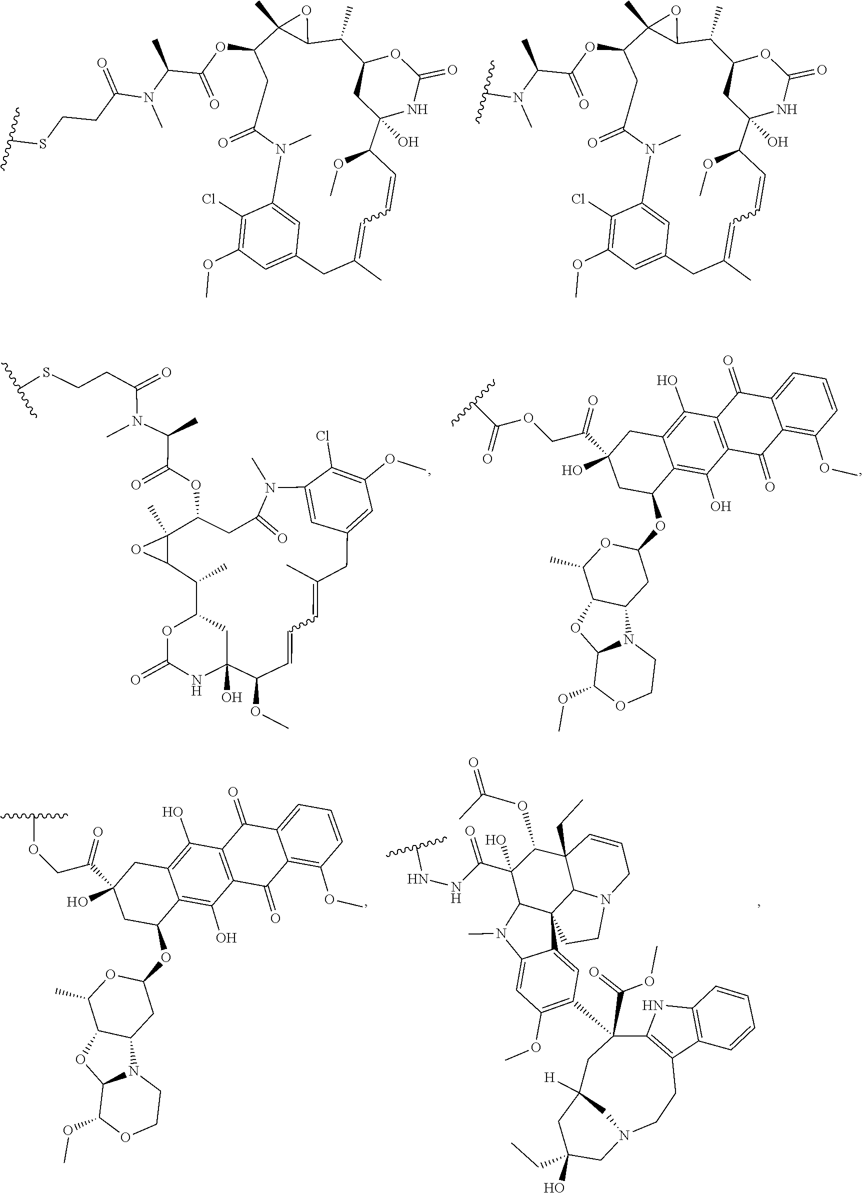

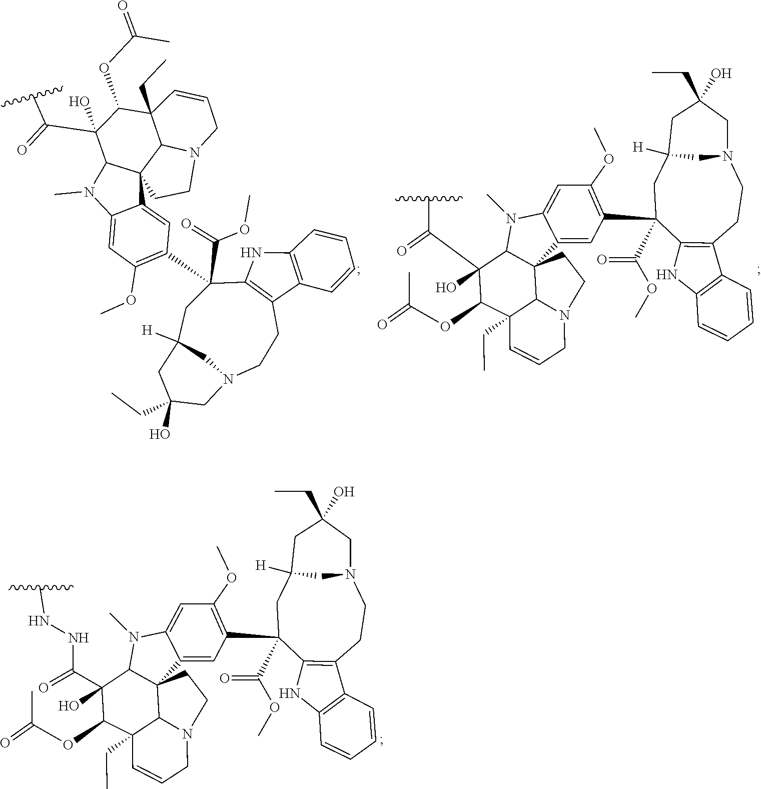

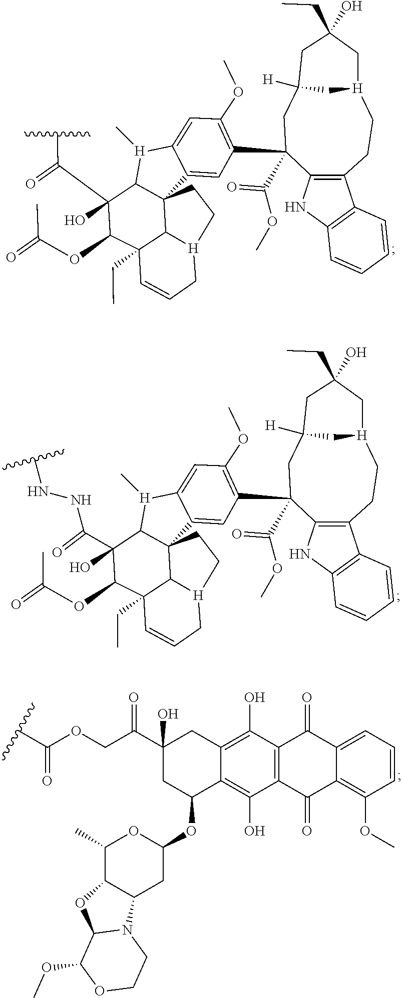

or
moiety C is an auristatin, preferably having a structure according to the following formula:

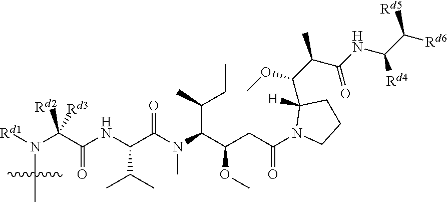

wherein:
R^{1d} is independently H or C₁-C₆ alkyl; preferably H or CH₃;
R^{2d} is independently C₁-C₆ alkyl; preferably CH₃ or iPr;
R^{3d} is independently H or C₁-C₆ alkyl; preferably H or CH₃;
R^{4d} is independently H, C₁-C₆ alkyl, COO(C₁-C₆ alkyl), CON(H or C₁-C₆ alkyl), C₃-C₁₀ aryl or C₃-C₁₀ heteroaryl; preferably H, CH₃, COOH, COOCH₃ or thiazolyl;
R^{5d} is independently H, OH, C₁-C₆ alkyl; preferably H or OH; and
R^{6d} is independently C₃-C₁₀ aryl or C₃-C₁₀ heteroaryl; preferably optionally substituted phenyl or pyridyl,
wherein preferably, moiety C is derived from MMAE or MMAF;
(e) the immunomodulator therapeutic agent is selected from molecules known to be able to modulate the immune system, such as ligands of CD3, CD25, TLRs, STING, 4-1BBL, 4-1BB, PD-1, mTor, PDL-1, NKG-2D IMiDs, wherein ligands can be agonists and/or antagonist; or
(f) the protein is a therapeutic protein selected from cytokines, such as IL2, IL10, IL12, IL15, TNF, Interferon Gamma, or is a therapeutic antibody.

### Treatment

The compounds described herein may be used to treat disease. The treatment may be therapeutic treatment, with the aim being to prevent, reduce or stop an undesired physiological change or disorder. The treatment may prolong survival as compared to expected survival if not receiving treatment. The disease that is treated by the compound may be any disease that might benefit from treatment. This includes chronic and acute disorders or diseases including those pathological conditions which predispose to the disorder.

The term “cancer” and “cancerous” is used in its broadest sense as meaning the physiological condition in mammals that is typically characterized by unregulated cell growth. A tumor comprises one or more cancerous cells. When treating cancer, the therapeutically effect that is observed may be a reduction in the number of cancer cells; a reduction in tumor size; inhibition or retardation of cancer cell infiltration into peripheral organs; inhibition of tumor growth; and/or relief of one or more of the symptoms associated with the cancer.

In animal models, efficacy may be assessed by physical measurements of the tumor during the treatment, and/or by determining partial and complete remission of the cancer. For cancer therapy, efficacy can, for example, be measured by assessing the time to disease progression (TTP) and/or determining the response rate (RR).

Particularly preferred embodiments for the methods of treatment related to the present invention are shown in the items further below and appended claims.

Herein disclosed are also methods for treatment of the human or animal body, e.g., by therapy, practised on the human or animal body, the methods involving a step of administering a therapeutically effective amount of a compound or a pharmaceutical composition as described herein to a subject in need thereof. More specifically, herein disclosed are methods for treatment, e.g., by therapy, of a subject suffering from or having risk for a disease or disorder; method for targeted delivery of a therapeutic agent to a subject suffering from or having risk for a disease or disorder. In the aforementioned methods, said disease or disorder may be independently selected from cancer, inflammation, atherosclerosis, fibrosis, tissue remodelling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumor, oncogenic osteomalacia, sarcoma, CUP (carcinoma of unknown primary), thymus cancer, desmoid tumors, glioma, astrocytoma, cervix cancer, skin cancer, kidney cancer and prostate cancer. When used in the methods disclosed herein, the compound has a prolonged residence at the disease site at a therapeutically relevant level, e.g., for at least 5 min, 10 min, 20 min, 30 min, 45 min, 1 h, 2 h, 3 h, 4 h, 5 h or 6 h, 24 h, 48 h, 72 h, 96 h, preferably beyond 1 h, more preferably beyond 6 h, even more preferably beyond 24 h post injection.

The disclosure of all compounds, compositions or combinations for use in any methods for treatment of the human or animal body disclosed herein is also intended to extend to corresponding methods for treating a disease or disorder in subject in need thereof comprising administration of a therapeutically effective amount of the compound, composition or combination to the subject; and to corresponding uses of the compounds, compositions or combinations in the manufacture of a medicament for treating the respective disease or disorder.

### Dosage

The present inventors have surprisingly found that the compounds, when given at certain doses, can provide optimal tumor uptake and/or tumor-to-organ ratio, thus showing minimal trapping in other organs (i.e., particularly clean biodistribution profile).

The compounds, pharmaceutical compositions or combinations described herein may be administered to a subject at a dose of the compound of general Formula I, II, III, IV, V, VI or VII of 10-500 nmol/kg, preferably 30-250 nmol/kg, more preferably 90-250 nmol/kg, even more preferably 90-160 nmol/kg, most preferably 90-125 nmol/kg, expressed as a mouse dose; or a corresponding human equivalent dose. The compounds, pharmaceutical compositions or combinations described herein may be administered to a human subject at a dose of said compound of 0.8-40 nmol/kg, preferably 2-20 nmol/kg, more preferably 7-20 nmol/kg, even more preferably 7-13 nmol/kg, most preferably 7-10 nmol/kg.

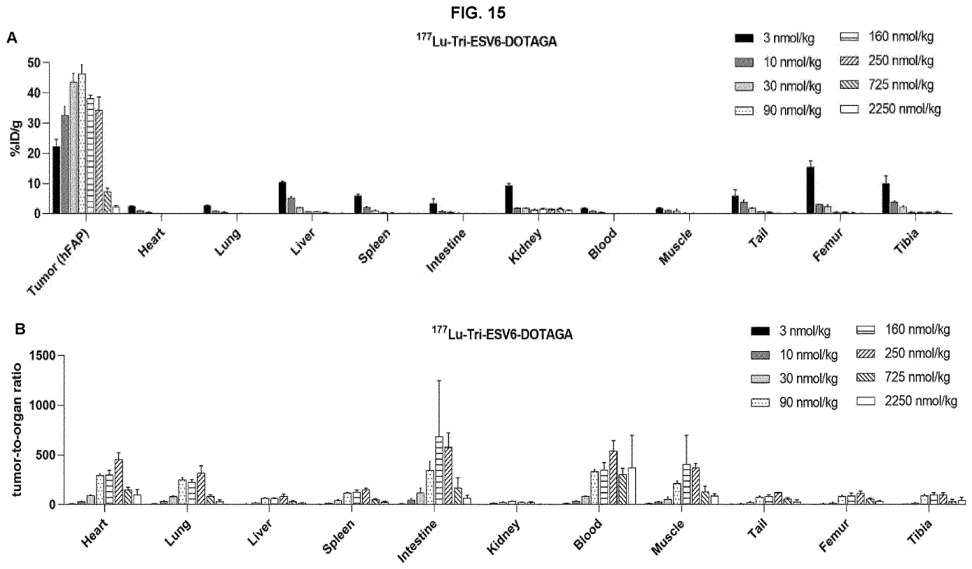

As demonstrated in FIG. 15, in mice, it was found that at doses in the range of preferably 10 to 500 nmol/kg, in particular at 90 nmol/kg or more, a particularly clean biodistribution profile and optimal tumor uptake can be achieved. Above 250 nmol/kg, saturation effects are observed. Accordingly, the range of 90 nmol/kg or more, preferably 90 to 250 nmol/kg (expressed as mouse dose) is particularly preferred. The range of 7 nmol/kg or more, preferably 7-20 nmol/kg (expressed as human dose) is particularly preferred.

Mouse doses can be converted to human equivalent doses (HED), e.g., considering the recommendation reported by FDA in the USFDA. Guidance for Industry: Estimating the Maximum Safe Starting Dose in Adult Healthy Volunteer. Rockville, MD: US Food and Drug Administration; 2005, for which the mouse dose (nmol/kg) has to be divided by a factor 12.3 to obtain the corresponding HED (nmol/kg). For a conversion to a human equivalent dose expressed in nmol or mg, a human reference body weight of 60, 70 or 80 kg, preferably 70 kg can be used. The conversion takes into consideration the differences in surface area between the two different species (Mus musculus vs. Homo sapiens sapiens).

For instance, for a compound having a molecular weight of 2246 g/mol, the range of 90 nmol/kg to 250 nmol/kg, expressed as mouse dose, corresponds to a HED of 0.016 mg/kg to 0.046 mg/kg or 1.1 mg to 3.2 mg, considering an average body weight of 70 kg.

In view of the above, a human dose of 1 mg or more, preferably 1 to 3 mg per administration is particularly advantageous.

### Pharmaceutical Compositions

The compounds described herein may be in the form of pharmaceutical compositions which may be for human or animal therapeutic use in human and/or veterinary medicine, and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilisers, dyes and even flavouring agents may be provided in the therapeutic pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the therapeutic pharmaceutical composition may be formulated to be administered using a mini-pump or by a mucosal route, for example, as a nasal spray or aerosol for inhalation or ingestible solution, or parenterally in which the composition is formulated by an injectable form, for delivery, by, for example, an intravenous, intramuscular or subcutaneous route. Alternatively, the therapeutic formulation may be designed to be administered by a number of routes.

If the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the therapeutic pharmaceutical compositions may be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or the pharmaceutical compositions can be injected parenterally, for example, intravenously, intramuscularly or subcutaneously. For parenteral administration, the therapeutic compositions may be best used in the form of a sterile aqueous solution which may contain other substances, for example, enough salts or monosaccharides to make the solution isotonic with blood. For buccal or sublingual administration, the therapeutic compositions may be administered in the form of tablets or lozenges which can be formulated in a conventional manner.

The therapeutic compound of the present invention may be administered in the form of a pharmaceutically acceptable or active salt. Pharmaceutically-acceptable salts are well known to those skilled in the art, and for example, include those mentioned by Berge et al, in J. Pharm. Sci., 66, 1-19 (1977). Salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts.

The routes for administration (delivery) may include, but are not limited to, one or more of oral (e.g. as a tablet, capsule, or as an ingestible solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Typically, a physician will determine the actual therapeutic dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

The therapeutic formulations may be packaged in unit-dose or multi-dose containers, for example sealed ampoules and vials, and may be stored in a freeze-dried (lyophilized) condition requiring only the addition of the sterile liquid carrier, for example water, for administration. Extemporaneous injection solutions and suspensions are prepared from sterile powders, granules and tablets of the kind previously described. Exemplary unit dosage formulations contain a daily dose or unit daily sub-dose, or an appropriate fraction thereof, of the active ingredient.

### Pharmaceutical Combinations

As used herein, the term “pharmaceutical combination” means, e.g., that the individual components comprised therein can be administered in the same dosage form or as separate dosage forms, concomitantly or sequentially; preferably, the subject is exposed, at a point in time, to a therapeutically or prophylactically effective amount of each of the individual components of the combination. For instance, the pharmaceutical combination may comprise the two or more individual components within the same formulation, or as separate formulations intended (e.g., as a part of a therapeutic dosage regimen/scheme, or by means of instructions for use) or arranged (e.g., as a kit of parts) for concomitant or sequential administration.

The compounds described herein may be administered to a patient in combination with a therapeutic agent, such as an immunoconjugate, preferably an immunocytokine, most preferably an immunocytokine comprising a human IL2 polypeptide conjugated to an scFv polypeptide sequence that is capable of binding to the epitope represented by SEQ ID NO:40 (and is further preferably an L19-IL2 immunocytokine further comprising linked VL and VH domains derived from the antibody L19, optionally capable of homodimerization in the scFv2 format), as described herein below.

Combinations with an Immunocytokine

Provided is a pharmaceutical combination comprising a compound or pharmaceutical composition as disclosed herein and an immunocytokine.

Herein, the term “immunocytokine” refers to a conjugate protein or fusion protein comprising a cytokine and an antibody, antibody fragment or antibody derivative. A fusion protein is a polypeptide that is a translation product resulting from the fusion of two or more genes or nucleic acid coding sequences into one open reading frame (ORF). The fused expression products of the two genes or ORFs may be conjugated by a linker. Herein, the terms conjugate protein or fusion protein are generally used interchangeably. The fusion protein may further comprise a signal peptide sequence, normally located upstream (5′) of the specific binding member and subunit.

### Immunocytokine Comprising IL2

Preferably, the immunocytokine comprises a sequence having IL2 activity, i.e., an IL2 polypeptide, i.e., the cytokine IL2 or a functional fragment thereof. Preferably, the immunocytokine comprises only one (i.e., a single) IL2 polypeptide per polypeptide chain. Herein the terms “IL2” and “IL2 polypeptide” are used interchangeably.

The IL2 may be derived from any animal, e.g. human, rodent (e.g. rat, mouse), horse, cow, pig, sheep, dog, etc. Human IL2 is preferred in conjugates for administration to humans. The amino acid sequence of human IL2 is set out in SEQ ID NO: 21. The immunocytokine conjugate preferably comprises a single IL2 polypeptide. An IL2 polypeptide in an immunocytokine of the invention retains a biological activity of IL2, e.g., an ability to promote proliferation and/or differentiation of activated T and B lymphocytes and natural killer (NK) cells, and/or to induce cytotoxic T cell (CTL) activity, and/or to induce NK/lymphokine-activated killer (LAK) cell antitumor cytotoxicity.

### Antibodies, Fragments or Derivatives Thereof Comprised in the Immunocytokine

Besides a cytokine (e.g., IL2), the immunocytokine comprises an antibody or an antibody fragment or an antibody derivative. Preferably the immunocytokine herein comprises an antibody derivative. The antibody derivative may, e.g., comprise a single-chain variable fragment (scFv), a diabody or a single chain diabody (scDb) or a Fab or a Fab2 or a nanobody or an “SIP” (WO2003/076469) or a “Crab” (Neri et al., (1995) J Mol Biol, 246, 367-73). The immunocytokine may comprise an IgG antibody or an IgG derivative.

Preferably the antibody derivate comprises an scFv. As is known in the art, an scFv comprises a VH domain and a VL domain, wherein the domains are linked by a linker that allows association of VH and VL domains to form an antigen binding site. The scFv may be stabilized by the incorporation of disulphide bridges linking the VH and VL domains

Single chain Fv (scFv) antibody polypeptide sequences are particularly preferred for incorporation in the immunocytokine (e.g., with a further polypeptide sequence having IL2 activity), owing to their small size of the scFv format, which provides physiological and therapeutic advantages for in vivo use of the immunocytokine conjugates. In addition, scFv lack an Fc region, potentially reducing anti-idiotypic reactions and also minimizing undesirable properties relating to activation of complement and interaction with Fc receptors that may hinder tumour targeting and cause non-specific cell activation.

The linker joining the VH and VL domains within an scFv chain may be a peptide linker sequence that is not long enough to allow pairing of the VH and VL domains within the same scFv polypeptide chain. Thus, a homodimer of scFvs may form instead, in which the VH of one scFv chain pairs with the VL of the other scFv chain (and vice versa). This general format may be referred to as an “scFv2” format, or, alternatively, in some cases, as a “diabody”. Examples of suitable short linker sequences are GSSGG (SEQ ID NO: 26) and GGSGG (SEQ ID NO: 27). Preferably the linker is such as the 12-residue linker SEQ ID NO: 22.

In a so-called single-chain diabody (“scDb”), the two sets of VH and VL domains (i.e., the two polypeptides that pair to form a dimer in a diabody or scFv2) are connected as a single-chain by a peptide linker (“scDb linker”) as follows:

(VH-VL)-linker-(VH-VL), wherein “(VH-VL)” indicates an scFv unit consisting of a set of a VH and a VL domain connected by a short linker, as described above.

The scDb linker sequence is sufficiently long and/or flexible to allow pairing of the VH domain of one scFv unit (i.e., the first VH and VL-containing polypeptide) with the VL domain of the other, complementary scFv unit (i.e., the second VH and VL-containing polypeptide), and vice versa, within a single polypeptide chain. Generally a long and/or flexible linker that allows two complementary VH and VL-containing polypeptides to dimerize within a single polypeptide chain in this manner is 10 to 20 amino acids in length.

### Antigen Binding Specificity of the Immunocytokine

The antibody, antibody fragment or antibody derivative suitably binds specifically to an extra-cellular matrix (ECM) component associated with neoplastic growth and/or angiogenesis. The antibody, fragment or derivative thereof (e.g., scFv, diabody or single-chain diabody) comprises an antigen-binding site having the complementarity determining regions (CDRs), or the VH and/or VL domains of an antibody capable of specifically binding to an antigen of interest. In particular, it may comprise one or more CDRs or VH and/or VL domains of an antibody capable of specifically binding to an antigen of the ECM.

The antigen-binding sites of the antibody, antibody fragment or antibody derivative (e.g., an scFv, a diabody or scFv2 or single-chain diabody) may be identical or different, but preferably are identical (e.g., the L19 antigen-binding sites of antibody L19, see below). Each of the antigen-binding sites may bind the same antigen or epitope. This can be achieved by providing two identical antigen-binding sites such as two identical VH-VL domain pairs, or by providing two different antigen-binding sites, for example comprising different VH and VL domains, which nevertheless both bind the same antigen or epitope. Alternatively, the antibody, antibody fragment or antibody derivative may be bispecific. By “bispecific” it is meant that each of the antigen-binding sites binds a different antigen. Optionally, two antigen-binding sites may bind two different antigens mentioned herein, e.g. two different antigens of the extracellular matrix, or two different domains of a particular antigen (e.g. fibronectin or tenascin-C).

The antigen may be an antigen preferentially expressed by cells of a tumour or tumour neovasculature or associated with the ECM. Such antigens include fibronectin and tenascin C, as described above.

Herein, the term “specific binding” means that one member of a specific binding pair will not show any significant binding to molecules other than its specific binding partner(s). The term is also applicable where, e.g., an antigen-binding site is specific for a particular epitope that is present on a number of different antigens, in which case the antibody, antibody fragment or antibody derivative carrying the antigen-binding site will be able to bind to the various antigens carrying the epitope.

The antibody, fragment or derivative thereof may specifically bind fibronectin. Fibronectin is an antigen subject to alternative splicing, and a number of alternative isoforms of fibronectin are known, including alternatively spliced isoforms A-FN and B-FN, comprising domains ED-A or ED-B, respectively, which are known markers of angiogenesis. Preferably, the antibody, fragment or derivative thereof binds to fibronectin isoform B-FN, e.g., most preferably it binds to the ED-B domain (extra domain B) of fibronectin isoform B-FN. The amino acid sequence of the ED-B domain of B-FN is provided by residues 1266-1356 of the UniProt database entry P02751 (human Fibronectin), and herein as SEQ ID NO: 39.

Thus, preferably, the antibody, fragment or derivative portion of the immunocytokine binds to an epitope of SEQ ID NO:39. Preferably, the antibody, fragment or derivative (e.g., an scFv polypeptide sequence) binds to the epitope represented by SEQ ID NO: 40.

Fibronectin isoform B-FN is one of the best-known markers for angiogenesis (WO1997/045544). The extra domain “ED-B” of 91 amino acids is found in the B-FN isoform and is identical in mouse, rat, rabbit, dog and man. B-FN accumulates around neovascular structures in aggressive tumours and other tissues undergoing angiogenesis, such as the endometrium in the proliferative phase and some ocular structures in pathological conditions, but is otherwise undetectable in normal adult tissues.

Preferably, the antibody derivative is a human monoclonal scFv sequence that specifically binds to alternatively spliced ED-B domain of fibronectin isoform B-FN, wherein preferably the scFv sequence binds to an epitope of SEQ ID NO:39, e.g., the epitope represented by SEQ ID NO: 40.

Also preferably, the antibody, antibody fragment or antibody derivative (e.g., scFv sequence) that specifically binds to alternatively spliced ED-B domain of fibronectin isoform B-FN comprises an antigen-binding site derived from the antibody L19. The antibody L19 binds specifically to the alternatively spliced ED-B domain of fibronectin isoform B-FN, wherein the epitope represented by SEQ ID NO: 40. The sequence of antibody L19 is disclosed in Pini et al. (1998) J. Biol. Chem. 273:21769-21776 or U.S. Pat. No. 8,097,254. An example of a most preferred antibody derivative is thus L19 scFv, which has been described previously (WO1999/058570; WO2006/119897 or WO2003/076469); see also the L19 scFv-comprising immunocytokine of SEQ ID NO: 15 and FIG. 1B in WO2020/070150).

Most preferably, the immunocytokine comprises (as the antibody derivative) a human monoclonal scFv polypeptide sequence specific for alternatively spliced ED-B domain of fibronectin isoform B-FN (e.g. the epitope represented by SEQ ID NO: 40) as described above and (as the cytokine) IL2 (see, e.g., SEQ ID NO: 15 and FIG. 1B in WO2020/070150, and SEQ ID NO: 12 herein).

### Antigen-Binding Site and Sequences

The antigen-binding site may, e.g., comprise one, two, three, four, five or six CDRs of antibody L19. Amino acid sequences of the CDRs of L19 are: SEQ ID NO: 13 (CDR1 VH); SEQ ID NO: 14 (CDR2 VH); SEQ ID NO: 15 (CDR3 VH); SEQ ID NO:16 (CDR1 VL); SEQ ID NO:17 (CDR2 VL), and/or SEQ ID NO:18 (CDR3 VL).

SEQ ID NOs 13-15 are the amino acid sequences of the VH CDR regions (1-3, respectively) of the human monoclonal antibody L19. SEQ ID NOs 16-18 are the amino acid of the VL CDR regions (1-3, respectively) of the human monoclonal antibody L19.

The antigen-binding site may be flanked by one, two, three, four, five, six, seven, or eight of the framework regions of antibody L19. The amino acid sequences of the framework regions of L19 are: SEQ ID NO: 30 (Framework region 1 VH); SEQ ID NO: 31 (Framework region 1 VH); SEQ ID NO: 32 (Framework region 1 VH); SEQ ID NO: 33 (Framework region 1 VH); SEQ ID NO: 34 (Framework region 1 VL); SEQ ID NO: 35 (Framework region 1 VL); SEQ ID NO: 36 (Framework region 1 VL); and/or SEQ ID NO: 37 (Framework region 1 VL).

The amino acid sequence of the VH and VL domains of antibody L19 correspond to SEQ ID NOs 19 and 20, respectively.

Preferably the antibody derivative comprises a VH domain with an amino acid sequence comprising VH CDR1, VH CDR2, and/or VH CDR3 of L19, and a VL domain with an amino acid sequence comprising VL CDR1, VL CDR2, and/or VL CDR3 of L19.

The antibody derivative as described above may comprise a VH domain having an amino acid sequence with at least 60%, 65%, 70%, 75%, 80%, 85%, 90%, 95% or 100% (preferably 80%) sequence identity with the amino acid sequence of the L19 VH domain as set out in SEQ ID NO: 22, and/or may comprise a VL domain having an amino acid sequence with at least 60%, 65%, 70%, 75%, 80%, 85%, 90%, 95% or 100% % (preferably 80%) sequence identity with the amino acid sequence of the L19 VL domain as set out in SEQ ID NO: 23.

Preferably the antibody derivative as described above is an L19 scFv (i.e., an scFv derived from and comprising one or more antigen-specific portions of antibody L19; interchangeably referred to as “scFv (L19)”). An L19 scFv may thus comprise one, two, three, four, five or six CDRs of antibody L19 (preferably all six CDRs). Optionally the L19 scFv may comprise an L19 VH domain (SEQ ID NO: 22) and/or an L19 VL domain (SEQ ID NO: 23), e.g., both the L19 VH and the L19 VL.

In an scFv unit, the VH and VL domains are joined by a linker. The linker may be a peptide linker sequence that is not long enough to allow pairing of the VH and VL domains. Thus, a homodimer of scFvs may form instead, in which the VH of one scFv chain pairs with the VL of the other scFv chain (and vice versa). This general format may be referred to as an “scFv2” format, or, alternatively, in some cases, as a “diabody”. Examples of suitable short linker sequences are GSSGG (SEQ ID NO: 26) and GGSGG (SEQ ID NO: 27). Preferably the linker is the 12-residue linker SEQ ID NO: 22.

The antibody derivative may be an L19 diabody having an amino acid sequence with at least 60%, 65%, 70%, 75%, 80%, 85%, 90%, 95% or 100% (preferably 80%) sequence identity with the amino acid sequence of as set out in SEQ ID NO: 28 or an L19 scDb having an amino acid sequence with at least 60%, 65%, 70%, 75%, 80%, 85%, 90%, 95% or 100% (preferably 80%) sequence identity with the amino acid sequence of as set out in SEQ ID NO: 29

In a preferred embodiment, the antibody derivative is an scFv (L19) having an amino acid sequence with at least 60%, 65%, 70%, 75%, 80%, 85%, 90%, 95% or 100% (preferably 80%) sequence identity with the amino acid sequence of as set out in SEQ ID NO: 38.

Linkage of Segments within the Immunocytokine

The immunocytokine comprises a cytokine (e.g., an IL2 polypeptide) and is conjugated to an antibody, an antibody fragment or an antibody derivative. This conjugation may be effected through any suitable covalent bond or linker moiety, e.g., a disulphide or peptide bond, most preferably a peptide linker sequence. A peptide linker sequence may be a short (2-30, preferably 10-20) residue stretch of amino acids. Suitable examples of peptide linker sequences are known in the art. Examples of suitable linker sequences are (G₄S)₃ (SEQ ID NO:24) or GSLDGAGGSAGADGG (SEQ ID NO: 25). One or more different linkers may be used. Preferably, the linker may be the 17-residue linker of SEQ ID NO: 23.

Thus, the antibody, antibody fragment, or antibody derivative and IL2 may be produced and/or secreted as a single-chain polypeptide.

The IL2 is preferably linked to the C-terminus of the antibody or the antibody fragment or the antibody derivative. Preferably, that linkage may be via a peptide linker sequence, as disclosed herein. Where the IL2 is conjugated to the C-terminus, the N-terminus of the antibody or the antibody fragment or the antibody derivative is preferably free. “Free” in this context refers to the N-terminus not being linked or otherwise conjugated to another moiety, such as IL2.

### Preferred L19-IL2 Immunoconjugates.

Preferably, the immunocytokine comprises an L19-derived scFv unit linked to an IL2 polypeptide in a conjugate polypeptide chain that that forms a homodimer due to complementary pairing between the VH and VL domains of the scFv unit in two polypeptide chains (in accordance with the scFv2 or diabody format). Thus, preferably, the immunocytokine comprises a sequence having at least 60%, 65%, 70%, 75%, 80%, 85%, 90%, 95% or 100% (preferably 80%) sequence identity with the amino acid sequence of the “L19-IL2” immunocytokine represented by SEQ ID NO: 12, which comprises an L19-derived scFv unit linked to IL2, possesses such functionality and forms a homodimer via the scFv units in this manner. More preferably, the immunocytokine comprises the sequence of SEQ ID NO: 12 (i.e., an amino acid sequence of an scFv-format L19-IL2 polypeptide).

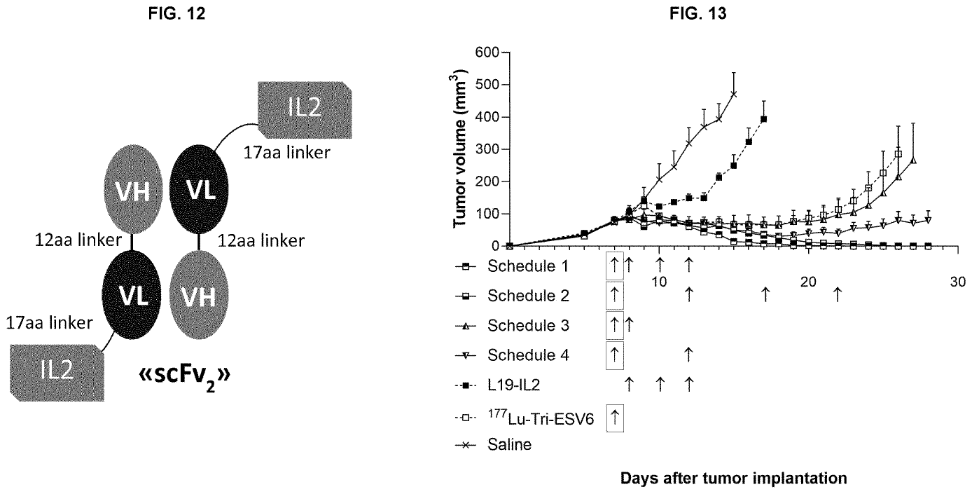

The production and purification of L19-IL2 constructs may be performed as described in WO01/062298. A schematic representation of the scFv-format L19-IL2 conjugate is shown in FIG. 12.

### Further Definitions Relating to the Immunocytokine

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215:405-410), FASTA (which uses the method of Pearson and Lipman (1988) PNAS USA 85: 2444-2448), or the Smith-Waterman algorithm (Smith and Waterman (1981) J. Mol Biol. 147:195-197), or the TBLASTN program, of Altschul et al. (1990) supra, generally employing default parameters. In particular, the psi-Blast algorithm (Nucl. Acids Res. (1997) 25 3389-3402) may be used.

Variants of these VH and VL domains and CDRs may also be employed in antibody molecules for use in conjugates as described herein. Suitable variants can be obtained by means of methods of sequence alteration, or mutation, and screening.

Particular variants for use as described herein may include one or more amino acid sequence alterations (addition, deletion, substitution and/or insertion of an amino acid residue), maybe less than about 20 alterations, less than about 15 alterations, less than about 10 alterations or less than about 5 alterations, 4, 3, 2 or 1.

### Precursor Compounds

In one aspect of the invention, herein disclosed is a compound, its individual diastereoisomers, its hydrates, its solvates, its crystal forms, its individual tautomers or a salt thereof, wherein the compound (precursor compound) comprises three moieties A and a reactive moiety G capable of reacting and forming a covalent bond with a conjugation partner. Upon conjugation (i.e., reacting and forming a covalent bond), the former precursor compound is bound to the former conjugation partner, which in turn to a therapeutic payload moiety C. The conjugation partner can be an atom, a molecule, a particle, a therapeutic agent. Preferably, the conjugation is a therapeutic agent, and can correspond to the payload moieties already described in detail above with respect to the therapeutic conjugates according to the invention.

Each moiety A preferably has the structure A1 or A2 as previously defined.

Preferably, the precursor compound is represented by the following formula:

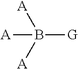

wherein B is a covalent bond or a multifunctional moiety covalently attaching the moieties A to G.

Preferably, G is capable of forming, upon reacting, an amide, ester, carbamate, hydrazone, thiazolidine, methylene alkoxy carbamate, disulphide, alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, aminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide or tetrapeptide linking group; and/or

Moiety B preferably has a structure as described in detail above with respect to the conjugates according to the invention.

Moiety G is preferably capable of forming, upon reacting, an amide, ester, carbamate, hydrazone, thiazolidine, methylene alkoxy carbamate, disulphide, alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, aminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide or tetrapeptide linking group. As will be appreciated by a person skilled in the art, multiple possibilities exist how to provide a reactive group capable of reacting with a conjugation partner to form a linking group according to the aforementioned list, and they are all encompassed by the present disclosure.

The moiety B may be cleavable or non-cleavable, multifunctional moiety which can be used to link one or more reactive and/or binder moieties to form the conjugate precursor of the invention. In some embodiments, the structure of the compound comprises, independently, more than one moiety A, preferably 2, 3, 4, 5, 6, 7, 8, 9 or 10 moieties A; and/or more than one moiety G, preferably 2, 3, 4, 5, 6, 7, 8, 9 or 10 moieties G per molecule. Preferably, the structure of the compound comprises 3 moieties A and 1 moiety G; or 3 moieties A and 2 moieties G per molecule.

Moiety G is preferably selected from: H, NH₂, OH, N₃, COOH, SH, Hal,

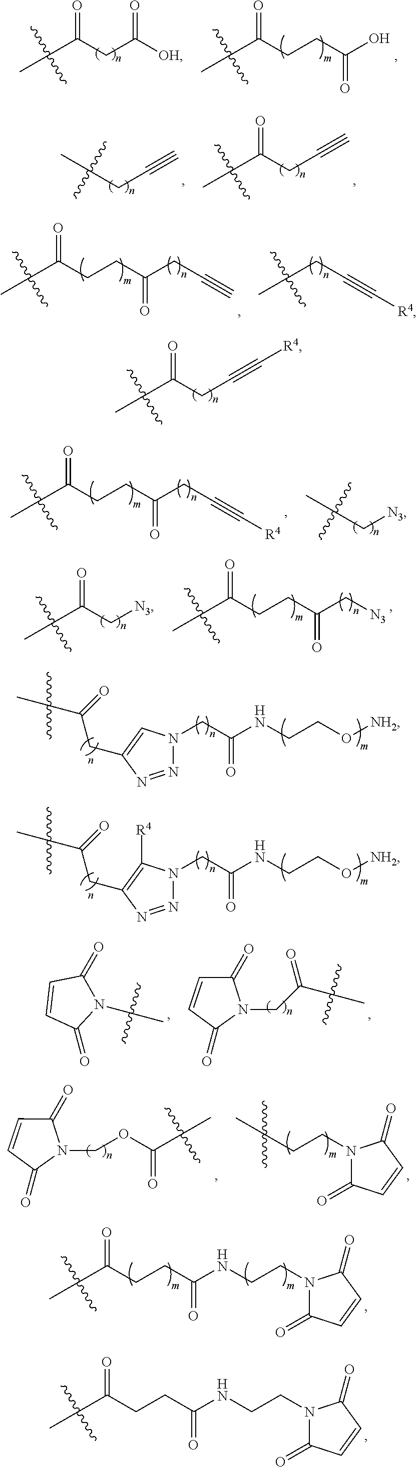

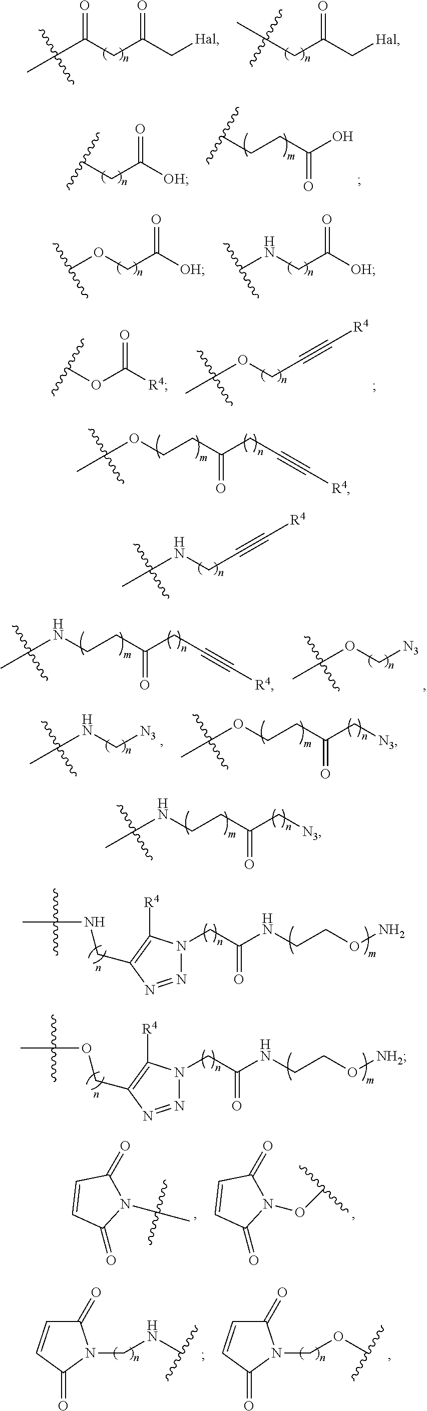

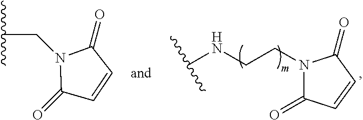

wherein each n is, independently, 0, 1, 2, 3, 4, 5, 6, 7, 8, 9 or 10; each m is, independently, 0, 1, 2, 3, 4 or 5; each Hal is F, Cl, Br or I; and each R⁴ is, independently selected from carboxy, alkyl, cycloalkyl, aryl and heteroaryl, wherein each of the foregoing is substituted or unsubstituted, halogen, and cyano.

In all structures, unless otherwise specified, all groups and variables are as defined further above throughout the present disclosure.

### Further Compounds

Herein disclosed are also novel tetravalent organic ligands of fibroblast activation protein (FAP) (“Tetra-ESV6”) suitable for therapeutic applications, comprising four small binding moieties A as defined above. A compound according to the present invention may be represented by following general Formula IV,

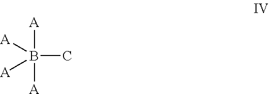

its individual diastereoisomers, its hydrates, its solvates, its crystal forms, its individual tautomers or a pharmaceutically acceptable salt thereof, wherein A is a binding moiety; B is a multifunctional moiety comprising a ramification point, and covalently connects the moieties A to C; and C is an atom, a molecule or a particle, and is a therapeutic agent.

Alternatively, moiety C comprises a ramification point, such that four moieties A are attached to C, and B is absent, i.e., moiety C can also serve as a ramification point therefore substituting B, preferably when C is a chelator. An exemplary structure is represented by general Formula V:

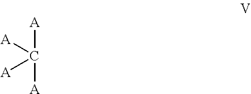

All exemplary structures, aspects, embodiments and definitions disclosed for the trivalent binders of general Formulae I and III disclosed in the present specification apply mutatis mutandis for general Formulae IV and V.

For instance, the tetravalent binder can be represented by Formulae VI or VII, wherein all groups and variables are otherwise the same as those defined for Formulae II or III:

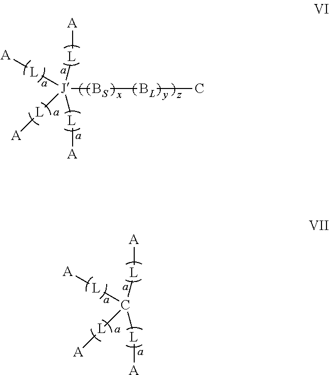

Exemplary structures for moiety J′ include:

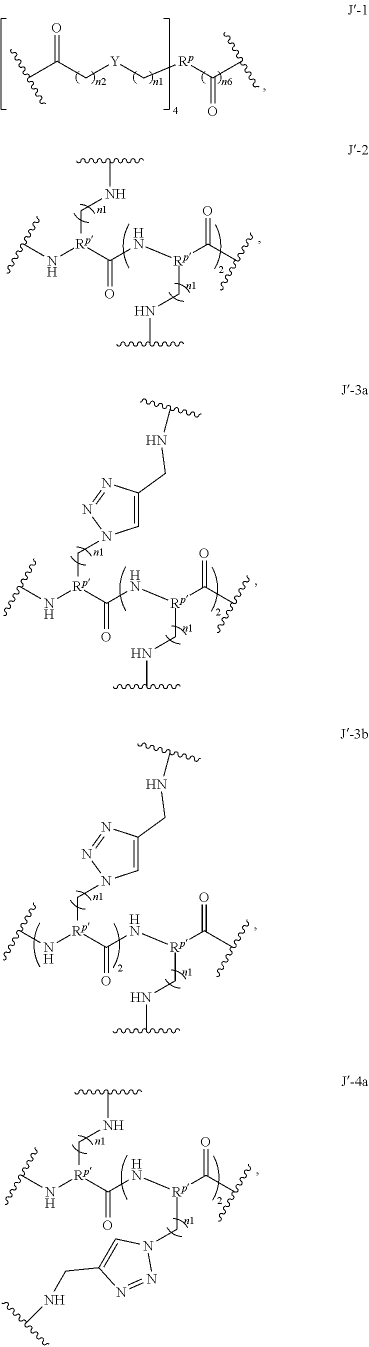

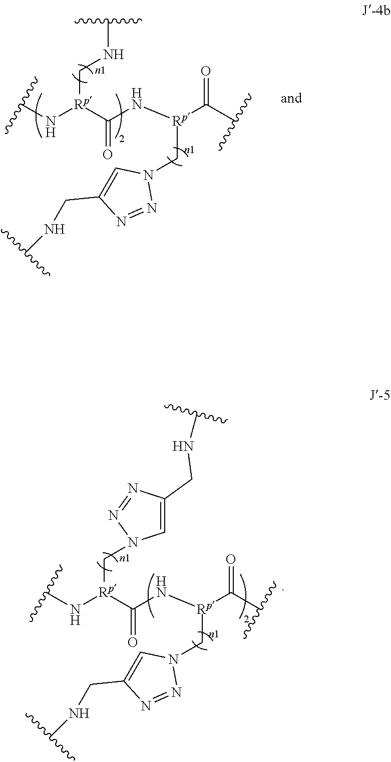

wherein all groups and variables are otherwise the same as defined herein.

Specific structures for moiety J′ include:

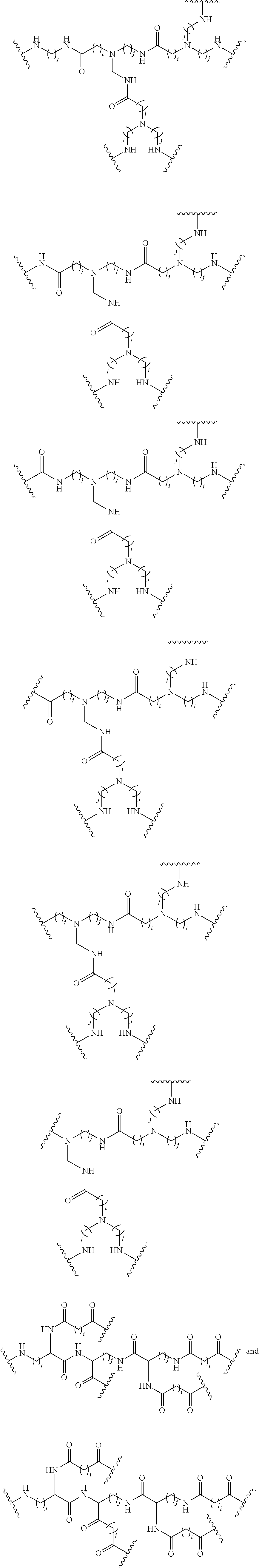

wherein each i and j is an integer independently selected from 0, 1, 2, 3 and 4; preferably wherein each i is 1 or 2, and each j is 1, 2 or 3.

Preferred structures for J′ include:

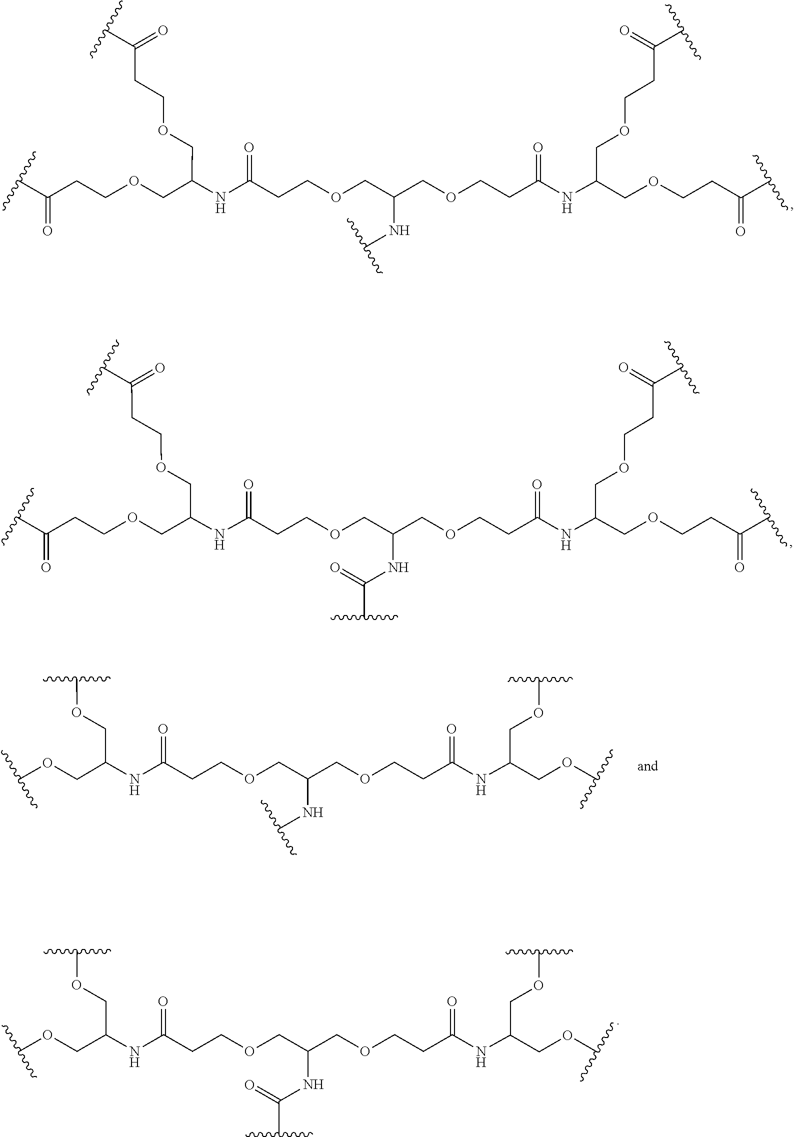 [SMILES: *NC(COCCC(=O)NC(COCCC(*)=O)COCCC(*)=O)COCCC(=O)NC(COCCC(*)=O)COCCC(*)=O ; *C(=O)CCOCC(COCCC(*)=O)NC(=O)CCOCC(COCCC(=O)NC(COCCC(*)=O)COCCC(*)=O)NC(*)=O ; *NC(COCCC(=O)NC(CO*)CO*)COCCC(=O)NC(CO*)CO* ; *OCC(CO*)NC(=O)CCOCC(COCCC(=O)NC(CO*)CO*)NC(*)=O]

Particularly preferred structures for fragment ((L)ₐ)₄J′ include:

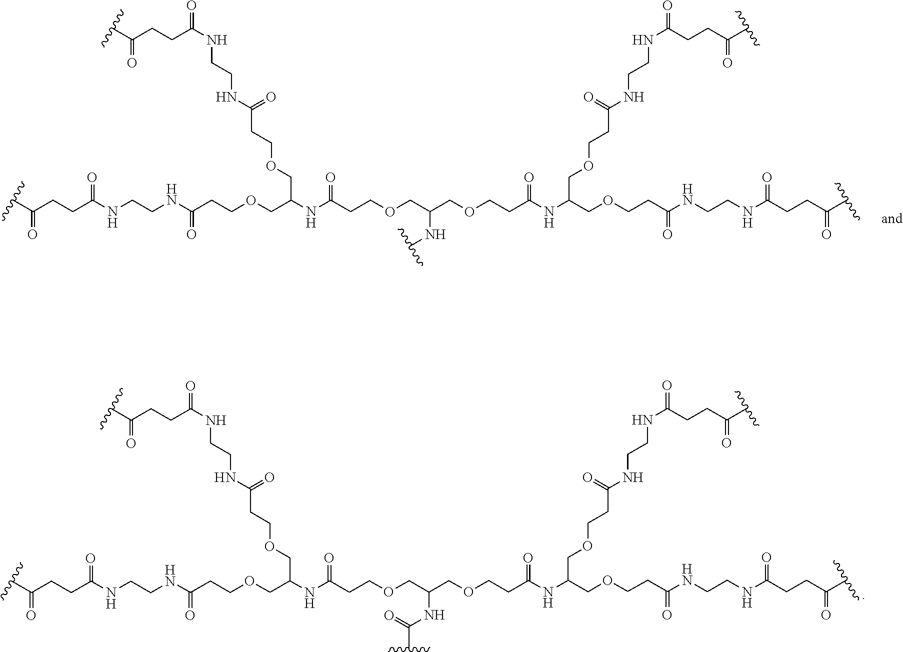 [SMILES: *NC(COCCC(=O)NC(COCCC(=O)NCCNC(=O)CCC(*)=O)COCCC(=O)NCCNC(=O)CCC(*)=O)COCCC(=O)NC(COCCC(=O)NCCNC(=O)CCC(*)=O)COCCC(=O)NCCNC(=O)CCC(*)=O ; *C(=O)CCC(=O)NCCNC(=O)CCOCC(COCCC(=O)NCCNC(=O)CCC(*)=O)NC(=O)CCOCC(COCCC(=O)NC(COCCC(=O)NCCNC(=O)CCC(*)=O)COCCC(=O)NCCNC(=O)CCC(*)=O)NC(*)=O]

Particularly preferred tetravalent compounds are shown in Table 17.

### Methods for Preparing a Therapeutic Conjugate

In one aspect of the invention, herein disclosed is a method for preparing a therapeutic conjugate comprising the step of conjugating with a precursor compound as described above with a conjugation partner. Preferably, the precursor compound is conjugated to the conjugation partner by reacting therewith to form a covalent bond. Preferably, the thus obtained conjugate is a therapeutic conjugate compound as described elsewhere in the present specification.

The conjugation partner can be an atom, a molecule or a particle which is a therapeutic agent, and can correspond to the therapeutic payload moieties already described in detail above with respect to the conjugates according to the invention.

Preferably, the method further comprises formulating the conjugate as a therapeutic pharmaceutical composition. The pharmaceutical compositions may be for human or animal therapy in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s). All formulation details and aspects disclosed above in the section “Pharmaceutical compositions” fully apply here too.

### General Techniques

The practice of the present invention employs, unless otherwise indicated, conventional methods of chemistry, biochemistry, molecular biology, cell biology, genetics, immunology and pharmacology, known to those of skill of the art. Such techniques are explained fully in the literature. See, e. g., Gennaro, A. R., ed. (1990) Remington's Pharmaceutical Sciences, 18th ed., Mack Publishing Co.; Hardman, J. G., Limbird, L. E., and Gilman, A. G., eds. (2001) The Pharmacological Basis of Therapeutics, 10th ed., McGraw-Hill Co.; Colowick, S. et al., eds., Methods In Enzymology, Academic Press, Inc.; Weir, D. M., and Blackwell, C. C., eds. (1986) Handbook of Experimental Immunology, Vols. I-IV, Blackwell Scientific Publications; Maniatis, T. et al., eds. (1989) Molecular Cloning: A Laboratory Manual, 2nd edition, Vols. I-III, Cold Spring Harbor Laboratory Press; Ausubel, F. M. et al., eds. (1999) Short Protocols in Molecular Biology, 4th edition, John Wiley & Sons; Ream et al., eds. (1998) Molecular Biology Techniques: An Intensive Laboratory Course, Academic Press; Newton, C. R., and Graham, A., eds. (1997) PCR (Introduction to Biotechniques Series), 2nd ed., Springer Verlag.

### Chemical Synthesis

The compounds described herein may be prepared by chemical synthesis techniques. It will be apparent to those skilled in the art that sensitive functional groups may need to be protected and deprotected during synthesis of a compound. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994). It is possible during some of the reactions that any stereocentres present could, under certain conditions, be epimerised, for example if a base is used in a reaction with a substrate having an optical centre comprising a base-sensitive group. It should be possible to circumvent potential problems such as this by choice of reaction sequence, conditions, reagents, protection/deprotection regimes, etc. as is well-known in the art.

### DEFINITIONS

Antibody. The term “antibody” is used in its broadest sense and covers monoclonal antibodies, polyclonal antibodies, dimers, multimers, multispecific antibodies (e.g., bispecific antibodies), veneered antibodies, antibody fragments and small immune proteins (SIPs) (see Int. J. Cancer (2002) 102, 75-85). An antibody is a protein generated by the immune system that is capable of recognizing and binding to a specific antigen. A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e. a molecule that contains an antigen binding site that immunospecifically binds an antigen of a target of interest or part thereof. The antibodies may be of any type—such as IgG, IgE, IgM, IgD, and IgA—any class—such as IgG1, IgG2, IgG3, IgG4, IgAQ1 and IgA2—or subclass thereof. The antibody may be or may be derived from murine, human, rabbit or from other species.

Antibody fragments. The term “antibody fragment” refers to a portion of a full length antibody, generally the antigen binding or variable region thereof. Examples of antibody fragments include, but are not limited to, Fab, Fab′, F(ab′)₂, and Fv fragments; diabodies; linear antibodies; single domain antibodies, including dAbs, camelid V_{HH} antibodies and the IgNAR antibodies of cartilaginous fish. Antibodies and their fragments may be replaced by binding molecules based on alternative non-immunoglobulin scaffolds, peptide aptamers, nucleic acid aptamers, structured polypeptides comprising polypeptide loops subtended on a non-peptide backbone, natural receptors or domains thereof.

Derivative. A derivative includes the chemical modification of a compound. Examples of such modifications include the replacement of a hydrogen by a halo group, an alkyl group, an acyl group or an amino group and the like. The modification may increase or decrease one or more hydrogen bonding interactions, charge interactions, hydrophobic interactions, van der Waals interactions and/or dipole interactions.

Analog. This term encompasses any enantiomers, racemates and stereoisomers, as well as all pharmaceutically acceptable salts and hydrates of such compounds.

Unless otherwise stated, the following definitions apply to chemical terms used in connection of compounds of the invention and compositions containing such compounds.

Alkyl refers to a branched or unbranched saturated hydrocarbyl radical. Suitably, the alkyl group comprises from 1 to 100, preferably 3 to 30, carbon atoms, more preferably from 5 to 25 carbon atoms. Preferably, alkyl refers to methyl, ethyl, propyl, butyl, pentyl, or hexyl.

Alkenyl refers to a branched or unbranched hydrocarbyl radical containing one or more carbon-carbon double bonds. Suitably, the alkenyl group comprises from 2 to 30 carbon atoms, preferably from 5 to about 25 carbon atoms.

Alkynyl refers to a branched or unbranched hydrocarbyl radical containing one or more carbon-carbon triple bonds. Suitably, the alkynyl group comprises from about 3 to about 30 carbon atoms, for example from about 5 to about 25 carbon atoms.

Halogen refers to fluorine, chlorine, bromine or iodine, preferably fluorine or chlorine.

Cycloalkyl refers to an alicyclic moiety, suitably having 3, 4, 5, 6, 7 or 8 carbon atoms. The group may be a bridged or polycyclic ring system. More often cycloalkyl groups are monocyclic. This term includes reference to groups such as cyclopropyl, cyclobutyl, cyclopentyl, cyclohexyl, norbornyl, bicyclo[2.2.2]octyl and the like.

Aryl refers to an aromatic carbocyclic ring system, suitably comprising 6, 7, 8, 9, 10, 11, 12, 13, 14, 15 or 16 ring carbon atoms. Aryl may be a polycyclic ring system, having two or more rings, at least one of which is aromatic. This term includes reference to groups such as phenyl, naphthyl fluorenyl, azulenyl, indenyl, anthryl and the like.

The prefix (hetero) herein signifies that one or more of the carbon atoms of the group may be substituted by nitrogen, oxygen, phosphorus, silicon or sulfur. Heteroalkyl groups include for example, alkyloxy groups and alkythio groups. Heterocycloalkyl or heteroaryl groups herein may have from 3, 4, 5, 6, 7, 8, 9, 10, 11, 12, 13, 14, 15 or 16 ring atoms, at least one of which is selected from nitrogen, oxygen, phosphorus, silicon and sulfur. In particular, a 3- to 10-membered ring or ring system and more particularly a 5- or 6-membered ring, which may be saturated or unsaturated. For example, selected from oxiranyl, azirinyl, 1,2-oxathiolanyl, imidazolyl, thienyl, furyl, tetrahydrofuryl, pyranyl, thiopyranyl, thianthrenyl, isobenzofuranyl, benzofuranyl, chromenyl, 2H-pyrrolyl, pyrrolyl, pyrrolinyl, pyrrolidinyl, imidazolyl, imidazolidinyl, benzimidazolyl, pyrazolyl, pyrazinyl, pyrazolidinyl, thiazolyl, isothiazolyl, dithiazolyl, oxazolyl, isoxazolyl, pyridyl, pyrazinyl, pyrimidinyl, piperidyl, piperazinyl, pyridazinyl, morpholinyl, thiomorpholinyl, especially thiomorpholino, indolizinyl, 1,3-Dioxo-1,3-dihydro-isoindolyl, 3H-indolyl, indolyl, benzimidazolyl, cumaryl, indazolyl, triazolyl, tetrazolyl, purinyl, 4H-quinolizinyl, isoquinolyl, quinolyl, tetrahydroquinolyl, tetrahydroisoquinolyl, decahydroquinolyl, octahydroisoquinolyl, benzofuranyl, dibenzofuranyl, benzothiophenyl, dibenzothiophenyl, phthalazinyl, naphthyridinyl, quinoxalyl, quinazolinyl, quinazolinyl, cinnolinyl, pteridinyl, carbazolyl, [beta]-carbolinyl, phenanthridinyl, acridinyl, perimidinyl, phenanthrolinyl, furazanyl, phenazinyl, phenothiazinyl, phenoxazinyl, chromenyl, isochromanyl, chromanyl, 3,4-dihydro-2H-isoquinolin-1-one, 3,4-dihydro-2H-isoquinolinyl, and the like.

“Substituted” signifies that one or more, especially up to 5, more especially 1, 2 or 3, of the hydrogen atoms in said moiety are replaced independently of each other by the corresponding number of substituents. The term “optionally substituted” as used herein includes substituted or unsubstituted. It will, of course, be understood that substituents are only at positions where they are chemically possible, the person skilled in the art being able to decide (either experimentally or theoretically) without inappropriate effort whether a particular substitution is possible. For example, amino or hydroxy groups with free hydrogen may be unstable if bound to carbon atoms with unsaturated (e.g. olefinic) bonds. Preferably, the term “substituted” signifies one or more, especially up to 5, more especially 1, 2 or 3, of the hydrogen atoms in said moiety are replaced independently of each other by the corresponding number of substituents selected from OH, SH, NH₂, halogen, cyano, carboxy, alkyl, cycloalkyl, aryl and heteroaryl. Additionally, the substituents described herein may themselves be substituted by any substituent, subject to the aforementioned restriction to appropriate substitutions as recognised by the skilled person. Preferably, any of the aforementioned substituents may be further substituted by any of the aforementioned substituents, each of which may be further substituted by any of the aforementioned substituents.

Substituents may suitably include halogen atoms and halomethyl groups such as CF₃ and CCl₃; oxygen containing groups such as oxo, hydroxy, carboxy, carboxyalkyl, alkoxy, alkoyl, alkoyloxy, aryloxy, aryloyl and aryloyloxy; nitrogen containing groups such as amino, alkylamino, dialkylamino, cyano, azide and nitro; sulfur containing groups such as thiol, alkylthiol, sulfonyl and sulfoxide; heterocyclic groups which may themselves be substituted; alkyl groups, which may themselves be substituted; and aryl groups, which may themselves be substituted, such as phenyl and substituted phenyl. Alkyl includes substituted and unsubstituted benzyl.

Where two or more moieties are described as being “each independently” selected from a list of atoms or groups, this means that the moieties may be the same or different. The identity of each moiety is therefore independent of the identities of the one or more other moieties.

### EXAMPLES

### 1. General Remarks and Procedures

Yields refer to chromatographically purified compounds, unless specified otherwise.

Mass Spectrometry (LC-ESI-MS) spectra were recorded on an Agilent 6100 Series Single Quadrupole MS System combined with an Agilent 1200 Series LC System, using an InfinityLab Poroshell 120 EC-C18 column, 4.6 mm×56 mm at a flow rate of 2 mL min⁻¹ with linear gradients of solvents A and B (A=Millipore water with 0.1% formic acid [FA], B=MeCN with 0.1% formic acid [FA]); or using an InfinityLab Poroshell 120 EC-C18 Column, 2.7 μm, 4.6×50 mm at a flow rate of 0.8 mL/min, 10% ACN in 0.1% aq. HCOOH to 100% ACN in 3 or 10 min.

High-Resolution Mass Spectrometry (HRMS) spectra and analytical Reversed-Phase Ultra Performance Liquid Chromatography (UPLC) were recorded on a Waters Xevo G2-XS QTOF coupled to a Waters Acquity UPLC H-Class System with PDA UV detector, using a ACQUITY UPLC BEH C18 Column, 130 Å, 1.7 μm, 2.1 mm×50 mm at a flow rate of 0.6 mL min⁻¹ with linear gradients of solvents A and B (A=Millipore water with 0.1% FA, B=MeCN with 0.1% FA).

Preparative reversed-phase high-pressure liquid chromatography (RP-HPLC) was performed on an Agilent 1200 Series System, using a Phenomenex Gemini® 5 μm NX-C18 semipreparative column, 110 Å, 150 mm×10 mm at a flow rate of 5 mL min⁻¹ with linear gradients of solvents A and B (A=Millipore water with 0.1% trifluoroacetic acid [TFA], B=MeCN with 0.1% trifluoroacetic acid [TFA]); or on an Agilent 1200 Series RP-HPLC with PDA UV detector, using a Synergi 10 μm, MAX-RP 80 Å 10×250 mm C18 column at a flow rate of 5 mL/min with linear gradients of solvents A and B (A=Millipore water with 0.1% TFA, B=ACN with 0.1% TFA).

### 2. Synthesis of Precursors and Reference Compounds

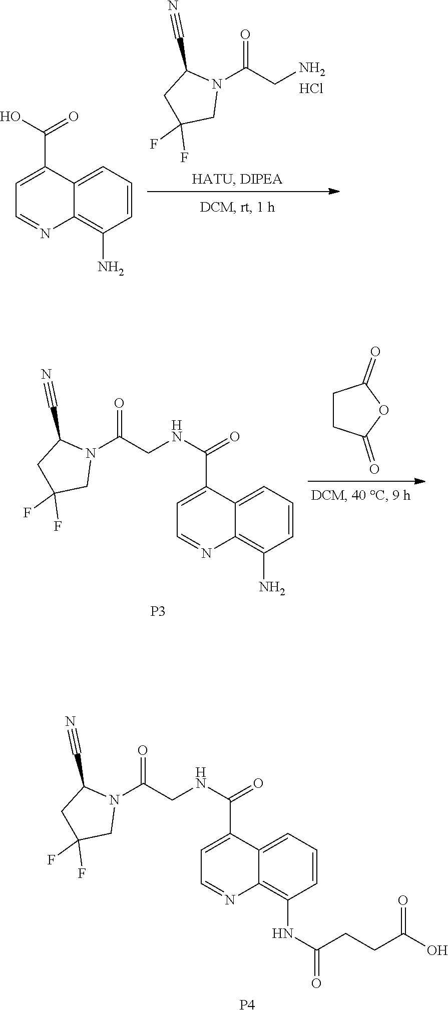

### Synthesis of ESV6-Succinic-COOH (P4) for Therapeutic Use

Step 1: (S)-8-amino-N-(2-(2-cyano-4,4-difluoropyrrolidin-1-yl)-2-oxoethyl) quinoline-4-carboxamide (P3). Commercially available 8-amino-quinoline-4-carboxylic acid (19.0 mg, 100 μmol, 1.0 eq), DIPEA (70.0 μL, 400 μmol, 4.0 eq) and HATU (38.0 mg, 100 μmol, 1.0 eq) were dissolved in a 1:1 DCM/DMF mixture (2 mL). After 15 min a solution of(S)-1-(2-aminoacetyl)-4,4-difluoropyrrolidine-2-carbonitrile trifluoroacetate (30.3 mg, 100 μmol, 1.0 eq) in DCM was added. The reaction mixture was stirred for 1 h at room temperature, washed with water, dried over Na₂SO₄, filtered and concentrated to obtain a brown crude as sticky oil. The residue was purified by flash chromatography (DCM/MeOH from 91:1 to 90:10) to yield the pure product as a brownish oil (24.8 mg, 68.9 μmol, 69% yield). MS (ES⁺) m/z 360 (M+H)⁺.

Step 2 (S)-4-((4-((2-(2-cyano-4,4-difluoropyrrolidin-1-yl)-2-oxoethyl)carbamoyl)quinolin-8-yl)amino) 4-oxobutanoic acid (P4). Triethylamine (20.8 μL, 150 μmol, 2.0 eq) and 4-dimethylaminopyridine (0.91 mg, 10.0 μmol, 0.1 eq) were added to a cooled solution (0 °C) of P3 (26.8 mg, 70.0 μmol, 1.0 eq) in DCM, followed by a dropwise addition of succinic anhydride (15.0 mg, 150 μmol, 2.0 eq). The reaction mixture was allowed to warm to room temperature. The reaction mixture was placed in a preheated 40 °C oil bath until full conversion was observed. The solvent was evaporated and the residue was purified by RP-HPLC to yield the pure product as a white powder (9.42 mg, 20.0 μmol, 28% yield). MS (ES⁺) m/z 460 (M+H)⁺.

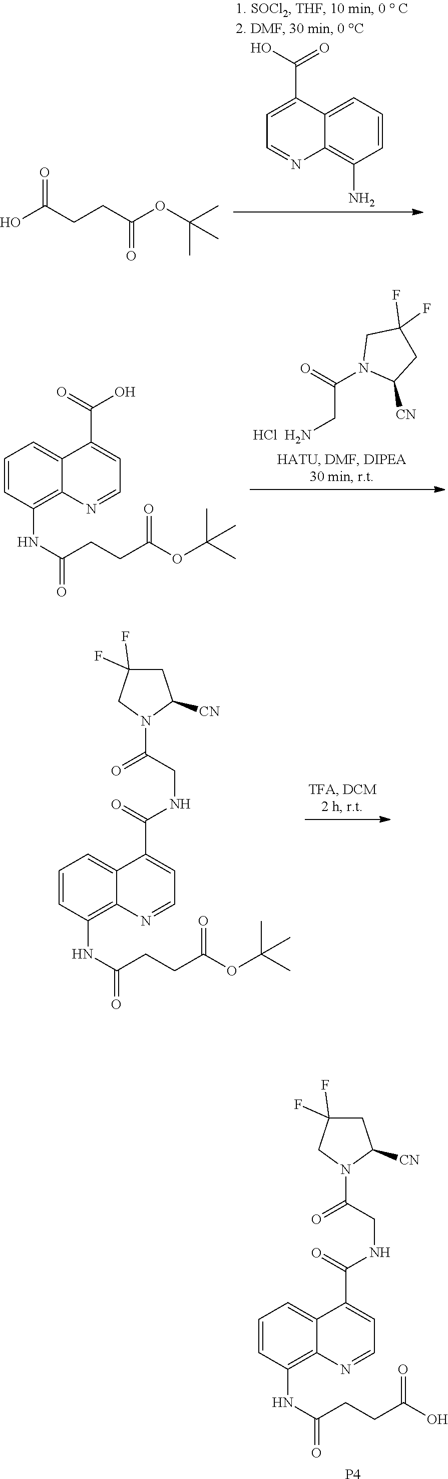

### Alternative Synthesis of ESV6-Succinic-COOH (P4) for Therapeutic Use

### Step 1:8-(4-(tert-butoxy)-4-oxobutanamido)quinoline-4-carboxylic acid

To a solution of 4-(tert-butoxy)-4-oxobutanoic acid (57 mg, 0.33 mmol, 1.2 eq) in dry THF (1.5 mL), SOCl2 (24 μL, 0.33 mmol, 1.2 eq) was slowly added at 0 °C. A solution of 8-aminoquinoline-4-carboxylic acid (50 mg, 0.25 mmol, 1 eq) in DMF (500 μL) was then added and the mixture was stirred for 30 min at 0 °C. The crude was concentrated under reduced pressure and purified by Reverse Phase MPLC (98:2 to 0:100 AcCN/water+0.1% HCOOH in 45 min). The fractions were collected and lyophilized to afford a white solid (50 mg, 0.15 mmol, 57%).

Step 2: tert-butyl(S)-4-((4-((2-(2-cyano-4,4-difluoropyrrolidin-1-yl)-2-oxoethyl)carbamoyl)quinolin-8-yl)amino)-4-oxobutanoate. 8-(4-(tert-butoxy)-4-oxobutanamido) quinoline-4-carboxylic acid (50 mg, 0.15 mmol, 1 eq), (S)-1-(2-aminoacetyl)-4,4-difluoropyrrolidine-2-carbonitrile hydrochloride (33 mg, 0.15 mmol, 1 eq) and HATU (55 g, 0.15 mmol, 1 eq) were suspended in 2.0 mL of DMF. DIPEA (100 μL, 0.6 mmol, 4 eq) was added dropwise and the reaction was stirred for 15 minutes. The crude was diluted with DCM, washed with water, dried over anhydrous Na₂SO₄, filtered and the solvent evaporated under vacuum. The crude was concentrated under reduced pressure and purified by Reverse Phase MPLC (98:2 to 0:100 AcCN/water+0.1% HCOOH in 45 min). The fractions were collected and lyophilized to afford a white solid (40 mg, 0.077 mmol, 52%).

Step 3: (S)-4-((4-((2-(2-cyano-4,4-difluoropyrrolidin-1-yl)-2-oxoethyl)carbamoyl)quinolin-8-yl)amino)-4-oxobutanoic acid (P4). tert-butyl(S)-4-((4-((2-(2-cyano-4,4-difluoropyrrolidin-1-yl)-2-oxoethyl)carbamoyl)quinolin-8-yl)amino)-4-oxobutanoate (40 mg, 0.077 mmol, 1 eq) was dissolved in DCM (600 μL) and TFA (285 μL, 3.85 mmol, 50 eq) was added dropwise at 0 °C. The mixture was stirred for 2 hours and then concentrated under reduced pressure. The crude was purified by Reverse Phase MPLC (98:2 to 0:100 AcCN/water+0.1% HCOOH in 45 min). The fractions were collected and lyophilized to afford a white solid (25 mg, 0.054 mmol, 71%).

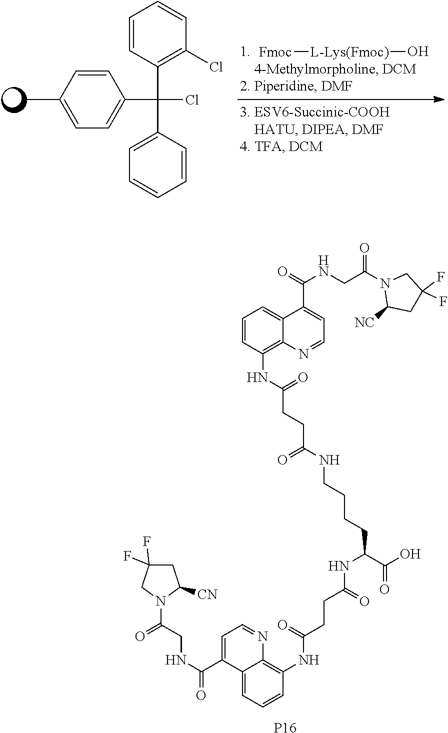

Synthesis of Bi-ESV6-COOH) (P16) for therapeutic use. To a solid-phase synthesis syringe, 2-chlorotrityl resin (300 mg) was added and then swollen with dry DCM for 15 min. Fmoc-L-Lys (Fmoc)-OH (89 mg, 0.15 mmol, 1 eq.) and 4-Methylmorpholine (45 μL, 0.40 mmol, 2.7 eq.) were sequentially added to the resin and the mixture was allowed to react for 3 h. Next, a capping step with methanol/4-Methylmorpholine/DCM (1:2:7 ratio, 5 mL, 30 min) was carried out, following by a wash with DMF and Fmoc-removal with 20% solution of Piperidine in DMF (10 mL). The resin was then treated with a solution of ESV6-Succinic-COOH (P4, 137 mg, 0.300 mmol, 2.0 eq.), HATU (86 mg, 0.22 mmol, 1.5 eq.) and DIPEA (97 μL, 0.75 mmol, 5.0 eq.) in DMF (5 mL) for 1 h. After multiple washing with DMF, the resin was submitted to the cleavage with 30% solution of TFA in DCM (10 mL) for 1 h. The cleaved solution was recovered, concentrated under vacuo and purified by RP-Chromatography (gradient: water/acetonitrile+0.1% FA 98:2 to 0:100 in 45 min). The fractions were collected and lyophilized to afford Bi-ESV6-COOH (P16) as a white solid (30 mg, 0.029 mmol, 19% yield). MS (ES+) m/z 1029.3 (M+H)⁺

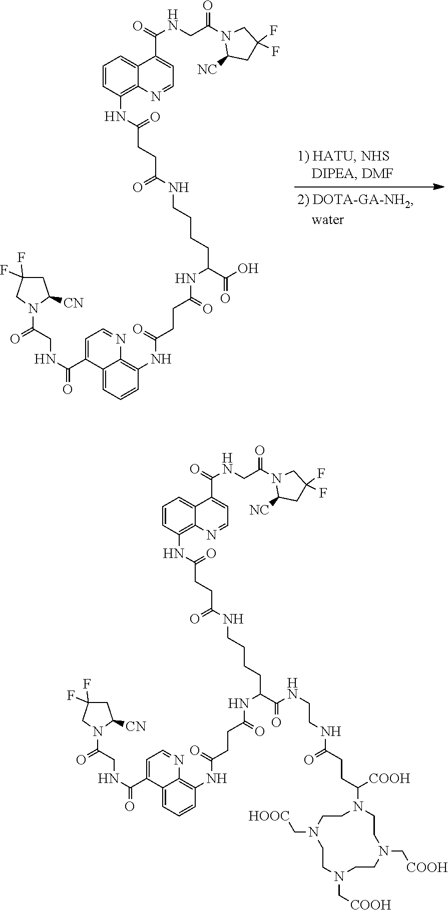 [SMILES: N#C[C@@H]1CC(F)(F)CN1C(=O)CNC(=O)c1ccnc2c(NC(=O)CCC(=O)NCCCCC(NC(=O)CCC(=O)Nc3cccc4c(C(=O)NCC(=O)N5CC(F)(F)C[C@H]5C#N)ccnc34)C(=O)O)cccc12 ; N#C[C@@H]1CC(F)(F)CN1C(=O)CNC(=O)c1ccnc2c(NC(=O)CCC(=O)NCCCCC(NC(=O)CCC(=O)Nc3cccc4c(C(=O)NCC(=O)N5CC(F)(F)C[C@H]5C#N)ccnc34)C(=O)NCCNC(=O)CCC(C(=O)O)N3CCN(CC(=O)O)CCN(CC(=O)O)CCN(CC(=O)O)CC3)cccc12]

Synthesis of Bi-ESV6-DOTAGA for therapeutic use. To a solution of Bi-ESV6-COOH) (P16, 12 mg, 0.012 mmol) in DMF (500 μL), N-hydroxysuccinimmide (2.0 mg, 0.017 mmol, 1.5 eq), HATU (6.7 mg, 0.017 mmol, 1.5 eq) and DIPEA (8 μL 0.05 mmol, 4.0 eq) were added. After 30 min, a solution of DOTA-GA-NH₂ (12 mg, 0.023 mmol, 2.0 eq) in water (500 μL) was added dropwise. The reaction mixture was stirred for further 30 min at room temperature, then purified by RP-HPLC (Agilent 1200 series system equipped with Synergi 4 μm Polar-RP 80 Å 10×150 mm C18 column using a gradient of 90:10 to 0:100 water/acetonitrile+0.1% TFA in 12 min). The fractions were collected and lyophilized to afford a white solid (10 mg, 0.007 mmol, 56% yield). MS (ES+) m/z 1530.5 (M+H)⁺.

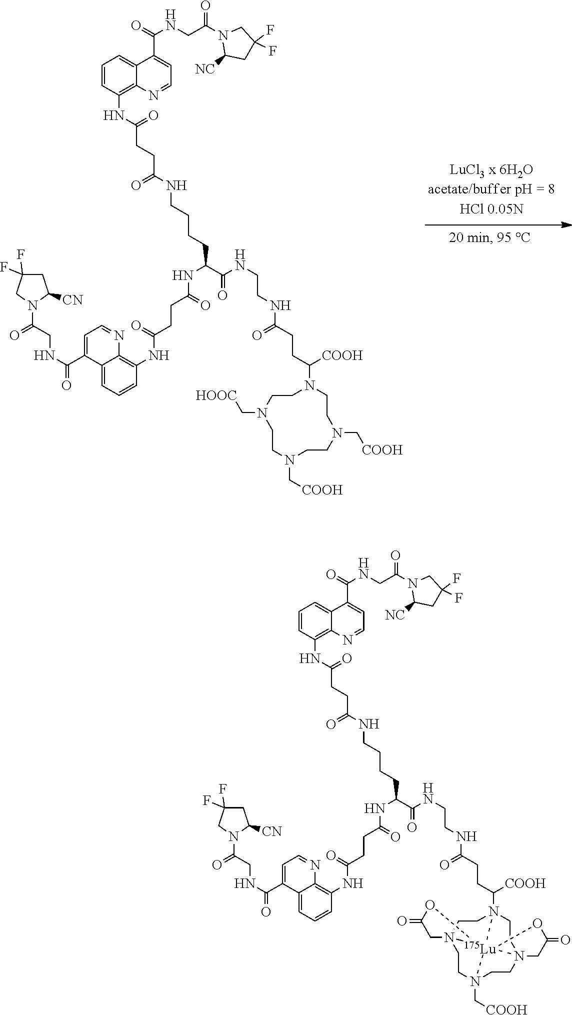

Synthesis of Bi-ESV6-DOTAGA-¹⁷⁵Lu for therapeutic use. Bi-ESV6-DOTAGA (4.0 mg, 2.6 μmol, 1 eq.) was dissolved in acetate buffer, pH=8 (300 μL). Subsequently a solution of LuCl₃ hexahydrate (2.0 mg, 5.2 μmol, 2 eq.) dissolved in 0.05 N HCl (1.50 mL) was added. The reaction was stirred at 90 °C for 20 min, then cooled down to r. t. and purified via RP-HPLC (Agilent 1200 series system equipped with Synergi 4 μm Polar-RP 80 Å 10×150 mm C18 column using a gradient of 90:10 to 50:50 water/acetonitrile+0.1% TFA in 7 min). The desired fractions were collected and lyophilized to afford a pale-yellow solid. (2.2 mg, 49%).

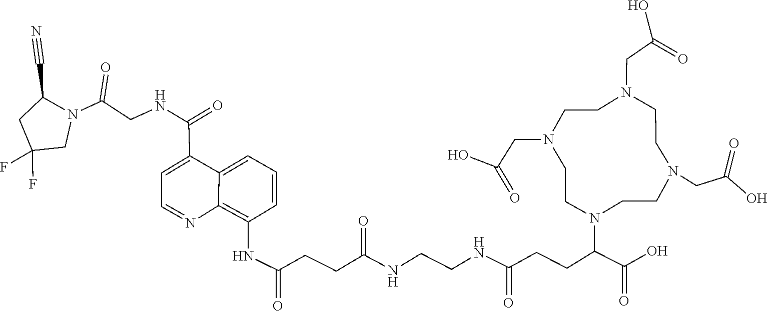 [SMILES: N#C[C@@H]1CC(F)(F)CN1C(=O)CNC(=O)c1ccnc2c(NC(=O)CCC(=O)NCCNC(=O)CCC(C(=O)O)N3CCN(CC(=O)O)CCN(CC(=O)O)CCN(CC(=O)O)CC3)cccc12]

Synthesis of ESV6-DOTAGA for therapeutic use. (S)-4-((4-((2-(2-cyano-4,4-difluoropyrrolidin-1-yl)-2-oxoethyl)carbamoyl)quinolin-8-yl)amino)-4-oxobutanoic acid (15 mg, 0.032 mmol, 1.0 eq) was dissolved in dry DMSO (400 μL). Dicyclohexylcarbodiimide (9 mg, 0.042 mmol, 1.3 eq) and N-hydroxysuccinimide (4.5 mg, 0.039 mmol, 1.3 eq) were added and the reaction was stirred overnight at room temperature, protected from light. 100 μL of PBS solution containing 2,2′,2″-(10-(4-((2-aminoethyl)amino)-1-carboxy-4-oxobutyl)-1,4,7,10-tetraazacyclododecane-1,4,7-triyl)triacetic acid (20 mg, 0.039 mmol, 1.2 eq) were added and the reaction was stirred for 2 h. The crude product was purified by reversed-phase HPLC (Water 0.1% TFA/Acetonitrile 0.1% TFA 9.5:0.5 to 2:8 in 20 min) and lyophilized, to obtain a white solid (2.4 mg, 8%). MS (ES+) m/z 960.39 (M+H)⁺

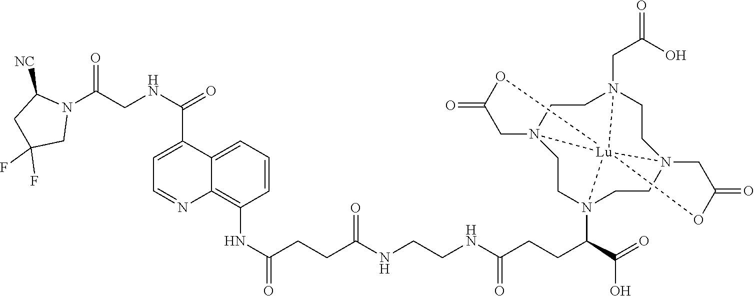

Synthesis of ESV6-DOTAGA-175Lu for therapeutic use. To a solution of ESV6-DOTAGA (0.96 mg, 1 μmol, 1 eq.) in 300 μL acetate buffer (aqueous solution, 1 M, pH 8), a freshly prepared solution of LuCl₃ hexahydrate (0.78 mg, 2 μmol, 2 eq.) in 0.05N HCl (1.5 mL) was added. The resulting mixture was stirred at 95 °C for 10-15 minutes, then purified via RP-HPLC (90:10 to 0:100 ACN/water+0.1% TFA in 12 min). The desired fractions were collected and lyophilized to afford a white solid. (0.8 mg, 71%). MS(ESI+) m/z 1133.3.

### 3. Chemical Structure of Tri-ESV6-DOTAGA (6)

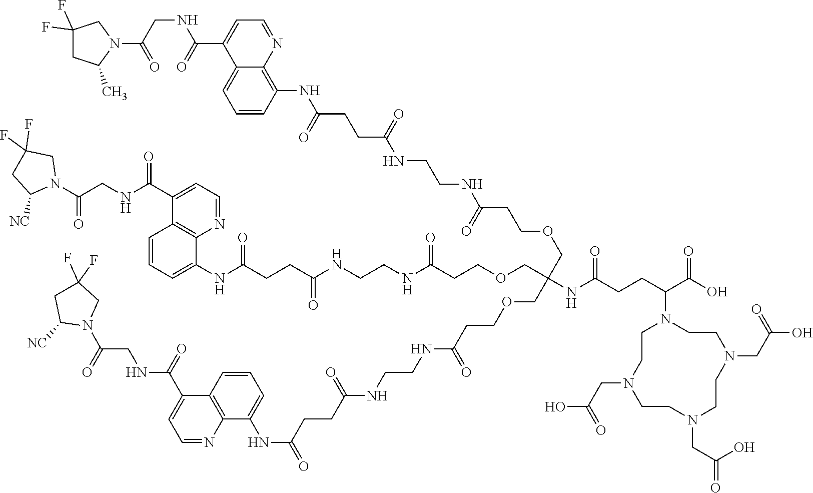 [SMILES: C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 2246.23 Da
Exact mass: 2244.8877 Da
Formula: C101H122F6N26O27
SMILES: O═C(NCCNC(CCOCC(COCCC(NCCNC(CCC(NC1=C(N═CC═C2C(NCC(N3CC(F)(F)C[C@H]3C #N)═O)═O)C2=CC═C1)=O)═O)═O)(NC(CCC(N4CCN(CC(O)═O)CCN(CC(O)═O)CCN(CC(O)═O)CC4)C(O)═O)═O)COCCC(N CCNC(CCC(NC5=CC═CC6=C5N═CC═C6C(NCC(N7CC(F)(F)C[C@H]7C #N)═O)═O)═O)═O)═O)═O)CCC(NC8=CC═CC9=C8N═CC═C9C(NCC(N % 10[C@H](C#N)CC(F)(F)C % 10)=O)═O)═O

### 4. Chemical Structure of Tri-ESV6-DOTA (7)

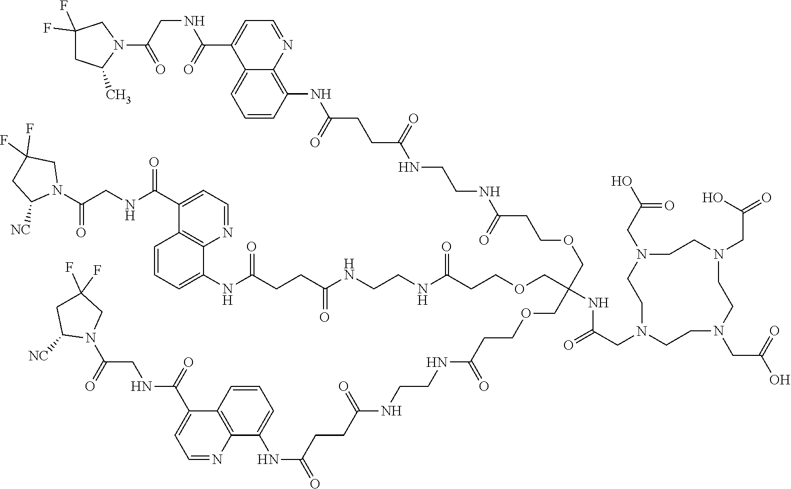 [SMILES: C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CN3CCN(CC(=O)O)CCN(CC(=O)O)CCN(CC(=O)O)CC3)cccc12]

Molecular weight: 2174.17 Da
Exact mass: 2177.87 Da
Formula: C98H118F6N26O25
SMILES: O—C(NCCNC(CCOCC(COCCC(NCCNC(CCC(NC1=C(N═CC═C2C(NCC(N3CC(F)(F)C[C@H]3C #N)═O)═O)C2=CC═C1)=O)═O)═O)(NC(CN4CCN(CC(O)═O)CCN(CC(O)═O)CCN(CC(O)═O)CC4)=O)COCCC(NCCNC(CCC(NC5=CC═CC6=C5N═CC═C6C(NCC(N7CC(F)(F)C[C@H]7C #N)═O)═O)═O)═O)═O)═O)CCC(NC8=CC═CC9=C8N═CC═C9C(NCC(N % 10 [C@H](C#N)CC(F)(F)C % 10)=O)═O)═O

### 5. Chemical Structure of Tri-ESV6-PEG12-DOTAGA

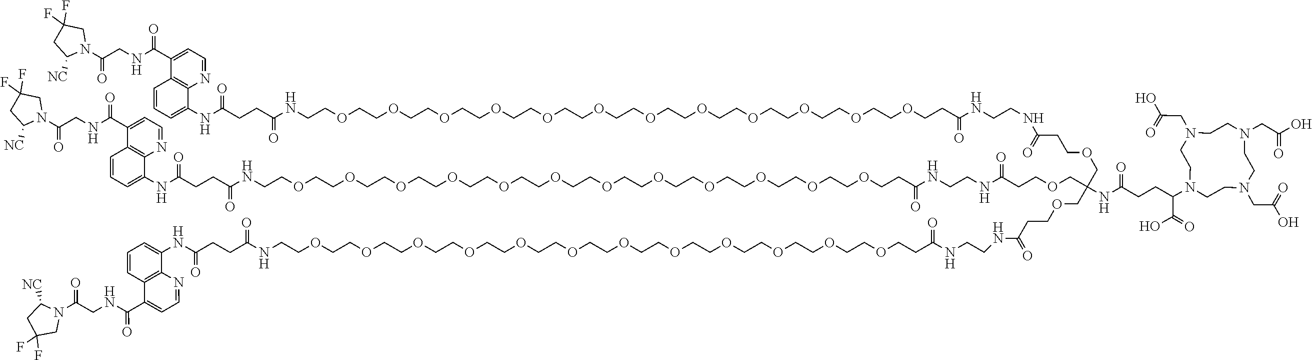 [SMILES: N#C[C@@H]1CC(F)(F)CN1C(=O)CNC(=O)c1ccnc2c(NC(=O)CCC(=O)NCCOCCOCCOCCOCCOCCOCCOCCOCCOCCOCCOCCOCCC(=O)NCCNC(=O)CCOCC(COCCC(=O)NCCNC(=O)CCOCCOCCOCCOCCOCCOCCOCCOCCOCCOCCOCCOCCNC(=O)CCC(=O)Nc3cccc4c(C(=O)NCC(=O)N5CC(F)(F)C[C@H]5C#N)ccnc34)(COCCC(=O)NCCNC(=O)CCOCCOCCOCCOCCOCCOCCOCCOCCOCCOCCOCCOCCNC(=O)CCC(=O)Nc3cccc4c(C(=O)NCC(=O)N5CC(F)(F)C[C@@H]5C#N)ccnc34)NC(=O)CCC(C(=O)O)N3CCN(CC(=O)O)CCN(CC(=O)O)CCN(CC(=O)O)CC3)cccc12]

Molecular weight: 4045.38 Da
Exact mass: 4042.94 Da
Formula: C182H118F6N26O25
SMILES: O═C(NCCOCCOCCOCCOCCOCCOCCOCCOCCOCCOCCOCCOCCC(NCCNC(CCOCC(COCCC(NCCNC(CC OCCOCCOCCOCCOCCOCCOCCOCCOCCOCCOCCOCCNC(CCC(NC1=CC═CC2=C1N═CC═C2C(NCC(N3 [C @H](C#N)CC(F)(F)C3)=O)═O)═O)═O)═O)═O)(NC(CCC(N4CCN(CC(O)═O)CCN(CC(O)═O)CCN(CC(O)═O)CC 4)C(O)═O)═O)COCCC(NCCNC(CCOCCOCCOCCOCCOCCOCCOCCOCCOCCOCCOCCOCCNC(CCC(NC5=CC═CC6=C5N═CC═C6C(NCC(N7 [C@H](C#N)CC(F)(F)C7)=O)═O)═O)═O)═O)═O)═O)═O)CCC(NC8=CC═CC9=C8N═CC═C9C(NCC(N % 10[C@H](C#N)CC(F)(F)C % 10)=O)═O)═O

### 6. Synthesis of Tri-ESV6-DOTAGA and Tri-ESV6-DOTA

### a) Synthetic Route

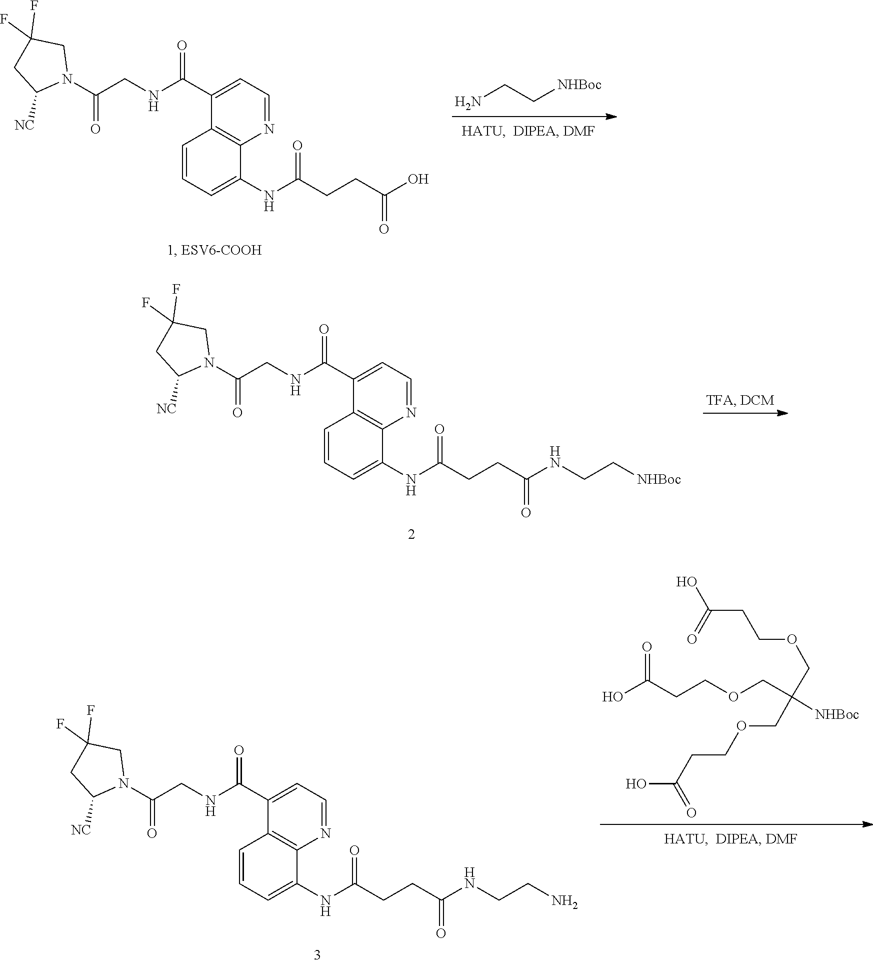 [SMILES: N#C[C@@H]1CC(F)(F)CN1C(=O)CNC(=O)c1ccnc2c(NC(=O)CCC(=O)O)cccc12 ; CC(C)(C)OC(=O)NCCN ; CC(C)(C)OC(=O)NCCNC(=O)CCC(=O)Nc1cccc2c(C(=O)NCC(=O)N3CC(F)(F)C[C@H]3C#N)ccnc12 ; N#C[C@@H]1CC(F)(F)CN1C(=O)CNC(=O)c1ccnc2c(NC(=O)CCC(=O)NCCN)cccc12 ; CC(C)(C)OC(=O)NC(COCCC(=O)O)(COCCC(=O)O)COCCC(=O)O]

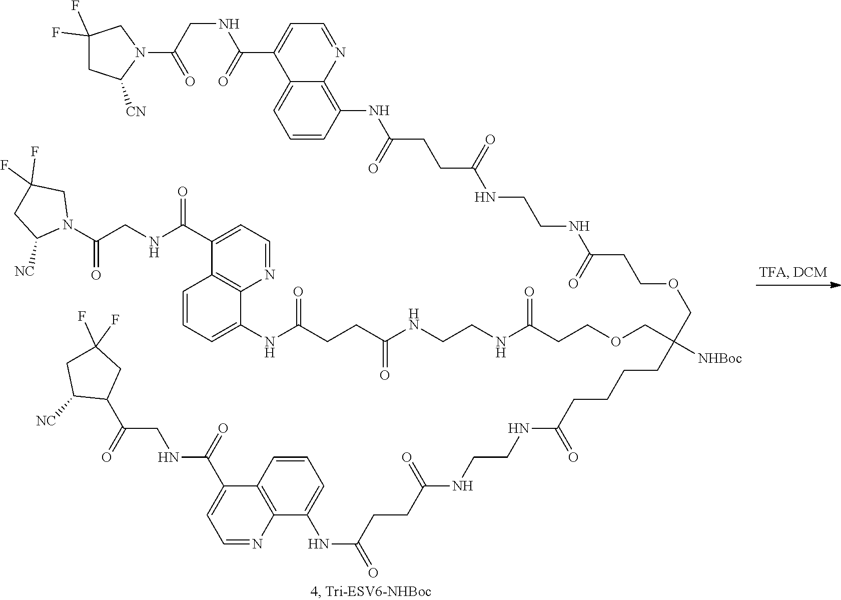 [SMILES: CC(C)(C)OC(=O)NC(CCCCC(=O)NCCNC(=O)CCC(=O)Nc1cccc2c(C(=O)NCC(=O)C3CC(F)(F)C[C@H]3C#N)ccnc12)(COCCC(=O)NCCNC(=O)CCC(=O)Nc1cccc2c(C(=O)NCC(=O)N3CC(F)(F)C[C@H]3C#N)ccnc12)COCCC(=O)NCCNC(=O)CCC(=O)Nc1cccc2c(C(=O)NCC(=O)N3CC(F)(F)C[C@H]3C#N)ccnc12]

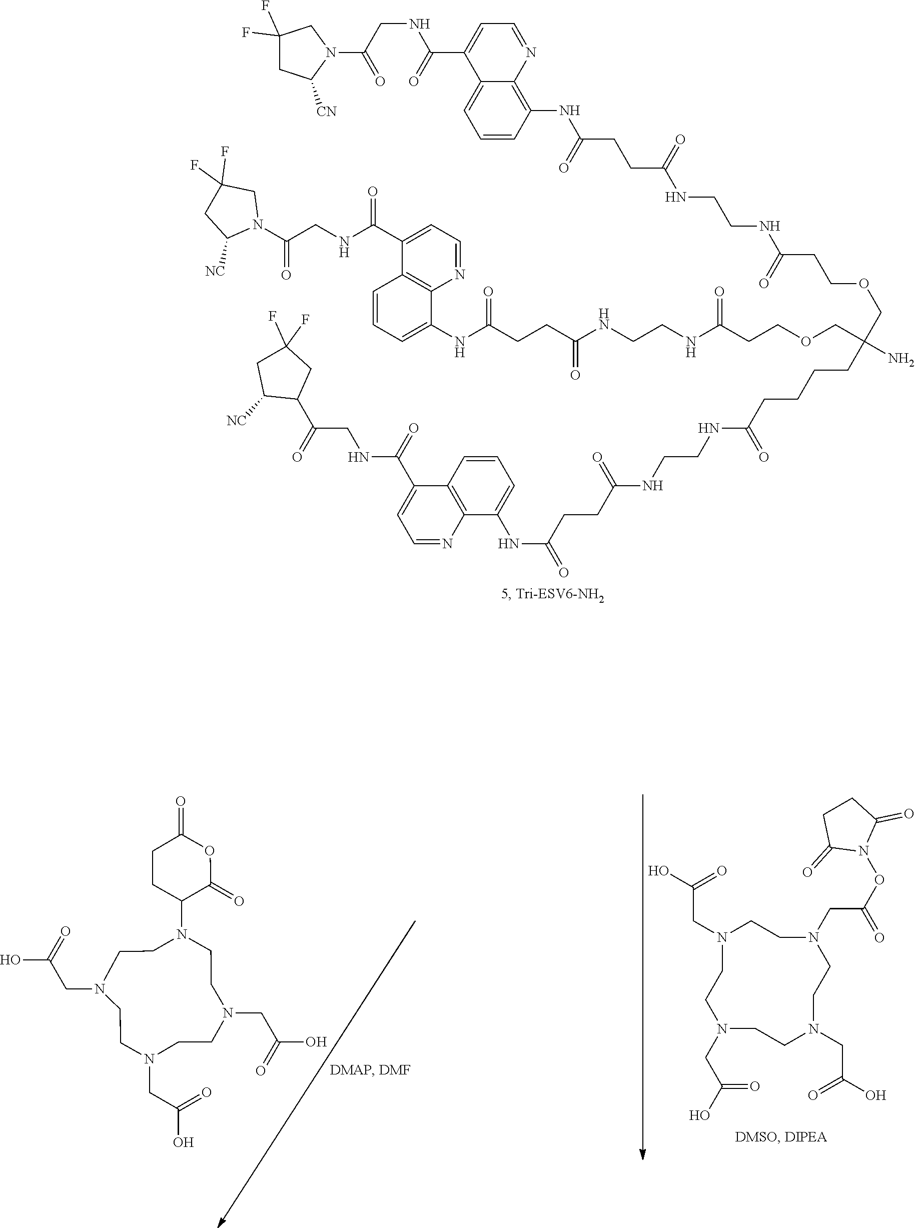 [SMILES: N#C[C@@H]1CC(F)(F)CC1C(=O)CNC(=O)c1ccnc2c(NC(=O)CCC(=O)NCCNC(=O)CCCCC(N)(COCCC(=O)NCCNC(=O)CCC(=O)Nc3cccc4c(C(=O)NCC(=O)N5CC(F)(F)C[C@H]5C#N)ccnc34)COCCC(=O)NCCNC(=O)CCC(=O)Nc3cccc4c(C(=O)NCC(=O)N5CC(F)(F)C[C@H]5C#N)ccnc34)cccc12 ; O=C(O)CN1CCN(CC(=O)O)CCN(C2CCC(=O)OC2=O)CCN(CC(=O)O)CC1 ; O=C(O)CN1CCN(CC(=O)O)CCN(CC(=O)ON2C(=O)CCC2=O)CCN(CC(=O)O)CC1]

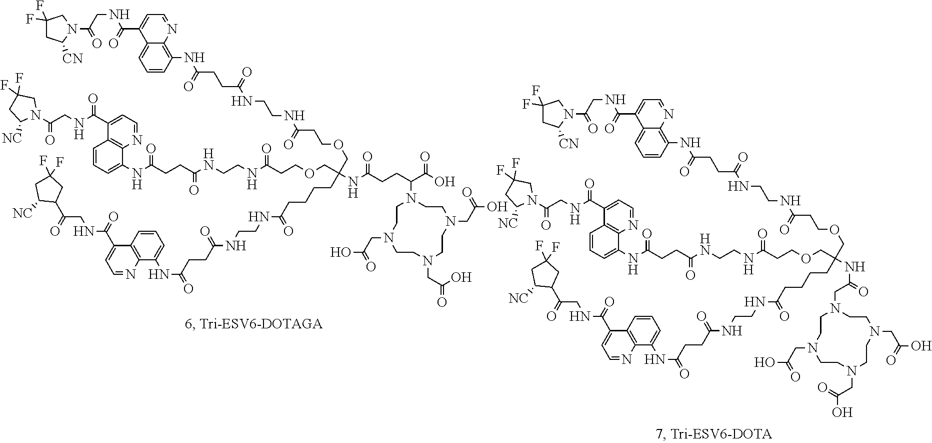

### b) Synthetic Experimental Procedures

### Step 1) tert-butyl(S)-(2-(4-((4-((2-(2-cyano-4,4-difluoropyrrolidin-1-yl)-2-oxoethyl)carbamoyl)quinolin-8-yl)amino)-4-oxobutanamido)ethyl)carbamate (2)

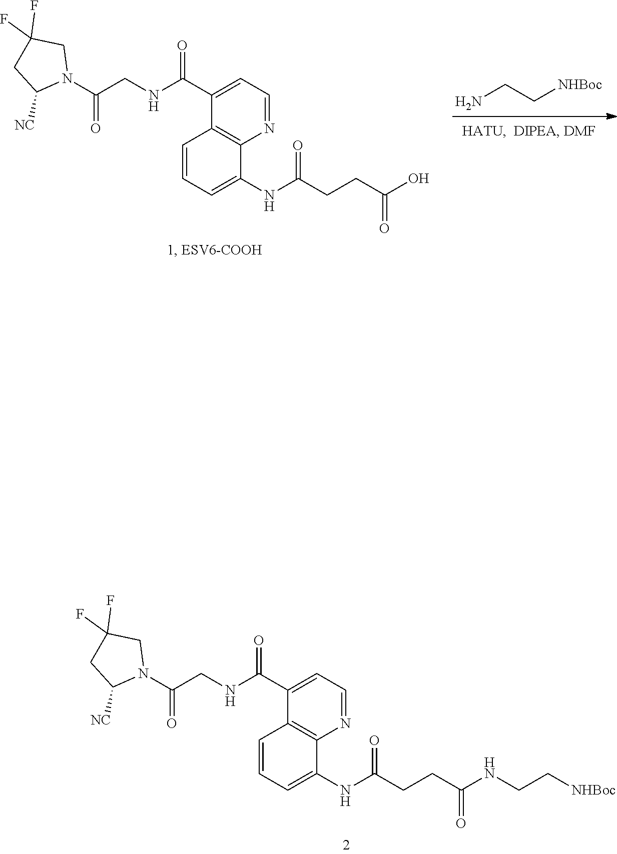 [SMILES: N#C[C@@H]1CC(F)(F)CN1C(=O)CNC(=O)c1ccnc2c(NC(=O)CCC(=O)O)cccc12 ; CC(C)(C)OC(=O)NCCN ; CC(C)(C)OC(=O)NCCNC(=O)CCC(=O)Nc1cccc2c(C(=O)NCC(=O)N3CC(F)(F)C[C@H]3C#N)ccnc12]

(S)-4-((4-((2-(2-cyano-4,4-difluoropyrrolidin-1-yl)-2-oxoethyl)carbamoyl)quinolin-8-yl)amino)-4-oxobutanoic acid (ESV6-COOH), 1, 100 mg, 217 μmol, 1 eq.), tert-butyl(2-aminoethyl)carbamate (42 mg, 261 μmol, 1.2 eq.) and HATU (100 mg, 261 μmol, 1.2 eq.) were suspended in 2.0 mL of DMF. DIPEA (150 μL, 868 μmol, 4 eq.) was added dropwise and the reaction was stirred for 15 minutes. The crude was purified via CombiFlash Nextgen 300+ (parameters: flow 30 mL/min, 24 gr C18 column, Water/Acetonitrile+0.1% Formic Acid 98:2 to 0:100 in 30 minutes) and the collected fractions were lyophilized to obtain a white solid (104 mg, 176 μmol, 80% yield).

### Step 2) (S)—N¹-(2-aminoethyl)-N⁴-(4-((2-(2-cyano-4,4-difluoropyrrolidin-1-yl)-2-oxoethyl)carbamoyl)quinolin-8-yl)succinimide (3)

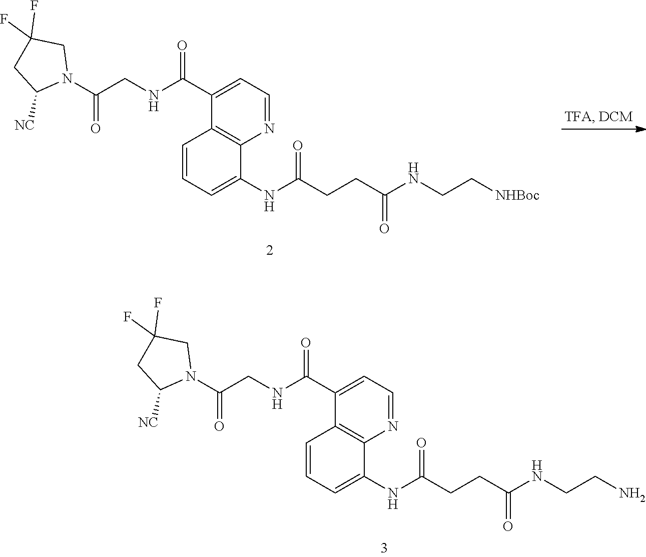 [SMILES: CC(C)(C)OC(=O)NCCNC(=O)CCC(=O)Nc1cccc2c(C(=O)NCC(=O)N3CC(F)(F)C[C@H]3C#N)ccnc12 ; N#C[C@@H]1CC(F)(F)CN1C(=O)CNC(=O)c1ccnc2c(NC(=O)CCC(=O)NCCN)cccc12]

tert-butyl(S)-(2-(4-((4-((2-(2-cyano-4,4-difluoropyrrolidin-1-yl)-2-oxoethyl)carbamoyl)quinolin-8-yl)amino)-4-oxobutanamido)ethyl)carbamate (104 mg, 176 μmol) was dissolved in 2 mL of a 30% v/v solution of TFA in DCM and stirred at room temperature for 1 h. The crude was purified via CombiFlash Nextgen 300+ (parameters: flow 30 mL/min, 24 gr C18 column, Water/Acetonitrile+0.1% Formic Acid 98:2 to 0:100 in 30 minutes) and the collected fractions were lyophilized to obtain a white solid (66 mg, 133 μmol, 76% yield).

### Step 3) tert-butyl (1,27-bis((4-((2-((S)-2-cyano-4,4-difluoropyrrolidin-1-yl)-2-oxoethyl)carbamoyl)quinolin-8-yl)amino)-14-((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-1,4,9,19,24,27-hexaoxo-12,16-dioxa-5,8,20,23-tetraazaheptacosan-14-yl)carbamate (4, Tri-ESV6-NHBoc)

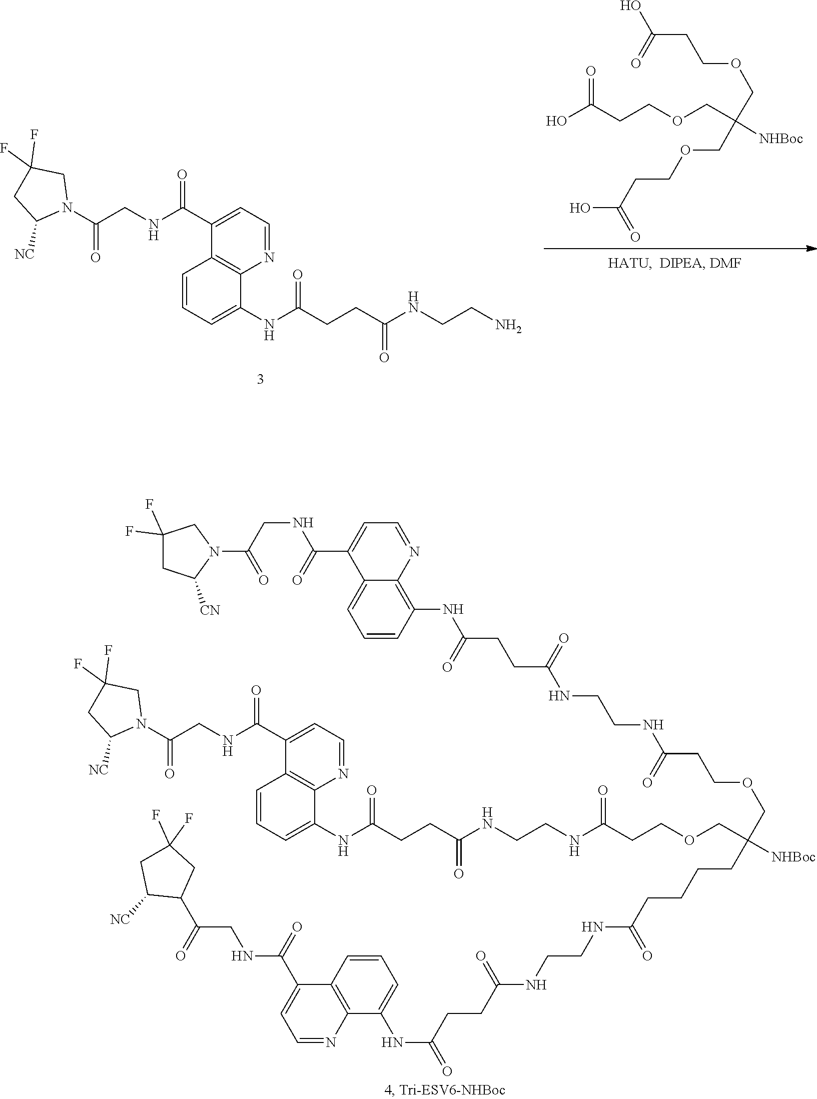 [SMILES: N#C[C@@H]1CC(F)(F)CN1C(=O)CNC(=O)c1ccnc2c(NC(=O)CCC(=O)NCCN)cccc12 ; CC(C)(C)OC(=O)NC(COCCC(=O)O)(COCCC(=O)O)COCCC(=O)O ; CC(C)(C)OC(=O)NC(CCCCC(=O)NCCNC(=O)CCC(=O)Nc1cccc2c(C(=O)NCC(=O)C3CC(F)(F)C[C@H]3C#N)ccnc12)(COCCC(=O)NCCNC(=O)CCC(=O)Nc1cccc2c(C(=O)NCC(=O)N3CC(F)(F)C[C@H]3C#N)ccnc12)COCCC(=O)NCCNC(=O)CCC(=O)Nc1cccc2c(C(=O)NCC(=O)N3CC(F)(F)C[C@H]3C#N)ccnc12]

(S)—N¹-(2-aminoethyl)-N⁴-(4-((2-(2-cyano-4,4-difluoropyrrolidin-1-yl)-2-oxoethyl)carbamoyl)quinolin-8-yl)succinimide (3, 66 mg, 133 μmol, 5 eq.), 3,3′-((2-((tert-butoxycarbonyl)amino)-2-((2-carboxyethoxy)methyl) propane-1,3-diyl)bis(oxy))dipropionic acid (11 mg, 26 μmol, 1 eq.) and HATU (34 mg, 91 μmol, 3.5 eq.) were suspended in 1.0 mL of DMF. DIPEA (40 μL, 260 μmol, 10 eq.) was added to the mixture and the reaction was stirred for 4 h at room temperature. The crude was purified via CombiFlash Nextgen 300+ (parameters: flow 15 mL/min, 12 gr C18 column, Water/Acetonitrile+0.1% Formic Acid 98:2 to 0:100 in 30 minutes) and the collected fractions were lyophilized to obtain a white solid (16 mg, 8.5 μmol, 32% yield).

### Step 4) N¹,N^{1′}-(9-amino-9-((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-4,14-dioxo-7,11-dioxa-3,15-diazaheptadecane-1,17-diyl)bis(N⁴-(4-((2-((S)-2-cyano-4,4-difluoropyrrolidin-1-yl)-2-oxoethyl)carbamoyl)quinolin-8-yl) succinamide) (5, Tri-ESV6-NH₂)

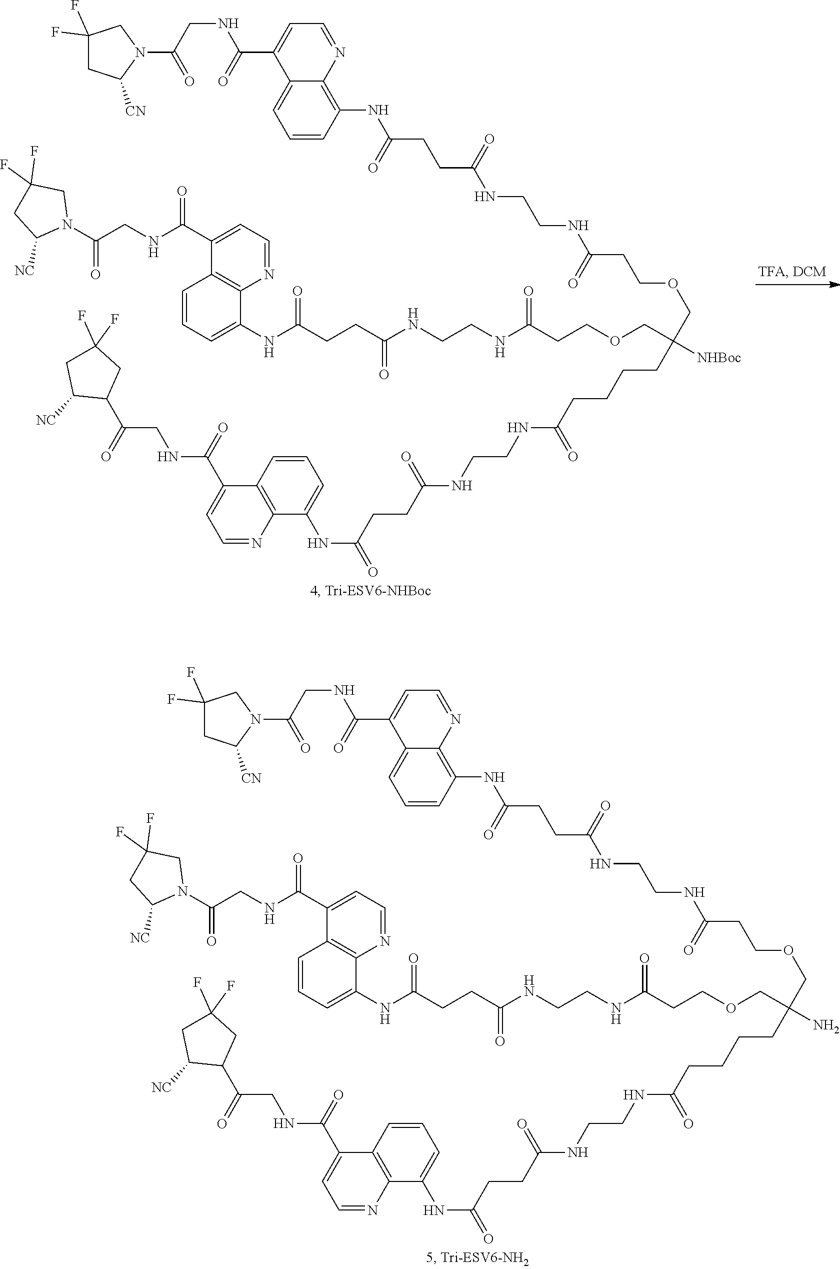 [SMILES: CC(C)(C)OC(=O)NC(CCCCC(=O)NCCNC(=O)CCC(=O)Nc1cccc2c(C(=O)NCC(=O)C3CC(F)(F)C[C@H]3C#N)ccnc12)(COCCC(=O)NCCNC(=O)CCC(=O)Nc1cccc2c(C(=O)NCC(=O)N3CC(F)(F)C[C@H]3C#N)ccnc12)COCCC(=O)NCCNC(=O)CCC(=O)Nc1cccc2c(C(=O)NCC(=O)N3CC(F)(F)C[C@H]3C#N)ccnc12 ; N#C[C@@H]1CC(F)(F)CC1C(=O)CNC(=O)c1ccnc2c(NC(=O)CCC(=O)NCCNC(=O)CCCCC(N)(COCCC(=O)NCCNC(=O)CCC(=O)Nc3cccc4c(C(=O)NCC(=O)N5CC(F)(F)C[C@H]5C#N)ccnc34)COCCC(=O)NCCNC(=O)CCC(=O)Nc3cccc4c(C(=O)NCC(=O)N5CC(F)(F)C[C@H]5C#N)ccnc34)cccc12]

tert-butyl (1,27-bis((4-((2-((S)-2-cyano-4,4-difluoropyrrolidin-1-yl)-2-oxoethyl)carbamoyl)quinolin-8-yl)amino)-14-((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-1,4,9,19,24,27-hexaoxo-12,16-dioxa-5,8,20,23-tetraazaheptacosan-14-yl)carbamate (4, Tri-ESV6-NHBoc, 16 mg, 8.5 μmol) was dissolved in 500 μL of a 30% v/v solution of TFA in DCM and stirred at room temperature for 1 h. The crude was purified via CombiFlash Nextgen 300+ (parameters: flow 15 mL/min, 4 gr C18 column, Water/Acetonitrile+0.1% Formic Acid 98:2 to 0:100 in 30 minutes) and the collected fractions were lyophilized to obtain a white solid (13 mg, 7.2 μmol, 85% yield).

### Step 5a) 2,2′,2″-(10-(19-carboxy-1-((4-((2-((S)-2-cyano-4,4-difluoropyrrolidin-1-yl)-2-oxoethyl)carbamoyl)quinolin-8-yl)amino)-14,14-bis((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-1,4,9,16-tetraoxo-12-oxa-5,8,15-triazanonadecan-19-yl)-1,4,7,10-tetraazacyclododecane-1,4,7-triyl)triacetic acid (6, Tri-ESV6-DOTAGA)

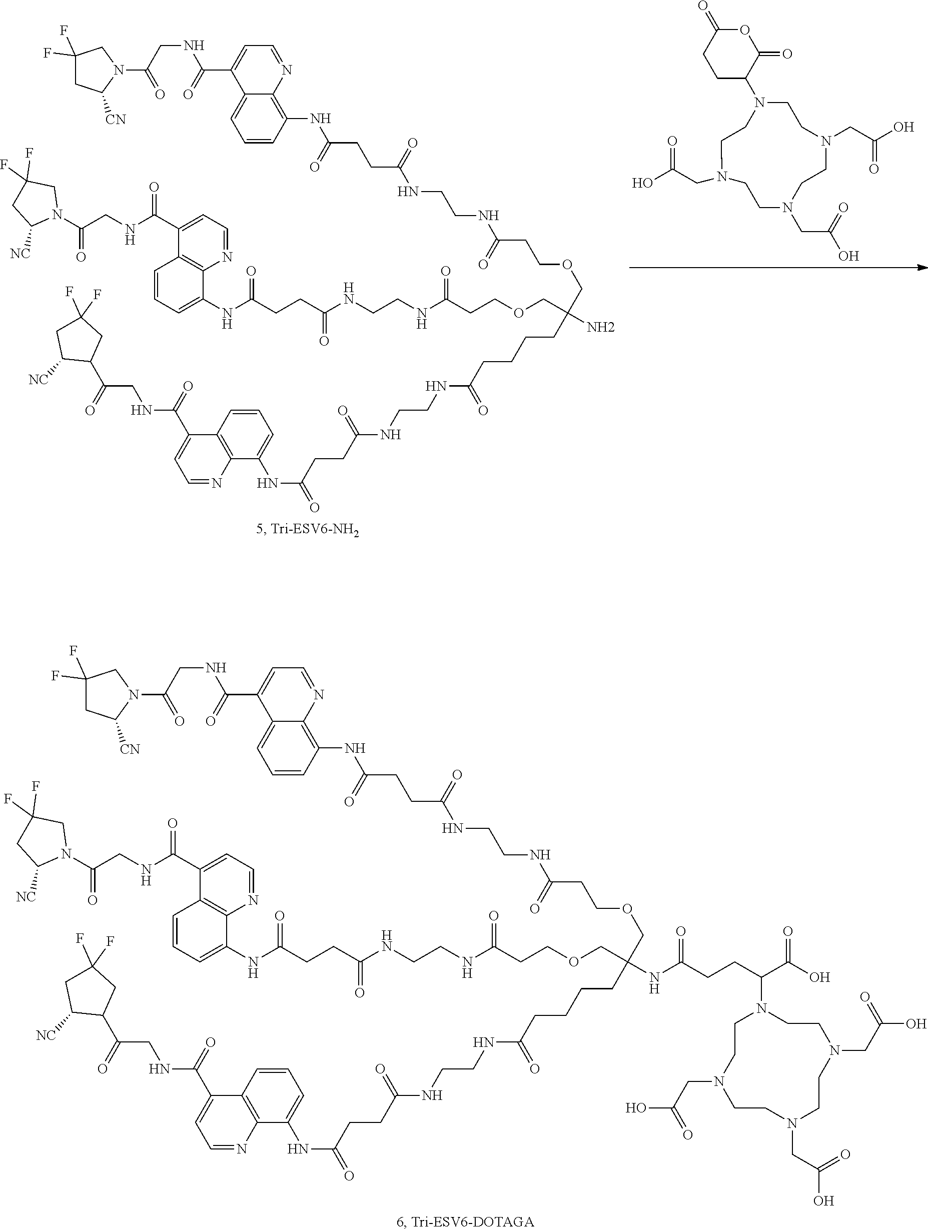

N¹,N^{1′}-(9-amino-9-((3-((2-(4-((4-((2-((S)-2-cyano-4,4-difluoropyrrolidin-1-yl)-2-oxoethyl)carbamoyl) quinolin-8-yl)amino)-4-oxobutanamido)ethyl)amino)-3-oxopropoxy)methyl)-4,14-dioxo-7,11-dioxa-3,15-diazaheptadecane-1,17-diyl)bis(N4-(4-((2-((S)-2-cyano-4,4-difluoropyrrolidin-1-yl)-2-oxoethyl)carbamoyl)quinolin-8-yl) succinamide) (5, Tri-ESV6-NH₂, 13 mg, 7.2 μmol, 1 eq.), 2,2′,2″-(10-(2,6-dioxotetrahydro-2H-pyran-3-yl)-1,4,7,10-tetraazacyclododecane-1,4,7-triyl)triacetic acid (DOTA-GA anhydride, 16 mg, 36 μmol, 5 eq.) and DMAP (5 mg, 36 μmol, 5 eq.) were suspended in dry DMF (500 μL). The resulting mixture was stirred at 50 °C for 6 hours, then diluted with milliQ water (2 mL) and purified via RP-HPLC (linear gradient of ACN/water+0.1% TFA from 90:10 to 0:100). The desired fractions were collected and lyophilized to afford a white solid. (6 mg, 2.7 μmol, 38% yield).

### Step 5b) 2,2′,2″-(10-(17-((4-((2-((S)-2-cyano-4,4-difluoropyrrolidin-1-yl)-2-oxoethyl)carbamoyl)quinolin-8-yl)amino)-4,4-bis((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-2,9,14,17-tetraoxo-6-oxa-3,10,13-triazaheptadecyl)-1,4,7,10-tetraazacyclododecane-1,4,7-triyl)triacetic acid (7, Tri-ESV6-DOTA)

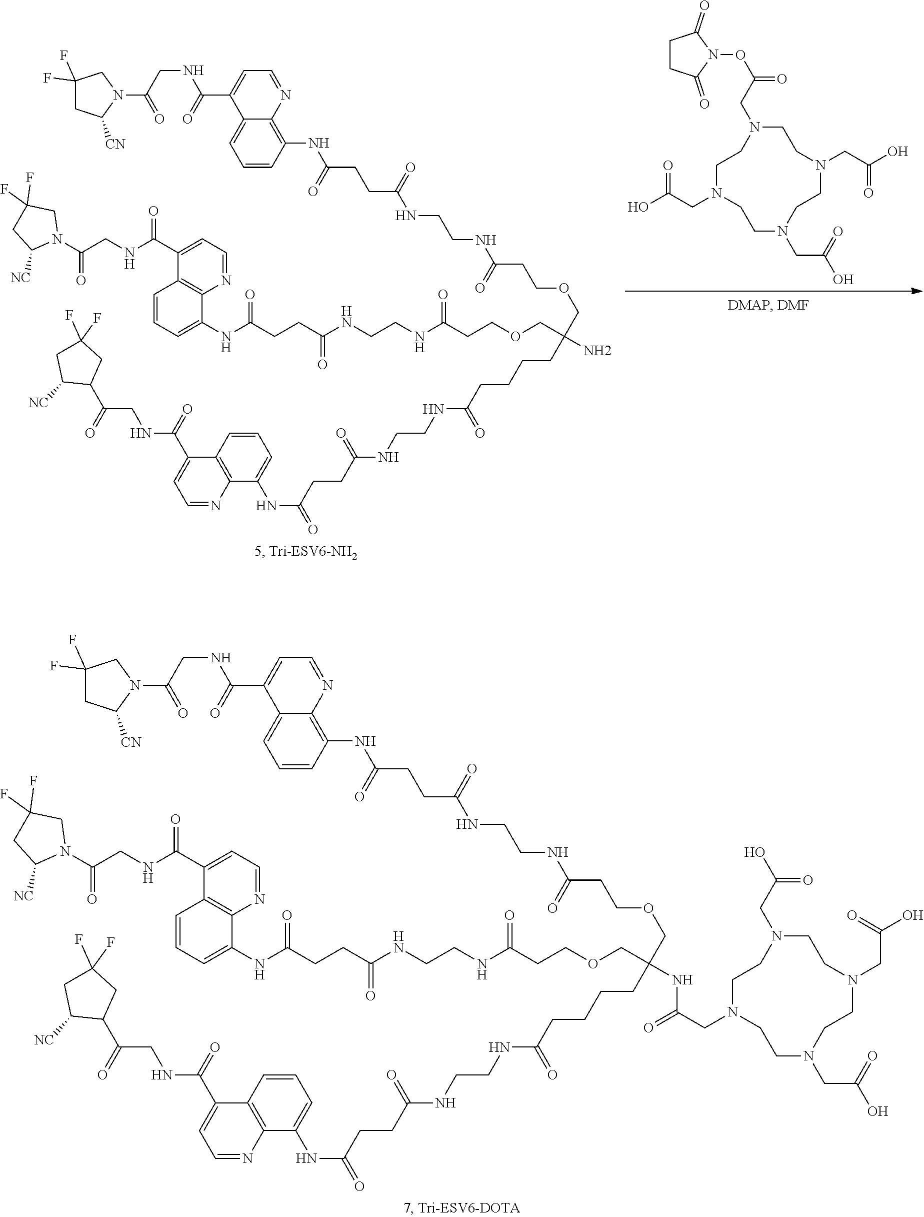

N¹,N^{1′}-(9-amino-9-((3-((2-(4-((4-((2-((S)-2-cyano-4,4-difluoropyrrolidin-1-yl)-2-oxoethyl)carbamoyl)quinolin-8-yl)amino)-4-oxobutanamido)ethyl)amino)-3-oxopropoxy)methyl)-4,14-dioxo-7,11-dioxa-3,15-diazaheptadecane-1,17-diyl)bis(N4-(4-((2-((S)-2-cyano-4,4-difluoropyrrolidin-1-yl)-2-oxoethyl)carbamoyl)quinolin-8-yl) succinamide) (5, Tri-ESV6-NH₂, 13 mg, 7.2 μmol, 1 eq.), 2,2′,2″-(10-(2,6-dioxotetrahydro-2H-pyran-3-yl)-1,4,7,10-tetraazacyclododecane-1,4,7-triyl)triacetic acid (DOTA-GA anhydride, 16 mg, 36 μmol, 5 eq.) and DMAP (5 mg, 36 μmol, 5 eq.) were suspended in dry DMF (500 μL). The resulting mixture was stirred at 50 °C for 6 hours, then diluted with milliQ water (2 mL) and purified via RP-HPLC (linear gradient of ACN/water+0.1% TFA from 90:10 to 0:100). The desired fractions were collected and lyophilized to afford a white solid. (6 mg, 2.7 μmol, 38% yield). 10

### 7. Synthesis of Tri-ESV6-PEG12-DOTAGA

### a) Synthetic Route

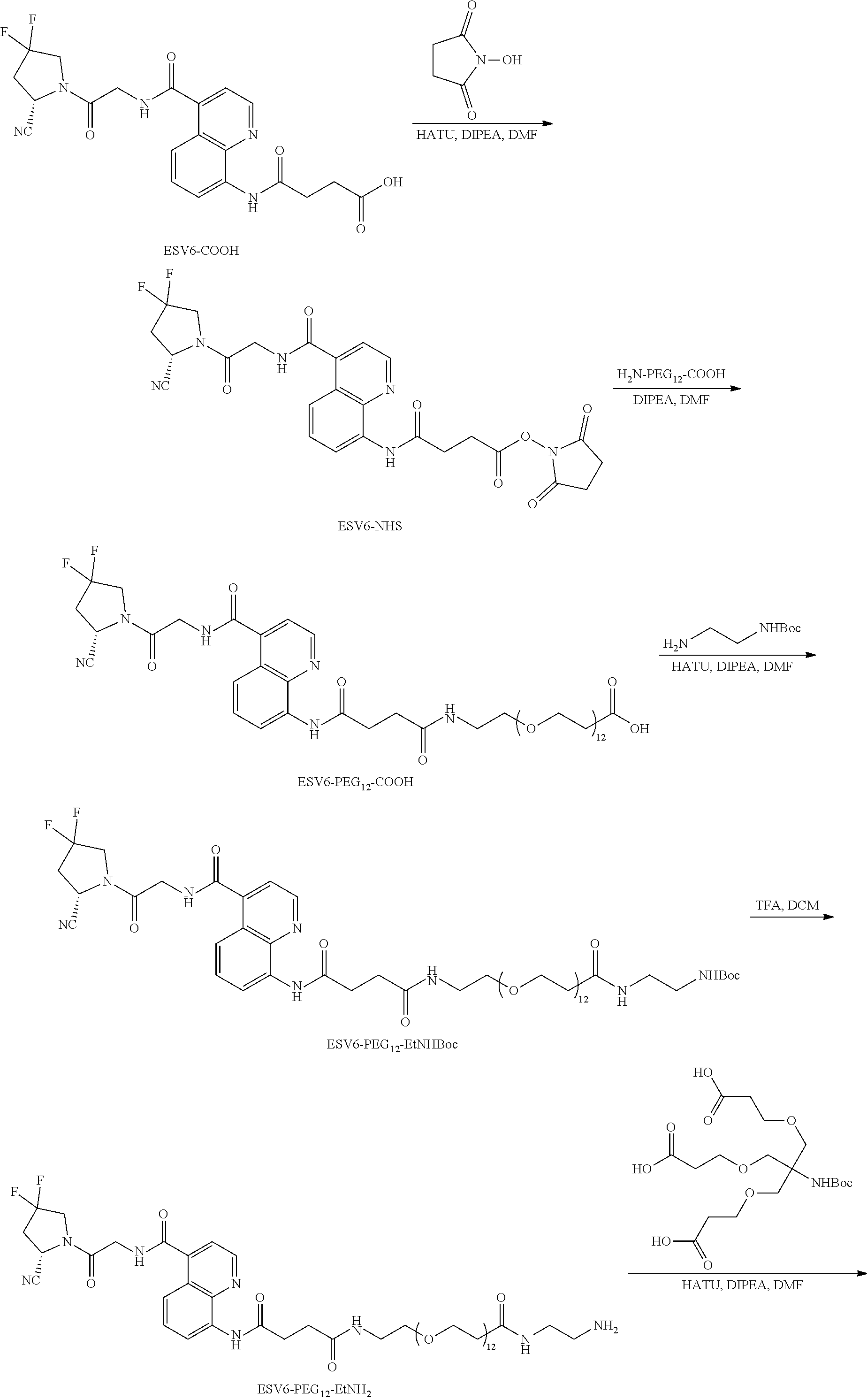

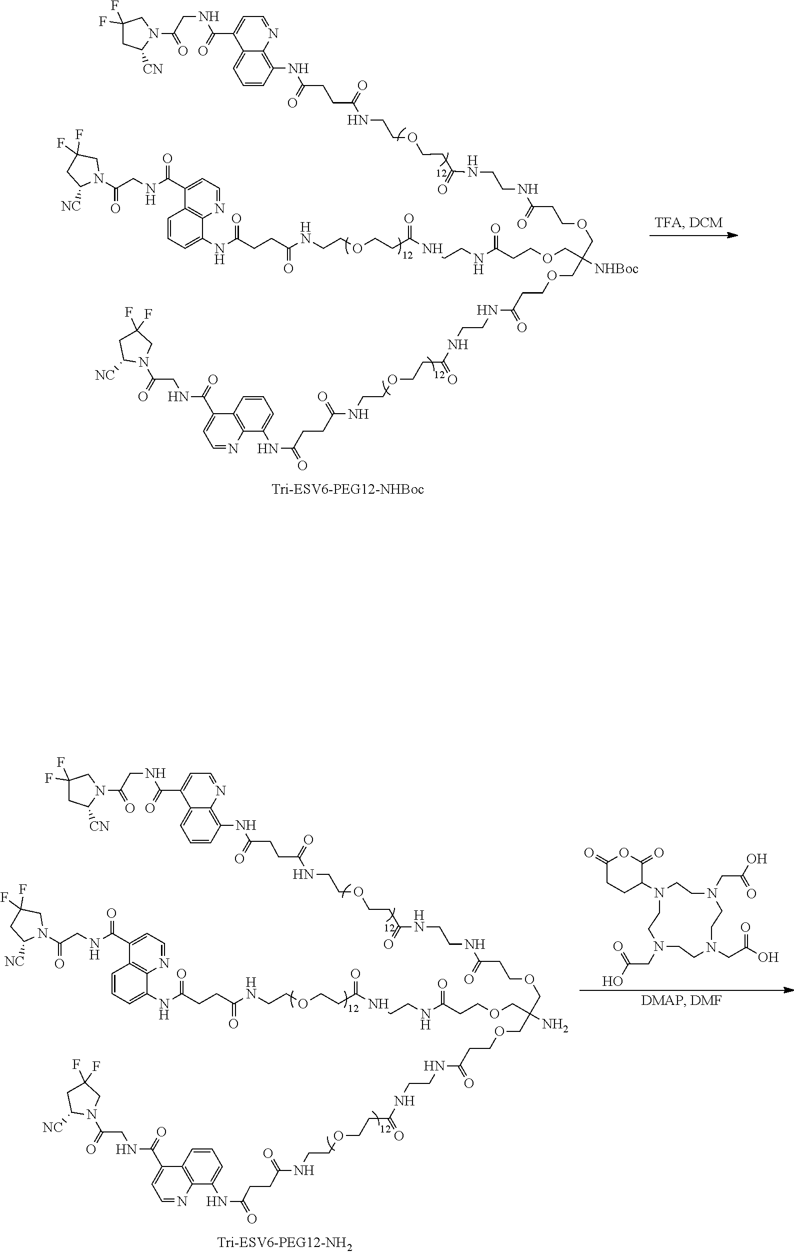

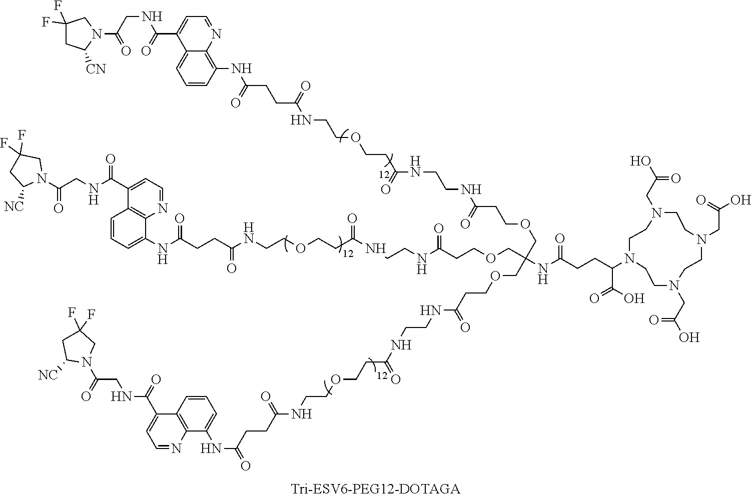

### b) Synthetic Experimental Procedures

### Step 1) 2,5-dioxopyrrolidin-1-yl(S)-4-((4-((2-(2-cyano-4,4-difluoropyrrolidin-1-yl)-2-oxoethyl)carbamoyl)quinolin-8-yl)amino)-4-oxobutanoate

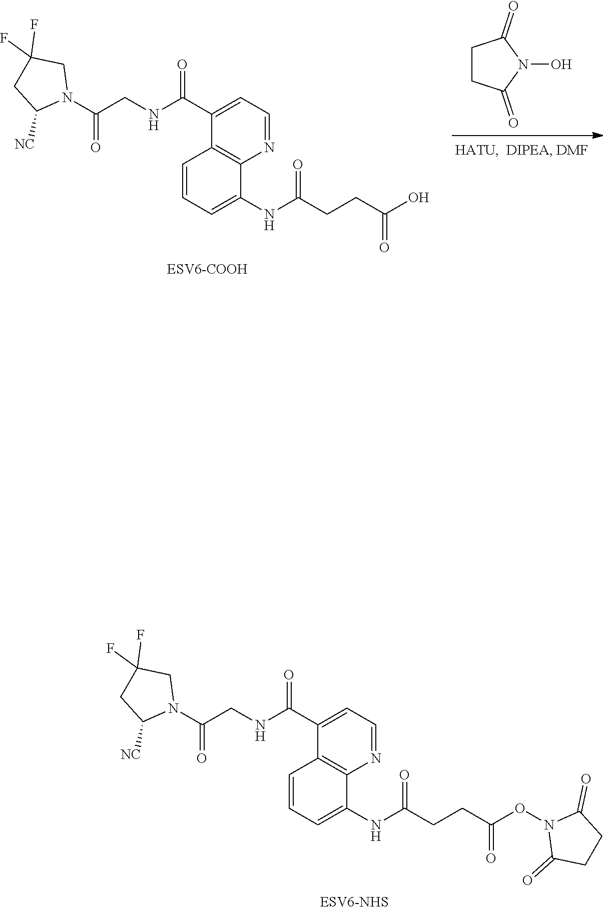 [SMILES: N#C[C@@H]1CC(F)(F)CN1C(=O)CNC(=O)c1ccnc2c(NC(=O)CCC(=O)O)cccc12 ; O=C1CCC(=O)N1O ; N#C[C@@H]1CC(F)(F)CN1C(=O)CNC(=O)c1ccnc2c(NC(=O)CCC(=O)ON3C(=O)CCC3=O)cccc12]

(S)-4-((4-((2-(2-cyano-4,4-difluoropyrrolidin-1-yl)-2-oxoethyl)carbamoyl)quinolin-8-yl)amino)-4-oxobutanoic acid (ESV6-COOH), 1, 100 mg, 217 μmol, 1 eq.), 1-hydroxypyrrolidine-2,5-dione (25 mg, 217 μmol, 1 eq.) and HATU (100 mg, 261 μmol, 1.2 eq.) were suspended in 1.0 mL of DMF. DIPEA (150 μL, 868 μmol, 4 eq.) was added dropwise and the reaction was stirred for 15 minutes. The crude was purified via CombiFlash Nextgen 300+ (parameters: flow 30 mL/min, 24 gr C18 column, Water/Acetonitrile+0.1% Formic Acid 98:2 to 0:100 in 30 minutes) and the collected fractions were lyophilized to obtain a white solid (90 mg, 162 μmol, 75% yield).

### Step 2) (S)-44-((4-((2-(2-cyano-4,4-difluoropyrrolidin-1-yl)-2-oxoethyl)carbamoyl)quinolin-8-yl)amino)-41,44-dioxo-4,7,10,13,16,19,22,25,28,31,34,37-dodecaoxa-40-azatetratetracontanoic acid

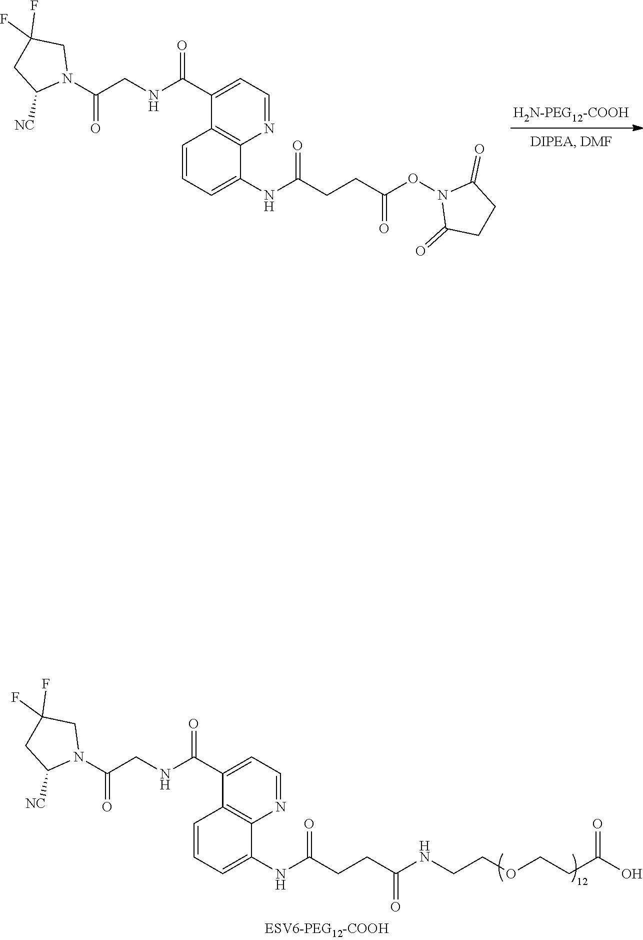

2,5-dioxopyrrolidin-1-yl(S)-4-((4-((2-(2-cyano-4,4-difluoropyrrolidin-1-yl)-2-oxoethyl)carbamoyl) quinolin-8-yl)amino)-4-oxobutanoate (ESV6-NHS, 90 mg, 162 μmol) and H₂N-PEG12-COOH) (CAS: 1415408-69-3, 60 mg, 97 μmol) and were suspended in 1.0 mL of DMF. DIPEA (200 μL) was added dropwise and the reaction was stirred for 15 minutes. The crude was purified via CombiFlash Nextgen 300+ (parameters: flow 30 mL/min, 24 gr C18 column, Water/Acetonitrile+0.1% Formic Acid 98:2 to 0:100 in 30 minutes) and the collected fractions were lyophilized to obtain a yellow oil (100 mg, 94 μmol, 97% yield).

### Step 3) tert-butyl(S)-(47-((4-((2-(2-cyano-4,4-difluoropyrrolidin-1-yl)-2-oxoethyl)carbamoyl)quinolin-8-yl)amino)-4,44,47-trioxo-7,10,13,16,19,22,25,28,31,34,37,40-dodecaoxa-3,43-diazaheptatetracontyl)carbamate

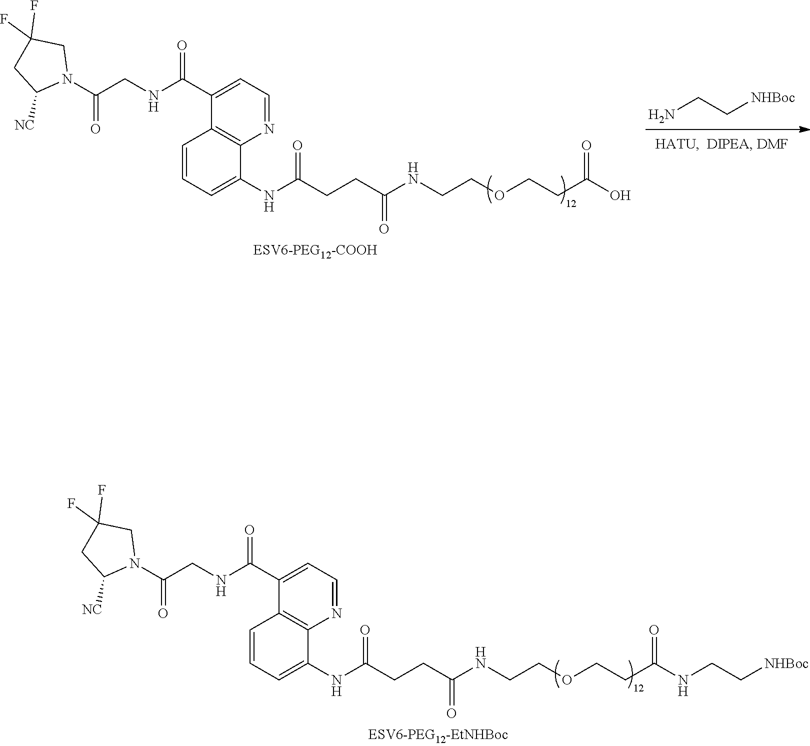

(S)-44-((4-((2-(2-cyano-4,4-difluoropyrrolidin-1-yl)-2-oxoethyl)carbamoyl)quinolin-8-yl)amino)-41,44-dioxo-4,7,10,13,16,19,22,25,28,31,34,37-dodecaoxa-40-azatetratetracontanoic acid (ESV6-PEG12-COOH, 100 mg, 94 μmol), tert-butyl(2-aminoethyl)carbamate (24 mg, 113 μmol, 1.2 eq.) and HATU (57 mg, 148 μmol, 1.5 eq.) were suspended in 2.0 mL of DMF. DIPEA (75 μL) was added dropwise and the reaction was stirred for 15 minutes. The crude was purified via CombiFlash Nextgen 300+ (parameters: flow 30 mL/min, 24 gr C18 column, Water/Acetonitrile+0.1% Formic Acid 98:2 to 0:100 in 30 minutes) and the collected fractions were lyophilized to obtain a yellow oil (95 mg, 79 μmol, 84% yield).

### Step 4) (S)—N¹-(42-amino-39-oxo-3,6,9,12,15,18,21,24,27,30,33,36-dodecaoxa-40-azadotetracontyl)-N4-(4-((2-(2-cyano-4,4-difluoropyrrolidin-1-yl)-2-oxoethyl)carbamoyl)quinolin-8-yl) succinamide

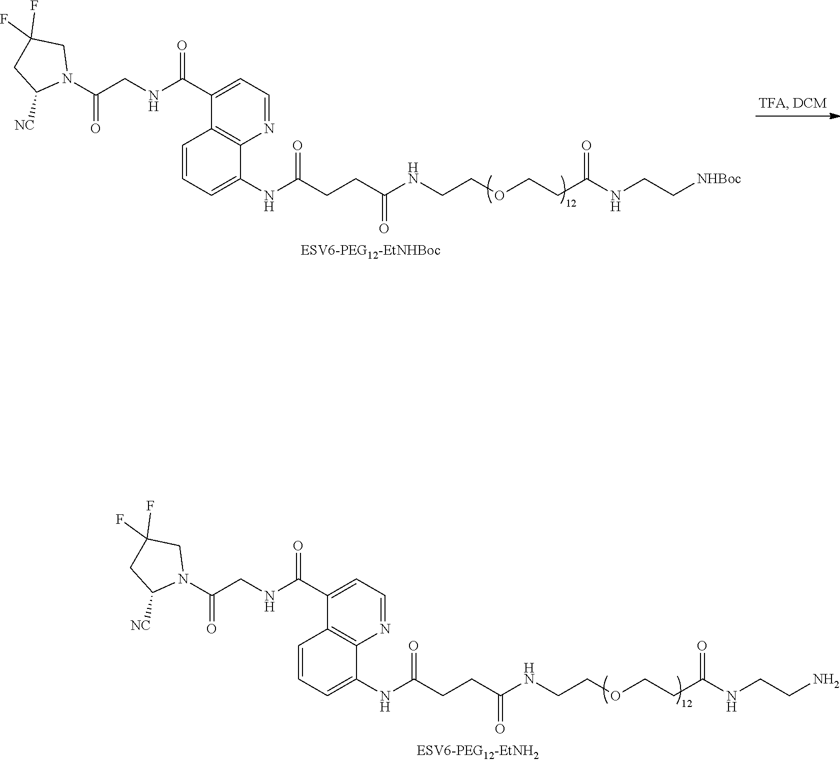

tert-butyl(S)-(47-((4-((2-(2-cyano-4,4-difluoropyrrolidin-1-yl)-2-oxoethyl)carbamoyl)quinolin-8-yl)amino)-4,44,47-trioxo-7,10,13,16,19,22,25,28,31,34,37,40-dodecaoxa-3,43-diazaheptatetracontyl)carbamate (95 mg, 79 μmol) was dissolved in 2 mL of a 50% v/v solution of TFA in DCM and stirred at room temperature for 1 h. The crude was purified via CombiFlash Nextgen 300+(parameters: flow 30 mL/min, 24 gr C18 column, Water/Acetonitrile+0.1% Formic Acid 98:2 to 0:100 in 30 minutes) and the collected fractions were lyophilized to obtain a yellow oil (67 mg, 61 μmol, 64% yield).

### Step 5) tert-butyl (1,107-bis((4-((2-((S)-2-cyano-4,4-difluoropyrrolidin-1-yl)-2-oxoethyl)carbamoyl)quinolin-8-yl)amino)-54-(53-((4-((2-((S)-2-cyano-4,4-difluoropyrrolidin-1-yl)-2-oxoethyl)carbamoyl)quinolin-8-yl)amino)-5,10,50,53-tetraoxo-2,13,16,19,22,25,28,31,34,37,40,43,46-tridecaoxa-6,9,49-triazatripentacontyl)-1,4,44,49,59,64,104,107-octaoxo-8,11,14,17,20,23,26,29,32,35,38,41,52,56,67,70,73,76,79,82,85,88,91,94,97,100-hexacosaoxa-5,45,48,60,63,103-hexaazaheptahectan-54-yl)carbamate

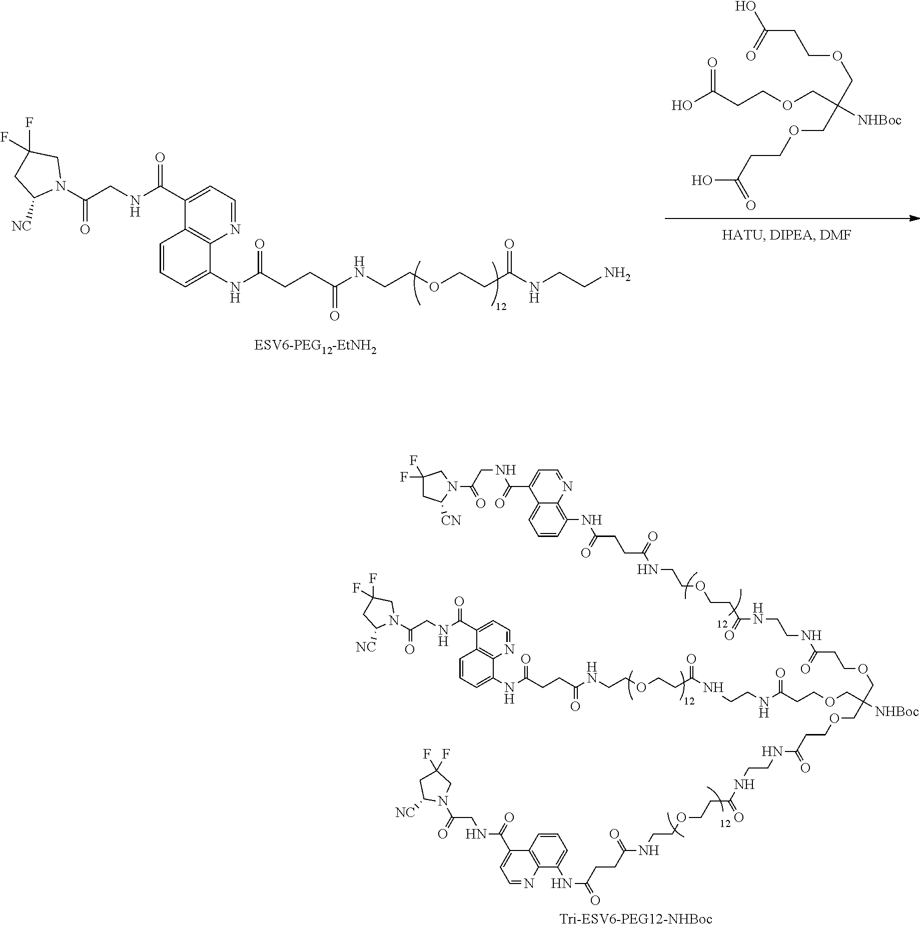

(S)—N¹-(42-amino-39-oxo-3,6,9,12,15,18,21,24,27,30,33,36-dodecaoxa-40-azadotetracontyl)-N⁴-(4-((2-(2-cyano-4,4-difluoropyrrolidin-1-yl)-2-oxoethyl)carbamoyl)quinolin-8-yl) succinamide (ESV6-PEG12-EtNH₂, 60 mg, 54 μmol, 4.5 eq.), 3,3′-((2-((tert-butoxycarbonyl)amino)-2-((2-carboxyethoxy)methyl) propane-1,3-diyl)bis(oxy))dipropionic acid (5.3 mg, 12 μmol, 1 eq.) and HATU (21 mg, 54 μmol, 4.5 eq.) were suspended in 0.5 mL of DMF. DIPEA (40 μL, 260 μmol, 10 eq.) was added to the mixture and the reaction was stirred for 4 h at room temperature. The crude was purified via CombiFlash Nextgen 300+ (parameters: flow 15 mL/min, 12 gr C18 column, Water/Acetonitrile+0.1% Formic Acid 98:2 to 0:100 in 30 minutes) and the collected fractions were lyophilized to obtain a yellow oil (35 mg, 9.4 μmol, 78% yield).

### Step 6) N¹,N^{1′}-(49-amino-49-(53-((4-((2-((S)-2-cyano-4,4-difluoropyrrolidin-1-yl)-2-oxoethyl)carbamoyl)quinolin-8-yl)amino)-5,10,50,53-tetraoxo-2,13,16,19,22,25,28,31,34,37,40,43,46-tridecaoxa-6,9,49-triazatripentacontyl)-39,44,54,59-tetraoxo-3,6,9,12,15,18,21,24,27,30,33,36,47,51,62,65,68,71,74,77,80,83,86,89,92,95-hexacosaoxa-40,43,55,58-tetraazaheptanonacontane-1,97-diyl)bis(N4-(4-((2-((5)-2-cyano-4,4-difluoropyrrolidin-1-yl)-2-oxoethyl)carbamoyl)quinolin-8-yl)succinamide)

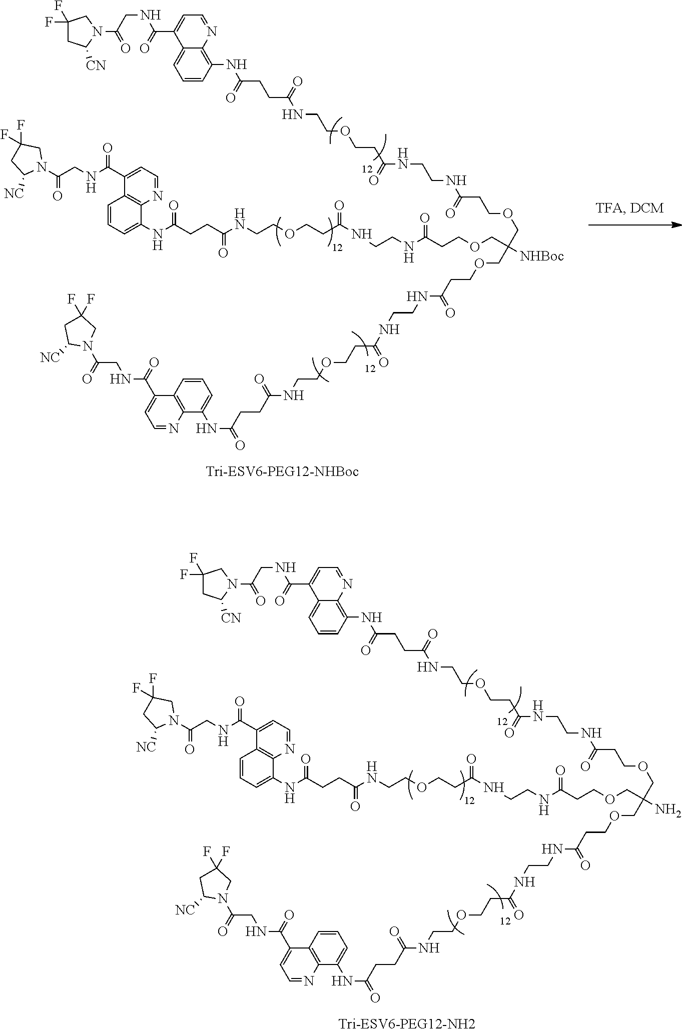

tert-butyl (1,107-bis((4-((2-((S)-2-cyano-4,4-difluoropyrrolidin-1-yl)-2-oxoethyl)carbamoyl)quinolin-8-yl)amino)-54-(53-((4-((2-((S)-2-cyano-4,4-difluoropyrrolidin-1-yl)-2-oxoethyl)carbamoyl)quinolin-8-yl)amino)-5,10,50,53-tetraoxo-2,13,16,19,22,25,28,31,34,37,40,43,46-tridecaoxa-6,9,49-triazatripentacontyl)-1,4,44,49,59,64, 104,107-octaoxo-8,11,14,17,20,23,26,29,32,35,38,41,52,56,67,70,73,76,79,82,85,88,91,94,97,100-hexacosaoxa-5,45,48,60,63,103-hexaazaheptahectan-54-yl)carbamate (Tri-ESV6-PEG12-NHBoc, 30 mg, 8.1 μmol) was dissolved in 500 μL of a 50% v/v solution of TFA in DCM and stirred at room temperature for 1 h. The crude was purified via CombiFlash Nextgen 300+ (parameters: flow 15 mL/min, 4 gr C18 column, Water/Acetonitrile+0.1% Formic Acid 98:2 to 0:100 in 30 minutes) and the collected fractions were lyophilized to obtain a white solid (15 mg, 4.2 μmol, 52% yield).

### Step 7) 2,2′,2″-(10-(1-carboxy-26-((4-((2-((S)-2-cyano-4,4-difluoropyrrolidin-1-yl)-2-oxoethyl)carbamoyl)quinolin-8-yl)amino)-6,6-bis(20-((4-((2-((S)-2-cyano-4,4-difluoropyrrolidin-1-yl)-2-oxoethyl)carbamoyl)quinolin-8-yl)amino)-5,10,17,20-tetraoxo-2,13-dioxa-6,9,16-triazaicosyl)-4,11,16,23,26-pentaoxo-8,19-dioxa-5,12,15,22-tetraazahexacosyl)-1,4,7,10-tetraazacyclododecane-1,4,7-triyl)triacetic acid (Tri-ESV6-PEG12-DOTAGA)

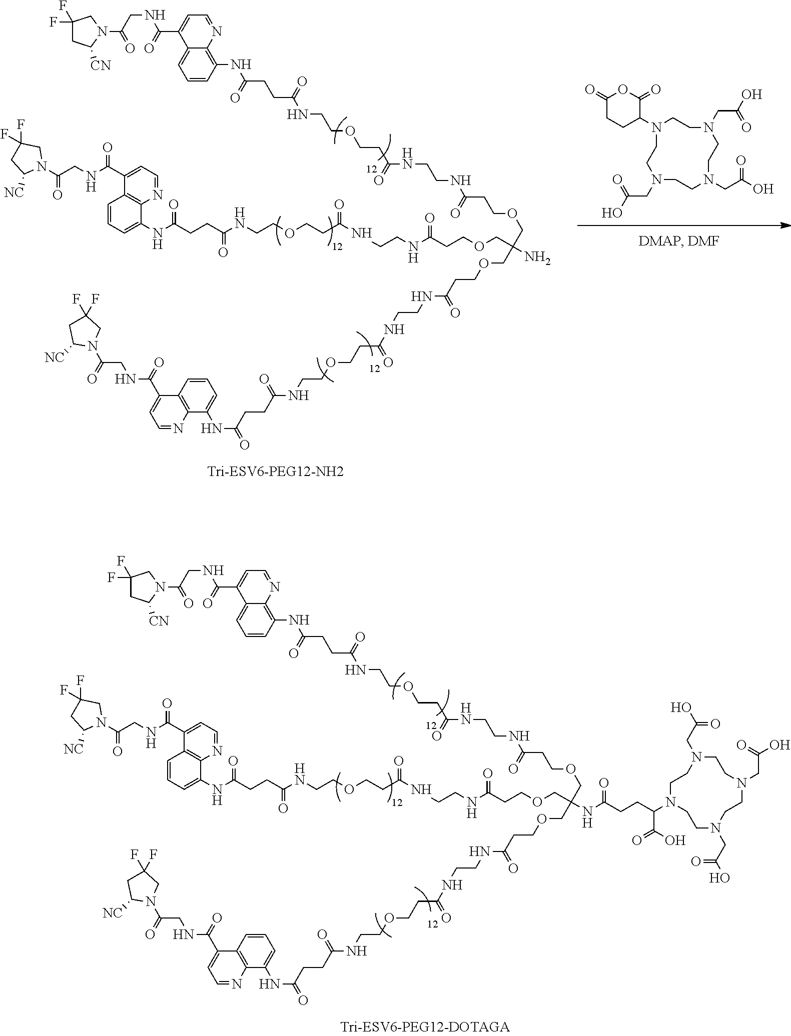

N¹,N^{1′}-(49-amino-49-(53-((4-((2-((S)-2-cyano-4,4-difluoropyrrolidin-1-yl)-2-oxoethyl)carbamoyl)quinolin-8-yl)amino)-5,10,50,53-tetraoxo-2,13,16,19,22,25,28,31,34,37,40,43,46-tridecaoxa-6,9,49-triazatripentacontyl)-39,44,54,59-tetraoxo-3,6,9,12,15,18,21,24,27,30,33,36,47,51,62,65,68,71,74,77,80,83,86,89,92,95-hexacosaoxa-40,43,55,58-tetraazaheptanonacontane-1,97-diyl)bis(N4-(4-((2-((S)-2-cyano-4,4-difluoropyrrolidin-1-yl)-2-oxoethyl)carbamoyl)quinolin-8-yl) succinamide) (Tri-ESV6-PEG12-NH₂, 10 mg, 2.8 μmol, 1 eq.), 2,2′,2″-(10-(2,6-dioxotetrahydro-2H-pyran-3-yl)-1,4,7,10-tetraazacyclododecane-1,4,7-triyl)triacetic acid (DOTA-GA anhydride, 6.5 mg, 14 μmol, 5 eq.) and DMAP (2 mg, 14 μmol, 5 eq.) were suspended in dry DMF (500 μL). The resulting mixture was stirred at 50 °C for 6 hours, then diluted with milliQ water (2 mL) and purified via RP-HPLC (linear gradient of ACN/water+0.1% TFA from 90:10 to 0:100). The desired fractions were collected and lyophilized to afford a white solid. (7 mg, 1.6 μmol, 56% yield).

### 8. Chemical Structures and Synthesis of Multivalent ESV6-DOTAGA Compounds

### a) Chemical Structure of Tetra-ESV6-DOTAGA (10)

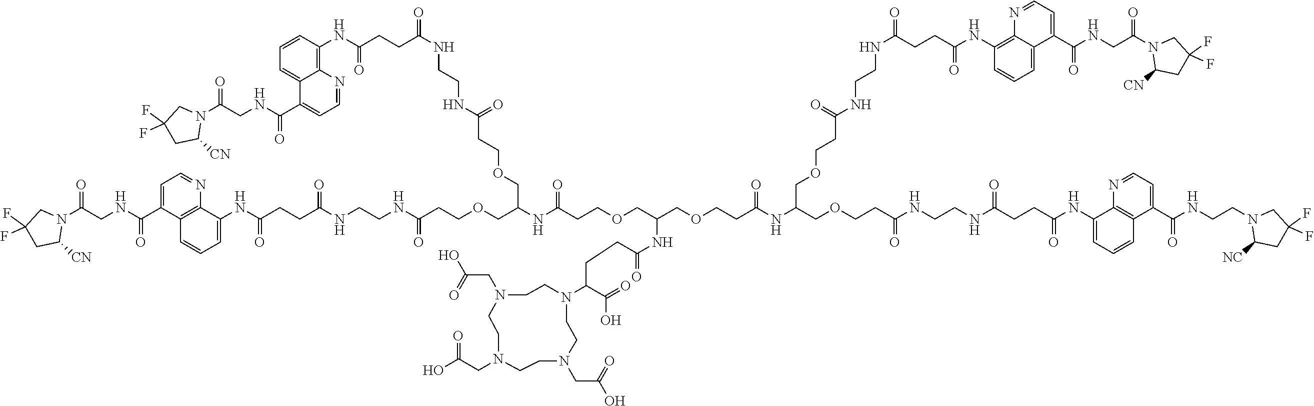

Molecular weight: 3062.0652 Da
Exact mass: 3060.2291 Da
Formula: C138H169F8N35037
SMILES: O═C(NCCNC(CCOCC(NC(CCOCC(NC(CCC(NICCN(CC(O)═O)CCN(CC(O)═O)CCN(CC(O)═O)CC1)C(O)═O)═O)COCCC(NC(COCCC(NCCNC(CCC(NC2=C(N═CC═C3C(NCC(N4CC(F)(F)C[C@H]4C #N)═O)═O)C3=CC═C2)=O)═O)═O)COCCC(NCCNC(CCC(NC5=CC═CC6=C5N═CC═C6C(NCC(N7[C@H](C#N)CC(F)(F)C7)=O)═O)═O)═O)═O)═O)═O)COCCC(NCCNC(CCC(NC8=C(N═CC═C9C(NCC(N % 10CC(F)(F)C[C@H] % 10C #N)═O)═O)C9=CC═C8)=O)═O)═O)═O)CCC(NC % 11=CC═CC % 12=C % 11N═CC═C % 12C(NCC(N %13 [C@H](C#N)CC(F)(F)C % 13)=O)═O)═O

### b) Chemical Structure of Hexa-ESV6-DOTAGA

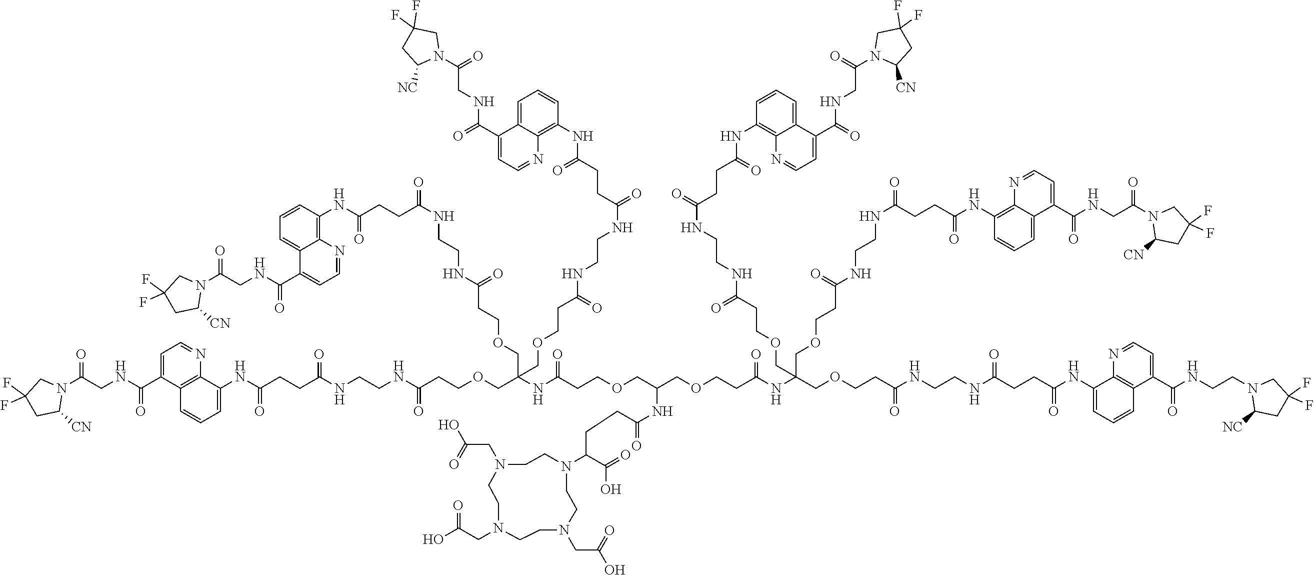

Molecular weight: 4233.2028 Da
Exact mass: 4230.6586 Da
Formula: C192H227F12N49049
SMILES: O═C(NCCNC(CCOCC(NC(CCOCC(NC(CCC(NICCN(CC(O)═O)CCN(CC(O)═O)CCN(CC(O)═O)CC1)C(O)═O)═O)COCCC(NC(COCCC(NCCNC(CCC(NC2=C(N═CC═C3C(NCC(N4CC(F)(F)C[C@H]4C #N)═O)═O)C3=CC═C2)=O)═O)═O)(COCCC(NCCNC(CCC(NC5=C(N═CC═C6C(NCC(N7CC(F)(F)C[C@H]7C #N)═O)═O)C6=CC═C 5)=O)═O)═O)COCCC(NCCNC(CCC(NC8=CC═CC9=C8N═CC═C9C(NCC(N % 10 [C@H](C#N)CC(F)(F)C % 10)=O)═O)═O)═O)═O)═O)═O) (COCCC(NCCNC(CCC(NC % 11=C(N═CC═C % 12C(NCC(N %13CC(F)(F)C[C@H] % 13C #N)═O)═O)C % 12=CC═C % 11)=O)═O)═O)COCCC(NCCNC(CCC(NC % 14=C(N═CC═C % 15C(NCC(N %16CC(F) (F)C[C@H] % 16C #N)═O)═O)C % 15=CC═C % 14)=O)═O)═O)═O)CCC(NC % 17=CC═CC % 18=C % 17N═CC—C % 18C(NCC(N % 19[C@H](C#N)CC(F)(F)C % 19)=O)═O)═O

### c) Chemical Structure of Octa-ESV6-DOTAGA

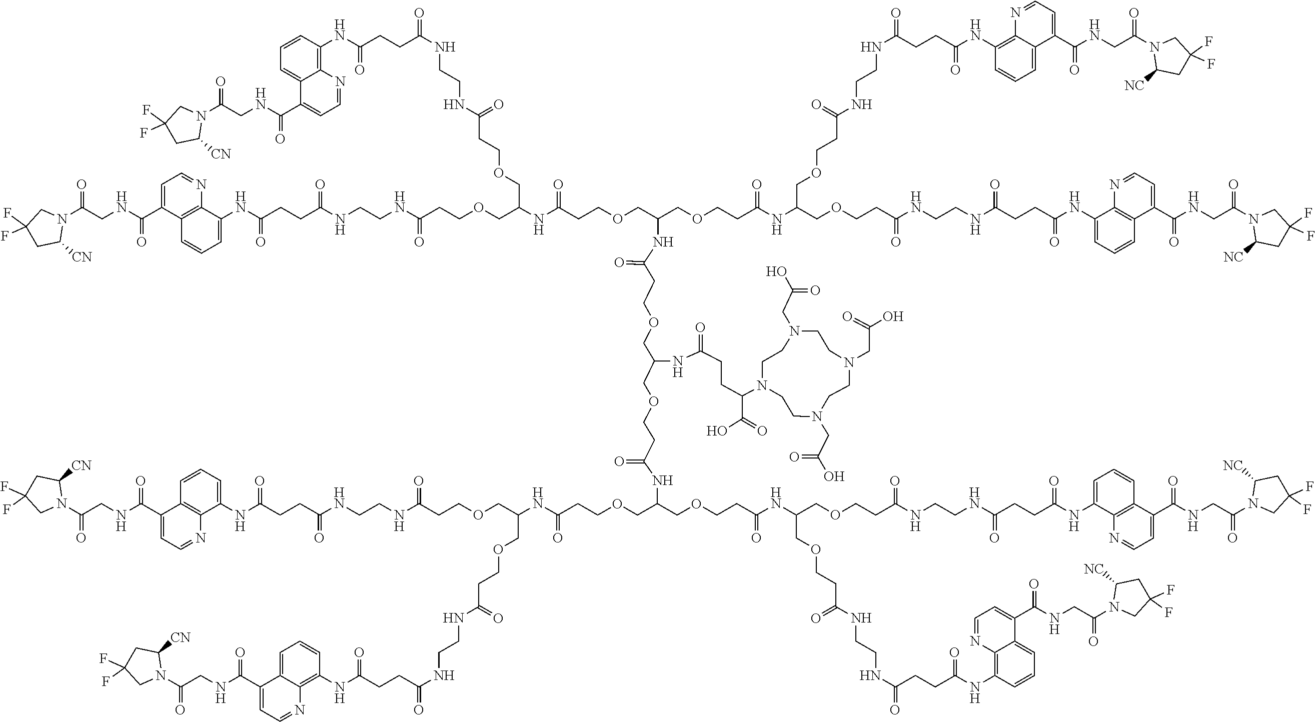

Molecular weight: 5864.8685 Da
Exact mass: 5861.3414 Da
Formula: C266H321F16N67069
SMILES: O═C(NCCNC(CCOCC(NC(CCOCC(NC(CCOCC(NC(CCC(NICCN(CC(O)═O)CCN(CC(O)═O)CCN(CC(O)═O) CC1)C(O)═O)═O)COCCC(NC(COCCC(NC(COCCC(NCCNC(CCC(NC2=C(N═CC═C3C(NCC(N4CC(F)(F)C[C @H]4C #N)═O)═O)C3=CC═C2)=O)═O)═O)COCCC(NCCNC(CCC(NC5=CC═CC6=C5N═CC═C6C(NCC(N7 [C@) H](C#N)CC(F)(F)C7)=O)═O)═O)═O)═O)═O)COCCC(NC(COCCC(NCCNC(CCC(NC8=C(N═CC═C9C(NCC(N % 10CC(F)(F)C[C@H] % 10C #N)═O)═O)C9=CC═C8)=O)═O)═O)COCCC(NCCNC(CCC(NC % 11=CC═CC % 12=C % 11N═CC═C % 12C(NCC(N %13 [C@H](C#N)CC(F)(F)C % 13)=O)═O)═O)═O)═O)═O)═O)═O)COCCC(NC(COCCC(NCCNC(CCC(NC % 14=C(N═CC═C % 15C(NCC(N %16CC(F)(F)C[C@H] % 16C #N)═O)═O)C % 15=CC═C % 14)=O)═O)═O)COCCC(NCCNC(CCC(NC % 17=CC═CC % 18=C % 17N═CC═C % 18C(NCC(N %19 [C@H](C#N)CC(F)(F) C % 19)=O)═O)═O)═O)═O)═O)═O)COCCC(NCCNC(CCC(NC % 20=C(N═CC═C % 21C(NCC(N %22CC(F)(F)C[C @H] % 22C #N)═O)═O)C % 21=CC═C % 20)=O)═O)═O)═O)CCC(NC % 23=CC═CC % 24=C % 23N═CC═C % 24C(NCC(N % 25 [C@H](C#N)CC(F)(F)C % 25)=O)═O)═O

### d) Synthetic Route for the Synthesis of Tetra-ESV6-DOTAGA and Octa-ESV6-DOTAGA

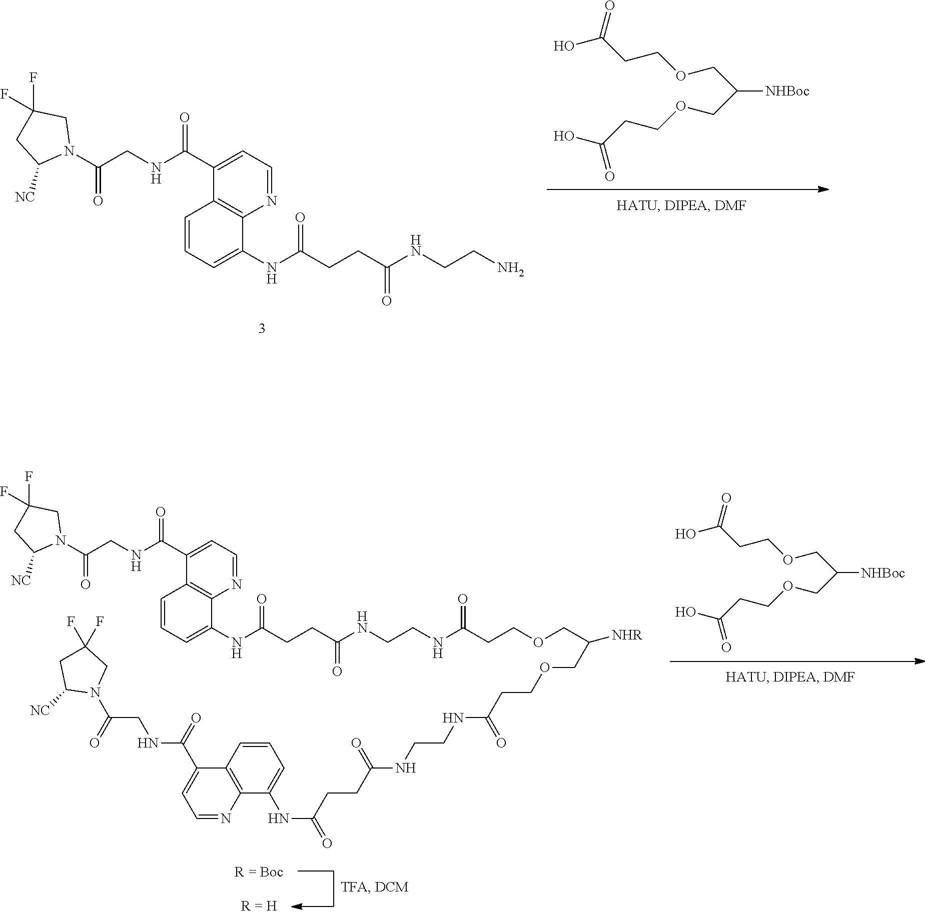

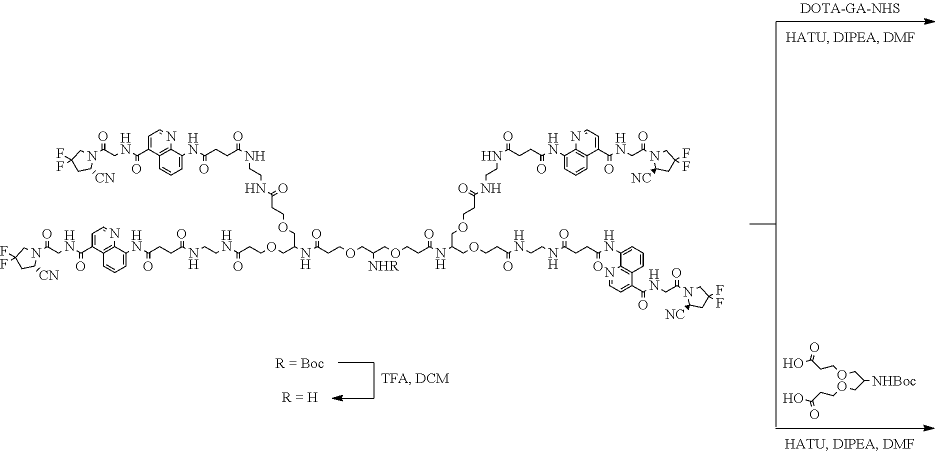

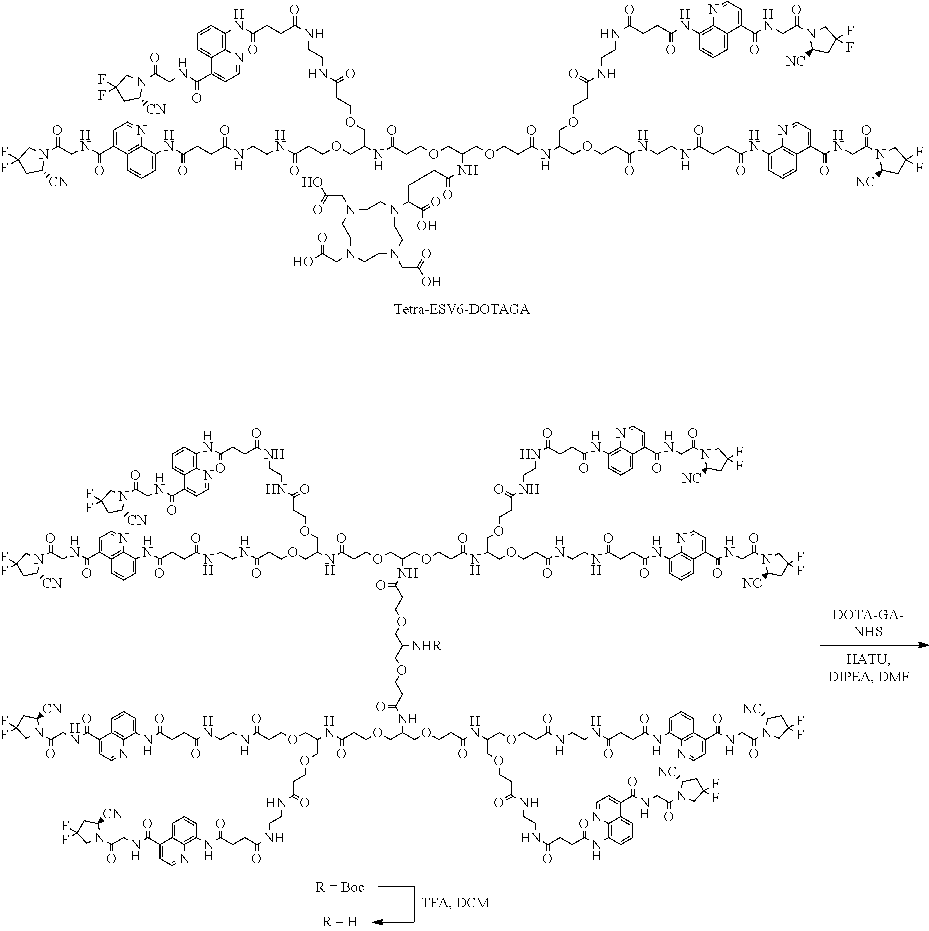

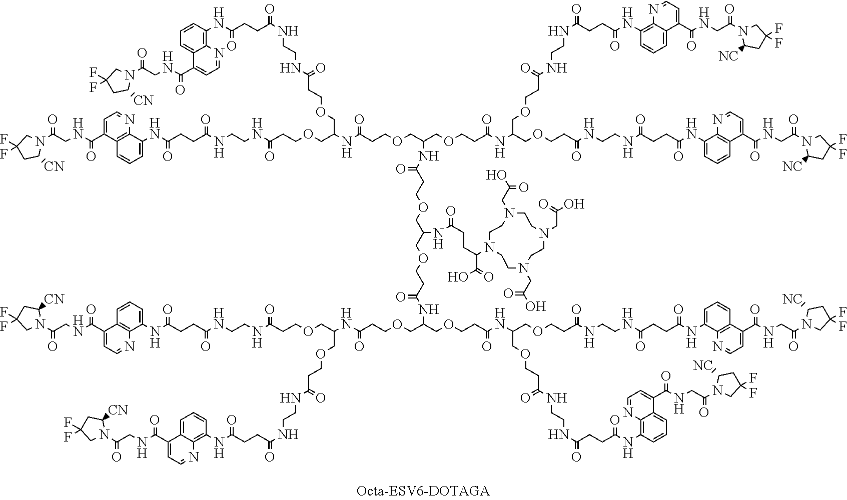

### e) Synthetic Route for the Synthesis of Hexa-ESV6-DOTAGA

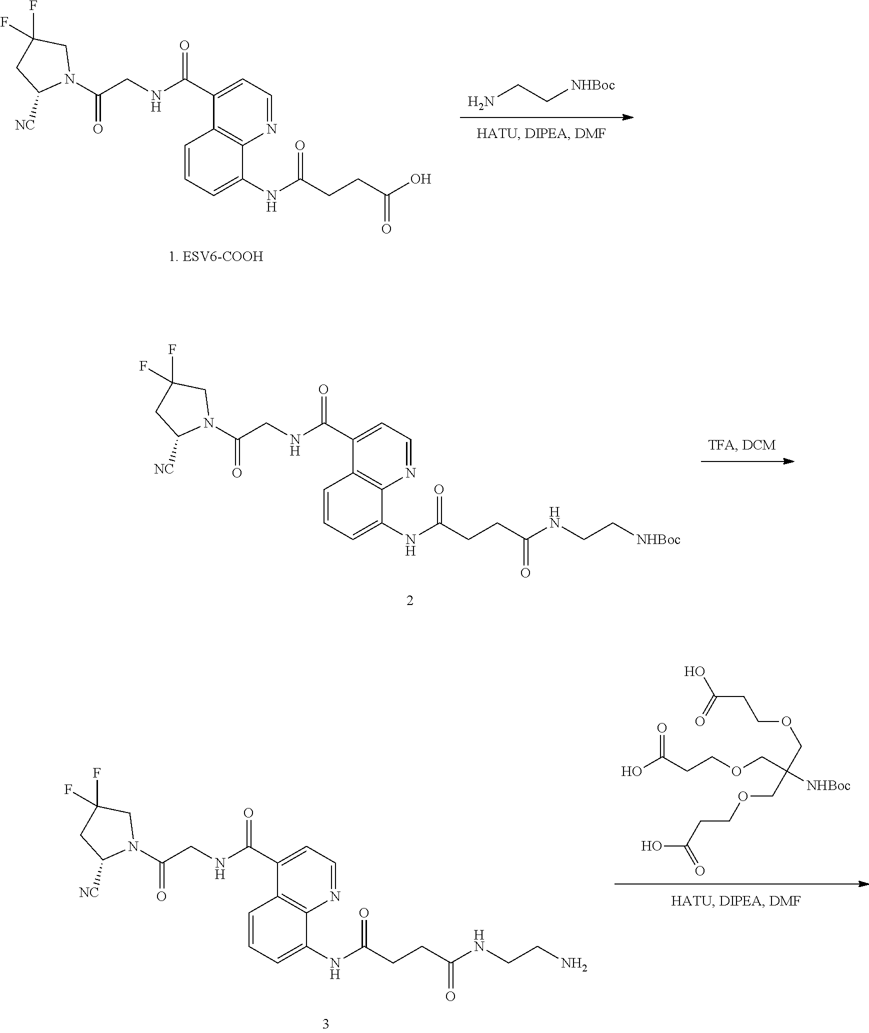 [SMILES: N#C[C@@H]1CC(F)(F)CN1C(=O)CNC(=O)c1ccnc2c(NC(=O)CCC(=O)O)cccc12 ; CC(C)(C)OC(=O)NCCN ; CC(C)(C)OC(=O)NCCNC(=O)CCC(=O)Nc1cccc2c(C(=O)NCC(=O)N3CC(F)(F)C[C@H]3C#N)ccnc12 ; N#C[C@@H]1CC(F)(F)CN1C(=O)CNC(=O)c1ccnc2c(NC(=O)CCC(=O)NCCN)cccc12 ; CC(C)(C)OC(=O)NC(COCCC(=O)O)(COCCC(=O)O)COCCC(=O)O]

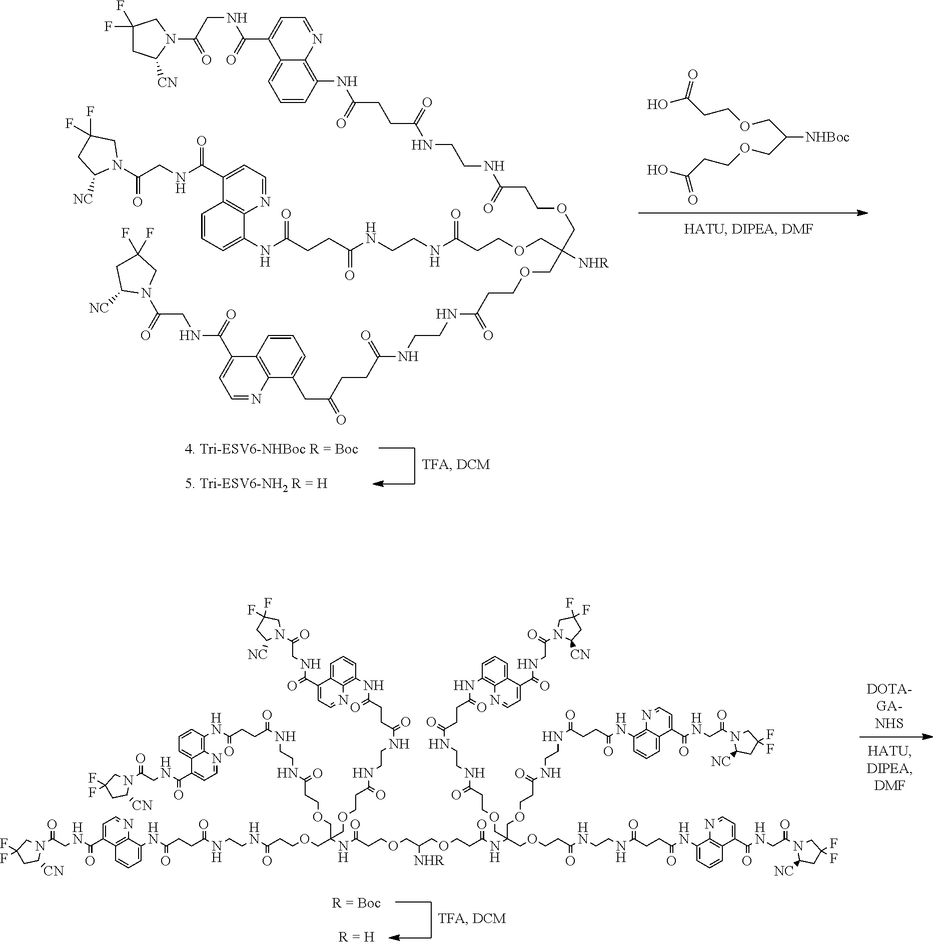

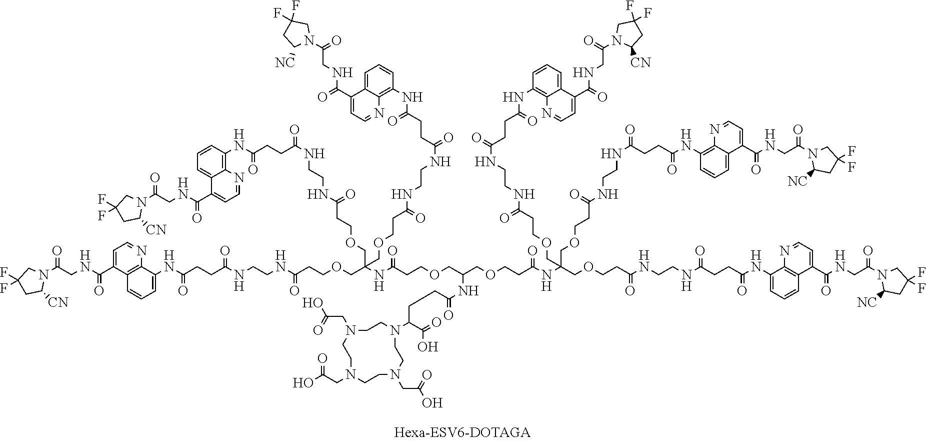

### 9. In Vitro Tests and Assays

### a) Inhibition Assay

Enzymatic activity of hFAP on the Z-Gly-Pro-AMC substrate was measured at room temperature on a microtiter plate reader, monitoring the fluorescence at an excitation wavelength of 360 nm and an emission wavelength of 465 nm. The reaction mixture contained substrate (20 μM), protein (66 μM, constant), assay buffer (50 mM Tris, 100 mM NaCl, and 1 mM EDTA, pH=7.4), and inhibitors with serial dilution from 167 nM to 80 fM, 1:2 in a total volume of 20 μL. Experiments were performed in triplicate, and the mean fluorescence values were fitted using Prism 7. The value is defined as the concentration of inhibitor required to reduce the enzyme activity by 50% after addition of the substrate [FIG. 1 and FIG. 8].

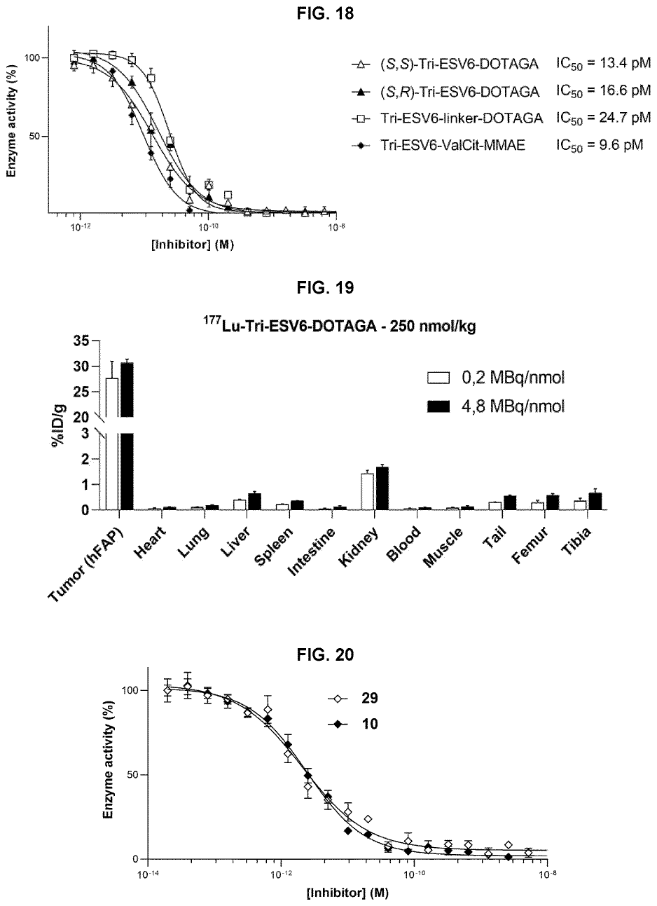

According to the same procedure, further comparative inhibition assays with trimeric binders having inter alia different lengths of linker group B and/or different payload groups C were performed with compounds Tri-ESV6-ValCit-MMAE (14), Tri-ESV6-linker-DOTAGA (13) and diastereoisomers of Tri-ESV6-DOTAGA (11, 12) [FIG. 18]. Comparative hFAP inhibition assays with tetravalent binders 10 and 29 were also performed [FIG. 20].

### b) Fluorescence Polarization Assay

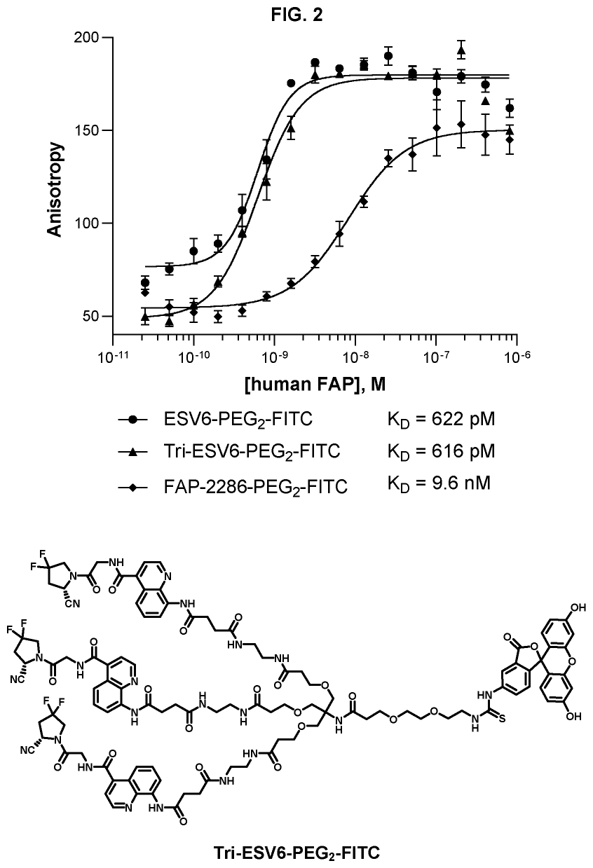

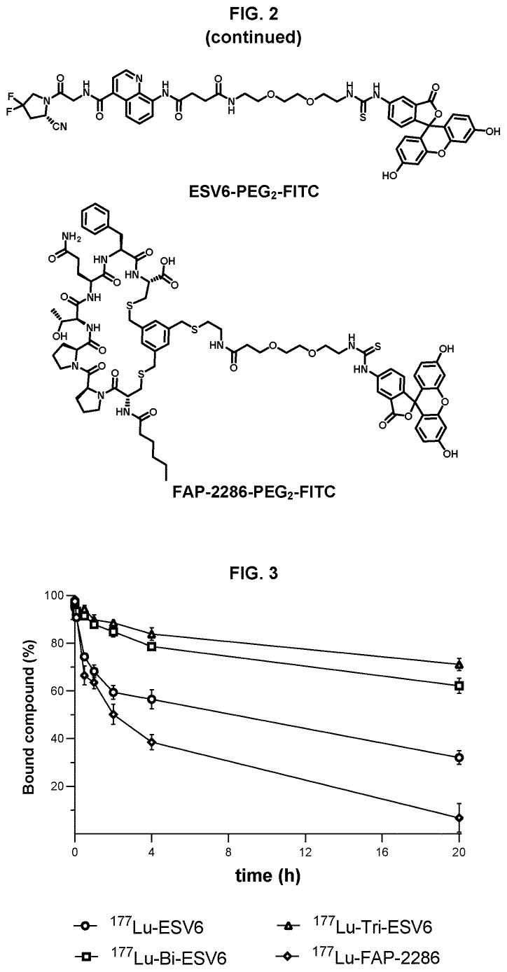

Fluorescence polarization experiments were performed in 384-well plates (nonbinding, ps, f-bottom, black, high volume, 30 μL final volume). Stock solutions of hFAP were serially diluted (1:2) with buffer (50 mM Tris, 100 mM NaCl, and 1 mM EDTA, pH=7.4), while the final concentration of the binders was kept constant at 1 nM. The fluorescence anisotropy was measured on a Tecan microtiter plate reader. Experiments were performed in triplicate, and the mean anisotropy values were fitted using Prism 7 ((Y=m1+m2×0.5×((X+k+m3)−sqrt((X+k+m3)\hat{ }2-4×X×k)), where k is the concentration of the fluorescent binder). Data are reported in FIG. 2.

### c) Cell Efflux

SK-RC-52.hFAP cells, were cultured using RPMI medium supplemented with 10% Fetal Bovine Serum (FBS) and 1% Antibiotic-Antimytotic. Cells were seeded in 24-well plates (200'000 cells/well, 0.5 mL/well) and incubated overnight at 37 °C, 5% CO₂. Then, media was removed, cells were washed with PBS (2×0.5 mL) and incubated with a solution of ¹⁷⁷Lu-ESV6, ¹⁷⁷Lu-Bi-ESV6, ¹⁷⁷Lu-Tri-ESV6 or ¹⁷⁷Lu-FAP-2286 (10 KBq, 1 μmol, 0.5 mL). After 1 h, media was removed, and cells were washed with PBS (2×0.5 mL) and incubated with fresh RPMI medium without supplements. Fractions of the culture media were measured with a gamma counter (Packard Cobra) at different time-points. Percentage of compound bound was calculated as fraction of the total activity and corrected for the ¹⁷⁷Lu decay, as shown in FIG. 3.

### d) Determination of Log D_{7.4}

The lipophilicity of ¹⁷⁷Lu-ESV6, ¹⁷⁷Lu-Bi-ESV6, ¹⁷⁷Lu-Tri-ESV6 and ¹⁷⁷Lu-FAP-2286 was determined as follows. 100 μL aliquots of the radioligand (~1 MBq) in PBS buffer were added to PBS buffer (500 μL, pH 7.4) and 1-octanol (600 μL). The two-layer mixture were vigorously shaken for 10 minutes on a vortex mixer and then centrifuged at 700 rpm for 5 min to facilitate the separation. 100 μL aliquots of both layers were measured in a Packard Cobra Gamma Counter and the partition coefficient was determined by dividing cpm (octanol) by cpm (PBS) and indicated as Log D_{7.4.}
Log D_{7.4} (¹⁷⁷Lu-ESV6): −4.13
Log D_{7.4} (¹⁷⁷Lu-Bi-ESV6): −3.75
Log D_{7.4} (¹⁷⁷Lu-Tri-ESV6): −3.10
Log D_{7.4} (¹⁷⁷Lu-FAP-2286): −3.05

### 10. Animal Studies

All animal experiments were conducted in accordance with Swiss animal welfare laws and regulations under the license number ZH006/2021 granted by the Veterinäramt des Kantons Zürich.

SK-RC-52.hFAP were grown to 80% confluence in RPMI-1640 with 10% fetal bovine serum (FBS) and 1% antibiotic-antimycotic and detached with Trypsin-EDTA (ethylenediaminetetraacetic acid) 0.05%. Tumor cells were resuspended in Hanks' Balanced Salt Solution medium. Aliquots of 5 million cells (100 μL of suspension) were injected subcutaneously in the right flank of female athymic Balb/c AnNRj-Foxn1 mice (6 to 8 wk of age).

a) Radiolabelling with ¹⁷⁷Lu

Radiolabeling of ESV6-DOTAGA, Bi-ESV6-DOTAGA, Tri-ESV6-DOTAGA, Tetra-ESV6-DOTAGA, Hexa-ESV6-DOTAGA, Octa-ESV6-DOTAGA and FAP-2286 with lutetium-177 (a therapeutic radionuclide) was performed with different specific activities for three different studies (biodistribution, therapy and dose optimization).

Prior to the biodistribution study, precursors (100 nmol) were dissolved in 100 μL of milliQ water and diluted with 200 μL of sodium acetate (1 M in water, pH=4.5). 20 MBq of ¹⁷⁷Lu solution were added and the mixture was heated at 90 °C for 10 minutes, followed by dilution with 1600 μL of PBS to achieve final volume of 2 mL (20 doses of 100 μL each).

Prior to the therapy studies, precursors (25 nmol) were dissolved in 25 μL of milliQ water, then sodium acetate buffer (75 μL, 1 M in water) and 25, 75 or 150 MBq of ¹⁷⁷Lu solution were added. The mixture was heated at 90 °C for 1 minutes followed by dilution with 400 μL of PBS to afford a final volume of 500 μL (5 doses of 100 μL each). Quality control of radiosynthesis was performed using radio-HPLC.

Prior to the dose optimization study for radiotherapeutic applications, precursors (0.24 to 180 nmol) were dissolved in milliQ water and diluted with sodium acetate (1 M in water, pH=4.5). ¹⁷⁷Lu solution (4 MBq) were added and the mixture was heated at 90 °C for 10 minutes, followed by dilution with PBS to achieve final volume of 400 μL (4 doses of 100 μL each).

Prior to the biodistribution study at different specific activities (FIG. 19), precursors (20 nmol) were dissolved in milliQ water and diluted with sodium acetate (1 M in water, pH=4.5). ¹⁷⁷Lu solution (4 or 96 MBq) were added and the mixture was heated at 90 °C for 10 minutes, followed by dilution with PBS to achieve final volume of 400 μL (4 doses of 100 μL each, corresponding to 0.2 MBq/nmol and 4.8MBq/nmol, respectively).

### b) Quantitative Biodistribution Analysis in Tumor Bearing Mice

ESV6-DOTAGA, Bi-ESV6-DOTAGA, Tri-ESV6-DOTAGA, Tetra-ESV6-DOTAGA, Hexa-ESV6-DOTAGA, Octa-ESV6-DOTAGA and FAP-2286 were radiolabeled with ¹⁷⁷Lu as described above. Tumors were allowed to grow to an average volume of 300 mm³. Mice were randomized and injected intravenously with radiolabeled preparations of ¹⁷⁷Lu-ESV6, ¹⁷⁷Lu-Bi-ESV6, ¹⁷⁷Lu-Tri-ESV6, Tetra-ESV6-DOTAGA, Hexa-ESV6-DOTAGA, Octa-ESV6-DOTAGA or ¹⁷⁷Lu-FAP-2286 (250 nmol/kg; 50 MBq/kg). Mice were euthanized at different time-points after the intravenous injection by CO₂ asphyxiation. Tumors, organs, and blood were collected, weighted, and radioactivity was measured with a Packard Cobra Gamma Counter. Values are expressed as percent ID/g±SD [FIG. 4]. The % ID/g in the tumors was corrected considering the SK-RC-52.hFAP tumor growth rate.

Results of the ¹⁷⁷Lu biodistribution study at different specific activities: low (0.2 MBq/nmol) and high (4.8 MBq/nmol) with ¹⁷⁷Lu Tri-ESV6-DOTAGA are shown in FIG. 19.

### Values (Average±Standard Deviation):

Quantitative in vivo biodistribution of ¹⁷⁷Lu-ESV6-DOTAGA, ¹⁷⁷Lu-Bi-ESV6-DOTAGA, ¹⁷⁷Lu-Tri-ESV6-DOTAGA, ¹⁷⁷Lu-FAP-2286, Tetra-ESV6-DOTAGA, Hexa-ESV6-DOTAGA and Octa-ESV6-DOTAGA at different time points after intravenous administration (250 nmol/kg, 50 MBq/kg) in mice bearing SK-RC-52.hFAP tumors, are shown in Tables 1 to 4 and 10 to 12. Data are reported as % ID/g±standard deviation.

### Tumor to Organ Ratios:

Tumor-to-organ ratios of ¹⁷⁷Lu-ESV6-DOTAGA, ¹⁷⁷Lu-Bi-ESV6-DOTAGA, ¹⁷⁷Lu-Tri-ESV6-DOTAGA, ¹⁷⁷Lu-FAP-2286, Tetra-ESV6-DOTAGA, Hexa-ESV6-DOTAGA and Octa-ESV6-DOTAGA, at different time points after intravenous administration (250 nmol/kg, 50 MBq/kg) in mice bearing SK-RC-52.hFAP tumors, are shown in Tables 5 to 8 and 13 to 15. Avg.=average; Std.d=standard deviation. Particularly advantageous results for Tri-ESV6-DOTAGA (e.g. kidney/tumor ratio) are reported in Table 7.

### c) Therapeutic Efficacy Evaluation in Tumor Bearing Mice

The anti-cancer efficacy of ¹⁷⁷Lu-ESV6, ¹⁷⁷Lu-Bi-ESV6 and ¹⁷⁷Lu-Tri-ESV6 was assessed in athymic Balb/c AnNRj-Foxn1 mice bearing SK-RC-52.hFAP tumor in the right flank. ¹⁷⁷Lu-ESV6, ¹⁷⁷Lu-Bi-ESV6 or ¹⁷⁷Lu-Tri-ESV6 were intravenously administered at a dose of 250 nmol/kg, 250 MBq/kg (single administration on day 9 after tumor implantation). Therapy experiments started when the average volume of established tumors had reached 100-150 mm³. Body weight of the animals and tumor volume were daily measured and recorded. Tumor dimensions were measured with an electronic caliper and tumor volume was calculated with the formula (long side, mm)×(short side, mm)×(short side, mm)×0.5. Animals were euthanized when one or more termination criterium indicated by the experimental license was reached. Prism 7 software (GraphPad Software) was used for data analysis. Results are shown in FIG. 5, FIG. 6, and FIG. 7.

### d) Dose Escalation for Radiotherapeutic Applications

Female athymic Balb/c AnNRj-Foxn1 mice bearing subcutis SK-RC-52.hFAP tumors were injected intravenously with eight different doses of ¹⁷⁷Lu-Tri-ESV6-DOTAGA ranging from 3 nmol/kg to 2250 nmol/kg. The mice were sacrificed after 24 h and tumor and healthy organs were harvested and measured with a gamma counter. Results reported as % ID/g are shown in FIG. 15A. Results reported as tumor to organ ratio are shown in FIG. 15B. Since the best tumor to organ ratios are obtained with high doses ranging between 90 and 250 nmol/kg, we anticipate that the ideal dose in human patients for radiotherapeutic applications will be between 1 and 3 milligram per patient. e) Therapeutic efficacy evaluation in tumor bearing mice in dose escalation and in combination with L19-IL2

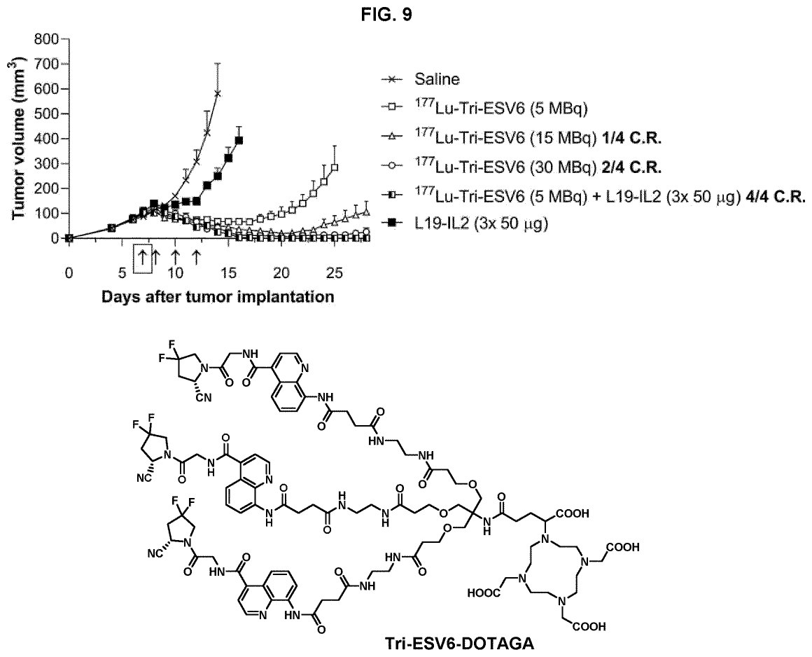

The anti-cancer efficacy of L19-IL2 as single agent (3×0.05 mg/mouse), ¹⁷⁷Lu-Tri-ESV6 as single agent at different doses (5 MBq/mouse, 15 MBq/mouse, 30 MBq/mouse), ¹⁷⁷Lu-Tri-ESV6+L19-IL2 combination (5 MBq/mouse of ¹⁷⁷Lu-Tri-ESV6 followed by three injections of L19-IL2 at 0.05 mg/mouse) or saline was assessed in athymic Balb/c AnNRj-Foxn1 mice bearing SK-RC-52.hFAP tumor in the right flank. Therapy experiments started when the average volume of established tumors had reached 100-150 mm³. Body weight of the animals and tumor volume were daily measured and recorded. Tumor dimensions were measured with an electronic caliper and tumor volume was calculated with the formula (long side, mm)×(short side, mm)×(short side, mm)×0.5. Animals were euthanized when one or more termination criterium indicated by the experimental license was reached. Prism 7 software (GraphPad Software) was used for data analysis. Results are shown in FIG. 9. All mice administrated with the ¹⁷⁷Lu-Tri-ESV6+L19-IL2 combination were cured after treatment.

The L19-IL2 immunocytokine conjugate used in the present Example comprises a human IL2 polypeptide having the sequence of SEQ ID NO: 21 fused at its N-terminus via a 17 amino acid linker (SEQ ID NO: 23) to the C-terminus of the VL domain of a single-chain variable fragment (scFv) molecule comprising the VH (SEQ ID NO: 19) and VL (SEQ ID NO: 20) domains of antibody L19, which specifically bind the extra-domain B of fibronectin (ED-B), as described in WO2001/062298. The epitope bound is the sequence of SEQ ID NO: 40 (cf. FIG. 4C in Fattorusso et al., Structure 7:381-390). In the immunocytokine employed in this present example, the pairing of the VH domain of one L19-IL2 molecule with the VL domain of another L19-IL2 molecule permits the formation of homodimers via the scFv portion of the immunocytokine, essentially in an “scFv2” or “diabody” format, as shown in FIG. 12.

The amino acid sequence of the L19-IL2 immunocytokine conjugate is set forth in SEQ ID NO 12:

EVQLLESGGGLVQPGGSLRLSCAASGFTFSSFS
MSWVRQAPGKGLEWVSSISGSSGTTYYADSVKG
RFTISRDNSKNTLYLQMNSLRAEDTAVYYCAKP
FPYFDYWGQGTLVTVSSGDGSSGGSGGASEIVL
TQSPGTLSLSPGERATLSCRASQSVSSSFLAWY
QQKPGQAPRLLIYYASSRATGIPDRFSGSGSGT
DFTLTISRLEPEDFAVYYCQQTGRIPPTFGQGT
KVEIKEFSSSSGSSSSGSSSSGAPTSSSTKKTQ
LQLEHLLLDLQMILNGINNYKNPKLTRMLTFKF
YMPKKATELKHLQCLEEELKPLEEVLNLAQSKN
FHLRPRDLISNINVIVLELKGSETTFMCEYADE
TATIVEFLNRWITFCQSIISTLT

The results demonstrate surprising synergistic increase in therapeutic activity as compared to the sum of the effects achieved by the individual components of the combination (the SMDC and the L19-IL2). Without wishing to be bound by any theory, it is believed that the mechanism of action of the combination involves activation of NK cells in the tumor, which boosts the antitumor efficacy.

f) Therapeutic Efficacy Evaluation in Tumor Bearing Mice (Schedule Optimization for Combination with L19-IL2)

The anti-cancer efficacy was assessed in athymic Balb/c AnNRj-Foxn1 mice bearing SK-RC-52.hFAP tumor in the right flank. Therapy experiments started when the average volume of established tumors had reached 100-150 mm³. Body weight of the animals and tumor volume were daily measured and recorded. Tumor dimensions were measured with an electronic caliper and tumor volume was calculated with the formula (long side, mm)×(short side, mm)×(short side, mm)×0.5. Animals were euthanized when one or more termination criterium indicated by the experimental license was reached. Prism 7 software (GraphPad Software) was used for data analysis. Different groups were treated with (i) L19-IL2 as single agent at a dose of 0.05 mg/mouse on day 8,10 and 12, (ii) ¹⁷⁷Lu-Tri-ESV6 as single agent at a dose of 5 MBq/mouse on day 7 (iii) ¹⁷⁷Lu-Tri-ESV6+L19-IL2 combination (¹⁷⁷Lu-Tri-ESV6 at a dose of 5 MBq/mouse on day 7 followed by three administrations of L19-IL2 at a dose 0.05 mg/mouse on day 8, 10 and 12—SCHEDULE 1—, or by three administrations of L19-IL2 at a dose 0.05 mg/mouse on day 12, 17 and 22—SCHEDULE 2—, or by a single administration of L19-IL2 at a dose 0.05 mg/mouse on day 8—SCHEDULE 3—, or by a single administration of L19-IL2 at a dose 0.05 mg/mouse on day 12—SCHEDULE 4—or (iv) saline or (iv) saline. Results are shown in FIG. 13.

g) Quantitative In Vivo MMAE Release from Different Therapeutic Conjugates

The ESV6 binding moiety in monomeric, dimeric or trimeric form was conjugated to an identical linker-payload structure based on a Glycine-Proline linker and an MMAE cytotoxic moiety. The three conjugates: (i) ESV6-GlyPro-MMAE—corresponding to Conjugate 58a of EP3891138B1—, (ii) Bi-ESV6-GlyPro-MMAE—corresponding to Conjugate 11 of WO2022/171811A1, and (iii) Tri-ESV6-GlyPro-MMAE—corresponding to Conjugate 9 of the present invention—, were compared in a tumor mouse model to study the efficacy of release of the MMAE cytotoxic moiety into the tumor.

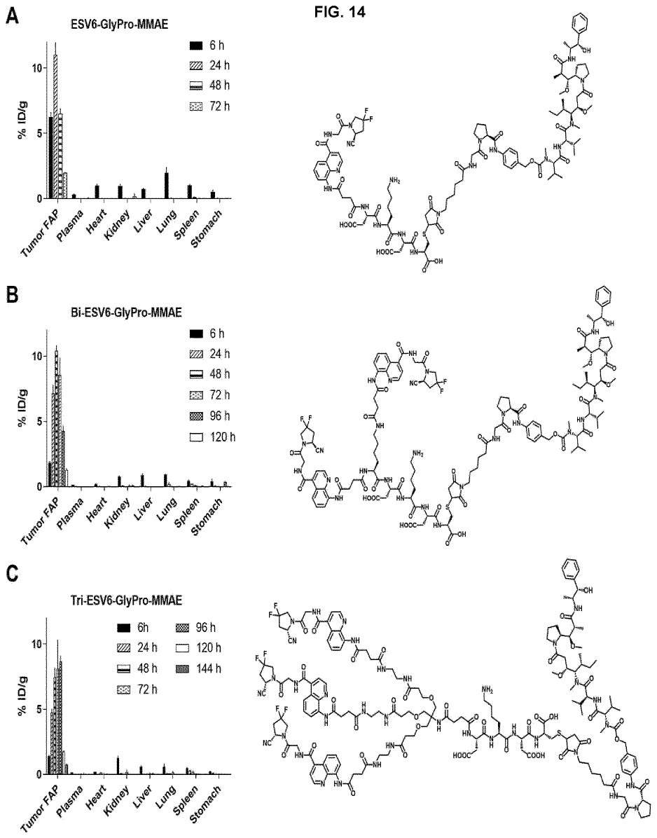

HT-1080.hFAP tumors were implanted into the right flank of athymic Balb/c AnNRj-Foxn1 mice and allowed to grow to an average volume of approximately 200 mm³. Mice were injected with the three conjugates at a dose of 250 nmol/kg and sacrificed at different timepoints after administration. Fresh blood was collected in lithium heparin tubes (BD Microcontainer LH Tubes), vortexed, and centrifuged (15,000 g, 15 minutes). Plasma was frozen and stored at −80 °C. Healthy organs and tumors were subsequently excised, frozen with dry ice, and stored at −80 °C Frozen plasma (50 μL) and mouse tissues (~50 mg) were thawed, and 500 μL of PBS was added. Samples were kept on ice, and 50 μL solution of internal standard (d8-MMAE, 50 nM) was added. Samples were then homogenized at 4 °C with a tissue lyser for 2 minutes at 30 Hz for 4 cycles. After homogenization, samples were centrifuged (21′000 g, 10 min). Subsequently, 100 μL of supernatants were collected and added to 900 μL of acetonitrile (ACN) to induce protein precipitation. After centrifugation (21′000 g, 10 min), 800 μL of supernatants were collected and dried at room temperature with a vacuum centrifuge. Pellets were then resuspended in 20 μL of an aqueous solution containing 3% acetonitrile and 0.1% of HCOOH, and 5 μL were injected into the UHPLC-MS system. Chromatographic separation was carried out on a Hypersil Gold C18 column (100 mm×2.1 mm, 1.9 μm particle size, 175 Å pore size) column temperature was set at 50 °C and a flow rate of 700 L/min with a gradient program from 95% A (water+0.1% HCOOH), 5% B (ACN+0.1% HCOOH) to 35% of A in 2.5 minutes, from 35% A to 5% A in 0.4 minutes and 5% A was kept for 1.3 minutes before reconditioning at 95% A. The LC system was coupled to a Q-Exactive mass spectrometer via an Ion Max HESI Source. Ionization was carried out with a spray voltage of 3.5 kV; Sheath gas 40 units; Aux gas 10 units; capillary temperature of 380 °C; Aux gas temperature 450 °C; S-lens RF level 60. The mass spectrometer was operating in targeted Single Ion Monitoring mode (t-SIM) following the molecular ion 718.5113 m/z. The detector was working in positive ionization mode with the following parameters: resolution 70'000 (FWHM at 200 m/z); AGC target of 5×104; maximum injection time of 200 ms; isolation window 14 m/z; isolation offset 5 m/z. Peak areas of analytes and internal standards were integrated, and corresponding ratios were calculated. The ratios were then transformed into pmol/g of wet tissue using single-concentration external calibration points and corrected by the total weight of the sample analysed. The percentage of injected dose per gram (% ID/g) was finally calculated by normalizing the value based on the total dose injected into the mouse. Data analysis was performed with Skyline v22.2.0.351. Results are shown in FIG. 14.

h) Therapeutic Efficacy Evaluation of ESV6-GlyPro-MMAE and Tri-ESV6-GlyPro-MMAE in HT-1080.hFAP Tumor Bearing Mice (Schedule Optimization)

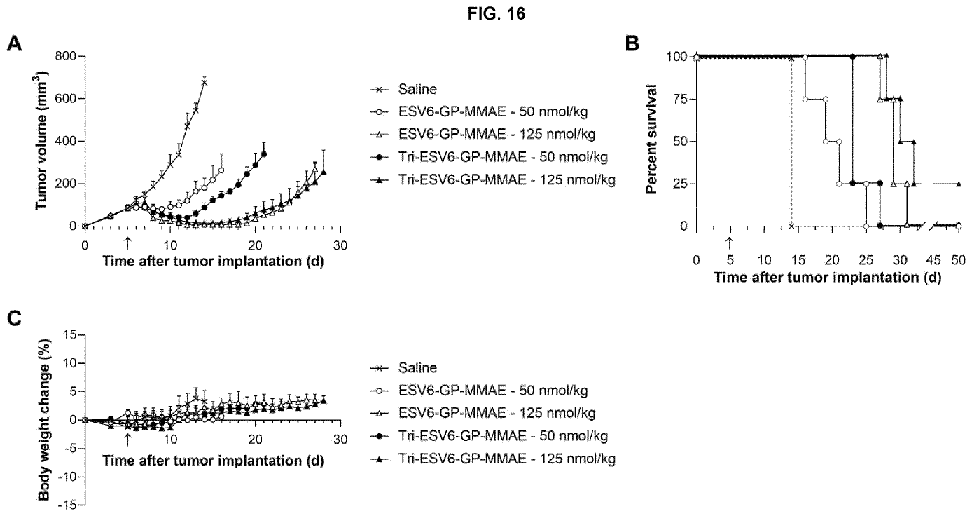

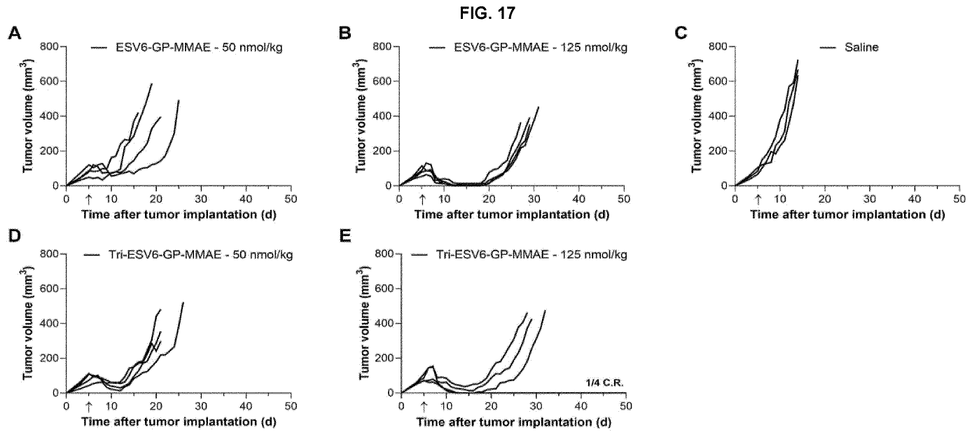

The anti-cancer efficacy of (i) ESV6-GlyPro-MMAE (50 or 125 nmol/kg) —corresponding to Conjugate 58a of EP3891138B1—, and (ii) Tri-ESV6-GlyPro-MMAE (50 or 125 nmol/kg) according to the present invention was assessed in athymic Balb/c AnNRj-Foxn1 mice bearing HT-1080.hFAP tumor in the right flank. Therapy experiments started when the average volume of established tumors had reached 80-100 mm3. Body weight of the animals and tumor volume were daily measured and recorded. Tumor dimensions were measured with an electronic caliper and tumor volume was calculated with the formula (long side, mm)×(short side, mm)×(short side, mm)×0.5. Animals were euthanized when one or more termination criterium indicated by the experimental license was reached. Prism 7 software (GraphPad Software) was used for data analysis. Results are shown in FIG. 16 and FIG. 17.

### 11. Chemical Structures and Synthesis of Tri-ESV6-DOTAGA Compounds with PEG Spacers

### a) Chemical Structure of Tri-ESV6-PEG₂-DOTAGA

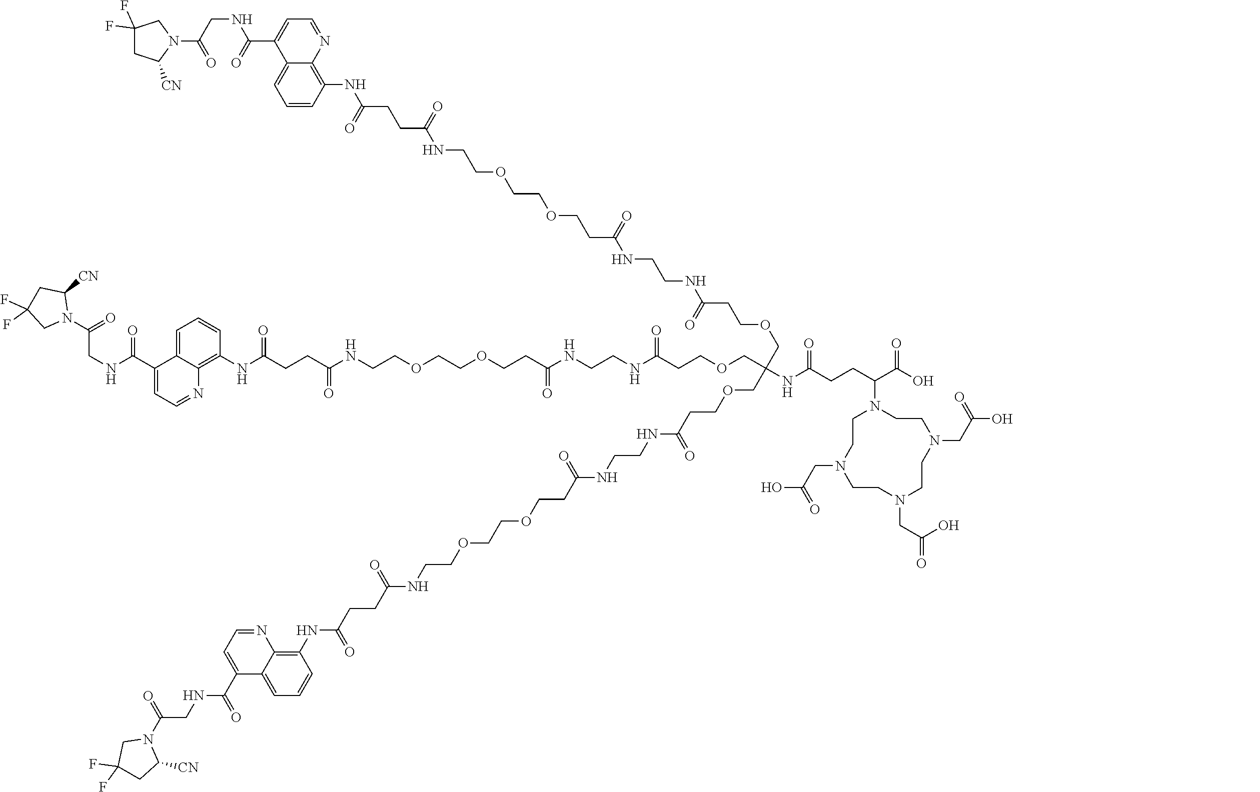 [SMILES: N#C[C@@H]1CC(F)(F)CN1C(=O)CNC(=O)c1ccnc2c(NC(=O)CCC(=O)NCCOCCOCCC(=O)NCCNC(=O)CCOCC(COCCC(=O)NCCNC(=O)CCOCCOCCNC(=O)CCC(=O)Nc3cccc4c(C(=O)NCC(=O)N5CC(F)(F)C[C@H]5C#N)ccnc34)(COCCC(=O)NCCNC(=O)CCOCCOCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 2723.7874 Da
Exact mass: 2722.1563 Da
Formula: C122H161F6N29036
SMILES: O═C(NC(COCCC(NCCNC(CCOCCOCCNC(CCC(NC1=C(N=CC=C2C(NCC(N3CC(F)(F)C[C@H]3C #N)═O)═O) C2=CC═C1)=O)═O)═O)═O)(COCCC(NCCNC(CCOCCOCCNC(CCC(NC4=C(N═CC—C5C(NCC(N6CC(F)(F)C[C@H]6C #N)═O)═O)C5=CC═C4)=O)═O)═O)═O)COCCC(NCCNC(CCOCCOCCNC(CCC(NC7=C(N═CC═C8C(N CC(N9CC(F)(F)C[C@H]9C #N)═O)═O)C8=CC═C7)=O)═O)═O)═O)CCC(N % 10CCN(CC(O)═O)CCN(CC(O)═O) CCN(CC(O)═O)CC % 10)C(O)═O

### b) Chemical Structure of Tri-ESV6-PEG4-DOTAGA

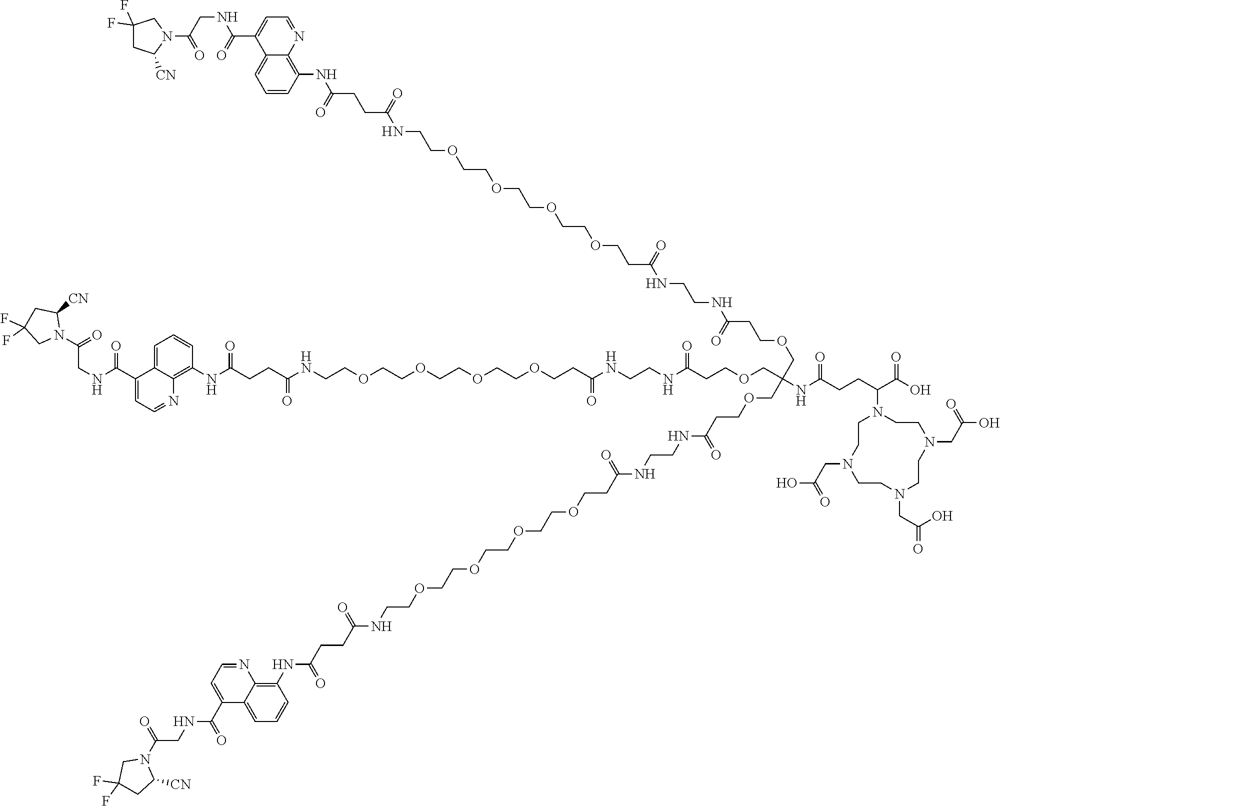 [SMILES: N#C[C@@H]1CC(F)(F)CN1C(=O)CNC(=O)c1ccnc2c(NC(=O)CCC(=O)NCCOCCOCCOCCOCCC(=O)NCCNC(=O)CCOCC(COCCC(=O)NCCNC(=O)CCOCCOCCOCCOCCNC(=O)CCC(=O)Nc3cccc4c(C(=O)NCC(=O)N5CC(F)(F)C[C@H]5C#N)ccnc34)(COCCC(=O)NCCNC(=O)CCOCCOCCOCCOCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 2988.1054 Da
Exact mass: 2986.3136 Da
Formula: C134H185F6N29042
SMILES: O═C(NC(COCCC(NCCNC(CCOCCOCCOCCOCCNC(CCC(NC1=C(N═CC═C2C(NCC(N3CC(F)(F)C[C@H]3C #N)═O)═O)C2=CC═C1)=O)═O)═O)═O)(COCCC(NCCNC(CCOCCOCCOCCOCCNC(CCC(NC4=C(N═CC═C5C(NCC(N6CC(F)(F)C[C@H]6C #N)═O)═O)C5=CC═C4)=O)═O)═O)═O)COCCC(NCCNC(CCOCCOCCOCCOCCN C(CCC(NC7=C(N═CC═C8C(NCC(N9CC(F)(F)C[C@H]9C #N)═O)═O)C8=CC═C7)=O)═O)═O)═O)CCC(N % 10C CN(CC(O)═O)CCN(CC(O)═O)CCN(CC(O)═O)CC % 10)C(O)═O

### c) Chemical Structure of Tri-ESV6-PEG6-DOTAGA

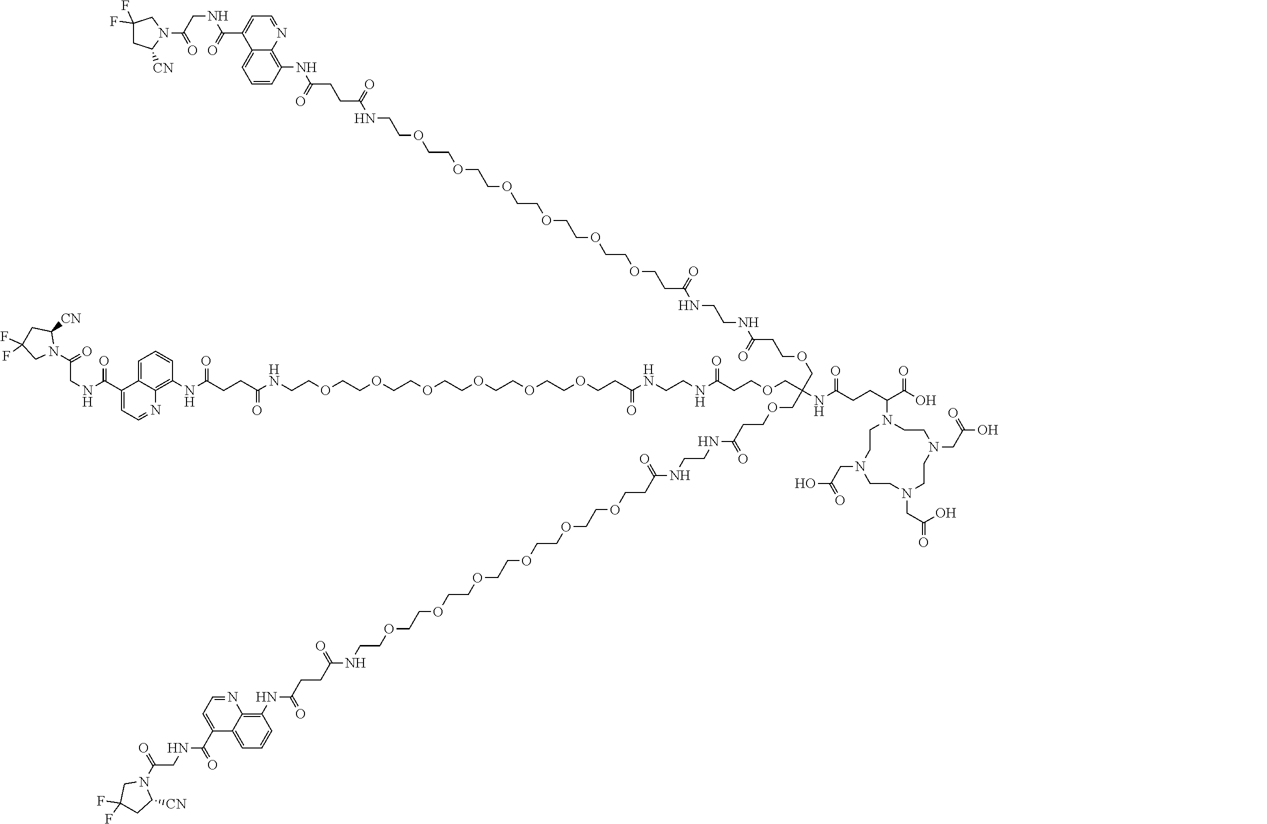 [SMILES: N#C[C@@H]1CC(F)(F)CN1C(=O)CNC(=O)c1ccnc2c(NC(=O)CCC(=O)NCCOCCOCCOCCOCCOCCOCCC(=O)NCCNC(=O)CCOCC(COCCC(=O)NCCNC(=O)CCOCCOCCOCCOCCOCCOCCNC(=O)CCC(=O)Nc3cccc4c(C(=O)NCC(=O)N5CC(F)(F)C[C@H]5C#N)ccnc34)(COCCC(=O)NCCNC(=O)CCOCCOCCOCCOCCOCCOCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 3252.4234 Da
Exact mass: 3250.4709 Da
Formula: C146H209F6N29048
SMILES: O═C(NC(COCCC(NCCNC(CCOCCOCCOCCOCCOCCOCCNC(CCC(NC1=C(N═CC═C2C(NCC(N3CC(F)(F)C[C@H]3C #N)═O)═O)C2=CC═C1)=O)═O)═O)═O)(COCCC(NCCNC(CCOCCOCCOCCOCCOCCOCCNC(CCC(N C4=C(N═CC═C5C(NCC(N6CC(F)(F)C[C@H]6C #N)═O)—O)C5=CC═C4)=O)═O)═O)═O)COCCC(NCCNC(CCO CCOCCOCCOCCOCCOCCNC(CCC(NC7=C(N═CC═C8C(NCC(N9CC(F)(F)C[C@H]9C #N)═O)═O)C8=CC═C7)=O)═O)═O)═O)CCC(N % 10CCN(CC(O)═O)CCN(CC(O)═O)CCN(CC(O)═O)CC % 10)C(O)═O

### d) Chemical Structure of Tri-ESV6-PEG8-DOTAGA

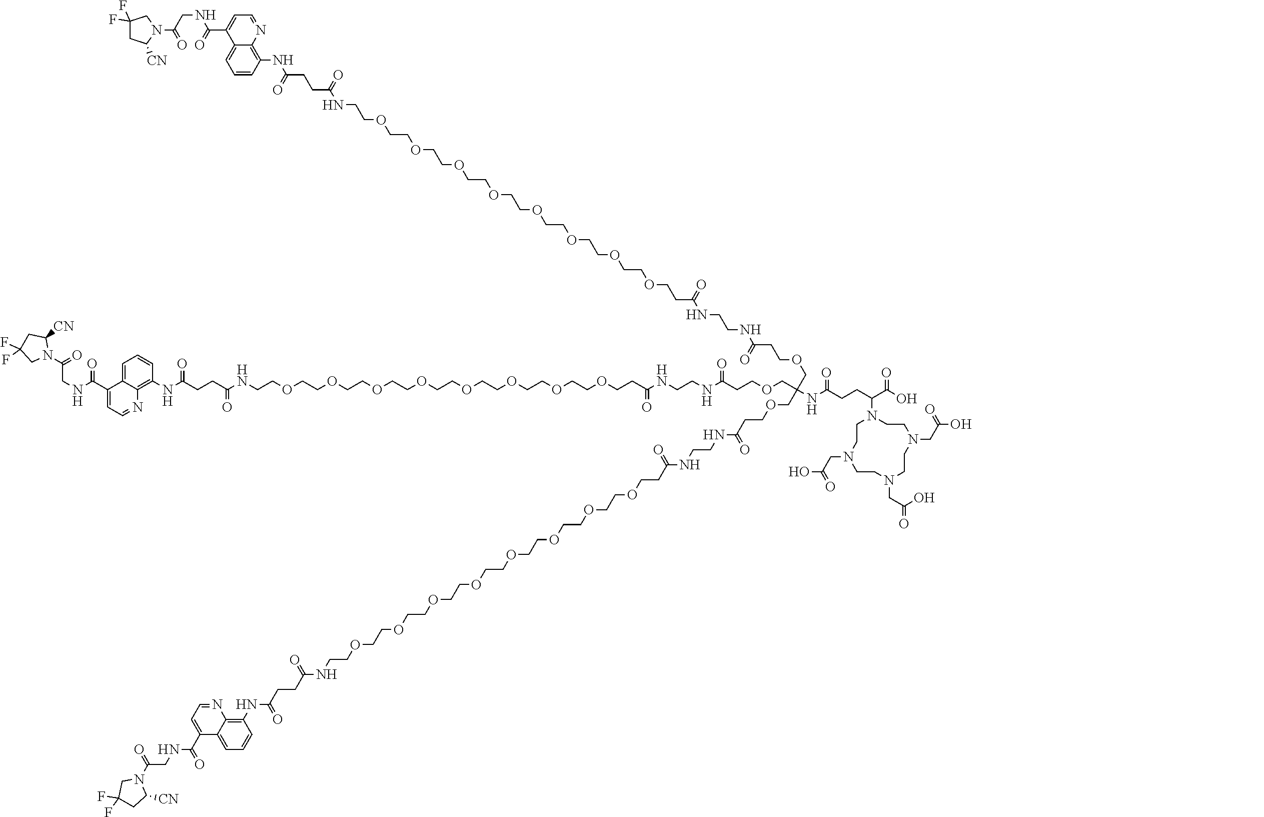 [SMILES: N#C[C@@H]1CC(F)(F)CN1C(=O)CNC(=O)c1ccnc2c(NC(=O)CCC(=O)NCCOCCOCCOCCOCCOCCOCCOCCOCCC(=O)NCCNC(=O)CCOCC(COCCC(=O)NCCNC(=O)CCOCCOCCOCCOCCOCCOCCOCCOCCNC(=O)CCC(=O)Nc3cccc4c(C(=O)NCC(=O)N5CC(F)(F)C[C@H]5C#N)ccnc34)(COCCC(=O)NCCNC(=O)CCOCCOCCOCCOCCOCCOCCOCCOCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 3516.7414 Da
Exact mass: 3514.6282 Da
Formula: C158H233F6N29054
SMILES: O═C(NC(COCCC(NCCNC(CCOCCOCCOCCOCCOCCOCCOCCOCCNC(CCC(NC1=C(N═CC—C2C(NCC(N3C C(F)(F)C[C@H]3C #N)═O)═O)C2=CC═C1)=O)═O)═O)═O)(COCCC(NCCNC(CCOCCOCCOCCOCCOCCOCCO CCOCCNC(CCC(NC4=C(N═CC═C5C(NCC(N6CC(F)(F)C[C@H]6C #N)═O)═O)C5=CC═C4)=O)═O)═O)═O)CO CCC(NCCNC(CCOCCOCCOCCOCCOCCOCCOCCOCCNC(CCC(NC7=C(N═CC═C8C(NCC(N9CC(F)(F)C[C@ H]9C #N)═O)═O)C8=CC═C7)=O)═O)═O)═O)CCC(N % 10CCN(CC(O)═O)CCN(CC(O)═O)CCN(CC(O)═O)CC % 10)C(O)═O

### e) Synthetic Route for the Synthesis of TriOncoFAP-PEG₂-DOTAGA, TriOncoFAP-PEG4-DOTAGA, TriOncoFAP-PEG6-DOTAGA and TriOncoFAP-PEG8-DOTAGA

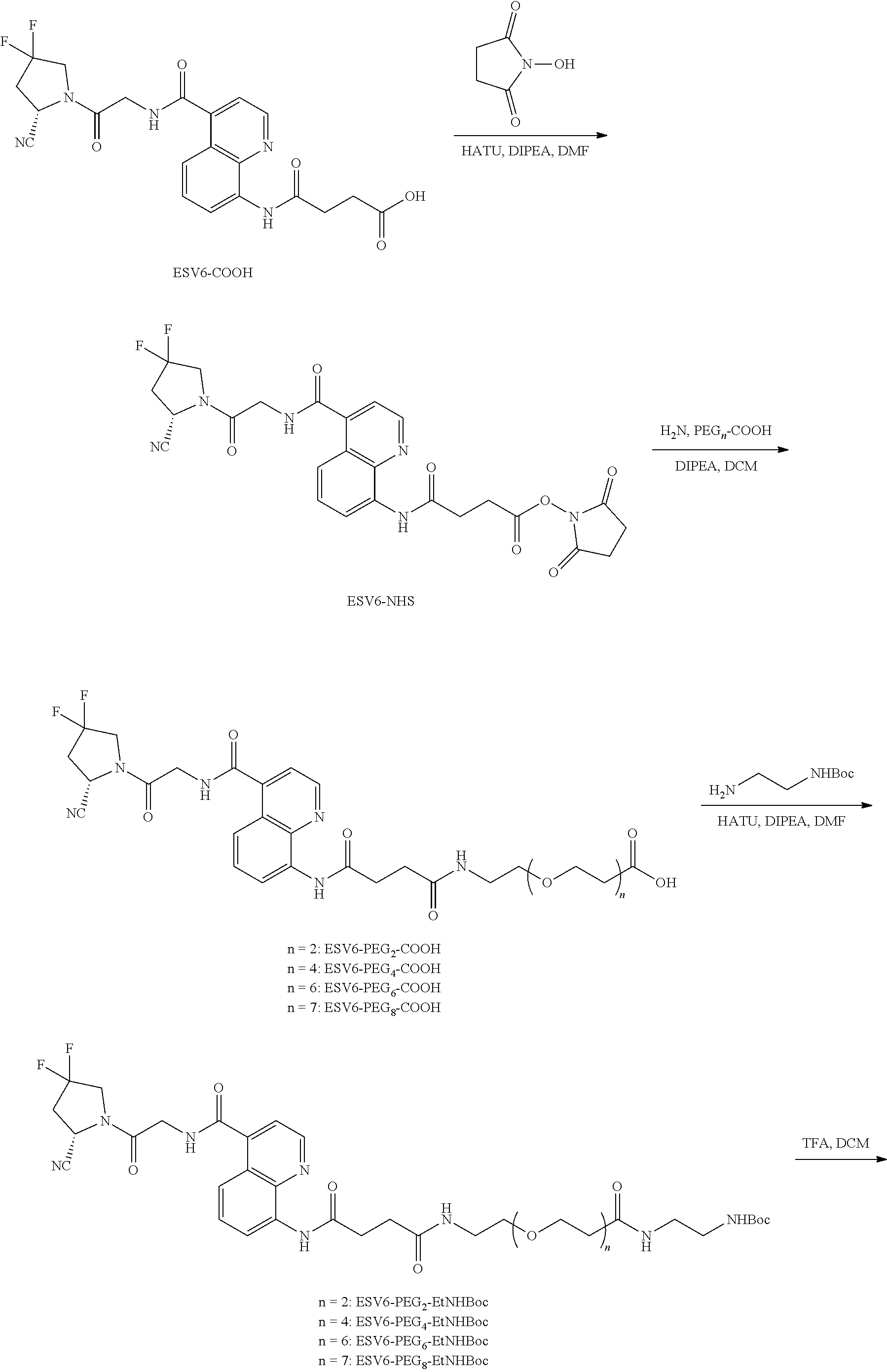

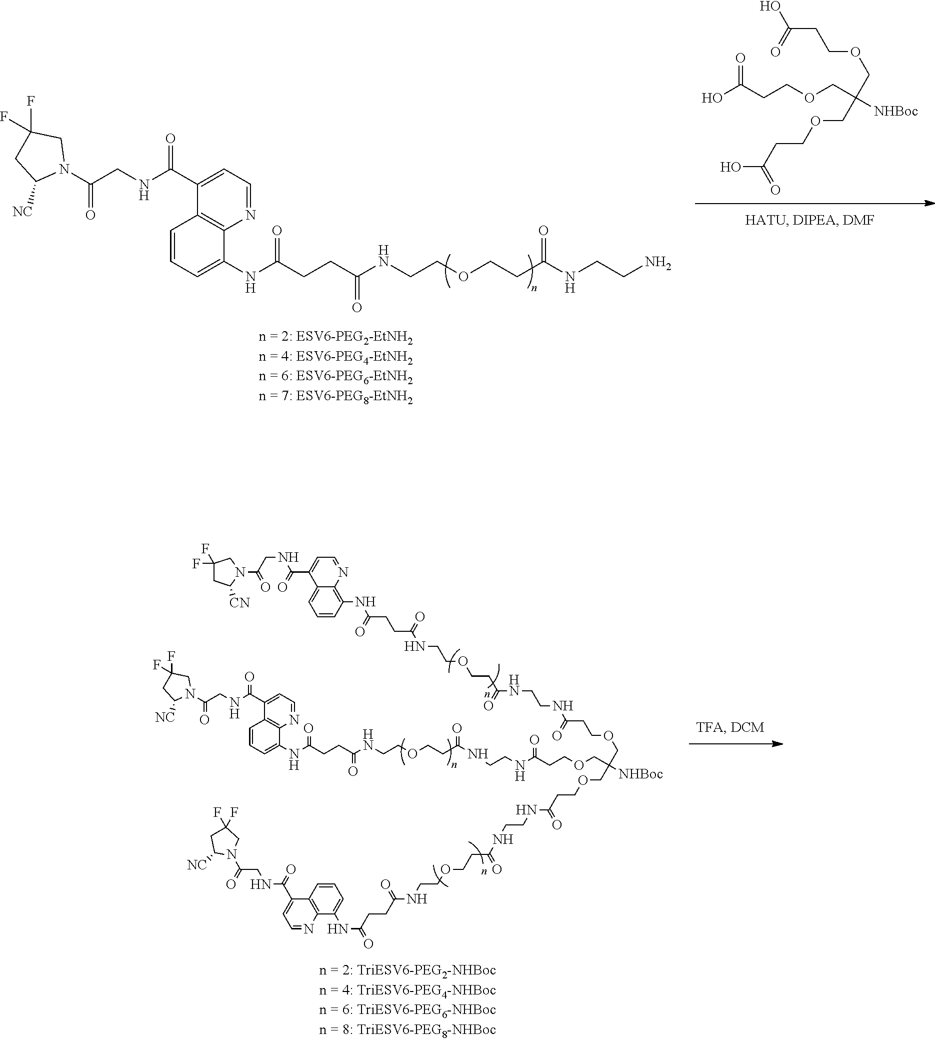

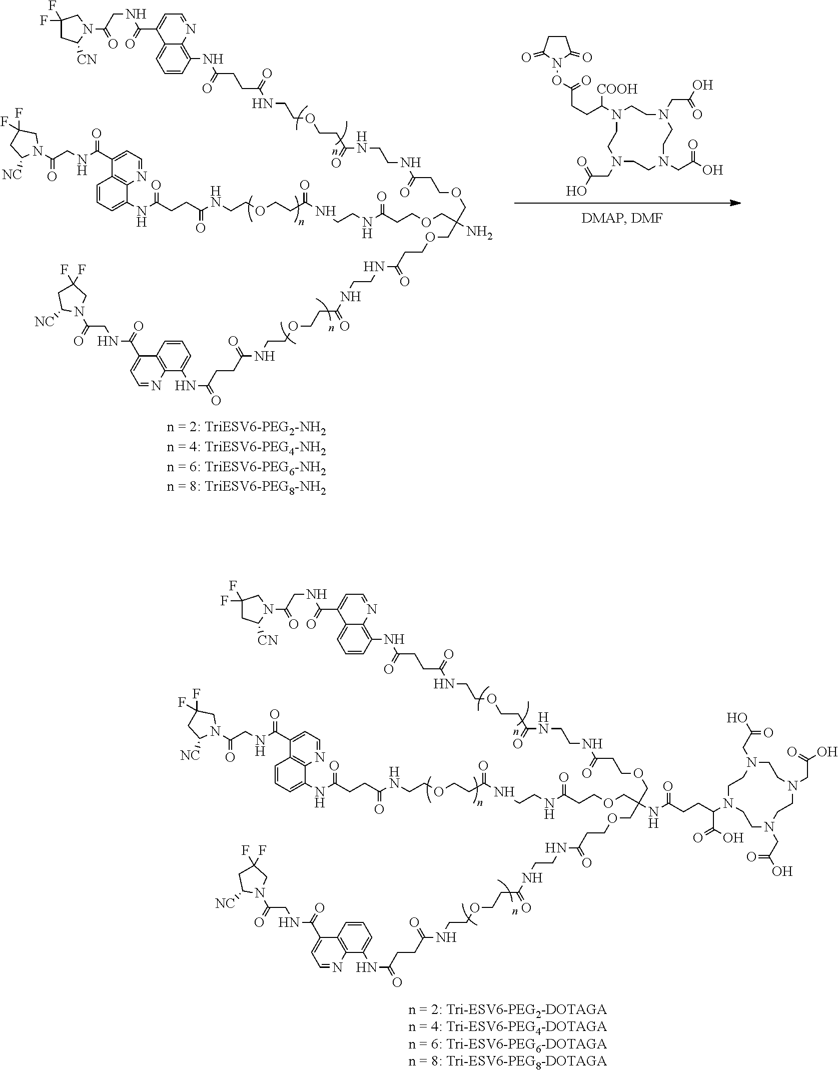

Enzymatic activity of hFAP on the Z-Gly-Pro-AMC substrate was measured at room temperature on a microtiter plate reader, monitoring the fluorescence at an excitation wavelength of 360 nm and an emission wavelength of 465 nm. The reaction mixture contained substrate (20 μM), protein (66 μM, constant), assay buffer (50 mM Tris, 100 mM NaCl, and 1 mM EDTA, pH=7.4), and inhibitors with serial dilution from 167 nM to 80 fM, 1:2 in a total volume of 20 μL. Experiments were performed in triplicate, and the mean fluorescence values were fitted using Prism 7. The value is defined as the concentration of inhibitor required to reduce the enzyme activity by 50% after addition of the substrate. The results are shown in FIG. 10. The compound with the shortest distance between the targeting moiety and the ramification point (PEG Unit=0) was surprisingly found to have the lowest IC50 [Table 16].

### 11. Chemical Structures and Synthesis of Compounds with Alternative Linkers

### a) Chemical Structure of ESV6-L-DOTAGA

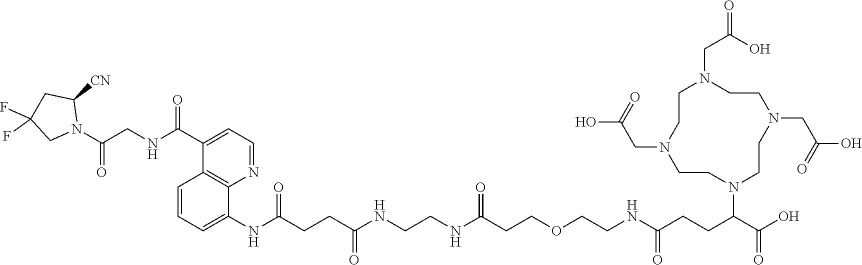 [SMILES: N#C[C@@H]1CC(F)(F)CN1C(=O)CNC(=O)c1ccnc2c(NC(=O)CCC(=O)NCCNC(=O)CCOCCNC(=O)CCC(C(=O)O)N3CCN(CC(=O)O)CCN(CC(=O)O)CCN(CC(=O)O)CC3)cccc12]

Molecular weight: 1075.0948 Da
Exact mass: 1074.4582 Da
Formula: C47H64F2N12015
SMILES: O═C(NCCNC(CCOCCNC(CCC(NICCN(CC(O)—O)CCN(CC(O)═O)CCN(CC(O)═O)CC1)C(O)═O)═O)═O)CCC(NC2=CC═CC3=C2N═CC═C3C(NCC(N4 [C@H](C#N)CC(F)(F)C4)=O)═O)═O

### b) Chemical Structure of Bi-ESV6-L-DOTAGA

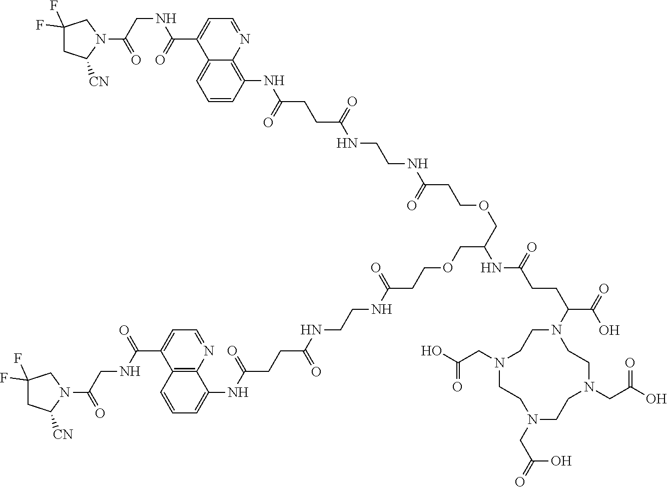 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12]

Molecular weight: 1660.6636 Da
Exact mass: 1659.6730 Da
Formula: C74H93F4N19021
SMILES: O═C(NCCNC(CCOCC(NC(CCC(NICCN(CC(O)—O)CCN(CC(O)—O)CCN(CC(O)═O)CC1)C(O)═O)═O)COCCC(NCCNC(CCC(NC2=C(N═CC═C3C(NCC(N4CC(F)(F)C[C@H]4C #N)═O)═O)C3=CC═C2)=O)═O)═O)═O)CCC(N C5=CC═CC6=C5N═CC═C6C(NCC(N7 [C@H](C#N)CC(F)(F)C7)=O)═O)═O

### c) Synthetic Route for the Synthesis of ESV6-L-DOTAGA and Bi-ESV6-L-DOTAGA

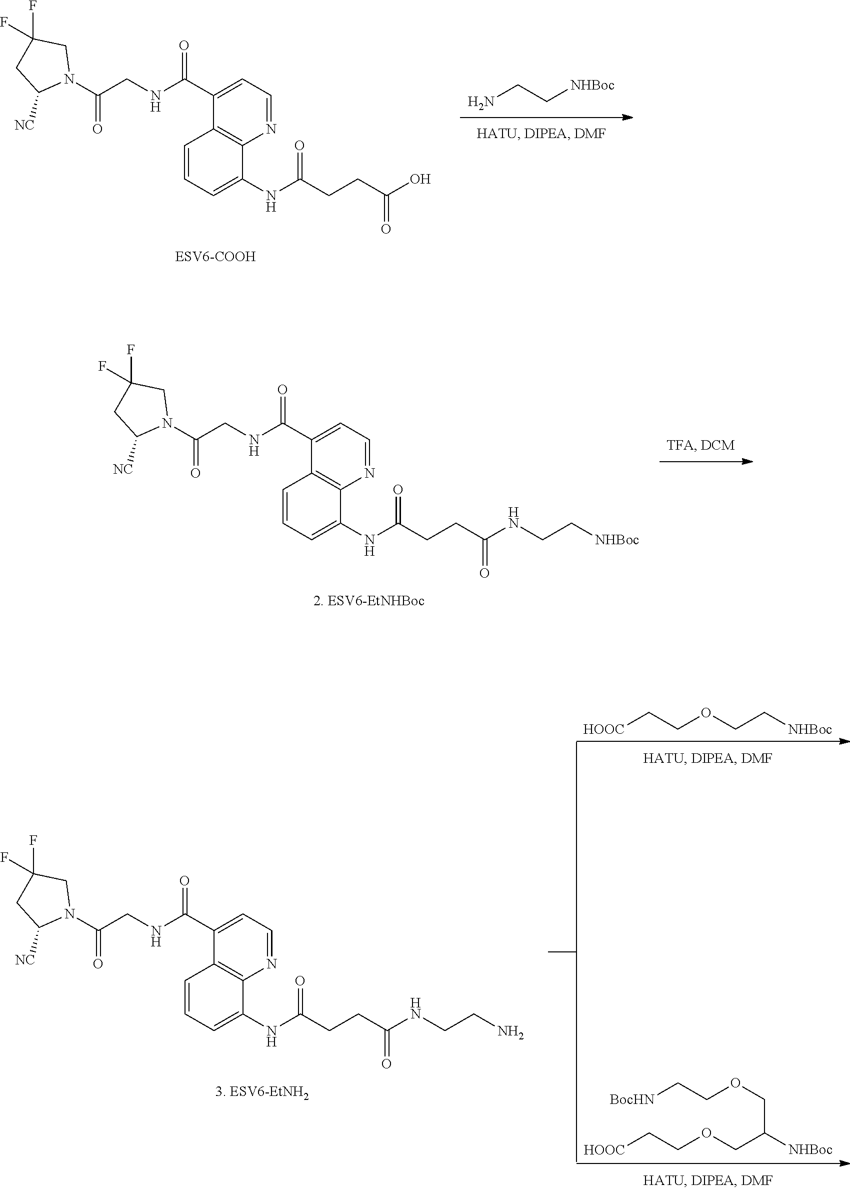 [SMILES: N#C[C@@H]1CC(F)(F)CN1C(=O)CNC(=O)c1ccnc2c(NC(=O)CCC(=O)O)cccc12 ; CC(C)(C)OC(=O)NCCN ; CC(C)(C)OC(=O)NCCNC(=O)CCC(=O)Nc1cccc2c(C(=O)NCC(=O)N3CC(F)(F)C[C@H]3C#N)ccnc12 ; CC(C)(C)OC(=O)NCCOCCC(=O)O ; N#C[C@@H]1CC(F)(F)CN1C(=O)CNC(=O)c1ccnc2c(NC(=O)CCC(=O)NCCN)cccc12 ; CC(C)(C)OC(=O)NCCOCC(COCCC(=O)O)NC(=O)OC(C)(C)C]

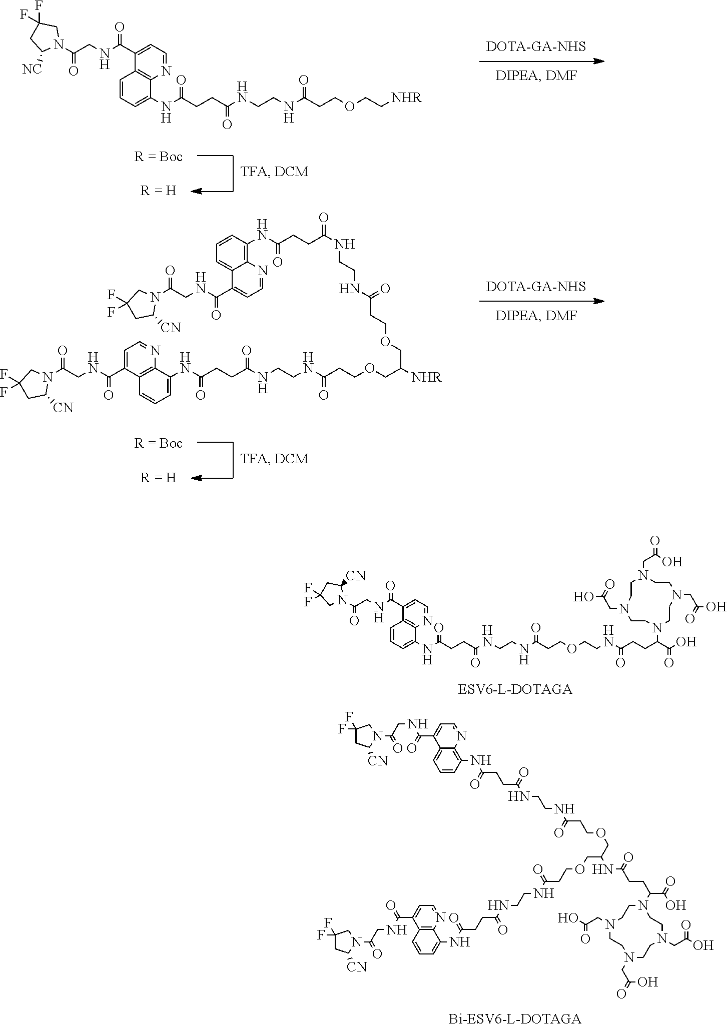

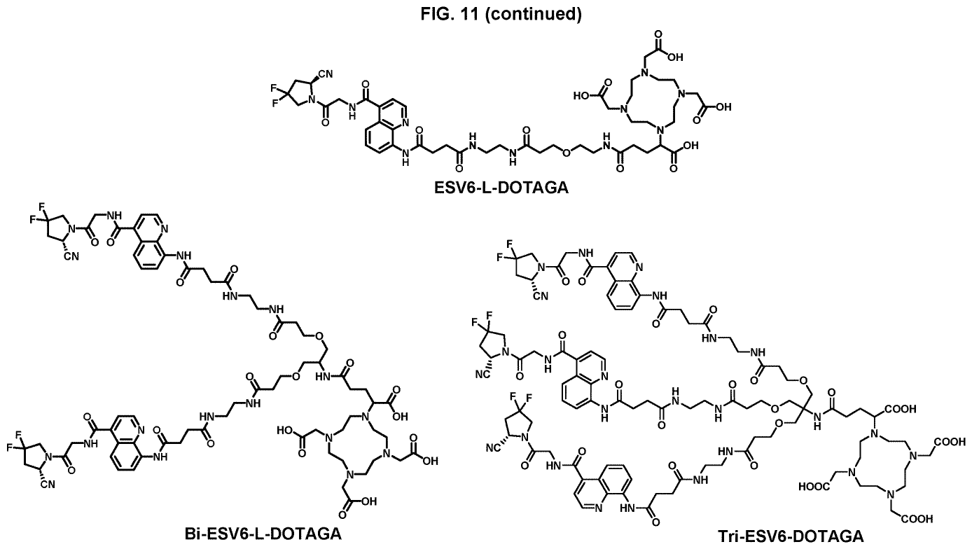

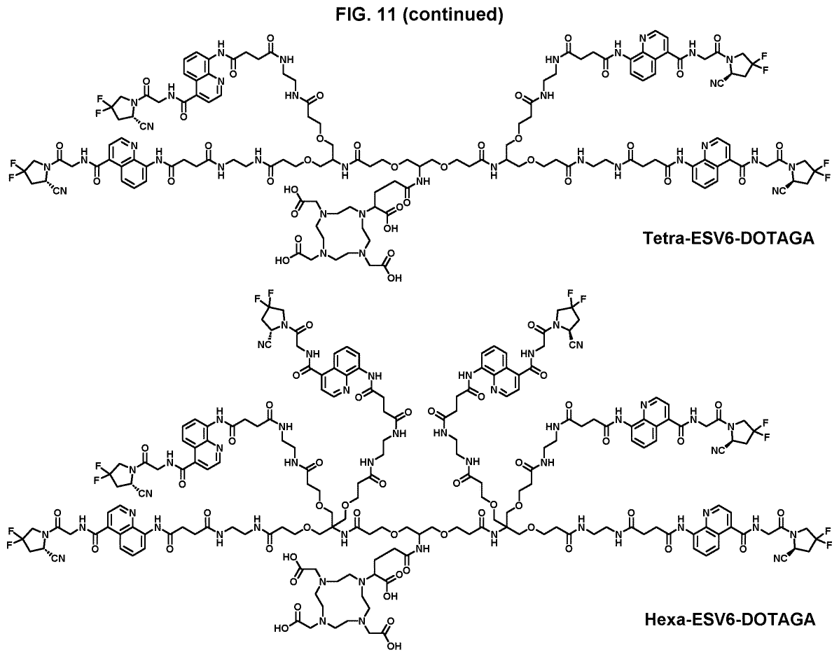

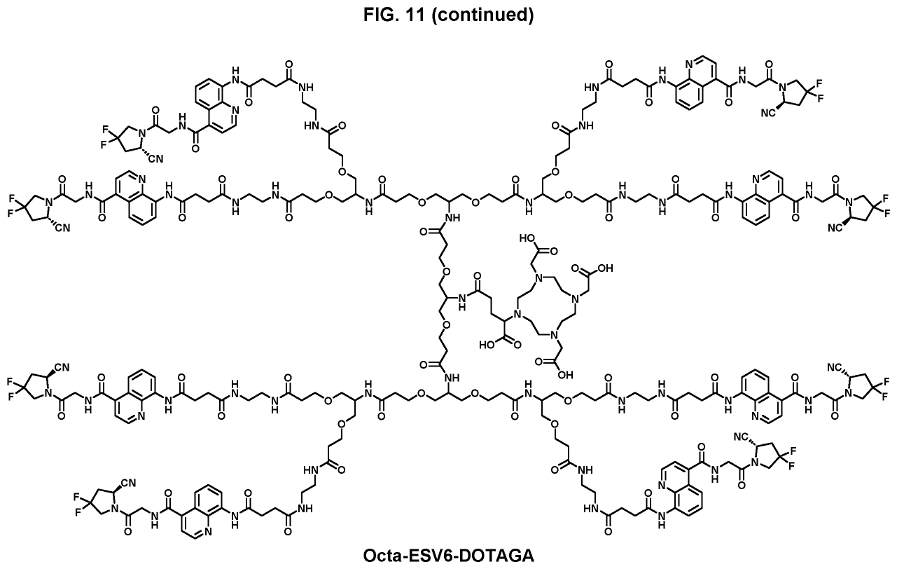

e) Inhibition assay with Tri-ESV6-DOTAGA, Tetra-ESV6-DOTAGA, Hexa-ESV6-DOTAGA, Octa-ESV6-DOTAGA, ESV6-L-DOTAGA and BI-ESV6-L-DOTAGA. Enzymatic activity of hFAP on the Z-Gly-Pro-AMC substrate was measured at room temperature on a microtiter plate reader, monitoring the fluorescence at an excitation wavelength of 360 nm and an emission wavelength of 465 nm. The reaction mixture contained substrate (20 μM), protein (66 μM, constant), assay buffer (50 mM Tris, 100 mM NaCl, and 1 mM EDTA, pH=7.4), and inhibitors with serial dilution from 167 nM to 80 fM, 1:2 in a total volume of 20 μL. Experiments were performed in triplicate, and the mean fluorescence values were fitted using Prism 7. The value is defined as the concentration of inhibitor required to reduce the enzyme activity by 50% after addition of the substrate. The results are shown in FIG. 11. The inhibitory activity is directly proportional to the valency until Tetra-ESV6-DOTAGA (tetravalent compound). Surprisingly, despite their superior valency, Hexa-ESV6-DOTAGA (hexavalent compound) and Octa-ESV6-DOTAGA (octavalent compound) lose over 10'000-fold activity compared to the tetravalent compound. Compounds with alternative linker (ESV6-L-DOTAGA and Bi-ESV6-DOTAGA) present IC50 comparable to the ones of original derivatives (ESV6-DOTAGA and Bi-ESV6-DOTAGA in FIG. 1).

### 13. Additional Compounds

### a) Chemical Structure of an Alternative Version of Tri-ESV6-DOTAGA (8)

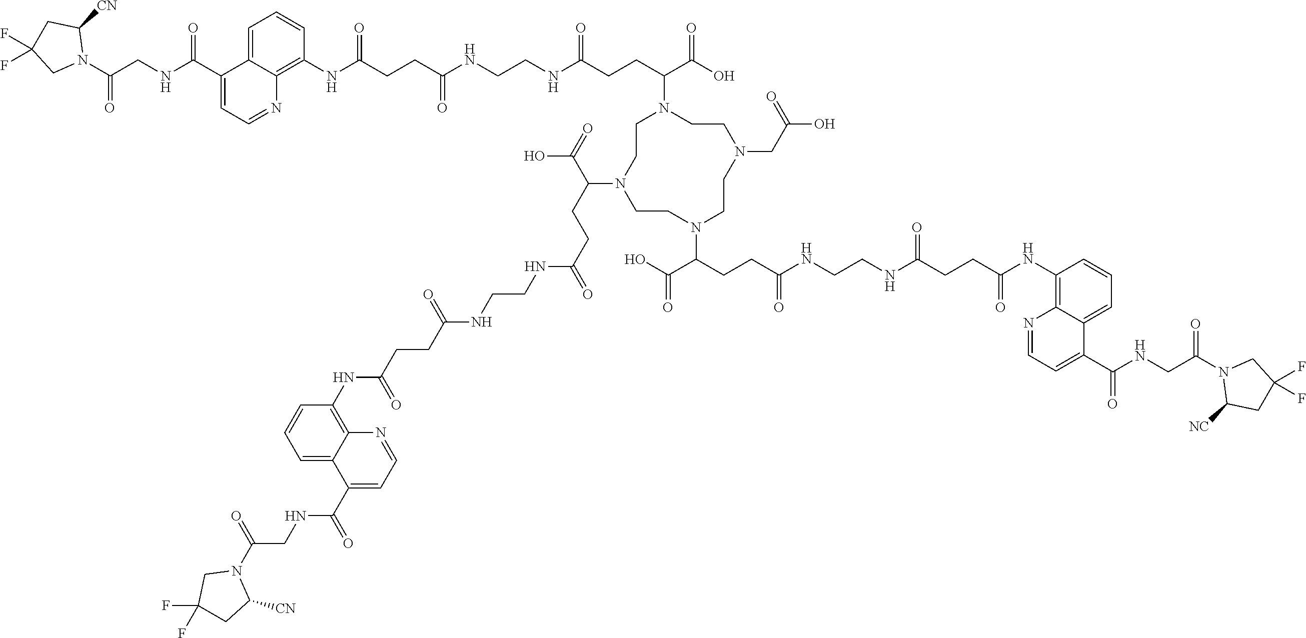 [SMILES: N#C[C@@H]1CC(F)(F)CN1C(=O)CNC(=O)c1ccnc2c(NC(=O)CCC(=O)NCCNC(=O)CCC(C(=O)O)N3CCN(CC(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CC3)cccc12]

Molecular weight: 2071.0484 Da
Exact mass: 2069.8032 Da
Formula: C94H109F6N25023
SMILES: O═C(CCC(NC1=C(N═CC═C2C(NCC(N3CC(F)(F)C[C@H]3C #N)═O)═O)C2=CC═C1)=O)NCCNC(CCC(N4CCN(C(C(O)═O)CCC(NCCNC(CCC(NC5=C(N═CC═C6C(NCC(N7CC(F)(F)C[C@H]7C #N)═O)═O)C6=CC═C5)=O)═O)═O)CCN(CC(O)═O)CCN(C(CCC(NCCNC(CCC(NC8=C(N═CC—C9C(NCC(N % 10CC(F)(F)C[C@H] % 10C #N)=O)═O)C9=CC═C8)-O)═O)═O)C(O)—O)CC4)C(O)—O)═O

### b) Synthetic Route for the Synthesis of the Alternative Version of TriOncoFAP-DOTAGA (8)

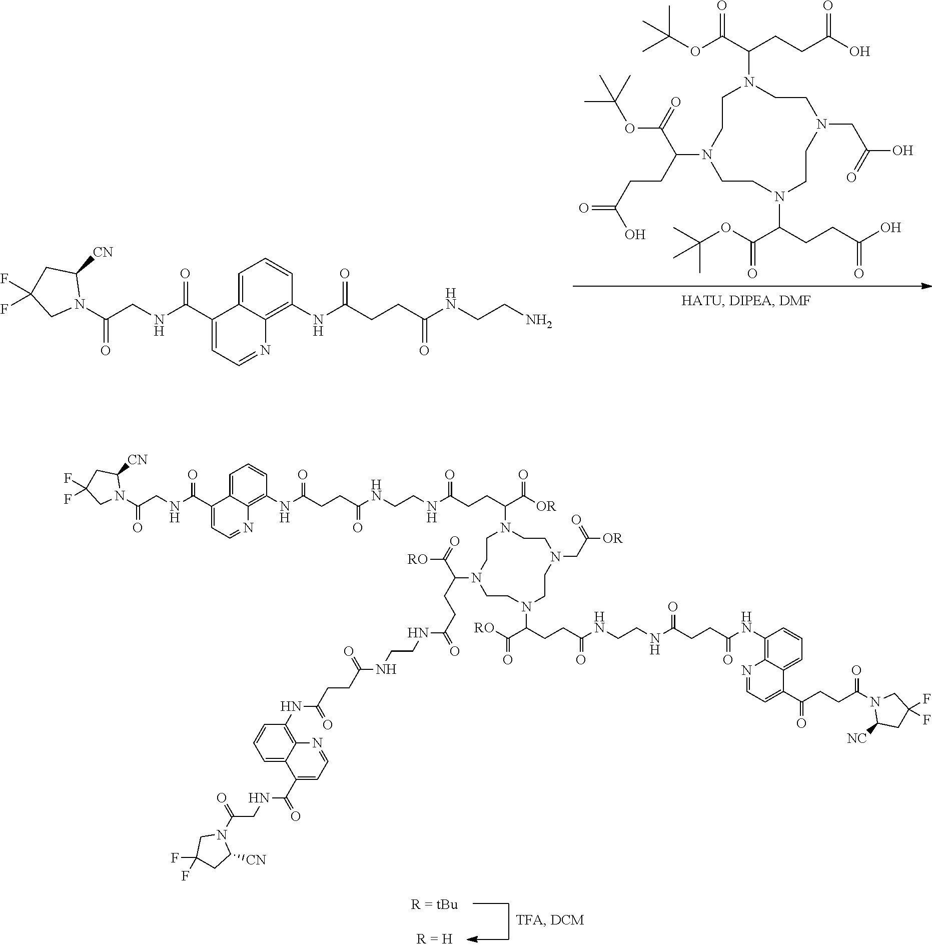

### c) Chemical Structure of Tri-ESV6-GlyPro-MMAE (9)

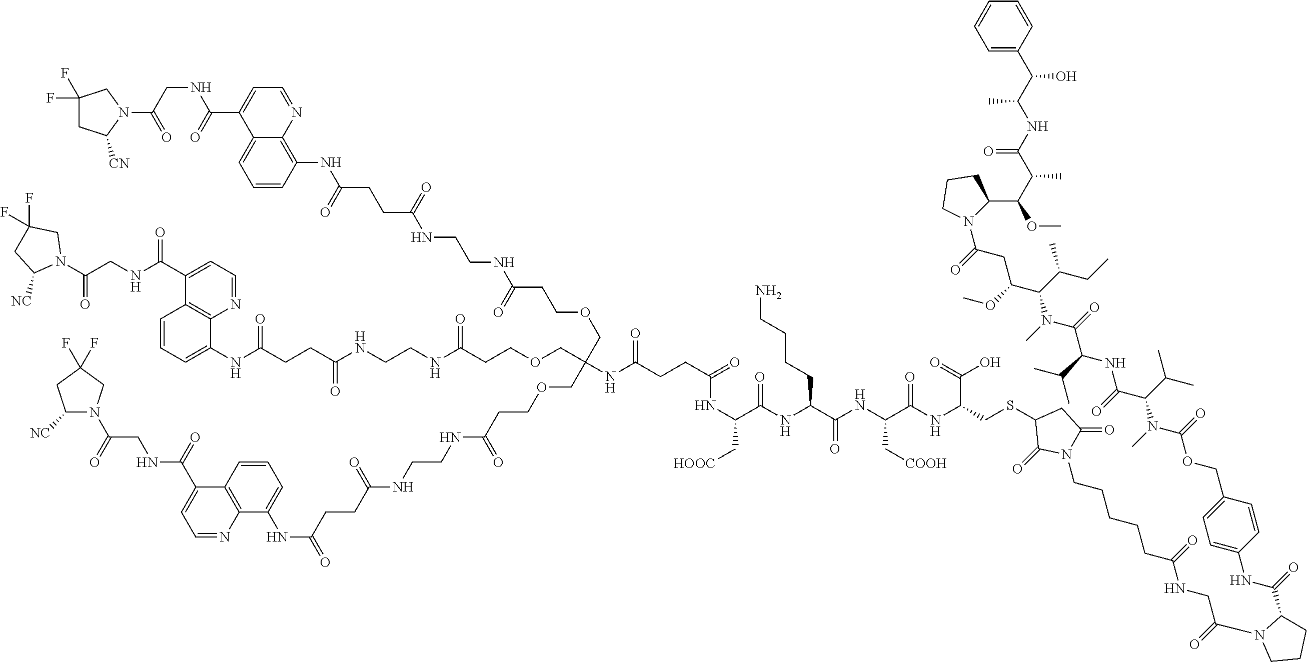

Molecular weight: 3563.8404 Da
Exact mass: 3561.5603 Da
Formula: C167H218F6N36043S
SMILES: O═CICC(SC[C@@H](C(O)═O)NC([C@H](CC(O)═O)NC([C@H](CCCCN)NC([C@H](CC(O)═O)NC(CCC(NC(COCCC(NCCNC(CCC(NC2=CC—CC3=C2N═CC═C3C(NCC(N4CC(F)(F)C[C@H]4C #N)—O)═O)═O)═O)═O) (COCCC(NCCNC(CCC(NC5=C(N═CC═C6C(NCC(N7CC(F)(F)C[C@H]7C #N)═O)═O) C6=CC═C5)=O)═O)═O)COCCC(NCCNC(CCC(NC8=CC═CC9=C8N═CC═C9C(NCC(N % 10 [C@H](C#N)CC(F)(F)C % 10)=O)═O)═O)═O)═O)═O)═O)═O)═O)═O)C(NICCCCCC(NCC(N % 11 [C@H](C(NC % 12=CC═C(COC(N(C)[C@@H](C(C)C)C(N [C@@H](C(C)C)C(N([C@@H]([C@H](C)CC)[C@H](OC) % 13)=O)C)=O)═O)═O)C═C % 12)=O)CCC % 11)=O)═O)═O

### Chemical Structure of (S,S)-Tri-ESV6-DOTAGA (11)

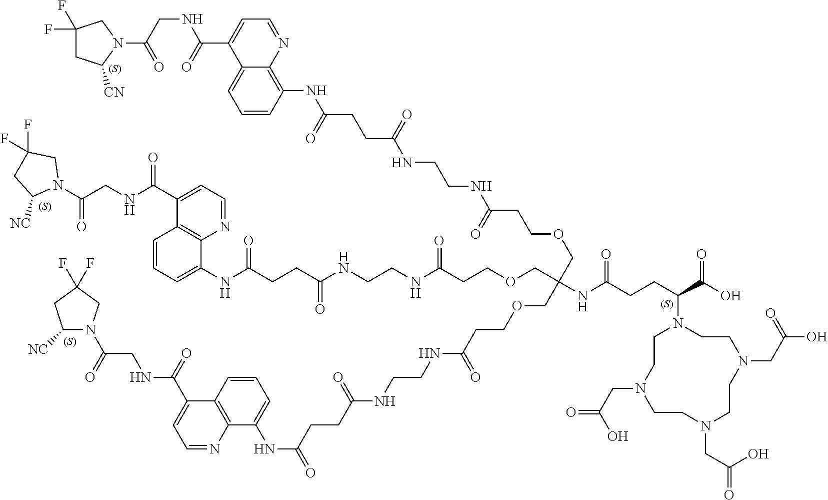

Molecular weight: 2246.2324 Da
Exact mass: 2244.8877 Da
Formula: C101H122F6N26027
SMILES: O═C(NCCNC(CCOCC(COCCC(NCCNC(CCC(NC1=C(N═CC═C2C(NCC(N3CC(F)(F)C[C@H]3C #N)═O)═O)C2=CC═C1)=O)═O)═O)(NC(CC[C@H](N4CCN(CC(O)═O)CCN(CC(O)═O)CCN(CC(O)═O)CC4)C(O)═O)═O)COC CC(NCCNC(CCC(NC5=CC═CC6=C5N═CC═C6C(NCC(N7CC(F)(F)C[C@H]7C #N)═O)═O)═O)═O)═O)═O)CCC(NC8=CC═CC9=C8N═CC═C9C(NCC(N % 10 [C@H](C#N)CC(F)(F)C % 10)=O)═O)═O

### Chemical Structure of (SR)-Tri-ESV6-DOTAGA (12)

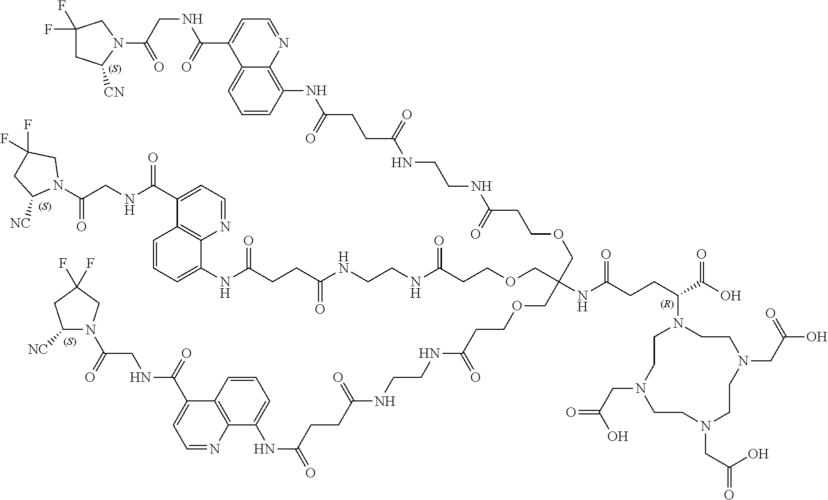

Molecular weight: 2246.2324 Da
Exact mass: 2244.8877 Da
Formula: C101H122F6N26027
SMILES: O═C(NCCNC(CCOCC(COCCC(NCCNC(CCC(NC1=C(N═CC—C2C(NCC(N3CC(F)(F)C[C@@H]3C #N)═O)═O) C2=CC═C1)=O)═O)═O)(NC(CC[C@@H](N4CCN(CC(O)═O)CCN(CC(O)═O)CCN(CC(O)═O)CC4)C(O)═O)═O) COCCC(NCCNC(CCC(NC5=CC═CC6=C5N═CC═C6C(NCC(N7CC(F)(F)C[C@@H]7C #N)═O)═O)═O)═O)═O)═O)CCC(NC8=CC═CC9=C8N═CC═C9C(NCC(N % 10 [C@@H](C#N)CC(F)(F)C % 10)=O)═O)═O

In all experiments described herein, unless indicated otherwise, Tri-ESV6-DOTAGA was used as its (S,R)-diastereomer.

### Chemical Structure of Tri-ESV6-Linker-DOTAGA (13)

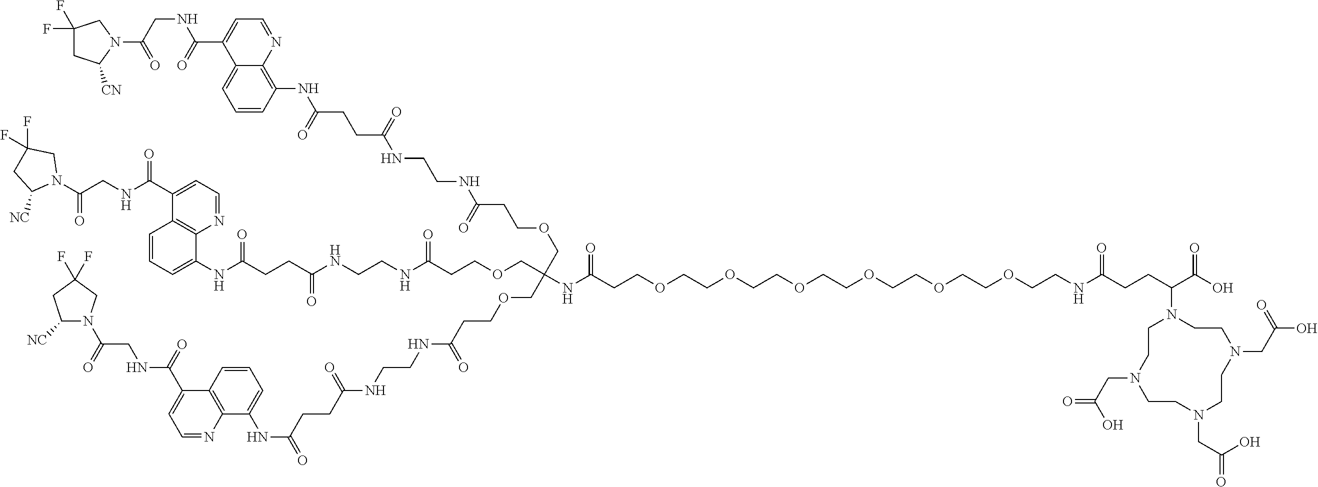 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CCOCCOCCOCCOCCOCCOCCNC(=O)CCC(C(=O)O)N3CCN(CC(=O)O)CCN(CC(=O)O)CCN(CC(=O)O)CC3)cccc12]

Molecular weight: 2581.6294 Da
Exact mass: 2580.0821 Da
Formula: C116H151F6N27034
SMILES: O═C(NCCNC(CCOCC(COCCC(NCCNC(CCC(NC1=C(N═CC═C2C(NCC(N3CC(F)(F)C[C@H]3C #N)═O)═O)C2=CC═C1)=O)═O)═O)(NC(CCOCCOCCOCCOCCOCCOCCNC(CCC(N4CCN(CC(O)═O)CCN(CC(O)═O)CCN(C C(O)═O)CC4)C(O)═O)═O)═O)COCCC(NCCNC(CCC(NC5=CC═CC6=C5N═CC═C6C(NCC(N7CC(F)(F)C[C@H]7C #N)═O)═O)═O)═O)═O)═O)CCC(NC8=CC═CC9=C8N═CC═C9C(NCC(N % 10 [C@H](C#N)CC(F)(F)C % 10)=O)═O)═O

### Chemical Structure of Tri-ESV6-ValCit-MMAE (14)

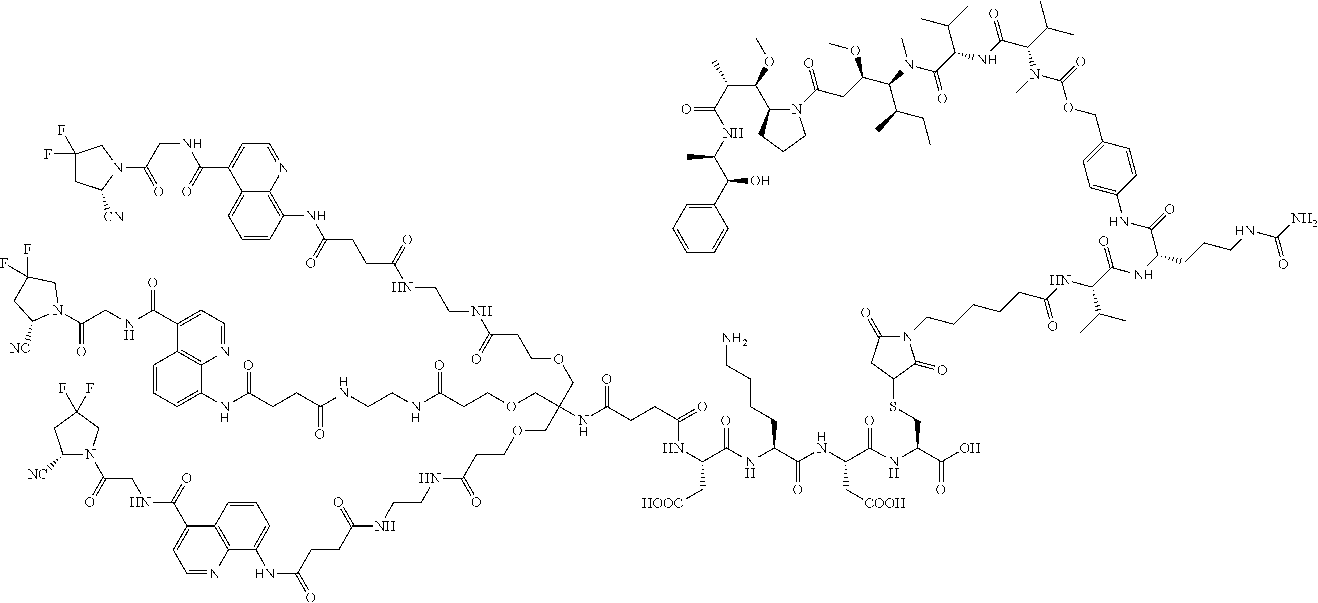

Molecular weight: 3665.9774 Da
Exact mass: 3663.6397 Da
Formula: C171H228F6N38044S
SMILES: O═C1CC(SC[C@@H](C(O)═O)NC([C@H](CC(O)═O)NC([C@H](CCCCN)NC([C@H](CC(O)═O)NC(CCC(NC(COCCC(NCCNC(CCC(NC2=CC═CC3=C2N═CC═C3C(NCC(N4CC(F)(F)C[C@H]4C #N)═O)═O)═O)═O)═O)(CO CCC(NCCNC(CCC(NC5=C(N═CC═C6C(NCC(N7CC(F)(F)C[C@H]7C #N)═O)═O)C6=CC═C5)=O)═O)═O)COC CC(NCCNC(CCC(NC8=CC═CC9=C8N═CC═C9C(NCC(N % 10 [C@H](C#N)CC(F)(F)C % 10)=O)═O)═O)═O)═O)═O)═O)═O)═O)═O)C(NICCCCCC(N [C@@H](C(C)C)C(N[C@H](C(NC % 11=CC═C(COC(N(C)[C@@H](C(C)C) C(N [C@@H](C(C)C)C(N([C@@H]([C@H](C)CC)[C@H](OC)CC(N % 12 [C@H]([C@H](OC)[C@@H](C)C(N[C@H](C)[C@@H](O)C % 13=CC═CC=C % 13)=O)CCC % 12)=O)C)=O)═O)═O)C=C % 11)=O)CCCNC(N)═O)═O)═O)═O

### 14. Preferred Conjugates

For ease of reference, numbers and structures of some compounds of the invention are summarized in Table 9 and Table 17 below.

TABLE 1
Injected dose per gram
¹⁷⁷Lu-ESV6-DOTAGA

| | |
|---|---|
| 24 h | 48 h |

| | Avg. | Std. d | Avg. | Std. d |
|---|---|---|---|---|
| Tumor | 2.53 | 0.32 | 1.19 | 0.23 |
| Heart | 0.01 | 0.00 | 0.00 | 0.00 |
| Lung | 0.01 | 0.01 | 0.01 | 0.01 |
| Liver | 0.07 | 0.01 | 0.06 | 0.01 |
| Spleen | 0.03 | 0.02 | 0.01 | 0.01 |
| Intestine | 0.01 | 0.01 | 0.01 | 0.00 |
| Kidney | 0.47 | 0.09 | 0.28 | 0.04 |
| Blood | 0.00 | 0.00 | 0.00 | 0.00 |
| Muscle | 0.03 | 0.02 | 0.01 | 0.00 |
| Tail | 0.05 | 0.01 | 0.03 | 0.00 |

TABLE 2
Injected dose per gram

| ¹⁷⁷Lu-Bi-ESV6-DOTAGA | | |
|---|---|---|
| 24 h | 48 h | 72 h |

| | Avg. | Std. d | Avg. | Std. d | Avg. | Std. d |
|---|---|---|---|---|---|---|
| Tumor | 14.29 | 0.77 | 8.10 | 0.44 | 8.00 | 1.14 |
| Heart | 0.05 | 0.01 | 0.01 | 0.01 | 0.04 | 0.01 |
| Lung | 0.08 | 0.03 | 0.03 | 0.00 | 0.06 | 0.07 |
| Liver | 0.29 | 0.03 | 0.16 | 0.03 | 0.29 | 0.06 |
| Spleen | 0.11 | 0.01 | 0.07 | 0.03 | 0.14 | 0.04 |
| Intestine | 0.05 | 0.02 | 0.03 | 0.02 | 0.06 | 0.08 |
| Kidney | 0.96 | 0.07 | 0.47 | 0.04 | 0.66 | 0.13 |
| Blood | 0.05 | 0.01 | 0.00 | 0.00 | 0.00 | 0.00 |
| Muscle | 0.12 | 0.04 | 0.05 | 0.03 | 0.08 | 0.07 |
| Tail | 0.22 | 0.06 | 0.07 | 0.05 | 0.12 | 0.00 |

TABLE 3
Injected dose per gram

| | ¹⁷⁷Lu-Tri-ESV6-DOTAGA | | | | | | | | | |
|---|---|---|---|---|---|---|---|---|---|---|
| | 6 h | | 24 h | | 48 h | | 72 h | | 96 h | |
| | Avg. | Std. d | Avg. | Std. d | Avg. | Std. d | Avg. | Std. d | Avg. | Std. d |
| Tumor | 48.96 | 5.82 | 19.78 | 2.06 | 16.64 | 1.54 | 20.19 | 1.92 | 16.26 | 1.68 |
| Heart | 0.23 | 0.02 | 0.08 | 0.02 | 0.03 | 0.01 | 0.07 | 0.03 | 0.03 | 0.01 |
| Lung | 0.38 | 0.05 | 0.10 | 0.01 | 0.06 | 0.01 | 0.11 | 0.15 | 0.06 | 0.04 |
| Liver | 0.65 | 0.04 | 0.46 | 0.06 | 0.31 | 0.05 | 0.34 | 0.24 | 0.40 | 0.06 |
| Spleen | 0.30 | 0.14 | 0.16 | 0.10 | 0.10 | 0.06 | 0.24 | 0.08 | 0.24 | 0.01 |
| Intestine | 0.26 | 0.03 | 0.10 | 0.06 | 0.06 | 0.01 | 0.07 | 0.07 | 0.02 | 0.01 |
| Kidney | 2.14 | 0.17 | 1.16 | 0.11 | 0.78 | 0.10 | 0.83 | 0.14 | 0.73 | 0.07 |
| Blood | 0.25 | 0.13 | 0.07 | 0.00 | 0.01 | 0.00 | 0.01 | 0.02 | 0.00 | 0.00 |
| Muscle | 0.45 | 0.45 | 0.18 | 0.15 | 0.04 | 0.03 | 0.04 | 0.02 | 0.03 | 0.01 |
| Tail | 1.06 | 0.25 | 0.31 | 0.04 | 0.14 | 0.06 | 0.19 | 0.05 | 0.17 | 0.02 |

TABLE 4
Injected dose per gram

| | FAP-2286 | | | | | | | | | |
|---|---|---|---|---|---|---|---|---|---|---|
| | 6 h | | 24 h | | 48 h | | 72 h | | 96 h | |
| | Avg. | Std. d | Avg. | Std. d | Avg. | Std. d | Avg. | Std. d | Avg. | Std. d |
| Tumor | 23.50 | 3.77 | 9.72 | 1.90 | 8.24 | 1.66 | 8.58 | 1.03 | 5.00 | 0.36 |
| Heart | 0.03 | 0.02 | 0.01 | 0.00 | 0.01 | 0.01 | 0.02 | 0.02 | 0.00 | 0.00 |
| Lung | 0.05 | 0.03 | 0.02 | 0.01 | 0.02 | 0.01 | 0.02 | 0.02 | 0.01 | 0.01 |
| Liver | 0.11 | 0.05 | 0.05 | 0.02 | 0.05 | 0.01 | 0.04 | 0.02 | 0.04 | 0.02 |
| Spleen | 0.06 | 0.06 | 0.02 | 0.01 | 0.01 | 0.01 | 0.06 | 0.07 | 0.05 | 0.06 |
| Intestine | 0.04 | 0.03 | 0.02 | 0.01 | 0.01 | 0.00 | 0.02 | 0.01 | 0.01 | 0.02 |
| Kidney | 4.46 | 0.20 | 2.10 | 0.24 | 1.14 | 0.18 | 1.11 | 0.16 | 0.84 | 0.15 |
| Blood | 0.01 | 0.01 | 0.00 | 0.00 | 0.00 | 0.00 | 0.00 | 0.00 | 0.00 | 0.00 |
| Muscle | 0.04 | 0.03 | 0.01 | 0.00 | 0.00 | 0.01 | 0.01 | 0.01 | 0.01 | 0.03 |
| Tail | 0.24 | 0.12 | 0.06 | 0.02 | 0.05 | 0.02 | 0.04 | 0.01 | 0.04 | 0.03 |

TABLE 5
Tumor to organs ratio
¹⁷⁷Lu-ESV6-DOTAGA

| | |
|---|---|
| 24 h | 48 h |

| | Avg. | Std. d | Avg. | Std. d |
|---|---|---|---|---|
| Heart | 497.97 | 188.20 | 314.15 | 128.34 |
| Lung | 977.01 | 862.42 | 805.04 | 665.19 |
| Liver | 37.66 | 3.86 | 20.54 | 3.21 |
| Spleen | 158.68 | 99.05 | 503.00 | 667.46 |
| Intestine | 240.54 | 146.77 | 232.61 | 101.19 |
| Kidney | 5.41 | 0.38 | 4.22 | 0.66 |
| Blood | 628.54 | 129.67 | 2173.00 | 1746.16 |
| Muscle | 159.12 | 146.22 | 142.88 | 58.96 |
| Tail | 48.95 | 1.57 | 45.61 | 11.75 |

TABLE 6
Tumor to organs ratio

| | ¹⁷⁷Lu-Bi-ESV6-DOTAGA | | | | | |
|---|---|---|---|---|---|---|
| | 24 h | | 48 h | | 72 h | |
| | Avg. | Std. d | Avg. | Std. d | Avg. | Std. d |
| Heart | 342.92 | 140.14 | 1206.24 | 1360.06 | 203.58 | 8.55 |
| Lung | 184.57 | 62.80 | 300.13 | 40.04 | 517.13 | 593.74 |
| Liver | 48.81 | 2.26 | 51.30 | 10.93 | 28.14 | 6.23 |
| Spleen | 125.34 | 12.99 | 133.66 | 78.48 | 61.08 | 11.91 |
| Intestine | 298.43 | 83.33 | 458.76 | 260.93 | 544.57 | 581.06 |
| Kidney | 14.96 | 1.32 | 17.41 | 2.09 | 12.21 | 0.73 |
| Blood | 270.81 | 60.15 | 4240.99 | 3054.91 | 2515.44 | 1141.18 |
| Muscle | 135.38 | 53.63 | 270.58 | 233.43 | 140.83 | 72.09 |
| Tail | 69.69 | 18.23 | 187.42 | 166.69 | 64.13 | 8.05 |

TABLE 7
Tumor to organs ratio

| | ¹⁷⁷Lu-Tri-ESV6-DOTAGA | | | | | | | | | |
|---|---|---|---|---|---|---|---|---|---|---|
| | 6 h | | 24 h | | 48 h | | 72 h | | 96 h | |
| | Avg. | Std. d | Avg. | Std. d | Avg. | Std. d | Avg. | Std. d | Avg. | Std. d |
| Heart | 215.4 | 38.7 | 269.1 | 82.6 | 559.6 | 163.1 | 344.2 | 159 | 492 | 99 |
| Lung | 128.8 | 7.2 | 196.9 | 37.1 | 299.5 | 62.8 | 477.1 | 428 | 344 | 158 |
| Liver | 76.3 | 12.6 | 43.9 | 7.6 | 56.2 | 14.2 | 1294.6 | 2497 | 41 | 6 |
| Spleen | 196.0 | 103.9 | 384.4 | 578.9 | 244.8 | 201.2 | 92.1 | 33 | 69 | 6 |
| Intestine | 188.9 | 41.0 | 243.1 | 119.3 | 282.6 | 60.9 | 498.0 | 320 | 763 | 193 |
| Kidney | 22.9 | 2.5 | 17.2 | 2.6 | 21.4 | 2.0 | 24.8 | 4 | 22 | 3 |
| Blood | 260.4 | 194.5 | 284.8 | 40.5 | 1925.8 | 665.8 | 7913.2 | 7252 | 3504 | 346 |
| Muscle | 187.0 | 123.1 | 159.9 | 84.0 | 1095.4 | 1292.9 | 536.6 | 177 | 514 | 146 |
| Tail | 48.4 | 16.1 | 65.3 | 15.5 | 136.9 | 55.6 | 108.9 | 22 | 98 | 13 |

TABLE 8
(Tumor to organs ratio)

| | FAP-2286 | | | | | | | | | |
|---|---|---|---|---|---|---|---|---|---|---|
| | 6 h | | 24 h | | 48 h | | 72 h | | 96 h | |
| | Avg. | Std. d | Avg. | Std. d | Avg. | Std. d | Avg. | Std. d | Avg. | Std. d |
| Heart | 1148.2 | 920 | 1445.4 | 850 | 1063.6 | 604 | 3262.7 | 3425 | 2163 | 1807 |
| Lung | 1314.6 | 1691 | 449.4 | 145 | 692.4 | 474 | 932.1 | 720 | 256 | 82 |
| Liver | 242.8 | 118 | 224.6 | 104 | 166.7 | 33 | 242.5 | 86 | 138 | 61 |
| Spleen | 674.4 | 446 | 438.7 | 173 | 589.3 | 163 | 398.2 | 341 | 238 | 249 |
| Intestine | 775.5 | 360 | 688.8 | 382 | 937.8 | 294 | 741.4 | 476 | 1064 | 1056 |
| Kidney | 5.3 | 1 | 4.6 | 1 | 7.5 | 3 | 7.9 | 2 | 6 | 1 |
| Blood | 3690.0 | 2264 | 2722.6 | 962 | 4800.4 | 3213 | 2985.6 | 1991 | 1394 | 786 |
| Muscle | 1018.5 | 981 | 6042.4 | 5671 | 1698.5 | 1358 | 1528.5 | 1759 | 201 | 150 |
| Tail | 118.2 | 63 | 195.8 | 84 | 172.3 | 44 | 266.2 | 88 | 145 | 69 |

TABLE 9

| # | Structure |
|---|---|
| 6 | 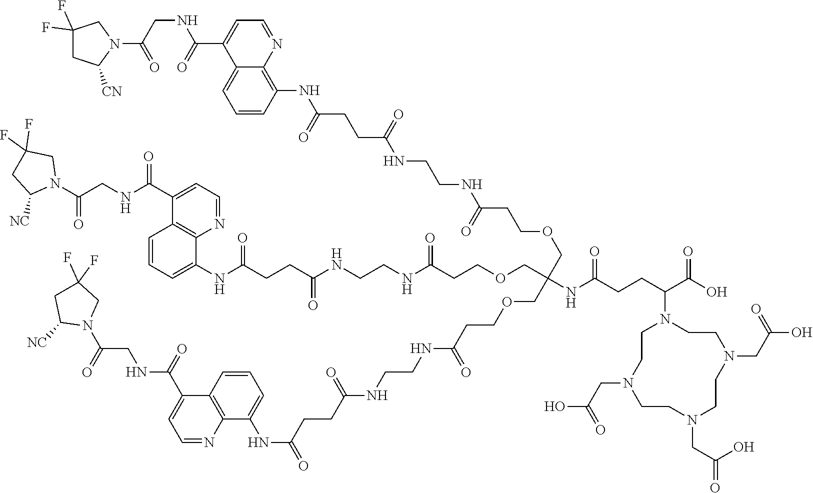 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12] |
| 6a | 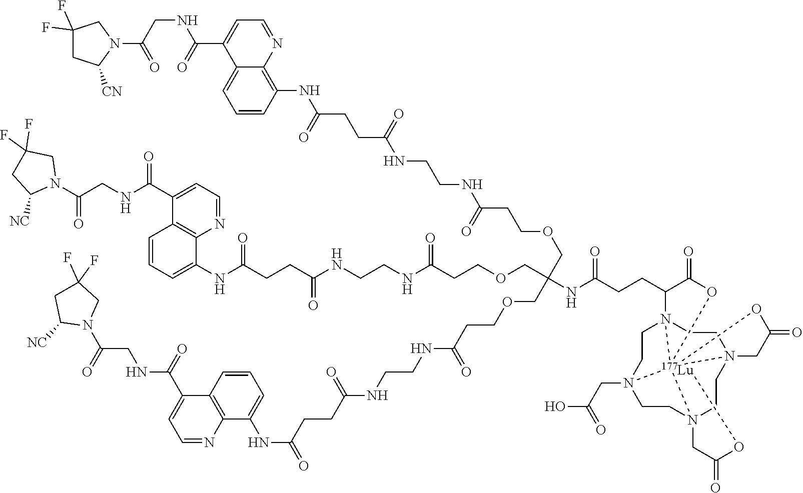 |
| 6b | 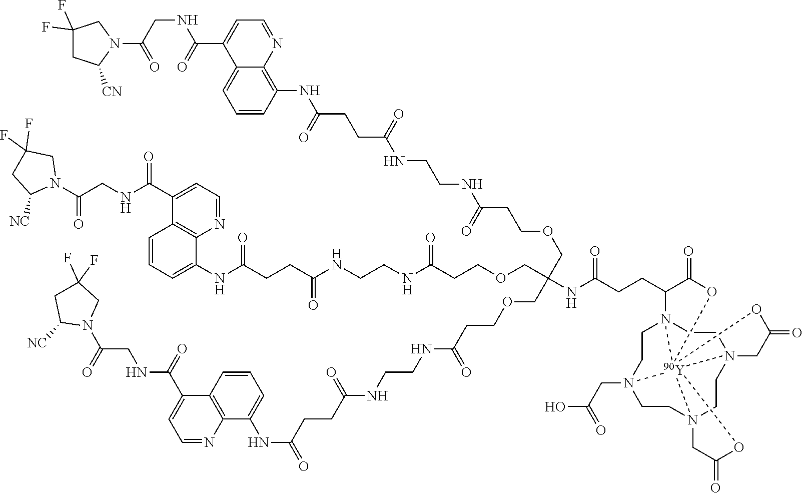 |
| 6c | 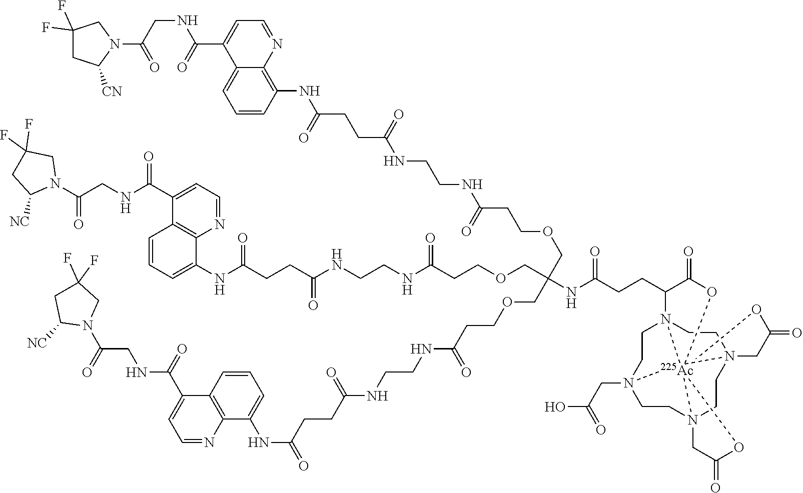 |
| 6d | 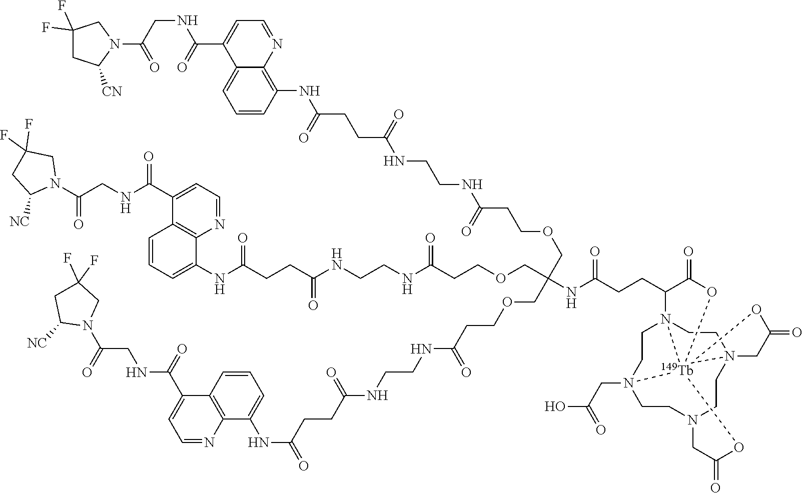 |
| 7 | 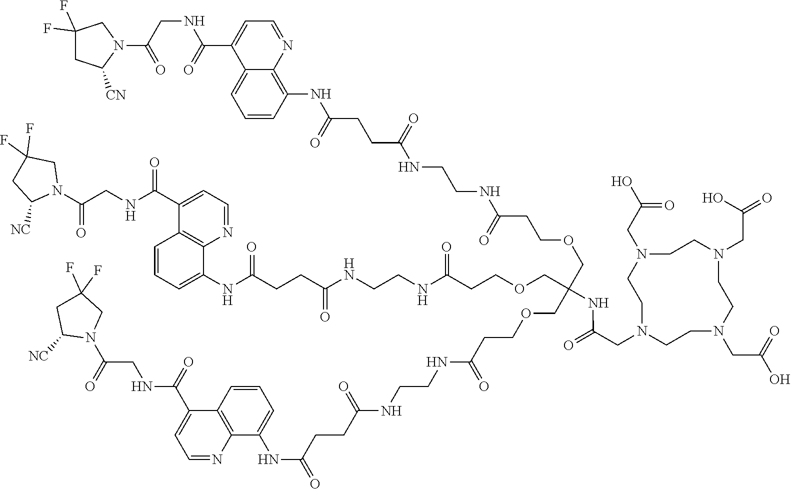 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CN3CCN(CC(=O)O)CCN(CC(=O)O)CCN(CC(=O)O)CC3)cccc12] |
| 7a | 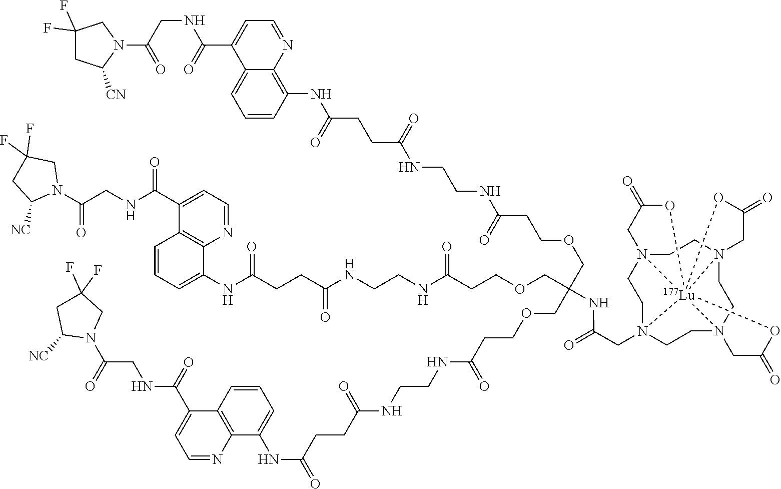 |
| 7b | 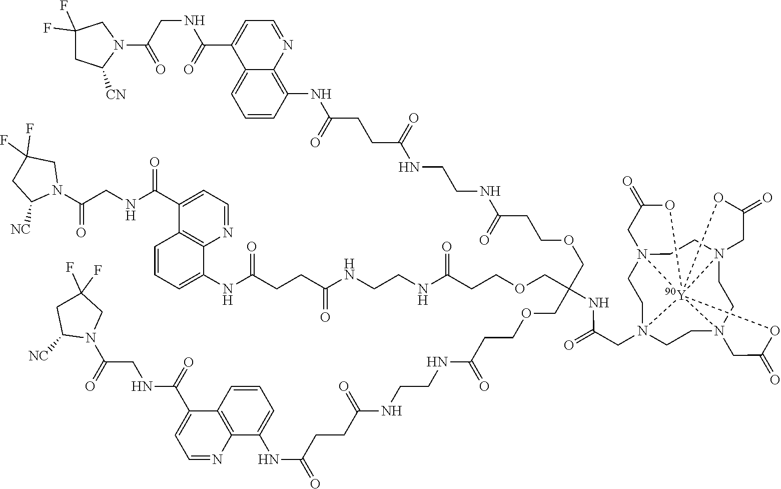 |
| 7c | 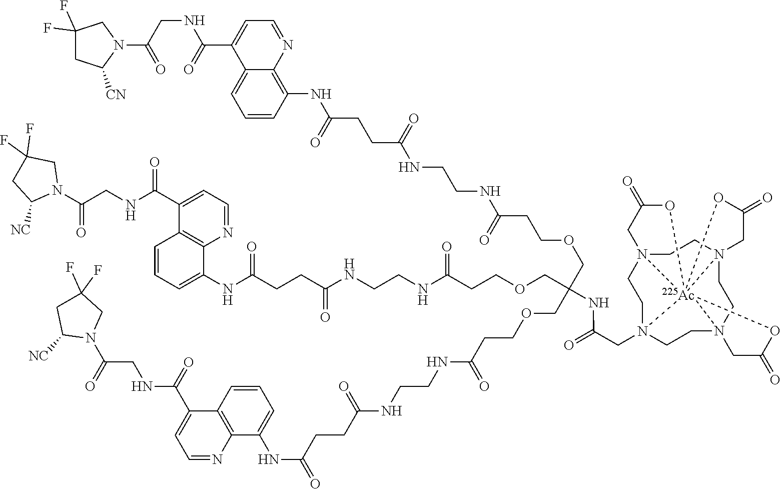 |
| 7d | 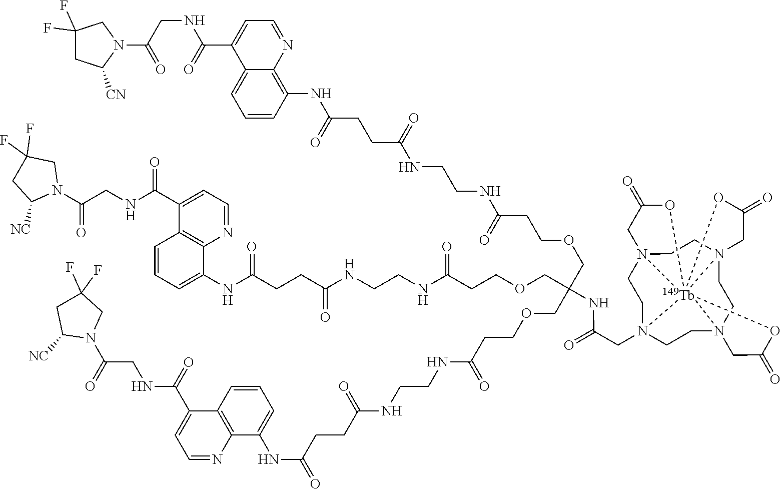 |
| 6e | 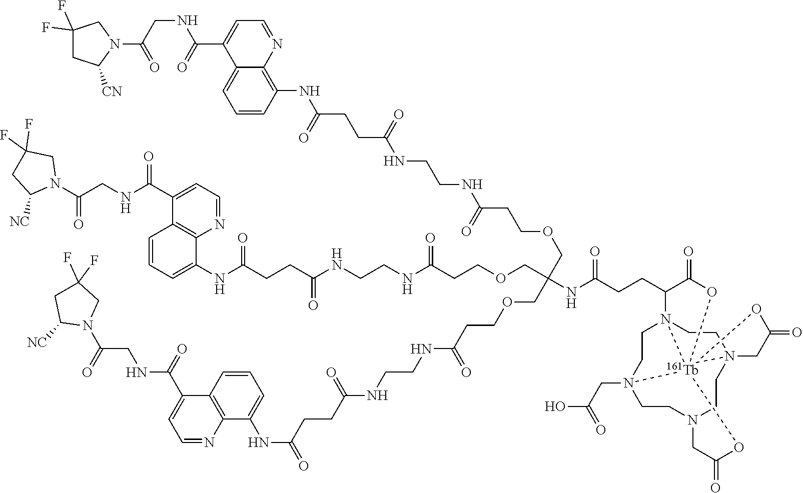 |
| 7e | 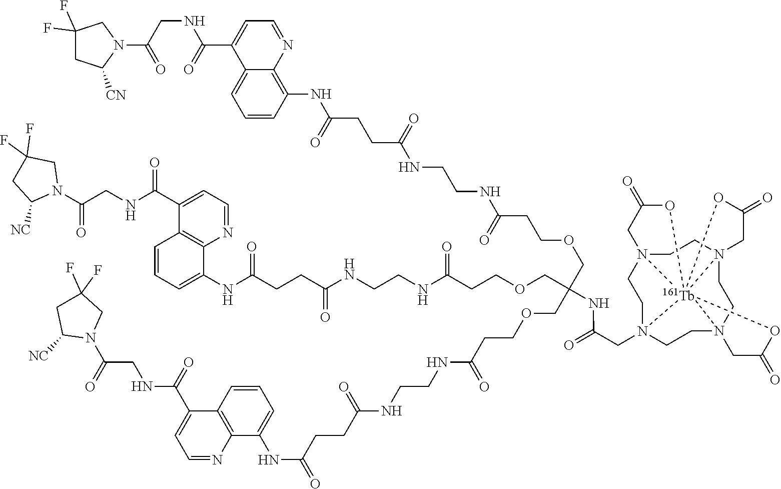 |
| 6f | 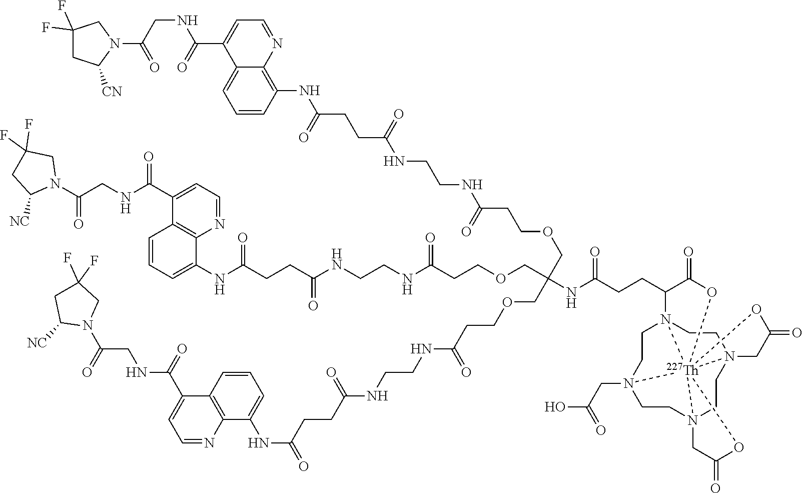 |
| 7f | 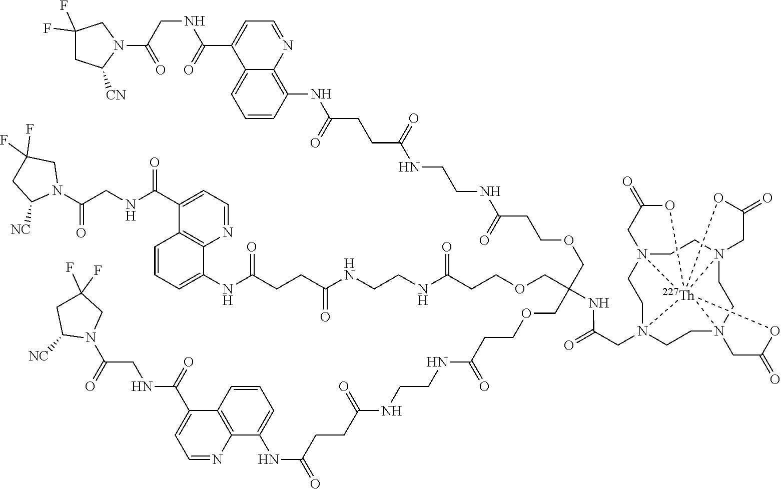 |
| 8 | 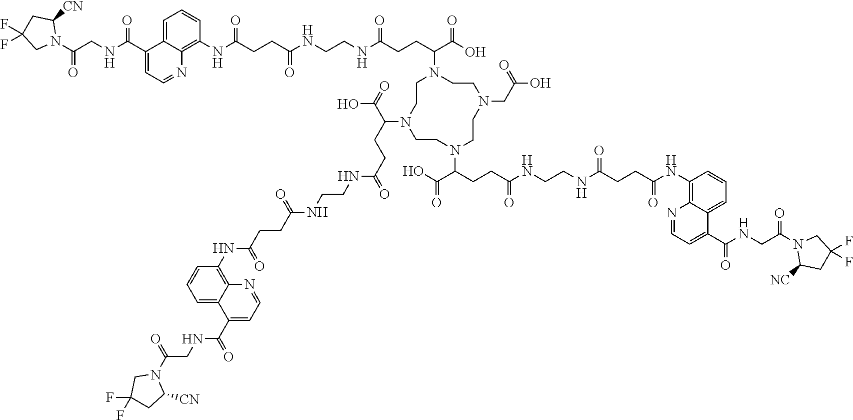 [SMILES: N#C[C@@H]1CC(F)(F)CN1C(=O)CNC(=O)c1ccnc2c(NC(=O)CCC(=O)NCCNC(=O)CCC(C(=O)O)N3CCN(CC(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CC3)cccc12] |
| 9 | 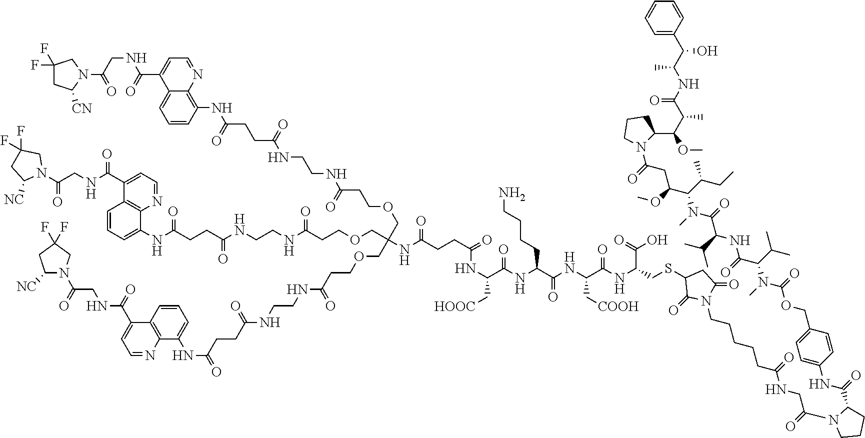 |
| 11 | 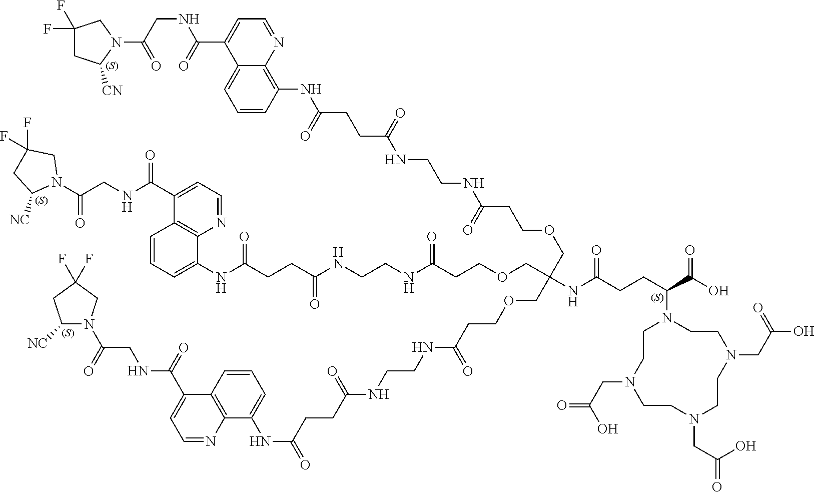 |
| 12 | 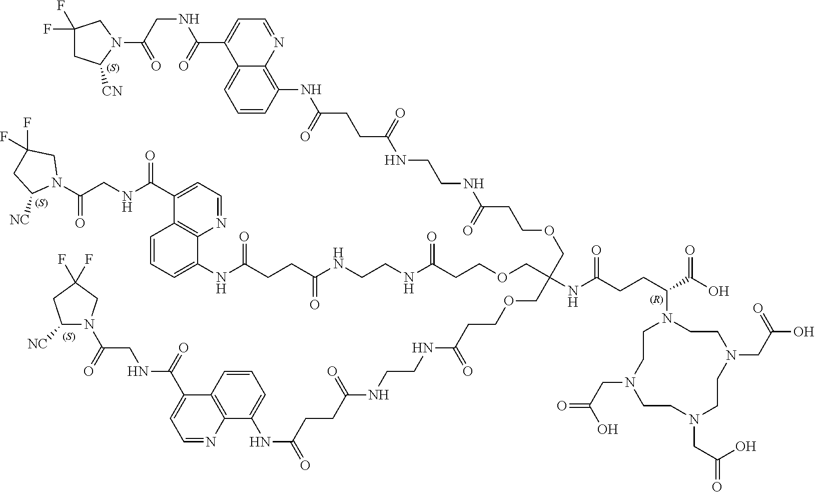 |
| 13 | 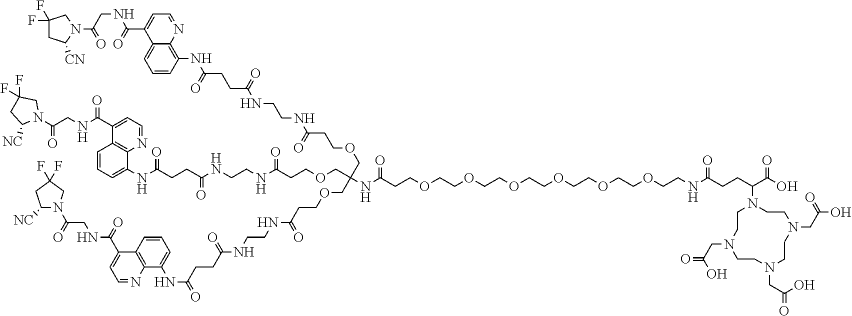 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)(COCCC(=O)NCCNC(=O)CCC(=O)Nc3cccc4c(C(=O)NCC(=O)N5CC(F)(F)C[C@H]5C#N)ccnc34)NC(=O)CCOCCOCCOCCOCCOCCOCCNC(=O)CCC(C(=O)O)N3CCN(CC(=O)O)CCN(CC(=O)O)CCN(CC(=O)O)CC3)cccc12] |
| 14 | 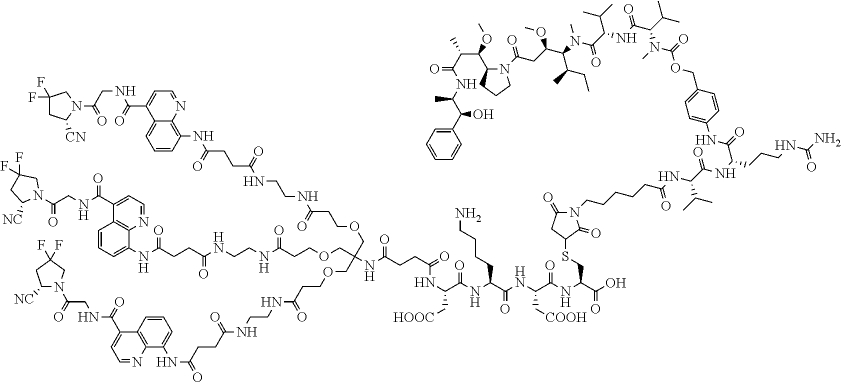 |

TABLE 10
Injected dose per gram

| ¹⁷⁷Lu-Tetra-ESV6-DOTAGA | | |
|---|---|---|
| 6 h | 24 h | 96 h |

| | Avg. | Std. d | Avg. | Std. d | Avg. | Std. d |
|---|---|---|---|---|---|---|
| Tumor | 57.25 | 4.61 | 42.15 | 6.62 | 13.36 | 0.85 |
| Heart | 0.50 | 0.03 | 0.26 | 0.03 | 0.12 | 0.07 |
| Lung | 1.09 | 0.11 | 0.35 | 0.10 | 0.24 | 0.12 |
| Liver | 3.81 | 0.46 | 4.52 | 0.60 | 6.64 | 1.14 |
| Spleen | 1.33 | 0.22 | 2.19 | 0.64 | 3.64 | 0.89 |
| Intestine | 0.70 | 0.16 | 0.15 | 0.03 | 0.07 | 0.03 |
| Kidney | 2.10 | 0.35 | 1.79 | 0.21 | 5.77 | 0.71 |
| Blood | 1.16 | 0.42 | 0.29 | 0.03 | 0.01 | 0.00 |
| Muscle | 0.94 | 0.14 | 0.44 | 0.09 | 0.06 | 0.04 |
| Tail | 1.85 | 1.24 | 1.16 | 0.12 | 0.54 | 0.06 |

TABLE 11
Injected dose per gram

| ¹⁷⁷Lu-Hexa-ESV6-DOTAGA | | |
|---|---|---|
| 6 h | 24 h | 96 h |

| | Avg. | Std. d | Avg. | Std. d | Avg. | Std. d |
|---|---|---|---|---|---|---|
| Tumor | 2.77 | 0.49 | 1.80 | 0.49 | 1.36 | 0.20 |
| Heart | 0.33 | 0.05 | 0.23 | 0.04 | 0.16 | 0.05 |
| Lung | 6.91 | 2.36 | 5.33 | 1.54 | 1.38 | 0.54 |
| Liver | 62.54 | 9.61 | 53.45 | 6.25 | 49.80 | 4.01 |
| Spleen | 41.26 | 5.05 | 47.44 | 1.99 | 38.61 | 5.73 |
| Intestine | 0.24 | 0.15 | 0.10 | 0.03 | 0.07 | 0.02 |
| Kidney | 0.53 | 0.07 | 0.57 | 0.16 | 1.37 | 0.05 |
| Blood | 0.36 | 0.06 | 0.10 | 0.01 | 0.02 | 0.01 |
| Muscle | 0.66 | 0.87 | 0.14 | 0.04 | 0.05 | 0.03 |
| Tail | 0.70 | 0.30 | 1.65 | 1.33 | 1.02 | 0.81 |

TABLE 12
Injected dose per gram

| ¹⁷⁷Lu-Octa-ESV6-DOTAGA | | |
|---|---|---|
| 6 h | 24 h | 96 h |

| | Avg. | Std. d | Avg. | Std. d | Avg. | Std. d |
|---|---|---|---|---|---|---|
| Tumor | 2.09 | 0.15 | 0.95 | 0.07 | 1.46 | 0.70 |
| Heart | 0.45 | 0.06 | 0.40 | 0.15 | 0.24 | 0.01 |
| Lung | 9.25 | 1.74 | 6.43 | 0.67 | 2.41 | 1.41 |
| Liver | 61.55 | 5.14 | 53.12 | 2.55 | 35.48 | 5.37 |
| Spleen | 47.13 | 2.89 | 62.69 | 3.25 | 65.61 | 3.05 |
| Intestine | 0.90 | 1.26 | 0.18 | 0.12 | 0.04 | 0.01 |
| Kidney | 0.49 | 0.29 | 0.66 | 0.12 | 1.19 | 0.22 |
| Blood | 0.14 | 0.02 | 0.08 | 0.02 | 0.02 | 0.01 |
| Muscle | 0.14 | 0.07 | 0.18 | 0.09 | 0.07 | 0.01 |
| Tail | 2.11 | 2.90 | 0.79 | 0.18 | 0.60 | 0.26 |

TABLE 13
Tumor to organs ratio

| | ¹⁷⁷Lu-Tetra-ESV6-DOTAGA | | | | | |
|---|---|---|---|---|---|---|
| | 6 h | | 24 h | | 96 h | |
| | Avg. | Std. d | Avg. | Std. d | Avg. | Std. d |
| Heart | 114.60 | 10.72 | 166.10 | 39.84 | 133.14 | 55.02 |
| Lung | 52.76 | 4.00 | 138.32 | 80.65 | 69.86 | 41.75 |
| Liver | 15.19 | 2.34 | 9.42 | 1.68 | 2.07 | 0.48 |
| Spleen | 44.00 | 9.24 | 20.72 | 7.27 | 3.88 | 1.21 |
| Intestine | 86.50 | 26.67 | 284.15 | 24.71 | 213.10 | 94.17 |
| Kidney | 27.69 | 3.85 | 23.62 | 2.74 | 2.36 | 0.45 |
| Blood | 53.01 | 13.51 | 146.80 | 40.55 | 1062.04 | 322.45 |
| Muscle | 61.99 | 12.05 | 98.07 | 14.83 | 288.08 | 188.49 |
| Tail | 24.06 | 0.78 | 36.71 | 7.37 | 24.96 | 4.18 |

TABLE 14
(Tumor to organs ratio)

| ¹⁷⁷Lu-Hexa-ESV6-DOTAGA | | |
|---|---|---|
| 6 h | 24 h | 96 h |

| | Avg. | Std. d | Avg. | Std. d | Avg. | Std. d |
|---|---|---|---|---|---|---|
| Heart | 8.50 | 1.93 | 7.90 | 1.09 | 8.99 | 2.14 |
| Lung | 0.43 | 0.12 | 0.34 | 0.05 | 1.10 | 0.43 |
| Liver | 0.04 | 0.00 | 0.03 | 0.01 | 0.03 | 0.01 |
| Spleen | 0.07 | 0.02 | 0.04 | 0.01 | 0.04 | 0.01 |
| Intestine | 13.86 | 4.93 | 20.76 | 10.96 | 20.47 | 6.69 |
| Kidney | 5.24 | 0.78 | 3.37 | 1.55 | 0.99 | 0.14 |
| Blood | 7.84 | 1.17 | 17.77 | 4.49 | 85.06 | 30.99 |
| Muscle | 9.52 | 5.74 | 13.70 | 3.76 | 35.84 | 21.86 |
| Tail | 4.76 | 2.68 | 1.39 | 0.58 | 2.03 | 1.35 |

TABLE 15
(Tumor to organs ratio)

| ¹⁷⁷Lu-Octa-ESV6-DOTAGA | | |
|---|---|---|
| 6 h | 24 h | 96 h |

| | Avg. | Std. d | Avg. | Std. d | Avg. | Std. d |
|---|---|---|---|---|---|---|
| Heart | 4.71 | 0.30 | 2.70 | 1.39 | 6.06 | 3.20 |
| Lung | 0.23 | 0.04 | 0.15 | 0.01 | 0.67 | 0.22 |
| Liver | 0.03 | 0.00 | 0.02 | 0.00 | 0.04 | 0.01 |
| Spleen | 0.04 | 0.00 | 0.02 | 0.00 | 0.02 | 0.01 |
| Intestine | 9.12 | 7.94 | 6.83 | 3.23 | 34.84 | 17.72 |
| Kidney | 6.40 | 5.49 | 1.46 | 0.19 | 1.20 | 0.40 |
| Blood | 15.71 | 3.39 | 12.42 | 3.46 | 96.13 | 17.47 |
| Muscle | 16.64 | 6.31 | 5.94 | 2.01 | 23.58 | 15.41 |
| Tail | 3.89 | 3.64 | 1.24 | 0.26 | 2.76 | 1.55 |

TABLE 16

| | PEG | Distance | bonds | |
|---|---|---|---|---|
| | units | d₁ + d₂ | between | IC₅₀ |
| Compound | in L | (Å) | A and R.P. | (pM) |
| Tri-ESV6-DOTAGA | 0 | 17 | 14 | 12 |
| Tri-ESV6-PEG₂-DOTAGA | 2 | 28 | 24 | 38 |
| Tri-ESV6-PEG₄-DOTAGA | 4 | 36 | 30 | 126 |
| Tri-ESV6-PEG₆-DOTAGA | 6 | 44 | 36 | 273 |
| Tri-ESV6-PEG₈-DOTAGA | 8 | 51 | 42 | 1105 |
| Tri-ESV6-PEG₁₂-DOTAGA | 12 | 66 | 54 | 16670 |

TABLE 17

| # | Structure |
|---|---|
| 10 | 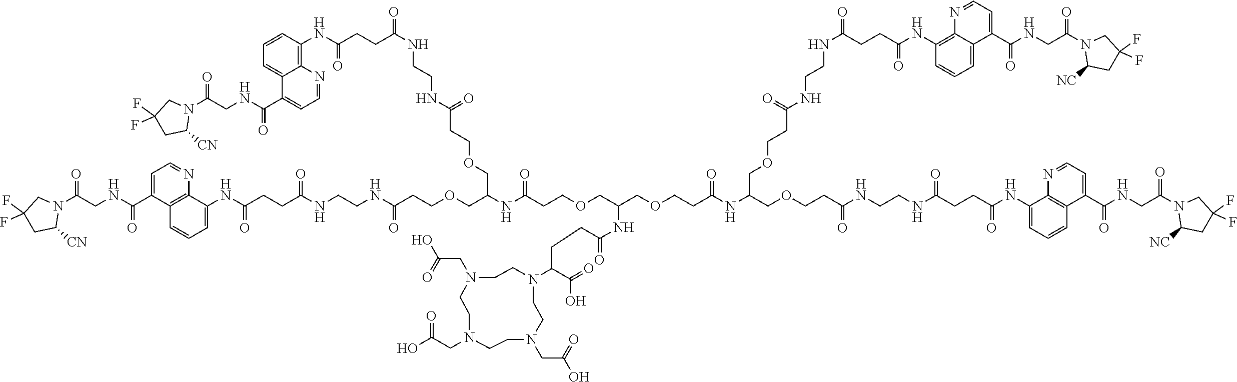 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)NC(=O)CCOCC(COCCC(=O)NC(COCCC(=O)NCCNC(=O)CCC(=O)Nc3cccc4c(C(=O)NCC(=O)N5CC(F)(F)C[C@H]5C#N)ccnc34)COCCC(=O)NCCNC(=O)CCC(=O)Nc3cccc4c(C(=O)NCC(=O)N5CC(F)(F)C[C@H]5C#N)ccnc34)NC(=O)CCC(C(=O)O)N3CCN(CC(=O)O)CCN(CC(=O)O)CCN(CC(=O)O)CC3)cccc12] |
| 15 | 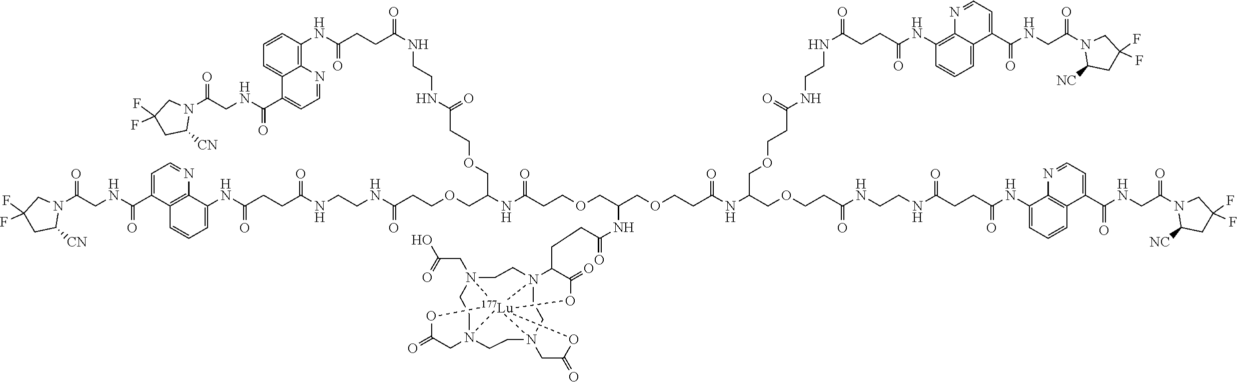 |
| 16 | 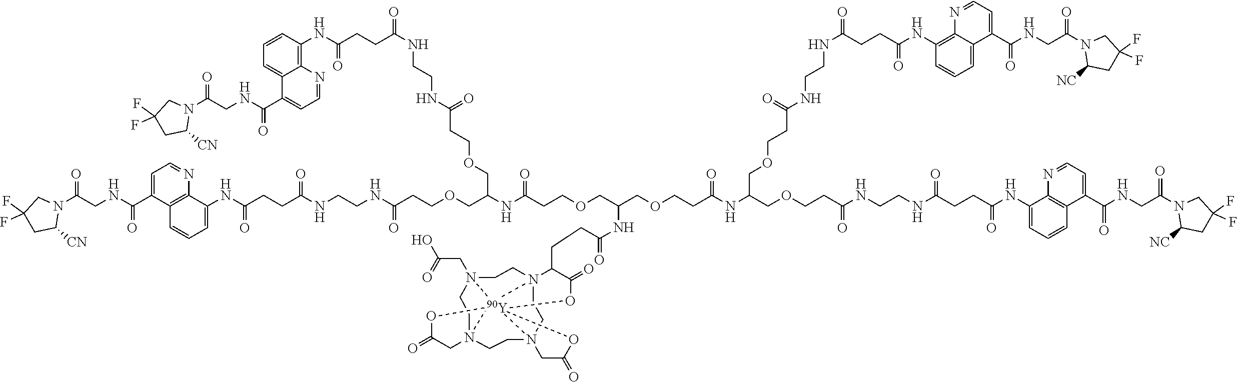 |
| 17 | 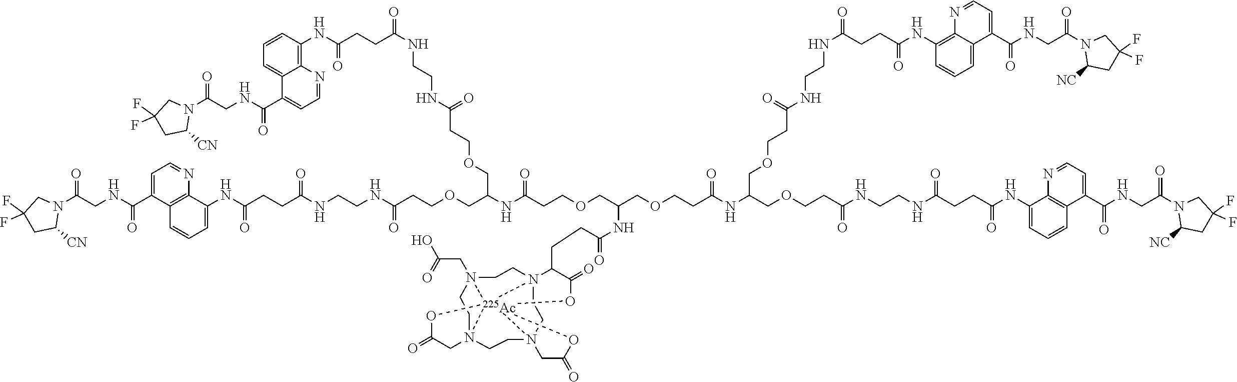 |
| 18 | 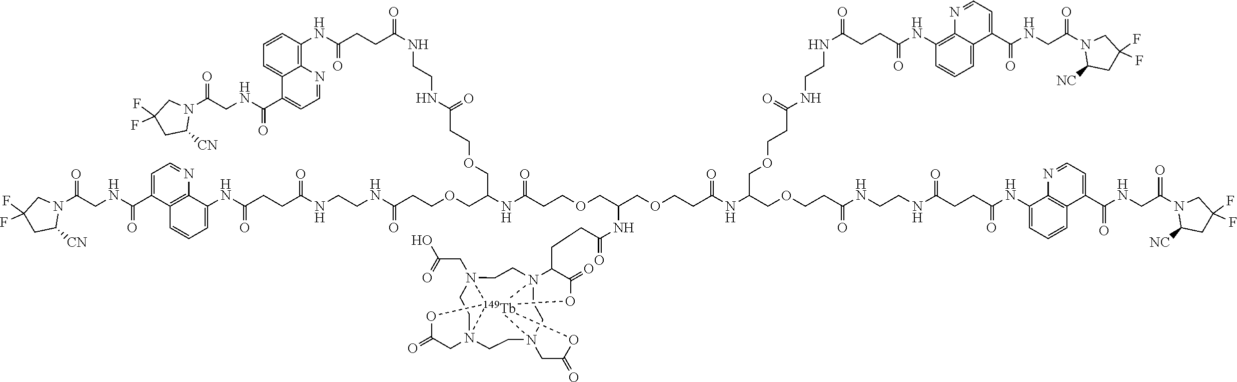 |
| 19 | 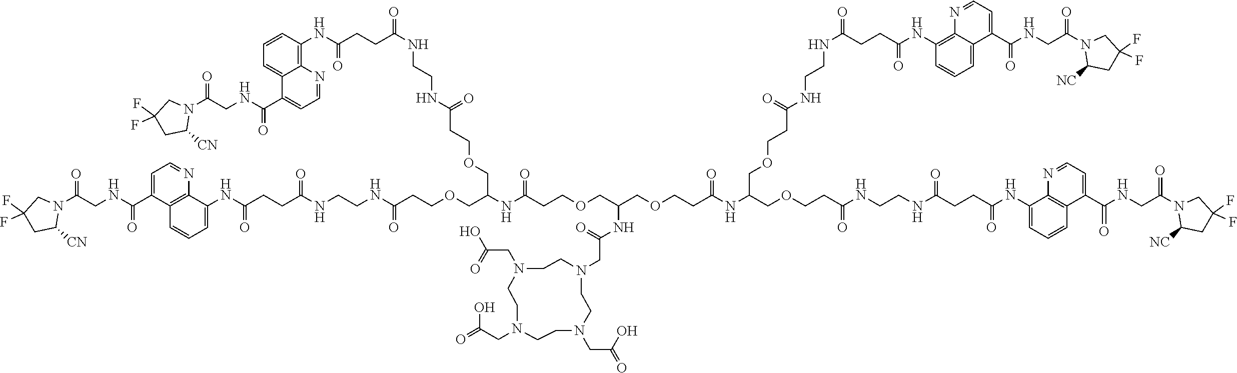 [SMILES: N#C[C@@H]1CC(F)(F)CN1C(=O)CNC(=O)c1ccnc2c(NC(=O)CCC(=O)NCCNC(=O)CCOCC(COCCC(=O)NCCNC(=O)CCC(=O)Nc3cccc4c(C(=O)NCC(=O)N5CC(F)(F)C[C@H]5C#N)ccnc34)NC(=O)CCOCC(COCCC(=O)NC(COCCC(=O)NCCNC(=O)CCC(=O)Nc3cccc4c(C(=O)NCC(=O)N5CC(F)(F)C[C@H]5C#N)ccnc34)COCCC(=O)NCCNC(=O)CCC(=O)Nc3cccc4c(C(=O)NCC(=O)N5CC(F)(F)C[C@H]5C#N)ccnc34)NC(=O)CN3CCN(CC(=O)O)CCN(CC(=O)O)CCN(CC(=O)O)CC3)cccc12] |
| 20 | 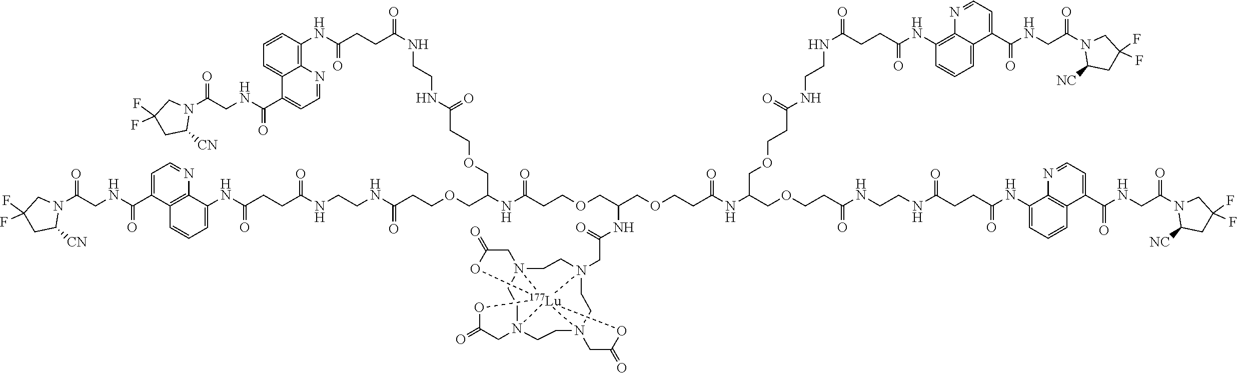 |
| 21 | 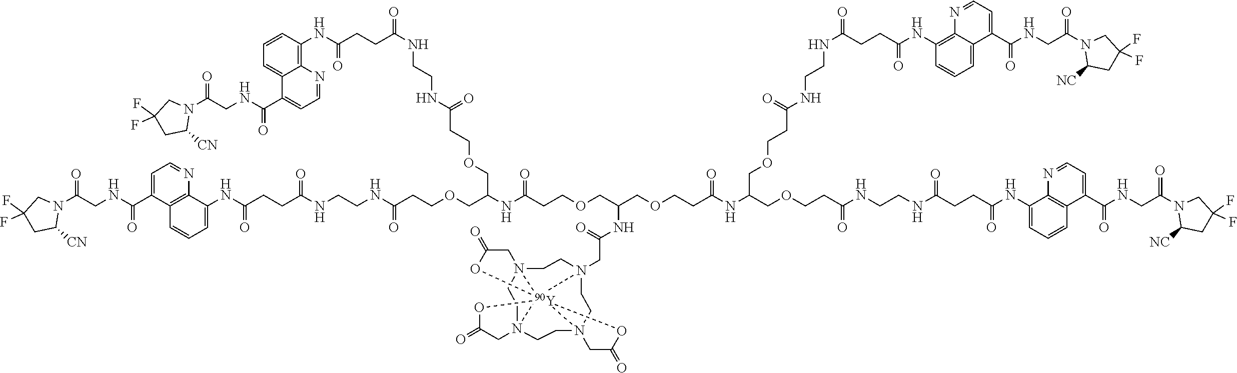 |
| 22 | 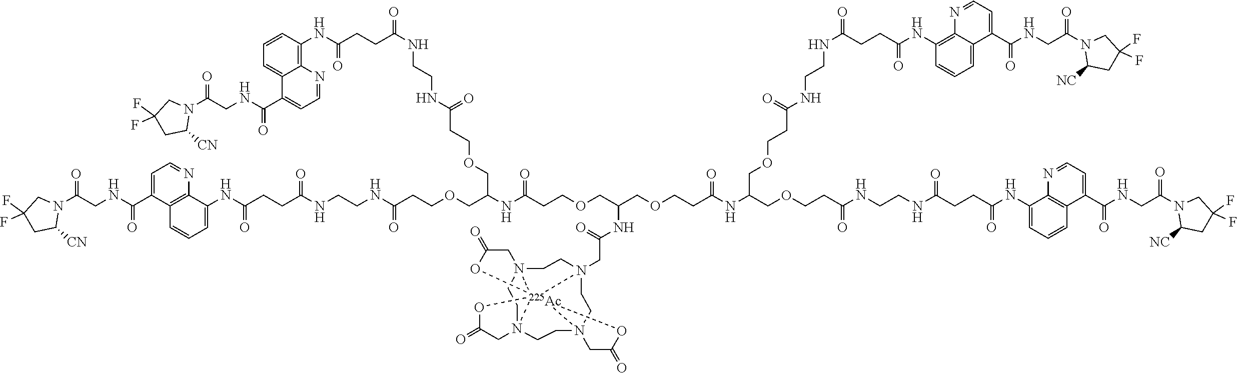 |
| 23 | 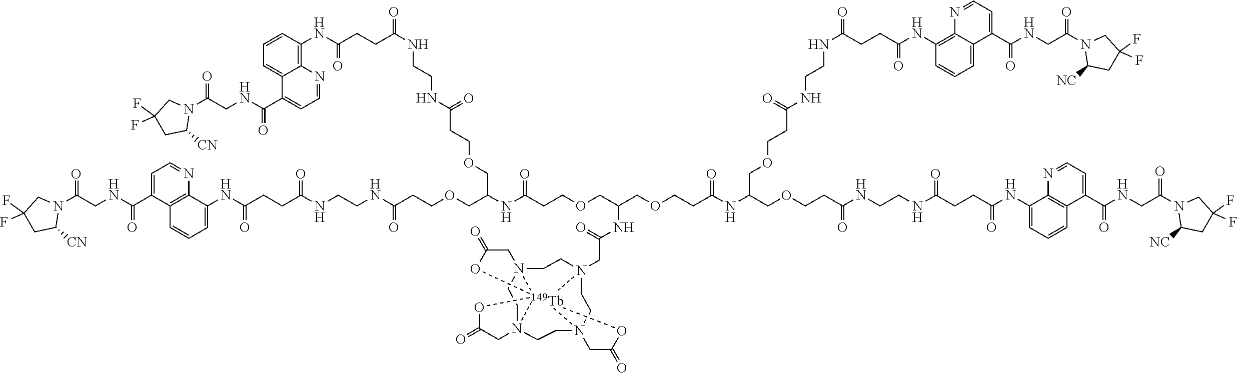 |
| 24 | 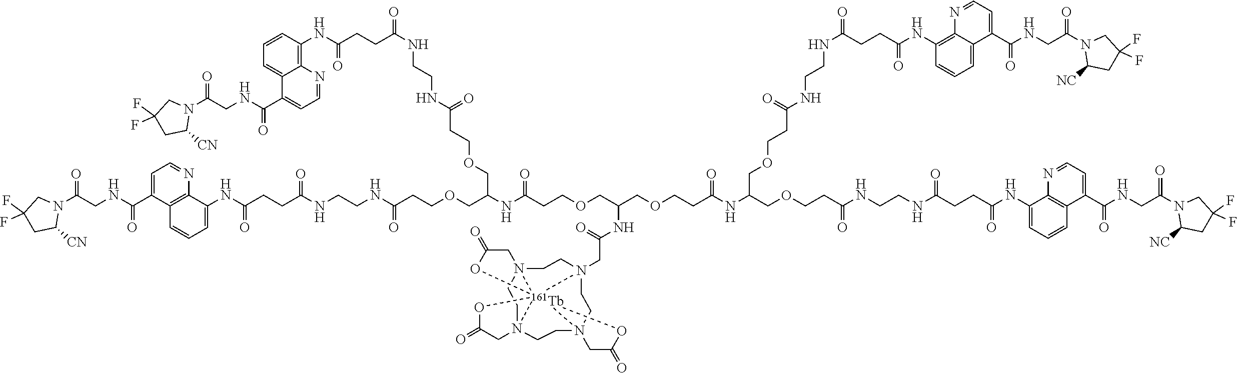 |
| 25 | 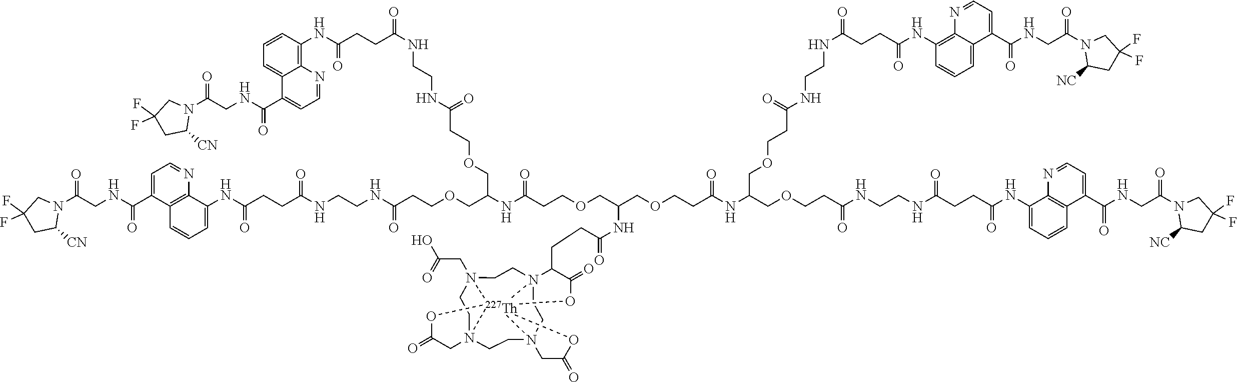 |
| 26 | 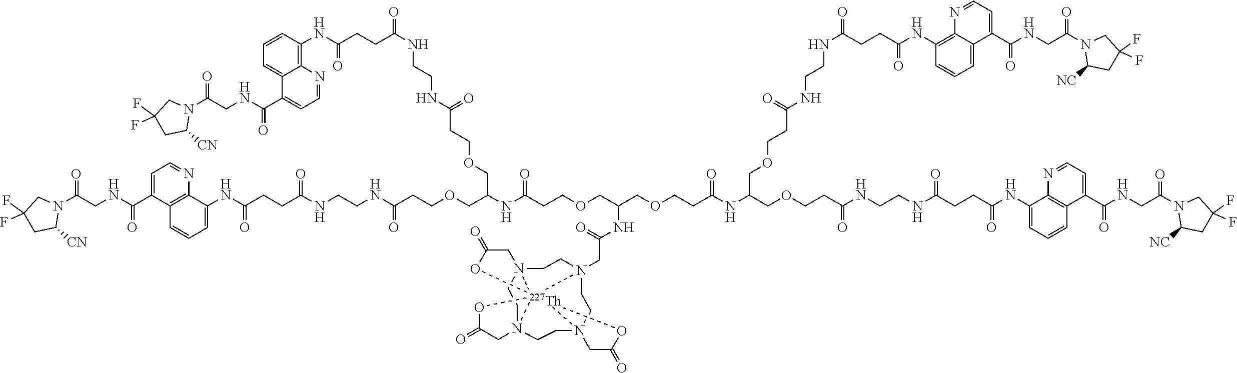 |
| 27 | 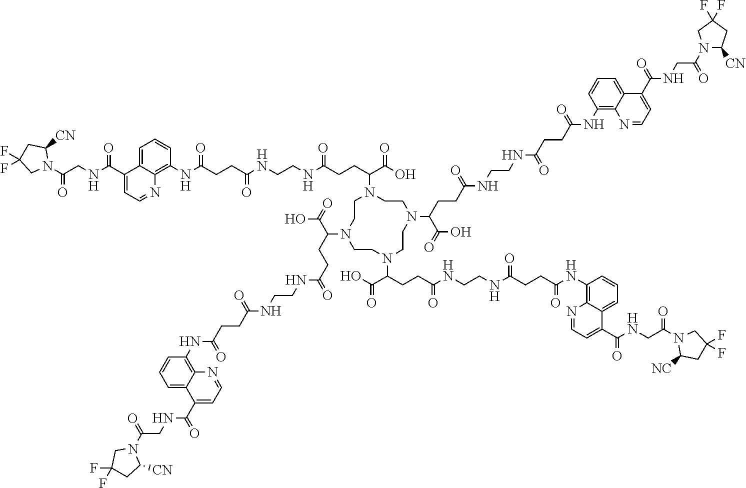 [SMILES: N#C[C@@H]1CC(F)(F)CN1C(=O)CNC(=O)c1ccnc2c(NC(=O)CCC(=O)NCCNC(=O)CCC(C(=O)O)N3CCN(C(CCC(=O)NCCNC(=O)CCC(=O)Nc4cccc5c(C(=O)NCC(=O)N6CC(F)(F)C[C@H]6C#N)ccnc45)C(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CCN(C(CCC(=O)NCCNC(=O)CCC(=O)Nc4cccc5c(C(=O)NCC(=O)N6CC(F)(F)C[C@H]6C#N)ccnc45)C(=O)O)CC3)cccc12] |
| 28 | 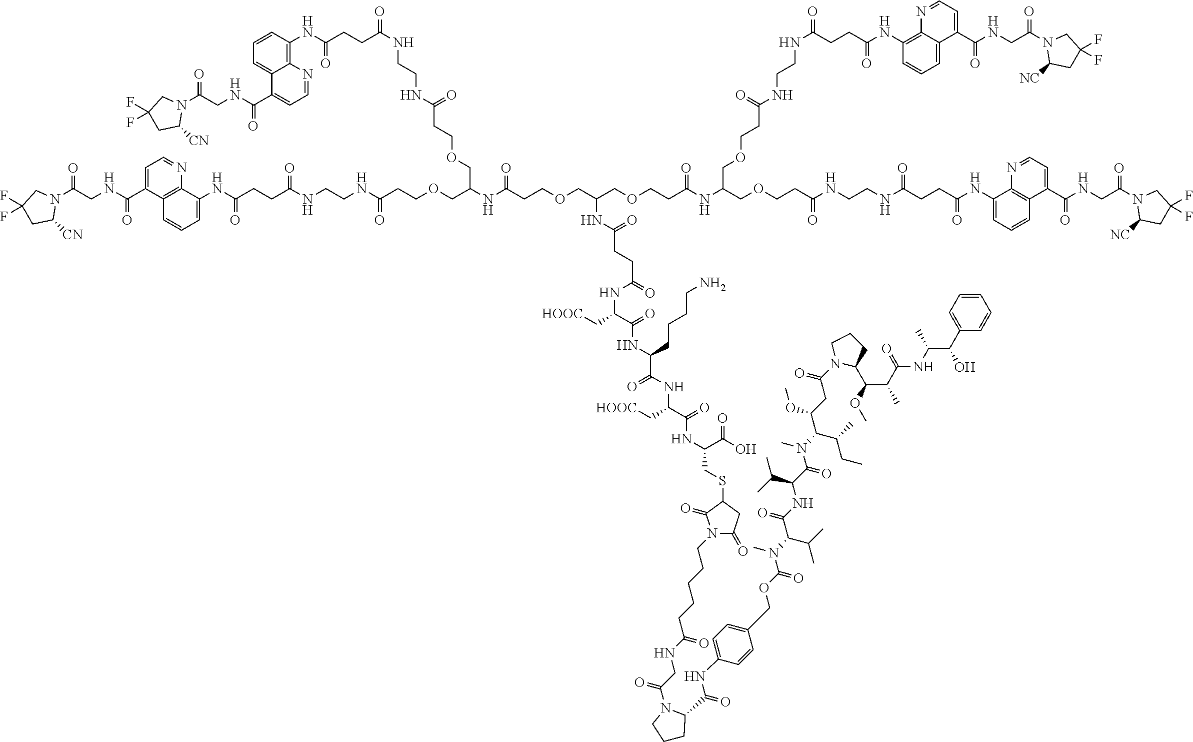 |
| 29 | 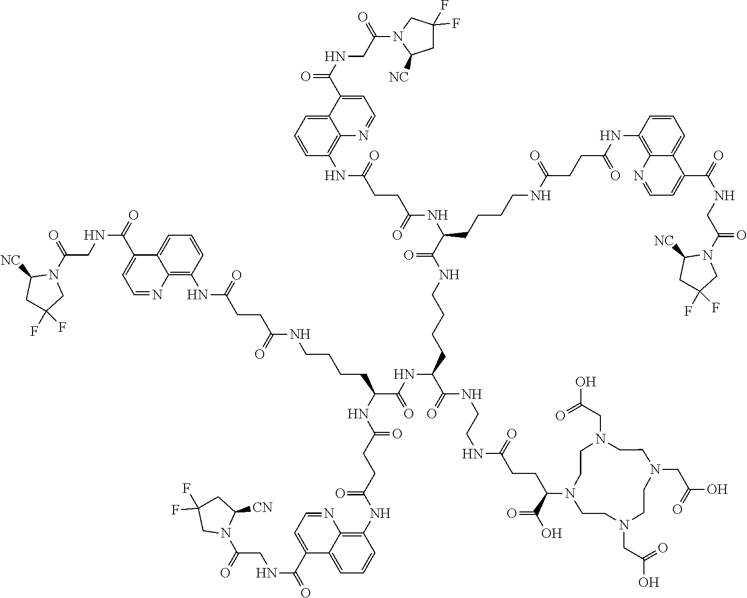 [SMILES: N#C[C@@H]1CC(F)(F)CN1C(=O)CNC(=O)c1ccnc2c(NC(=O)CCC(=O)NCCCC[C@H](NC(=O)CCC(=O)Nc3cccc4c(C(=O)NCC(=O)N5CC(F)(F)C[C@H]5C#N)ccnc34)C(=O)NCCCC[C@H](NC(=O)[C@H](CCCCNC(=O)CCC(=O)Nc3cccc4c(C(=O)NCC(=O)N5CC(F)(F)C[C@H]5C#N)ccnc34)NC(=O)CCC(=O)Nc3cccc4c(C(=O)NCC(=O)N5CC(F)(F)C[C@H]5C#N)ccnc34)C(=O)NCCNC(=O)CC[C@H](C(=O)O)N3CCN(CC(=O)O)CCN(CC(=O)O)CCN(CC(=O)O)CC3)cccc12] |

1. A compound, its individual diastereoisomers, its hydrates, its solvates, its crystal forms, its individual tautomers or a pharmaceutically acceptable salt thereof, wherein the compound is represented by the following Formula I: 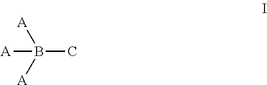
wherein each moiety A is independently represented by the following structure: 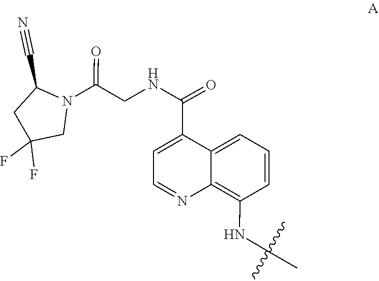 [SMILES: *Nc1cccc2c(C(=O)NCC(=O)N3CC(F)(F)C[C@H]3C#N)ccnc12]
B is a multifunctional moiety comprising a point of ramification, and covalently connects the moieties A to C; or wherein B is absent and C comprises a ramification point, such that three moieties A are attached to C;
and C is an atom, a molecule or a particle, and is a therapeutic agent.

2. The compound according to claim 1, represented by the following Formula II or III: 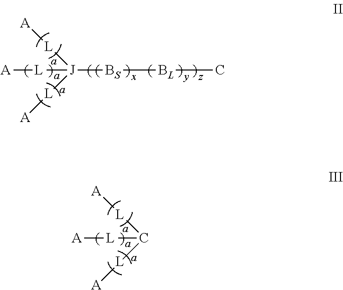
wherein each occurrence of L, B_{S} and B_{L} is independently a moiety comprising or consisting of a structural unit selected from alkylene, cycloalkylene, arylalkylene, heteroarylalkylene, heteroalkylene, heterocycloalkylene, cycloalkenylene, alkenylene, arylalkenylene, heteroarylalkenylene, heteroalkenylene, heterocycloalenkylene, alkynylene, heteroalkynylene, arylene, heteroarylene, aminoacyl, oxyalkylene, oxoalkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, and tetrapeptide, each of which is substituted or unsubstituted;
J is a moiety comprising a ramification point, comprising or consisting of a structural unit independently selected from the group consisting of alkylene, cycloalkylene, arylalkylene, heteroarylalkylene, heteroalkylene, heterocycloalkylene, alkenylene, cycloalkenylene, arylalkenylene, heteroarylalkenylene, heteroalkenylene, heterocycloalenkylene, alkynylene, heteroalkynylene, arylene, heteroarylene, aminoacyl, oxyalkylene, (oxo)alkylene, dioxoalkylene, aminoalkylene, diaminoalkylene, diacid ester, dialkylsiloxane, amide, thioamide, thioether, thioester, ester, carbamate, hydrazone, thiazolidine, methylene alkoxy carbamate, disulfide, vinylene, imine, imidamide, phosphoramide, saccharide, phosphate ester, phosphoramide, carbamate, dipeptide, tripeptide, tetrapeptide, each of which is substituted or unsubstituted;
each x is an integer independently selected from 0, 1, 2, 3, 4, 5, 6, 7, 8, 9, 10, 11, 12, 13, 14, 15, 16, 17, 18, 19 and 20;
each y is an integer independently selected from 0, 1, 2, 3, 4, 5, 6, 7, 8, 9, 10, 11, 12, 13, 14, 15, 16, 17, 18, 19 and 20;
each z is an integer independently selected from 0, 1, 2, 3, 4 and 5; and
each a is an integer independently selected from 0, 1, 2, 3, 4 and 5.

3. The compound according to claim 1 or 2, wherein the distance (d₁+d₂) between each moiety A and the closest point of ramification is ≤60, ≤51, ≤50, ≤44 or ≤36 Å; or
wherein the number of bonds separating the two respective groups, counted along the shortest path connecting them, is ≤50, ≤42, ≤41, ≤36 or ≤30.

4. The compound according to any one of the preceding claims, wherein the distance (d₁+d₂) between each moiety A and the closest point of ramification is ≤30, ≤28, ≤27, ≤26 or ≤25 Å; preferably ≤24, ≤22, ≤21, ≤19 or ≤18 Å; more preferably ≤17, ≤15, ≤13, ≤12, ≤11, ≤10, ≤8, ≤7, ≤6, ≤5, ≤4, ≤2 or ≤1.5 Å; or
wherein the number of bonds separating the two respective groups, counted along the shortest path connecting them, is ≤25, ≤24, ≤23, ≤22 or ≤21; preferably ≤20, ≤19, ≤18, ≤17, ≤16 or ≤15; more preferably ≤14, ≤13, ≤12, ≤11, ≤10, ≤9, ≤8, ≤7, ≤6, ≤5, ≤4, ≤3, ≤2 or 1.

5. The compound according to claim 1 or 2, wherein the distance (d₁+d₂) between each moiety A and the closest point of ramification is 30 Å or less, preferably 24 or less, more preferably 17 Å or less.

6. The compound according to any one of the preceding claims, wherein between each moiety A and the closest point of ramification, the distance (d₁+d₂) is 17 Å or less; or the number of bonds separating the two respective groups, counted along the shortest path connecting them is 14 or less.

7. The compound according to any one of the preceding claims, wherein the distance (d₁) between each moiety A and the closest moiety J is ≤55, ≤45, ≤38 or ≤30 Å; or
wherein the number of bonds separating the two respective groups, counted along the shortest path connecting them, is ≤45, ≤37, ≤31 or ≤25.

8. The compound according to any one of the preceding claims, wherein the distance (d₁) between each moiety A and the closest moiety J is ≤24, ≤23, ≤22, ≤21, ≤20 or ≤19 Å; preferably ≤18, ≤17, ≤16, ≤15, ≤13 or ≤12 Å; more preferably ≤11, ≤10, ≤8, ≤7, ≤6, ≤5, ≤4, ≤2 or ≤1.5 Å; or
wherein the number of bonds separating the two respective groups, counted along the shortest path connecting them, is ≤20, ≤19, ≤18, ≤17 or ≤16; preferably ≤15, ≤14, ≤13, ≤12, ≤11 or ≤10; more preferably ≤9, ≤8, ≤7, <6, ≤5, ≤4, ≤3, ≤2 or 1.

9. The compound according to any one of the preceding claims, wherein the distance (d₁) between each moiety A and J is 24 Å or less, preferably 18 Å or less, more preferably 11 Å or less.

10. The compound according to any one of the preceding claims, wherein between each moiety A and the closest moiety J, the distance (d₁) is 11 Å or less; or the number of bonds separating the two respective groups, counted along the shortest path connecting them, is 9 or less.

11. The compound according to any one of the preceding claims, wherein the distance (d₂) between each moiety (L)ₐ and the closest point of ramification is ≤19, ≤17, ≤16, ≤15 or ≤13 Å; preferably ≤12, ≤11, ≤10, ≤8 or ≤7 Å; more preferably ≤6, ≤5, ≤4, ≤2 or ≤1.5 Å; or
wherein the number of bonds separating the two respective groups, counted along the shortest path connecting them, is ≤16, ≤14, ≤13, ≤12, ≤11; preferably ≤10, ≤9, ≤8, ≤7 or ≤6; more preferably ≤5, ≤4, ≤3, ≤2 or 1.

12. The compound according to any one of the preceding claims, wherein the distance (d₂) between each moiety L and the closest point of ramification is 19 Å or less, preferably 12 Å or less, more preferably 6 Å or less.

13. The compound according to any one of the preceding claims, wherein the distance (d₂) between each moiety (L)ₐ and the closest point of ramification is 6 Å or less; or the number of bonds separating the two respective groups, counted along the shortest path connecting them, is 5 or less.

14. The compound according to claim 2, wherein the distance or separation between each moiety A and the closest point of ramification is as defined in claim 6.

15. The compound according to claim 14, wherein the distance or separation between each moiety A and the closest moiety J is as defined in claim 10.

16. The compound according to any one of the preceding claims, wherein L, B_{S} and/or B_{L} are each independently selected from:
(a) a group comprising or consisting of a structural unit independently selected from the group consisting of: 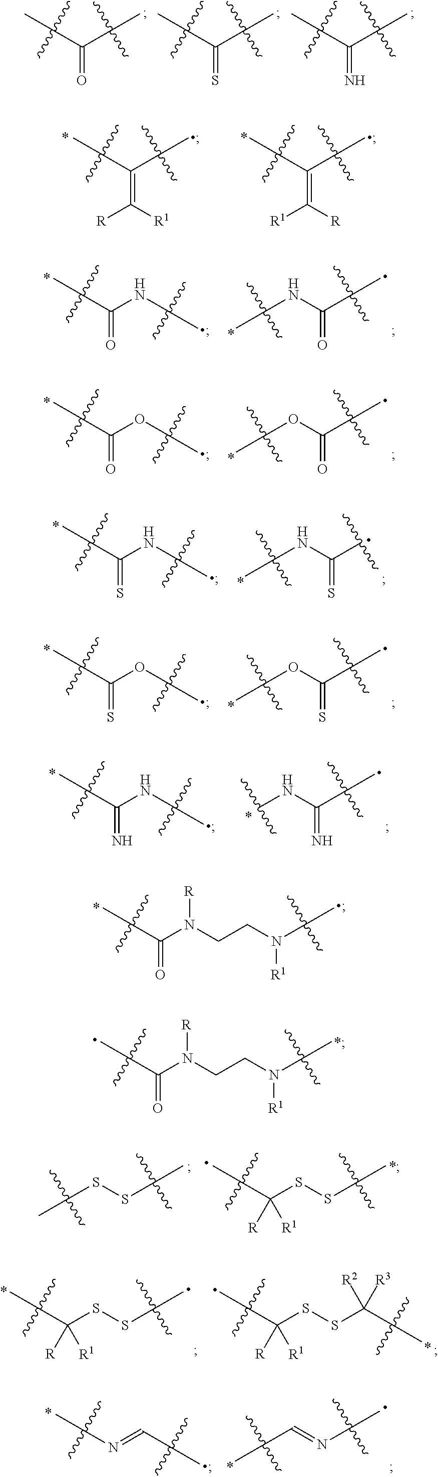 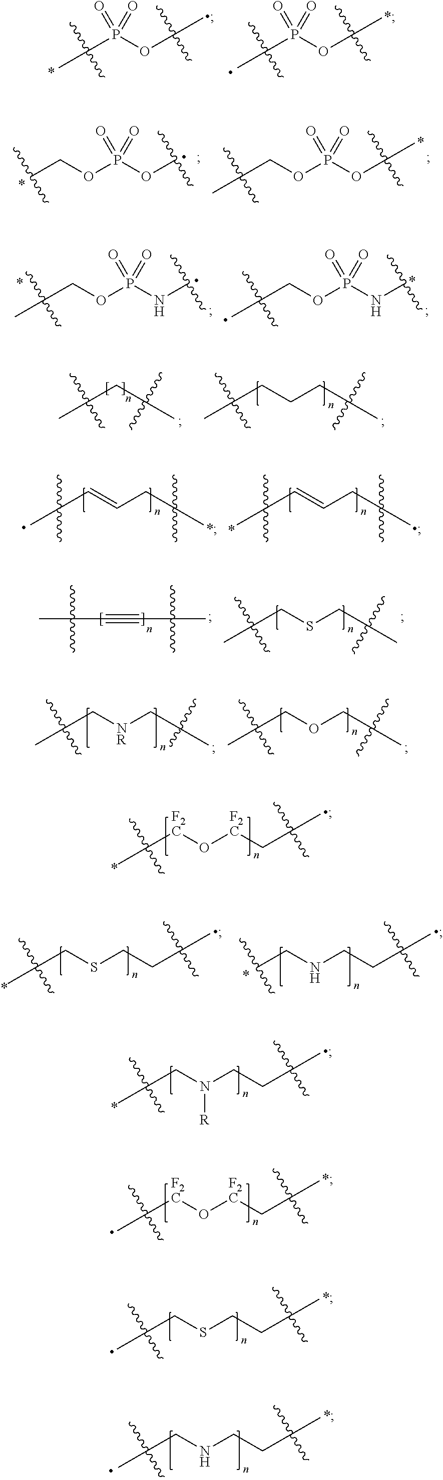 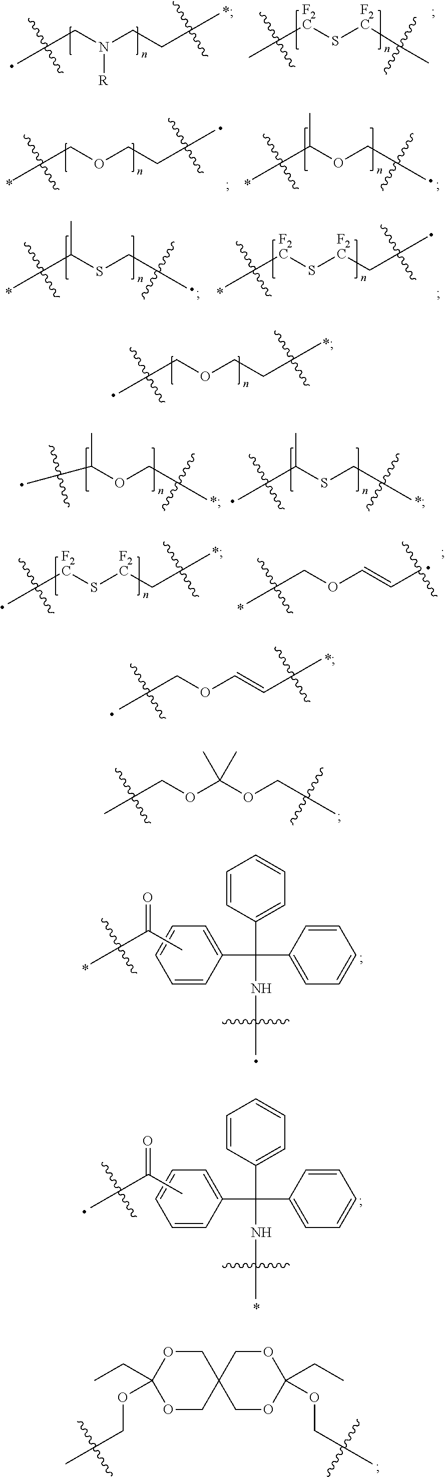 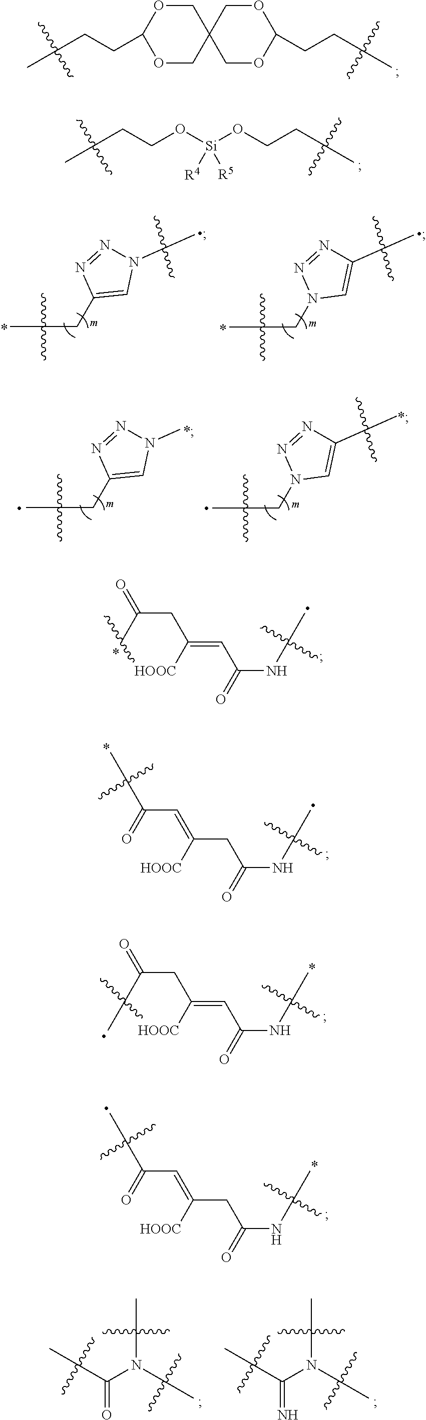 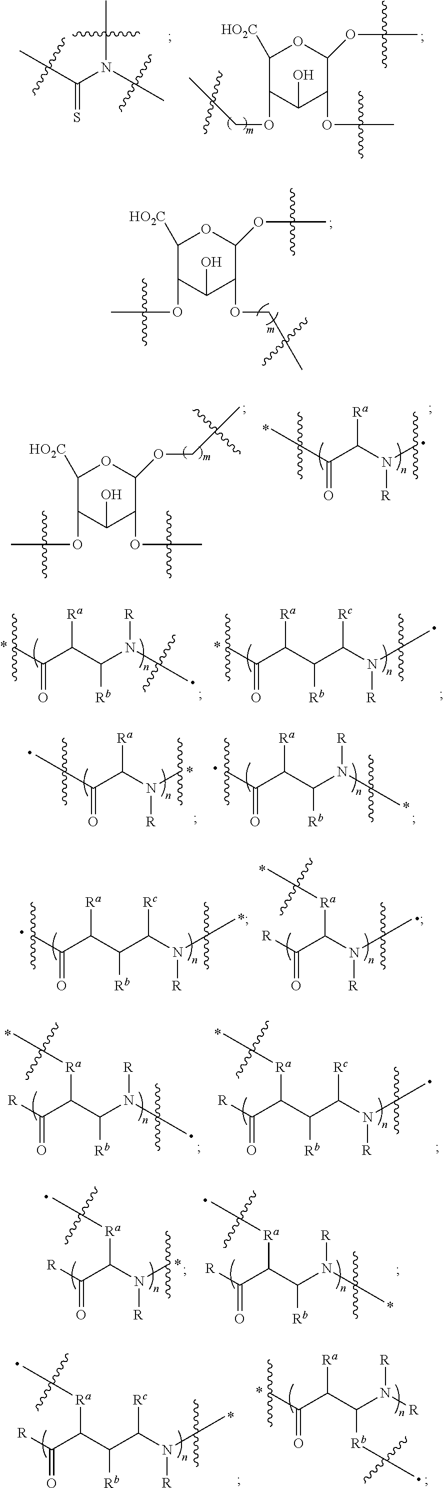 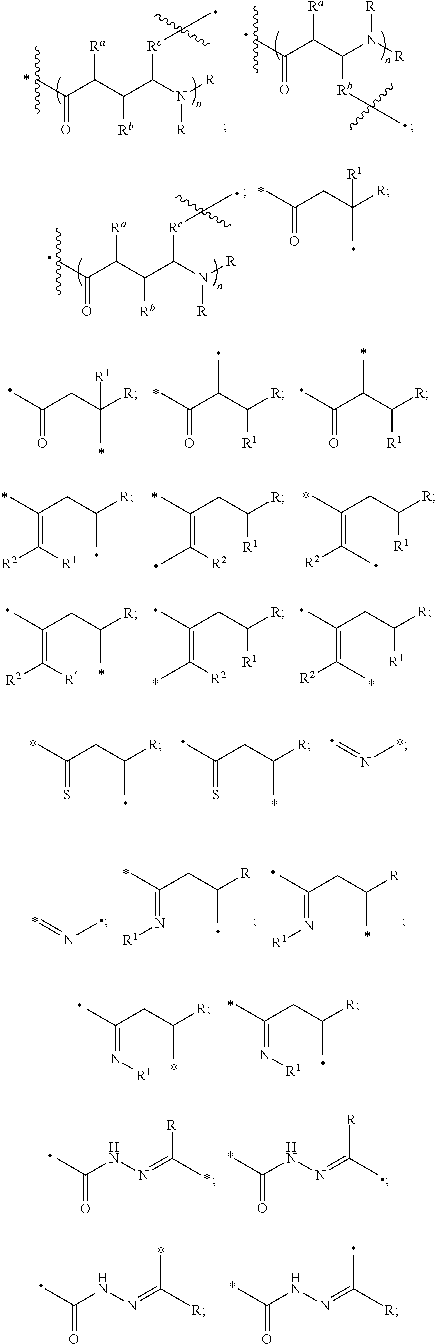 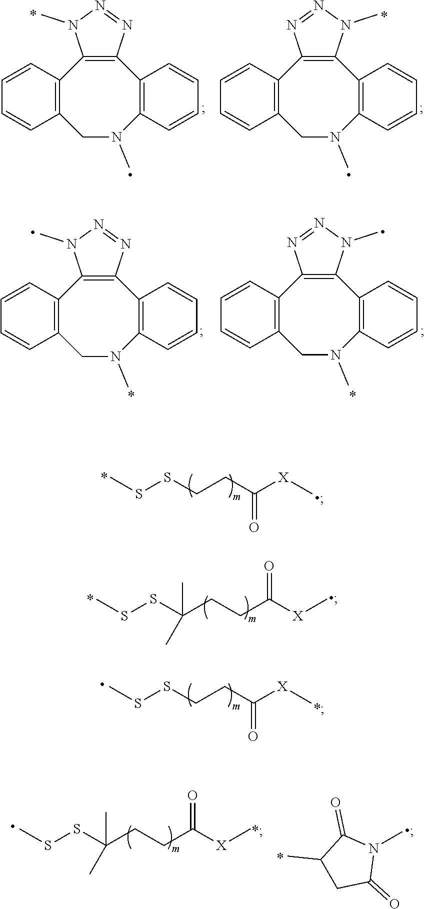 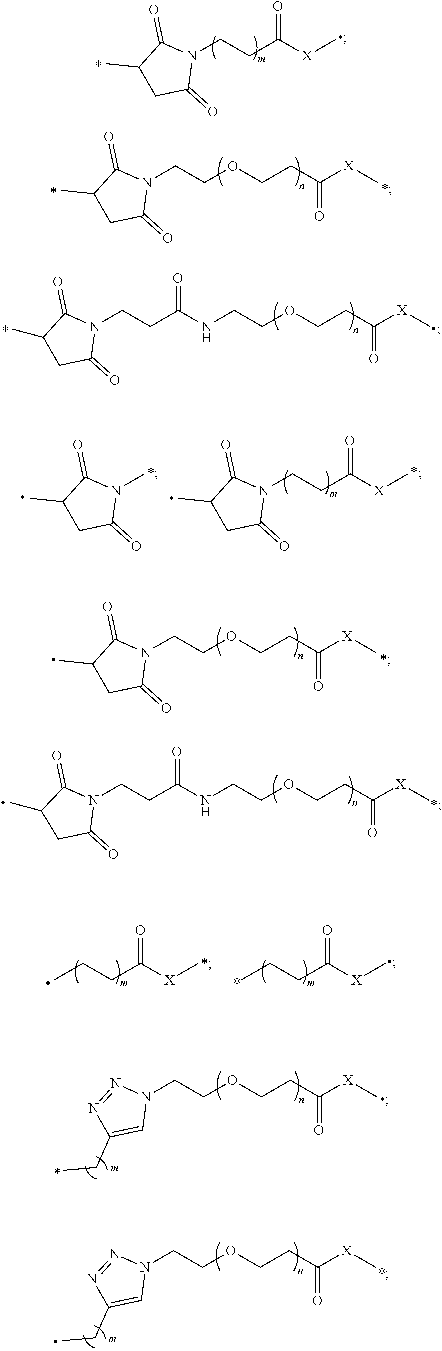
wherein each of R, R¹, R² and R³ is independently selected from H, OH, SH, NH₂, halogen, cyano, carboxy, alkyl, cycloalkyl, aryl and heteroaryl, each of which is substituted or unsubstituted;
each of R⁴ and R⁵ is independently selected from alkyl, cycloalkyl, aryl and heteroaryl, each of which is substituted or unsubstituted;
each of R^{a}, R^{b} and R^{c} is independently selected from side-chain residues of a proteinogenic or a non-proteinogenic amino acid, each of which can be further substituted;
each X is independently selected from NH, NR, S, O and CH₂, preferably NH;
each of n and m is independently an integer from 0 to 100, preferably 0 to 50, more preferably 0 to 30, yet more preferably selected from 0, 1, 2, 3, 4, 5, 6, 7, 8, 9, 10, 11, 12, 13, 14, 15, 16, 17, 18, 19 and 20; and
wherein each * represents a point of attachment for which the shortest path to a moiety A comprises less atoms than that for ·; and each · represents a point of attachment for which the shortest path to moiety C comprises less atoms than that for *;
(c) one or more of the following structural units: 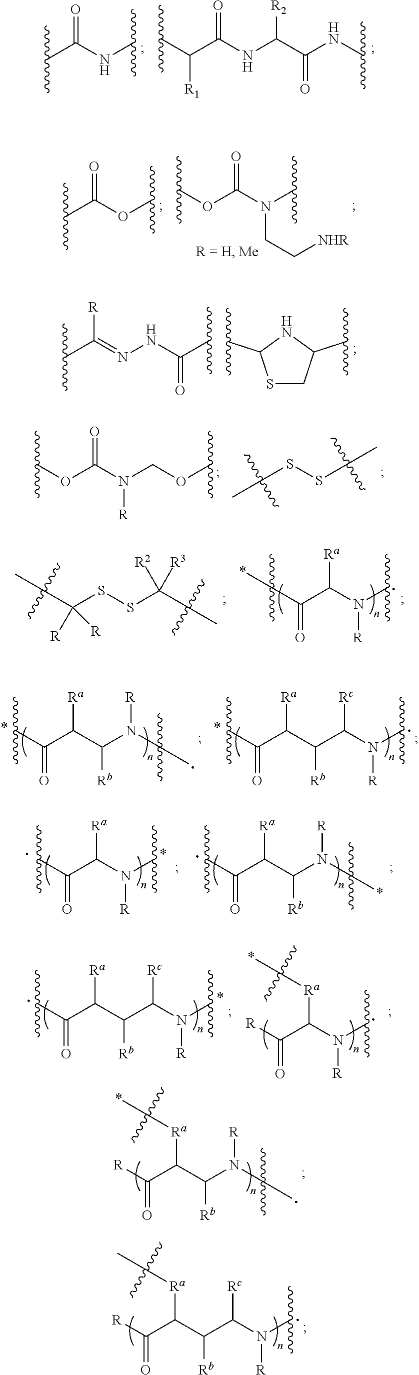 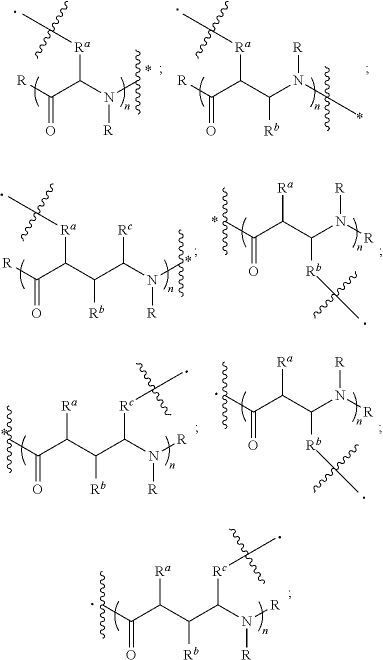
wherein in each of the above structures, n is 1, 2, 3 or 4; and
each * represents a point of attachment for which the shortest path to a moiety A comprises less atoms than that for ·; and each · represents a point of attachment for which the shortest path to moiety C comprises less atoms than that for *, with the proviso that when n is >1 and a respective point of attachment is indicated on any one of R^{a}, R^{b} and R^{c}, then it can be independently present in one or more of the peptide monomeric units, preferably in one peptide monomeric unit most distant from the other point of attachment indicated in the respective structure;
(d) one or more of B_{L} and B_{S} is independently selected from the following structures: 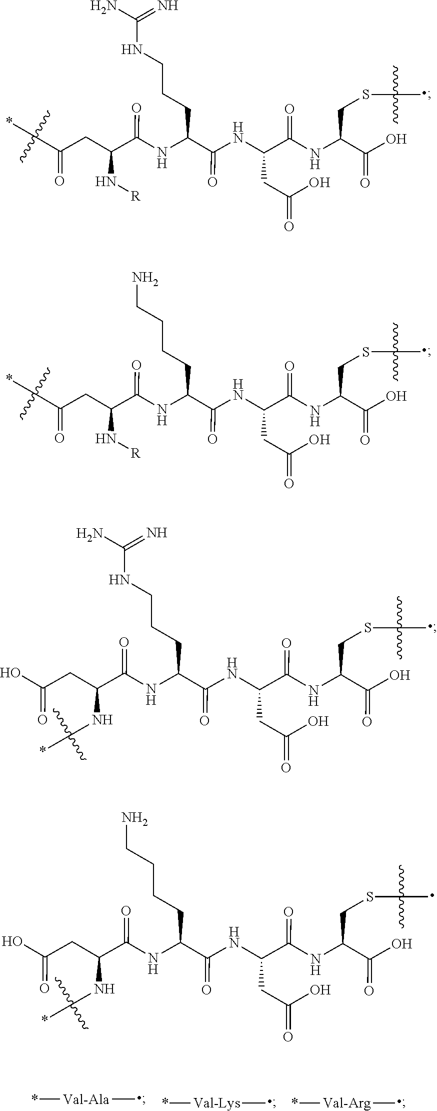
wherein each * represents a point of attachment for which the shortest path to moiety A comprises less atoms than that for ·; and each · represents a point of attachment for which the shortest path to moiety C comprises less atoms than that for *;
and/or
(e) y is 1, 2 or 3; and/or at least one B_{L} further comprises a cleavable linker group independently selected from the following structures: 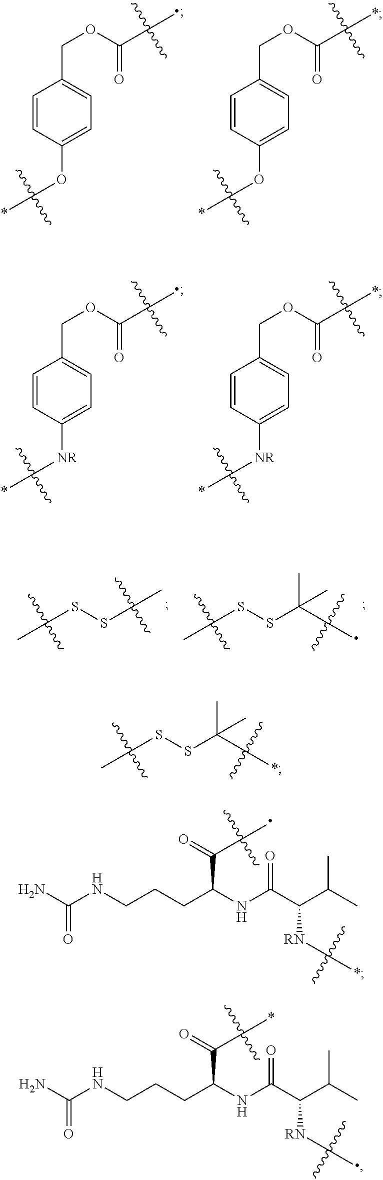
each * represents a point of attachment for which the shortest path to moiety A comprises less atoms than that for ·; and each · represents a point of attachment for which the shortest path to moiety C comprises less atoms than that for *; or
(f) wherein ((B_{S})ₓ(B_{L})_{y})_{z} is represented by the following structure: 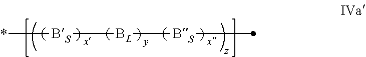
wherein B′_{S} and B″_{S} are each independently selected from the group consisting of: 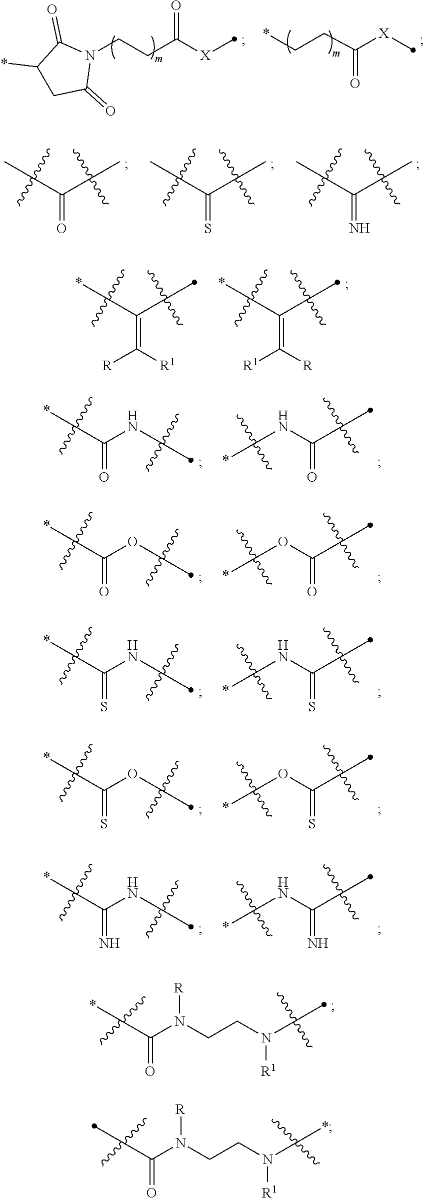
each B_{L} is independently selected from the group consisting of: 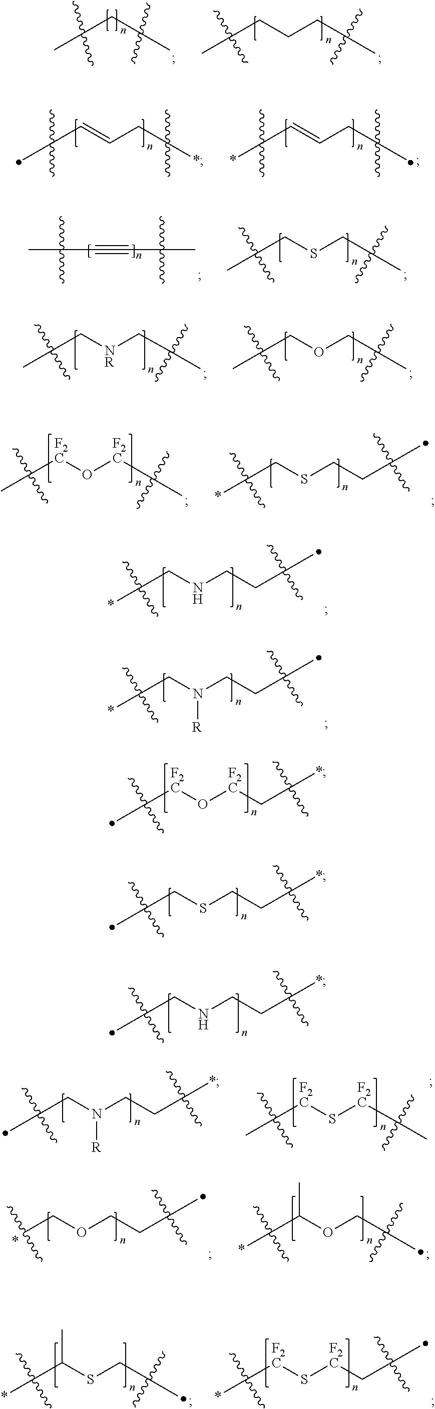 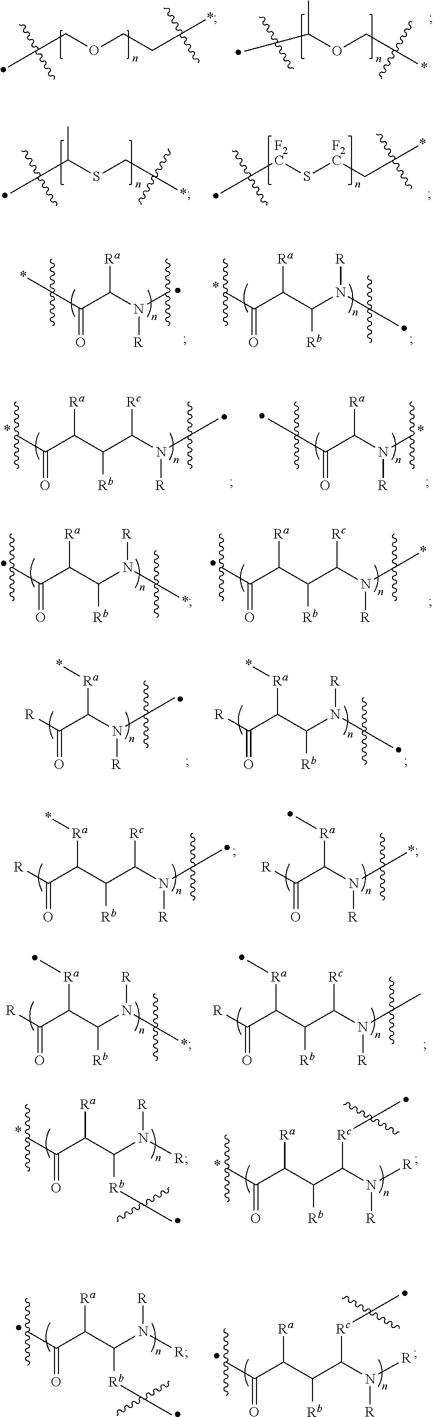 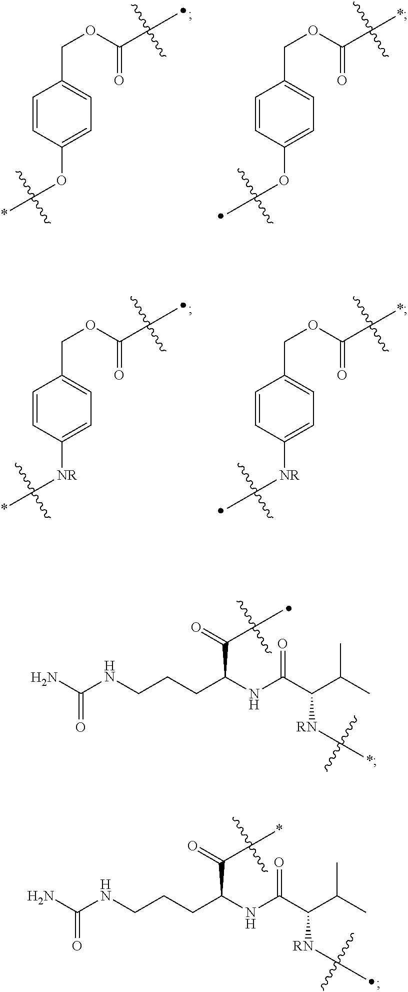
each n is 0, 1, 2, 3, 4 or 5;
each m is 0, 1, 2, 3, 4 or 5;
each x′ is 0, 1 or 2;
each x″ is 0, 1 or 2;
each y is 0, 1 or 2; and
z is 1 or 2,
wherein R, R¹, R², R³, R^{a}, R^{b}, R^{c}, X, * and · are defined as in any one of the preceding claims.

17. The compound according to any one of claims 2 to 16, wherein J has a structure represented by J-1, J-2, J-2, J-4 or J-5: 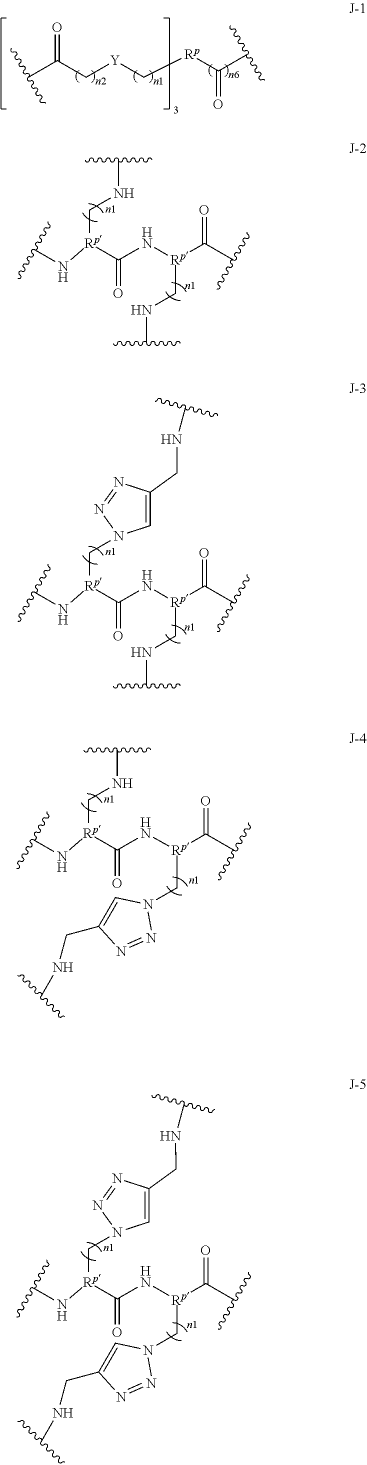
wherein each n₁ is independently 0, 1, 2, 3, 4, 5 or 6, preferably 4;
each n₂ is independently 0, 1, 2, 3 or 4, preferably 2;
each n₆ is independently 0 or 1, preferably 1;
each Y is independently O, NR, CR₂, preferably O;
each R^{p} is independently N⁺ or C, preferably C;
each R^{p′} is independently N or CR;
wherein each R is independently H or is selected from H, SH, NH₂, halogen, cyano, carboxy, C1-6-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C1-10 cycloheteroalkenyl, C₆₋₁₀ aryl, and (C₆₋₁₀ aryl)C₁₋₆ alkyl.

18. The compound according to any one of claims 2 to 16, wherein J has a structure selected from the following: 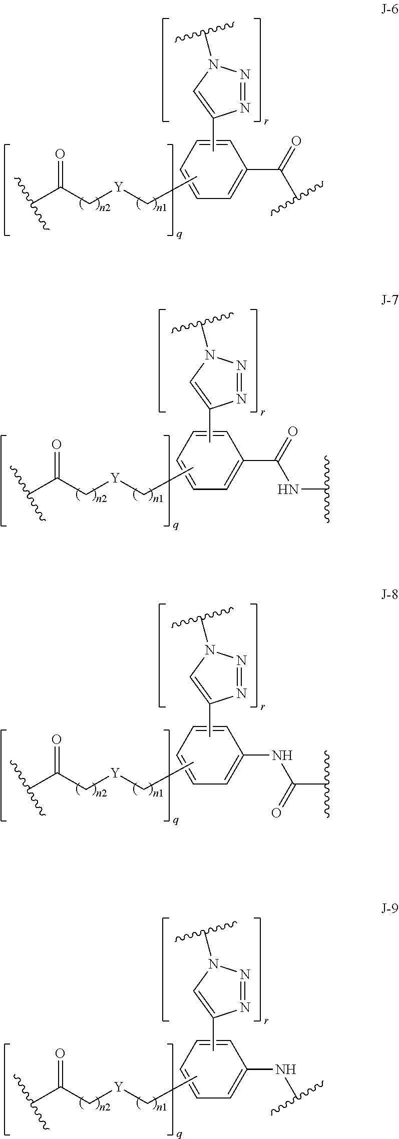
wherein each q is 0, 1, 2 or 3; each r is 0, 1, 2 or 3; q+r=3; and all other groups and variables are as defined in claim 6.

19. The compound according to any one of claims 2 to 17, wherein J has a structure represented by J-1a, J-1b, J-1c, J-1d, J-2a, J-2b, J2-c or J2-d:

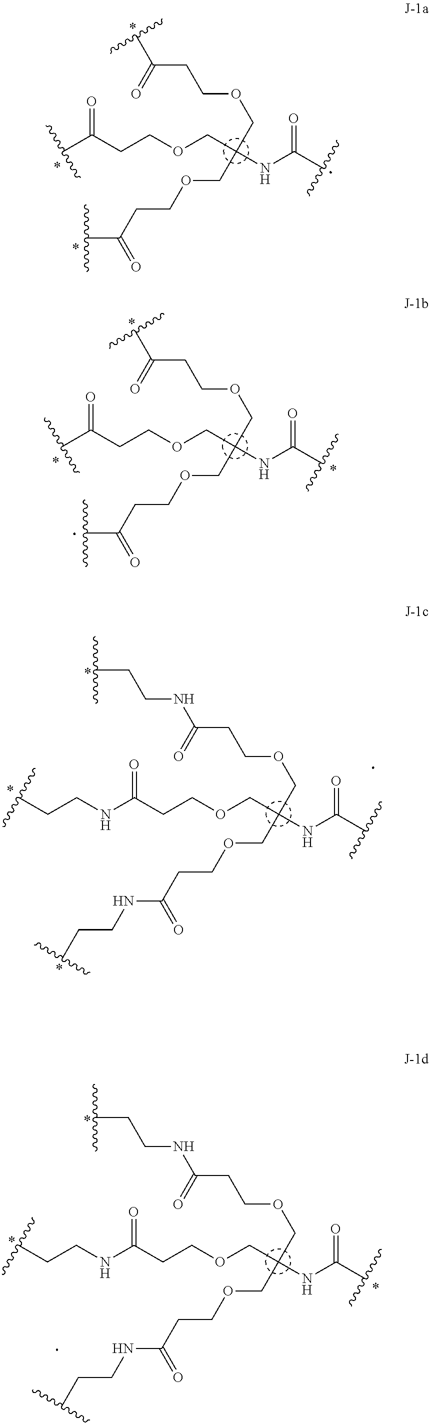

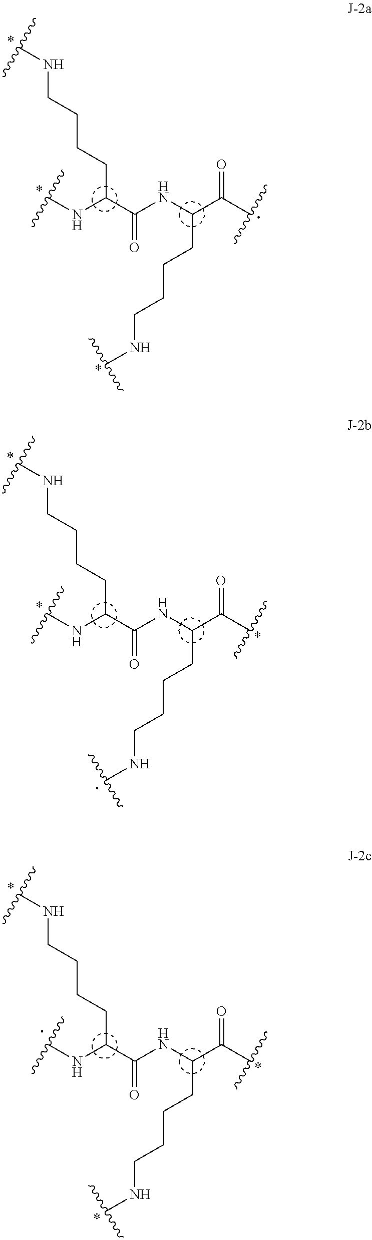

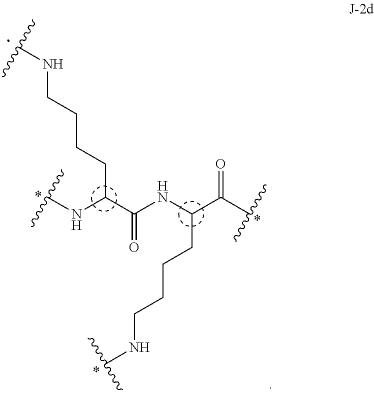

20. The compound according to any one of claims 2 to 16 and 18, wherein J has a structure selected from the following:

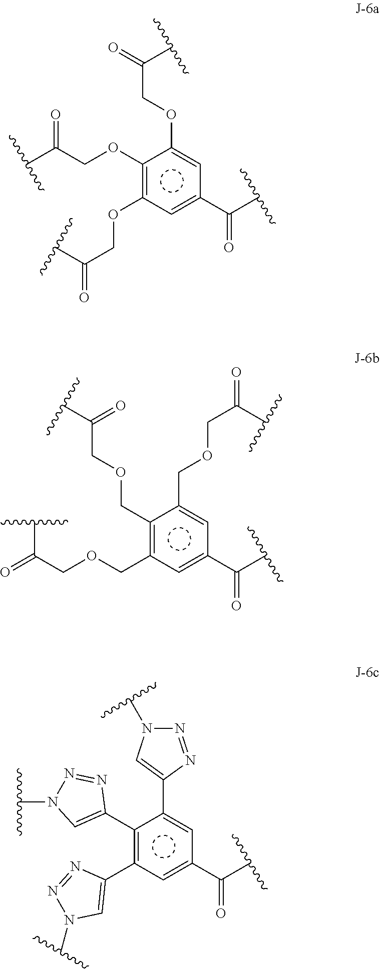 [SMILES: *C(=O)COc1cc(C(*)=O)cc(OCC(*)=O)c1OCC(*)=O ; *C(=O)COCc1cc(C(*)=O)cc(COCC(*)=O)c1COCC(*)=O ; *C(=O)c1cc(-c2cn(*)nn2)c(-c2cn(*)nn2)c(-c2cn(*)nn2)c1]

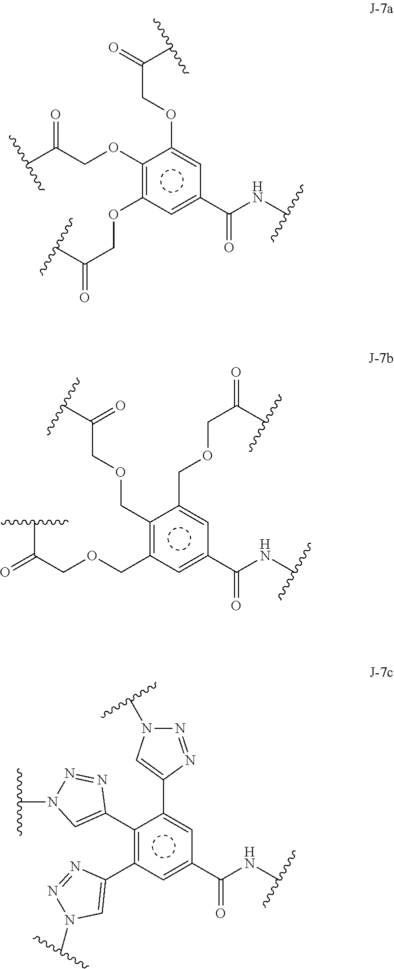 [SMILES: *NC(=O)c1cc(OCC(*)=O)c(OCC(*)=O)c(OCC(*)=O)c1 ; *NC(=O)c1cc(COCC(*)=O)c(COCC(*)=O)c(COCC(*)=O)c1 ; *NC(=O)c1cc(-c2cn(*)nn2)c(-c2cn(*)nn2)c(-c2cn(*)nn2)c1]

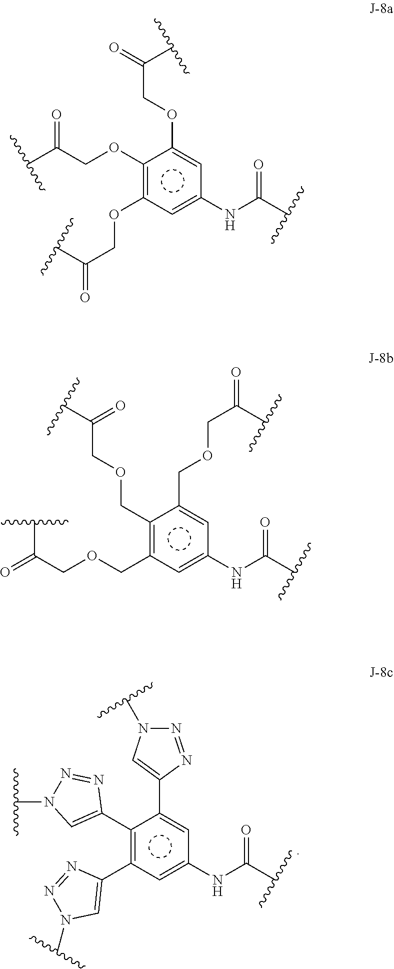 [SMILES: *C(=O)COc1cc(NC(*)=O)cc(OCC(*)=O)c1OCC(*)=O ; *C(=O)COCc1cc(NC(*)=O)cc(COCC(*)=O)c1COCC(*)=O ; *C(=O)Nc1cc(-c2cn(*)nn2)c(-c2cn(*)nn2)c(-c2cn(*)nn2)c1]

21. The compound according to any one of claims 2 to 20, wherein each L independently comprises or is represented by a structure selected from:
bond,

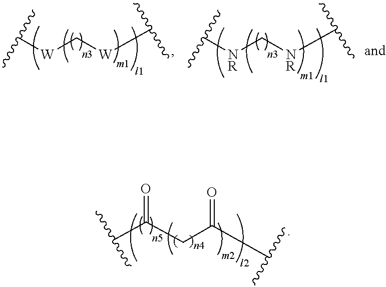

22. The compound according to any one of claims 2 to 21, wherein each (L)ₐ is represented by: 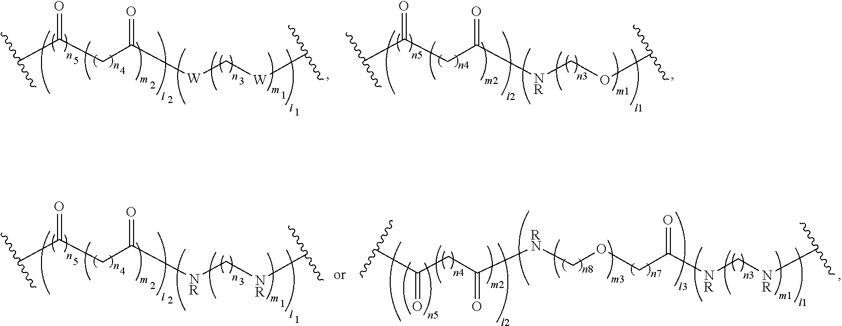
wherein each n₃, n₄, n₇ and n₈ is independently 0, 1, 2, 3, 4, 5 or 6, preferably 2;
each n₅ is independently 0 or 1, preferably 1:
each m₁, m₂ and m₃ is independently 0, 1, 2, 3, 4, 5, 6, 7, 8, 9, 10 or 11, preferably 1;
each l₁ and l₂ is independently 0 or 1, preferably 1;
each l₃ is independently 0, or 1, preferably 0;
each R is independently H or is selected from H, SH, NH₂, halogen, cyano, carboxy, C₁₋₆-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C₁₋₁₀ cycloheteroalkenyl, C₆₋₁₀ aryl, and (C₆₋₁₀ aryl)C₁₋₆ alkyl; and
each W is independently selected from NR and O.

23. The compound according to any one of claims 2 to 22, wherein the fragment ((L)ₐ) 3J is represented by the following structure K-1, K-2, K-3 or K-4, 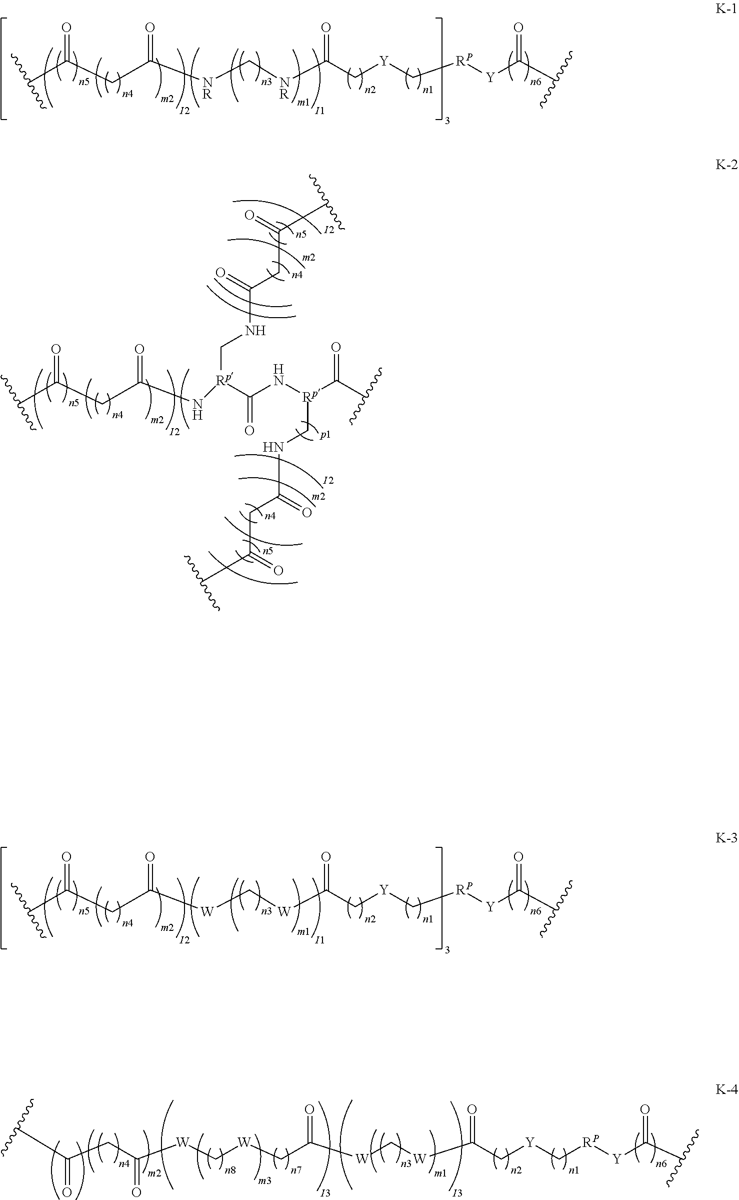
wherein each n₁ is independently 0, 1, 2, 3, 4, 5 or 6, preferably 4;
each n₂ is independently 0, 1, 2, 3 or 4, preferably 2;
wherein each n₃, n₄, n₇ and n₈ is independently 0, 1, 2, 3, 4, 5 or 6, preferably 2;
each n₅ and n₆ is independently 0 or 1, preferably 1;
each Y is independently O, NR, CR₂, preferably O;
each R^{p} is independently N⁺ or C, preferably C;
each R^{p′} is independently N or CR;
each m₁, m₂ and m₃ is independently 0, 1, 2, 3, 4, 5, 6, 7, 8, 9, 10 or 11, preferably 1;
each l₁ and l₂ is independently 0, or 1, preferably 1;
each l₃ is independently 0, or 1, preferably 0;
wherein each R is independently H or is selected from H, SH, NH₂, halogen, cyano, carboxy, C1-6-alkyl, O(C₁₋₆ alkyl), S(C₁₋₆-alkyl), C₂₋₆ alkenyl, C₂₋₆ alkynyl, C₁₋₆ heteroalkenyl, C₁₋₆ heteroalkynyl, C₃₋₁₀ cycloalkenyl, C1-10 cycloheteroalkenyl, C₆₋₁₀ aryl, and (C₆₋₁₀ aryl)C₁₋₆ alkyl; and
each W is independently selected from NR and O.

24. The compound according to any one of claims 2 to 23, wherein the fragment ((L)ₐ)₃J is selected from the following structures:

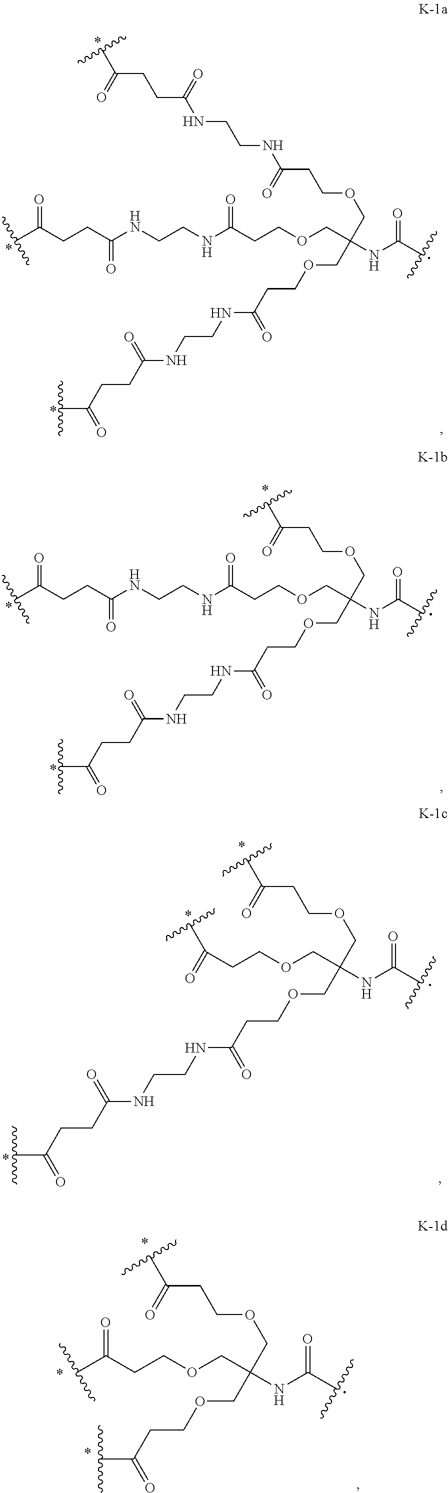

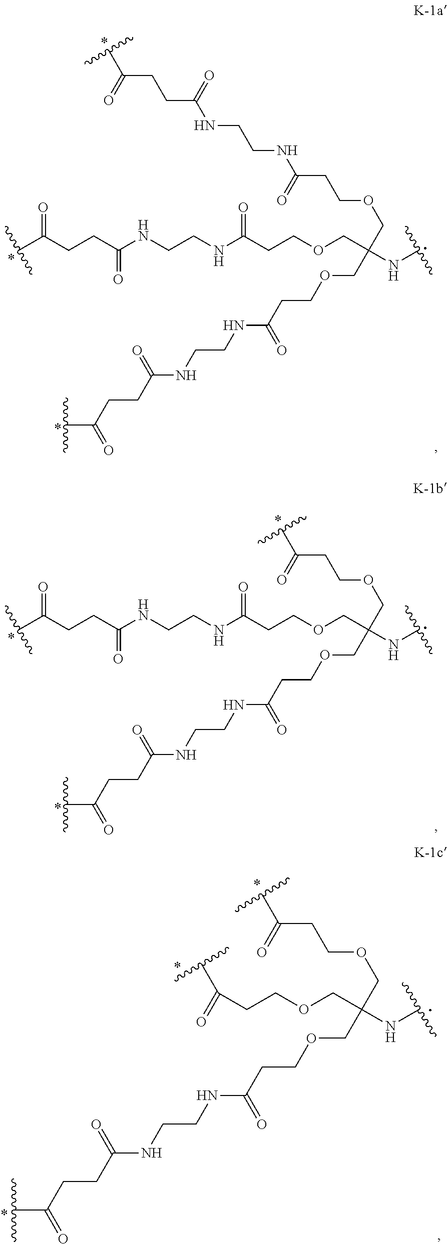

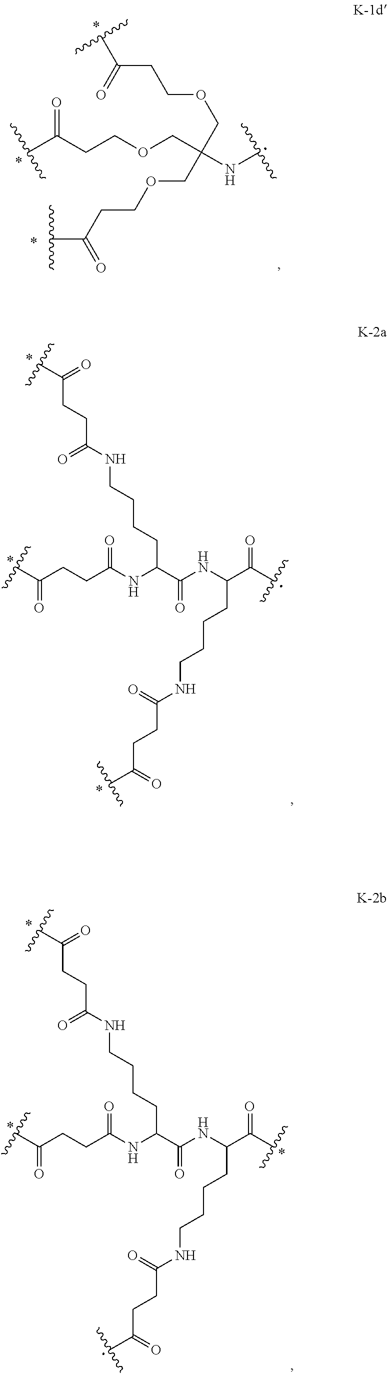

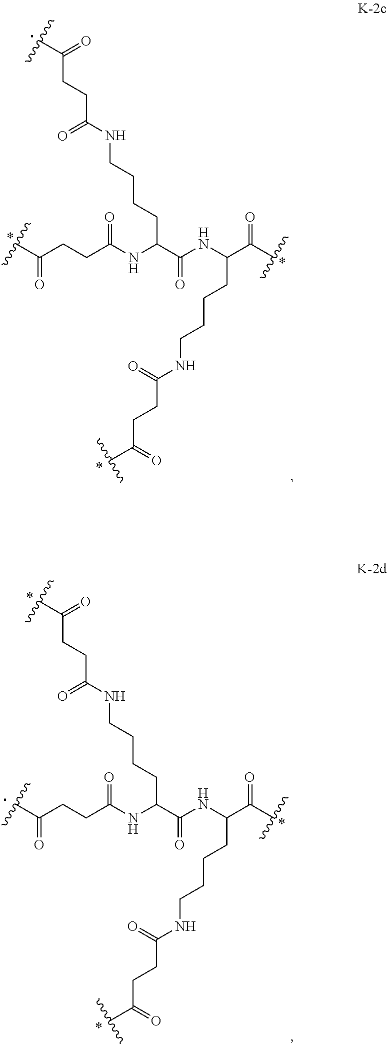

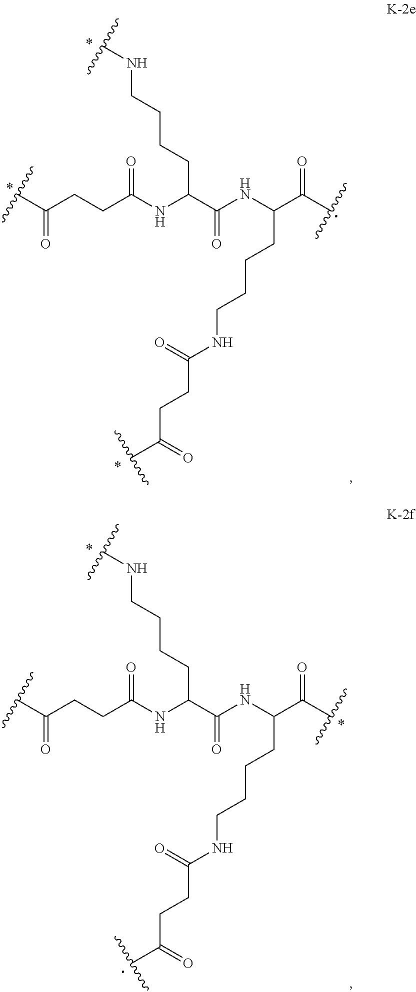

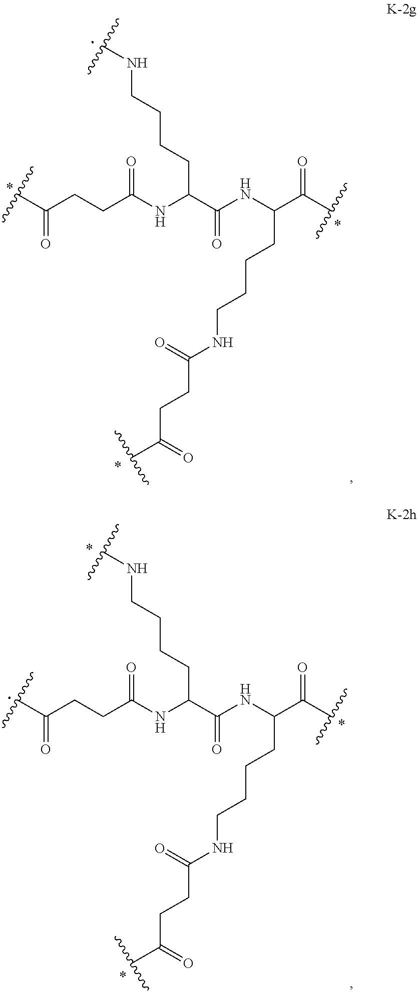

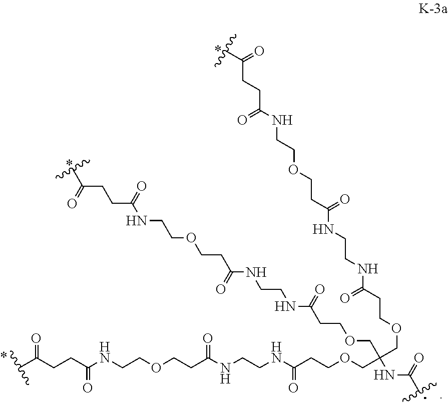

25. The compound according to any one of claims 2 to 23, wherein the fragment ((L)ₐ)₃J is selected from the following structures: 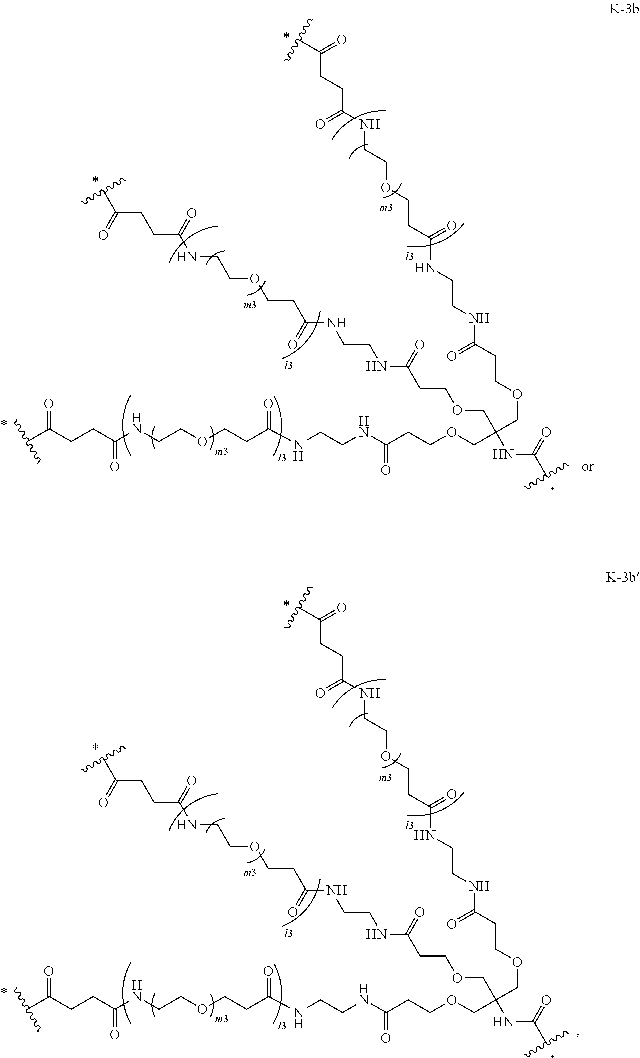
wherein each m₃ is independently 0, 1, 2, 3, 4, 5, 6, 7, 8, 9, 10 or 11, preferably 0, 1, 2, 3 or 4; and
each l₃ is independently 0 or 1, preferably 0.

26. The compound according to any one of the preceding claims, wherein the moiety C is selected from: (a) a chelating agent group suitable for radiolabelling with therapeutic nuclides; (b) a therapeutic radioactive group comprising a therapeutic radioisotope; (c) a chelate of a therapeutic radioactive isotope with a chelating agent; (d) a cytotoxic and/or cytostatic agent; (f) immunomodulator agent; or (g) a protein.

27. The compound according to any one of the preceding claims, wherein the moiety C is a chelating agent group suitable for radiolabelling with therapeutic nuclides selected from compounds having a structure according to any of the following formulae: 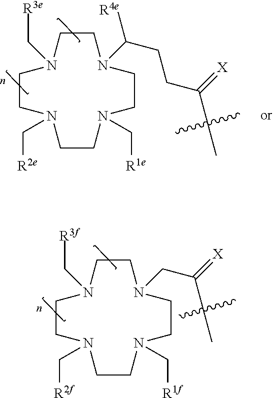
wherein:
n is 0, 1, 2, 3, 4 or 5; preferably 1;
R^{1e} is independently H, COOH, aryl-COOH or heteroaryl-COOH; preferably COOH;
R^{2e} is independently H, COOH, aryl-COOH or heteroaryl-COOH; preferably COOH;
each R^{3e} is independently H, COOH, aryl-COOH or heteroaryl-COOH; preferably COOH;
R^{4e} is independently H, COOH, aryl-COOH or heteroaryl-COOH; preferably COOH;
R^{1f} is independently H, COOH, aryl-COOH or heteroaryl-COOH; preferably COOH;
R^{2f} is independently H, COOH, aryl-COOH or heteroaryl-COOH; preferably COOH;
R^{3f} is independently H, COOH, aryl-COOH or heteroaryl-COOH; preferably COOH; and
X is O, NH or S; preferably O.

28. The compound according to any one of the preceding claims, wherein the moiety C is a chelating agent group suitable for radiolabelling with therapeutic nuclides selected from diethylenetriaminepentaacetic acid (DTPA), ethylenediaminetetraacetic acid (EDTA), 1,4,7,10-tetraazacyclododecane-N,N′,N″,N′″-tetraacetic acid (DOTA), 1,4,7-triazacyclononane-N,N′,N″-triacetic acid (NOTA), 1,4,8,11-tetraazacyclotetradecane-N,N′,N″,N′″-tetraacetic acid (TETA), iminodiacetic acid, bis(carboxymethylimidazole)glycine, 6-Hydrazinopyridine-3-carboxylic acid (HYNIC),

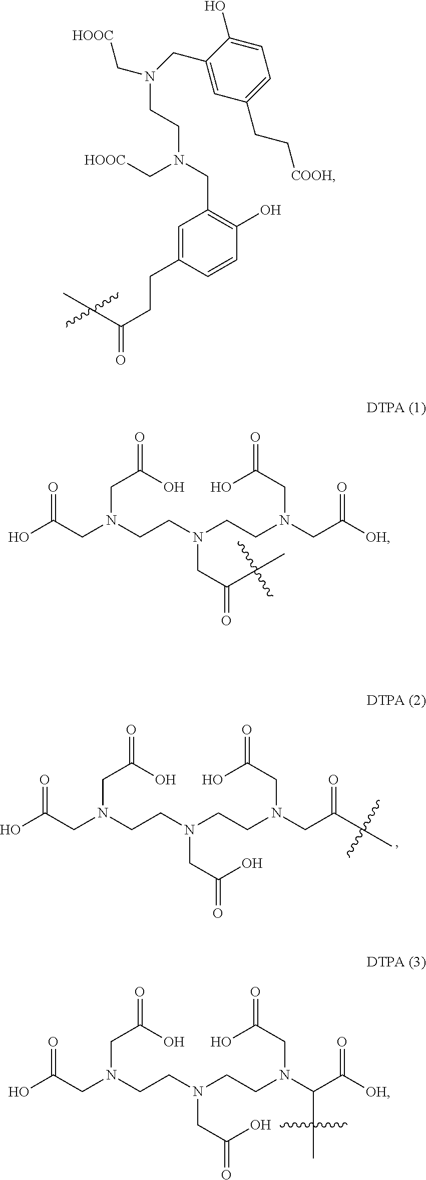 [SMILES: *C(=O)CCc1ccc(O)c(CN(CCN(CC(=O)O)Cc2cc(CCC(=O)O)ccc2O)CC(=O)O)c1 ; *C(=O)CN(CCN(CC(=O)O)CC(=O)O)CCN(CC(=O)O)CC(=O)O ; *C(=O)CN(CCN(CCN(CC(=O)O)CC(=O)O)CC(=O)O)CC(=O)O ; *C(C(=O)O)N(CCN(CCN(CC(=O)O)CC(=O)O)CC(=O)O)CC(=O)O]

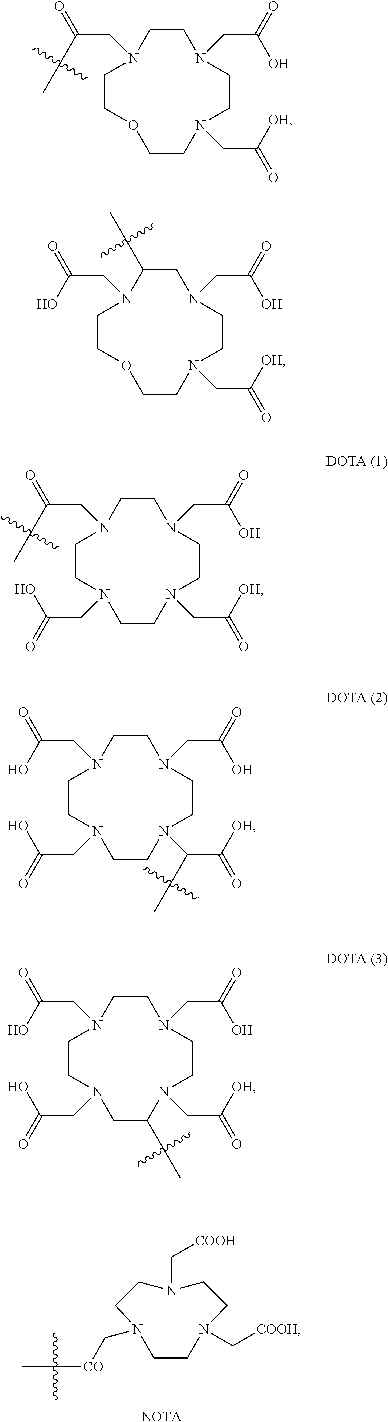

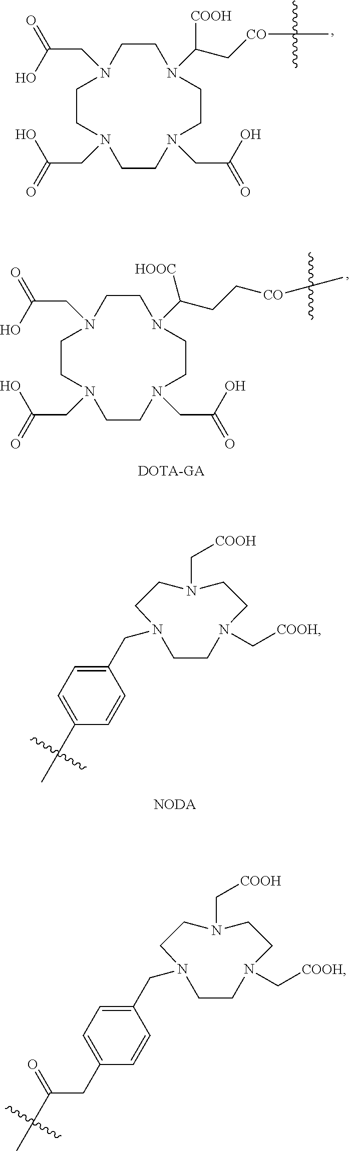 [SMILES: *C(=O)CC(C(=O)O)N1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *c1ccc(CN2CCN(CC(=O)O)CCN(CC(=O)O)CC2)cc1 ; "NODA" ; *C(=O)Cc1ccc(CN2CCN(CC(=O)O)CCN(CC(=O)O)CC2)cc1]

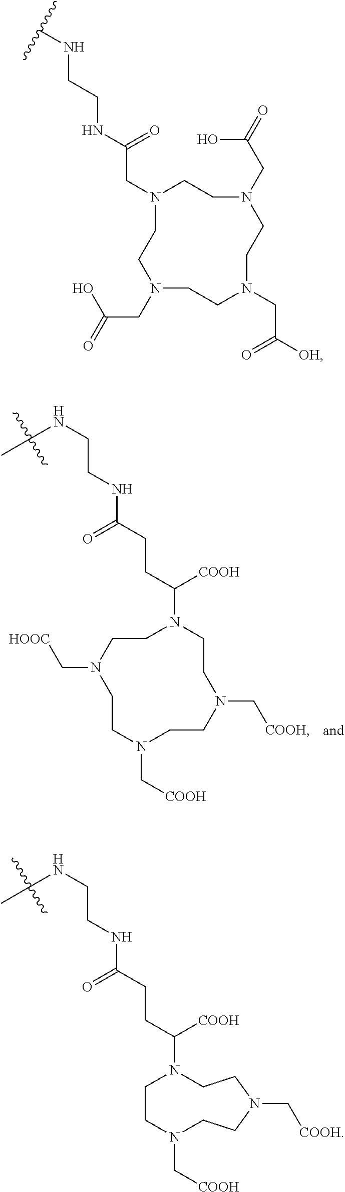 [SMILES: *NCCNC(=O)CN1CCN(CC(=O)O)CCN(CC(=O)O)CCN(CC(=O)O)CC1 ; *NCCNC(=O)CCC(C(=O)O)N1CCN(CC(=O)O)CCN(CC(=O)O)CCN(CC(=O)O)CC1 ; *NCCNC(=O)CCC(C(=O)O)N1CCN(CC(=O)O)CCN(CC(=O)O)CC1]

29. The compound according to any one of the preceding claims, wherein the moiety C is a therapeutic radioactive group comprising a therapeutic radioisotope selected from ²²³Ra, ⁸⁹Sr, ⁹⁰Y, ¹²¹Sn, ¹⁷⁷Lu, ¹³¹I, ²¹¹At, ²²⁵Ac, ¹⁸⁸Re, ¹⁴⁹Tb, ¹⁶¹Tb and ²²⁷Th, preferably ⁹⁰Y, ²²⁵Ac or ¹⁷⁷Lu, more preferably ¹⁷⁷Lu; which may not be used for diagnostic applications.

30. The compound according to any one of claims 26 to 29, wherein the chelate of a therapeutic radioactive isotope is a chelate of an isotope listed in claim 29 and/or with a chelating agent listed in claim 27 or 28.

31. The compound according to any one the preceding claims, wherein moiety C is a radioactive group selected from any of the following structures: 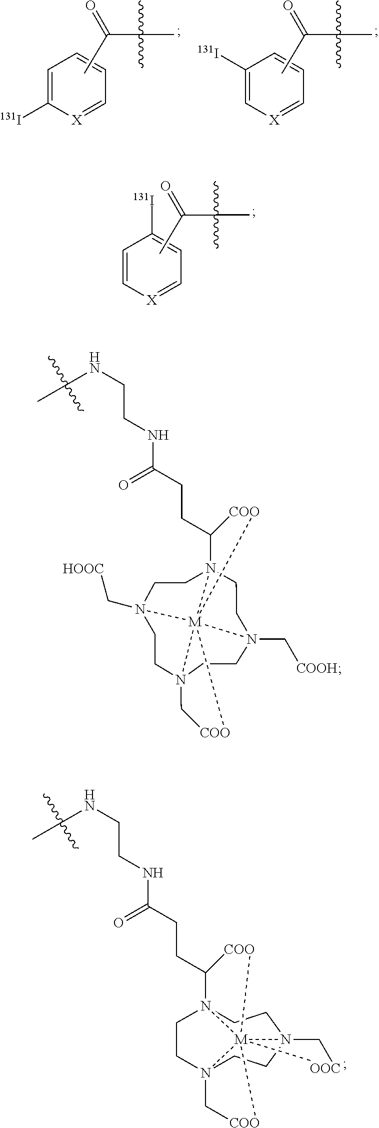 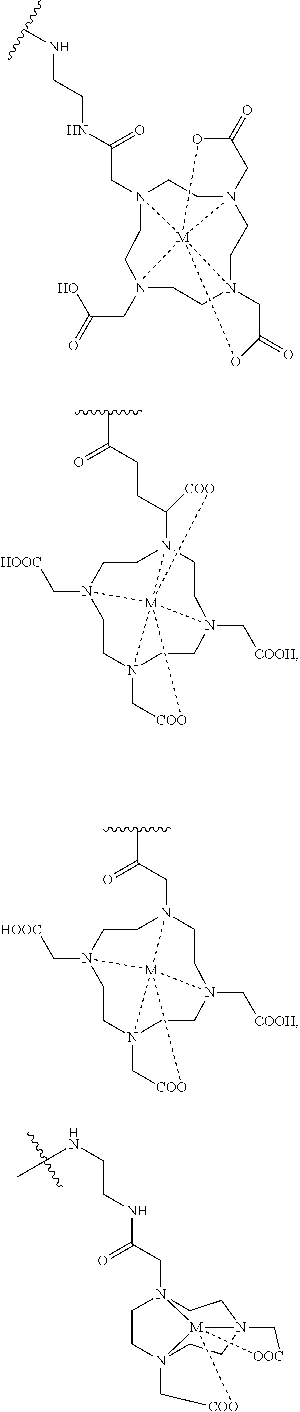 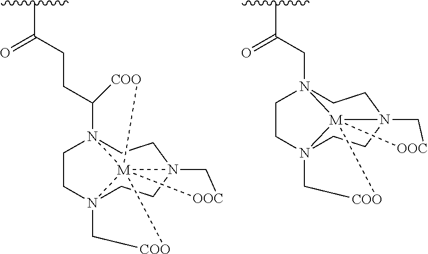
wherein M is a therapeutic radioactive isotope, preferably selected among the list recited in claim 29.

32. The compound according to any one of the preceding claims, wherein moiety C is selected from

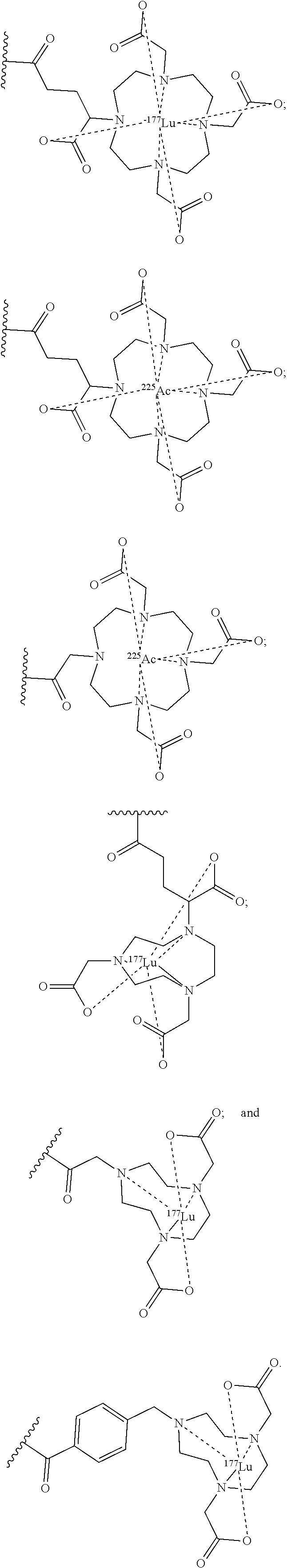

33. The compound according to any one of claims 1 to 26, wherein moiety C is a cytotoxic and/or cytostatic therapeutic agent selected from chemotherapeutic agent selected from the group consisting of topoisomerase inhibitors, alkylating agents, antimetabolites, antibiotics, mitotic disrupters, DNA intercalating agents, DNA synthesis inhibitors, DNA-RNA transcription regulator, enzyme inhibitors, gene regulators, hormone response modifiers, hypoxia-selective cytotoxins, epidermal growth factor inhibitors, anti-vascular agents and a combination of two or more thereof.

34. The compound according to any one of claims 1 to 26 and 33, wherein moiety C is an auristatin, having a structure according to the following formula: 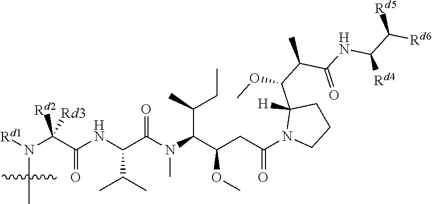
wherein:
R^{1d} is independently H or C₁-C₆ alkyl; preferably H or CH₃;
R^{2d} is independently C₁-C₆ alkyl; preferably CH₃ or iPr;
R^{3d} is independently H or C₁-C₆ alkyl; preferably H or CH₃;
R^{4d} is independently H, C₁-C₆ alkyl, COO(C₁-C₆ alkyl), CON(H or C₁-C₆ alkyl), C₃-C₁₀ aryl or C₃-C₁₀ heteroaryl; preferably H, CH₃, COOH, COOCH₃ or thiazolyl;
R^{5d} is independently H, OH, C₁-C₆ alkyl; preferably H or OH; and
R^{6d} is independently C₃-C₁₀ aryl or C₃-C₁₀ heteroaryl; preferably optionally substituted phenyl or pyridyl.

35. The compound according to any one of claims 1 to 26 and 33, wherein moiety Cis a topoisomerase inhibitor selected from camptothecin (CPT), and a derivative thereof derived by replacing a hydrogen atom from topotecan, irinotecan, silatecan, cositecan, exatecan, lurtotecan, gimatecan, belotecan, rubitecan, deruxtecan and DXd.

36. The compound according to any one of claims 1 to 26 and 33, wherein moiety C is a cytotoxic and/or cytostatic therapeutic agent selected from the following structures:

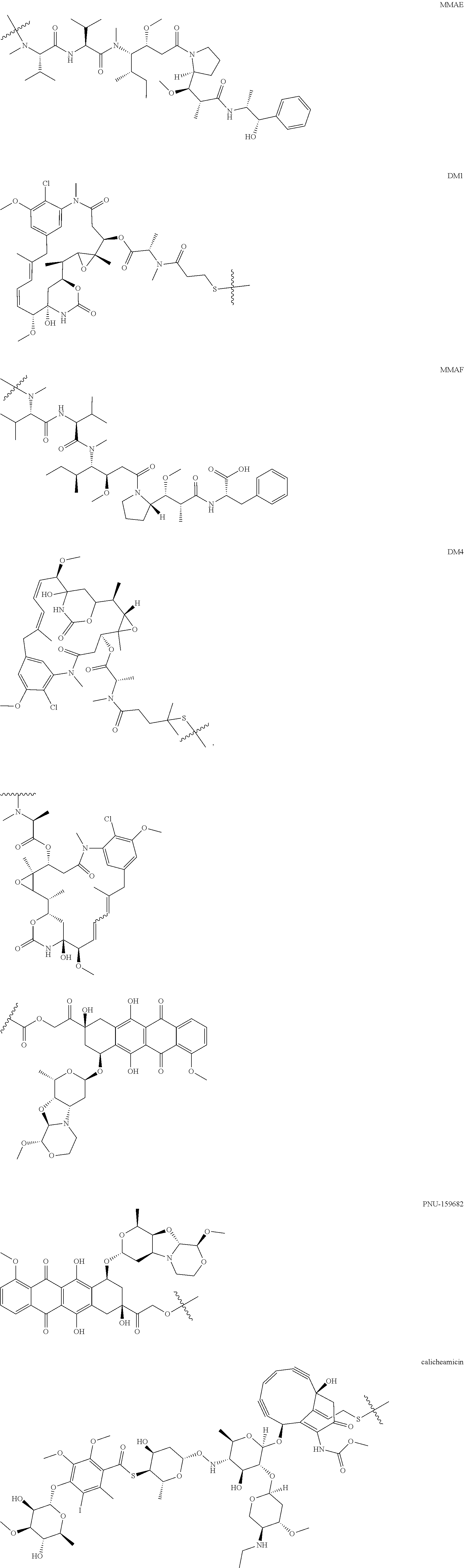

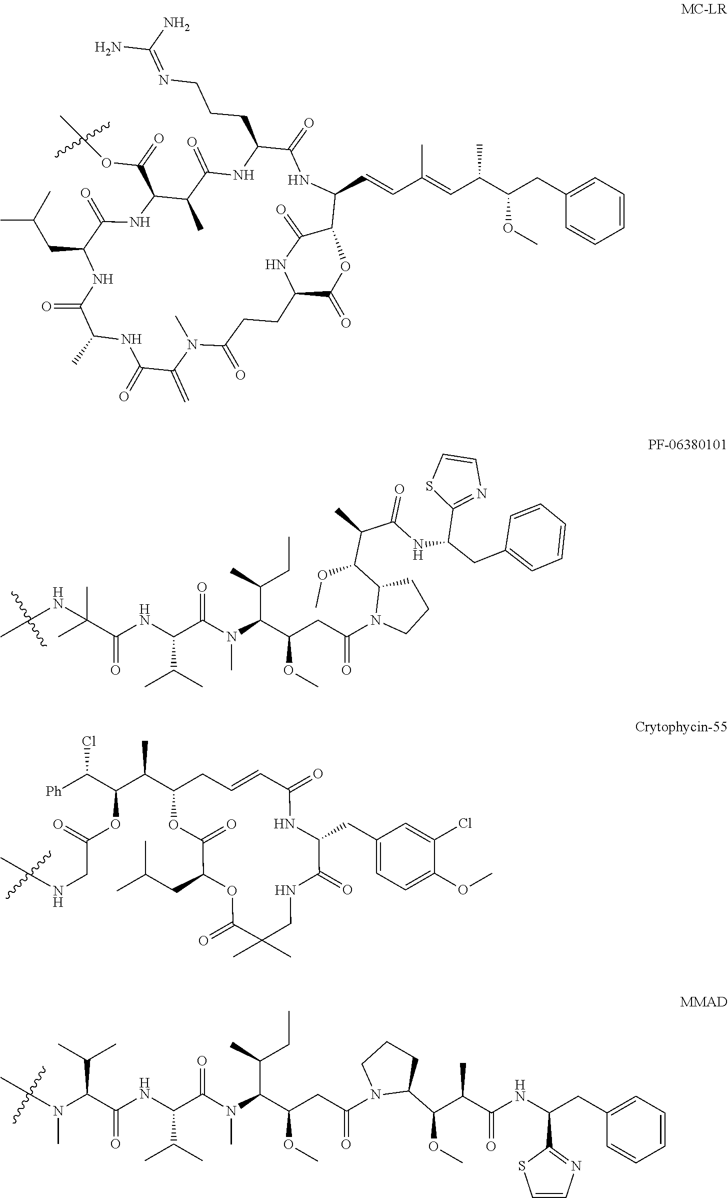

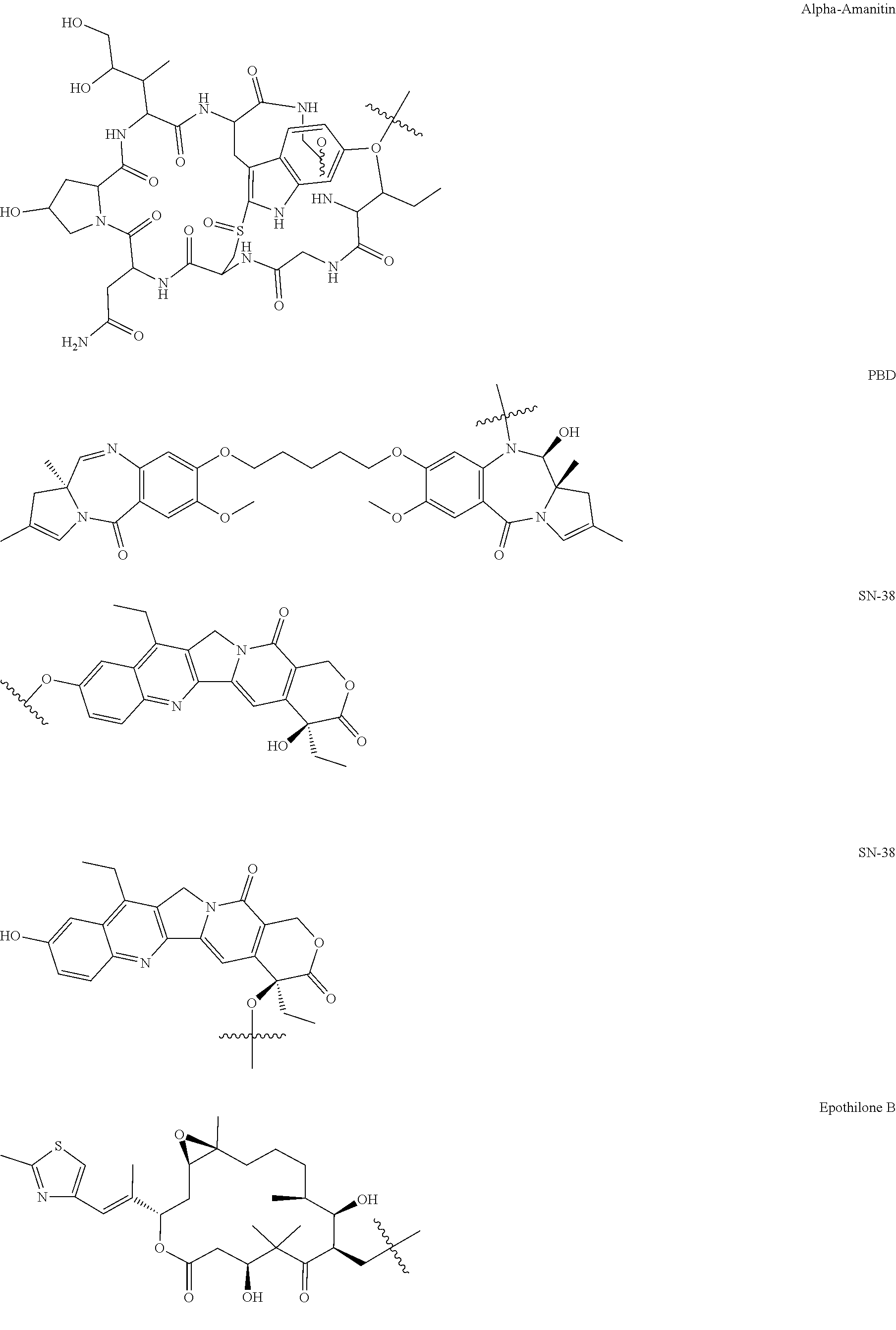

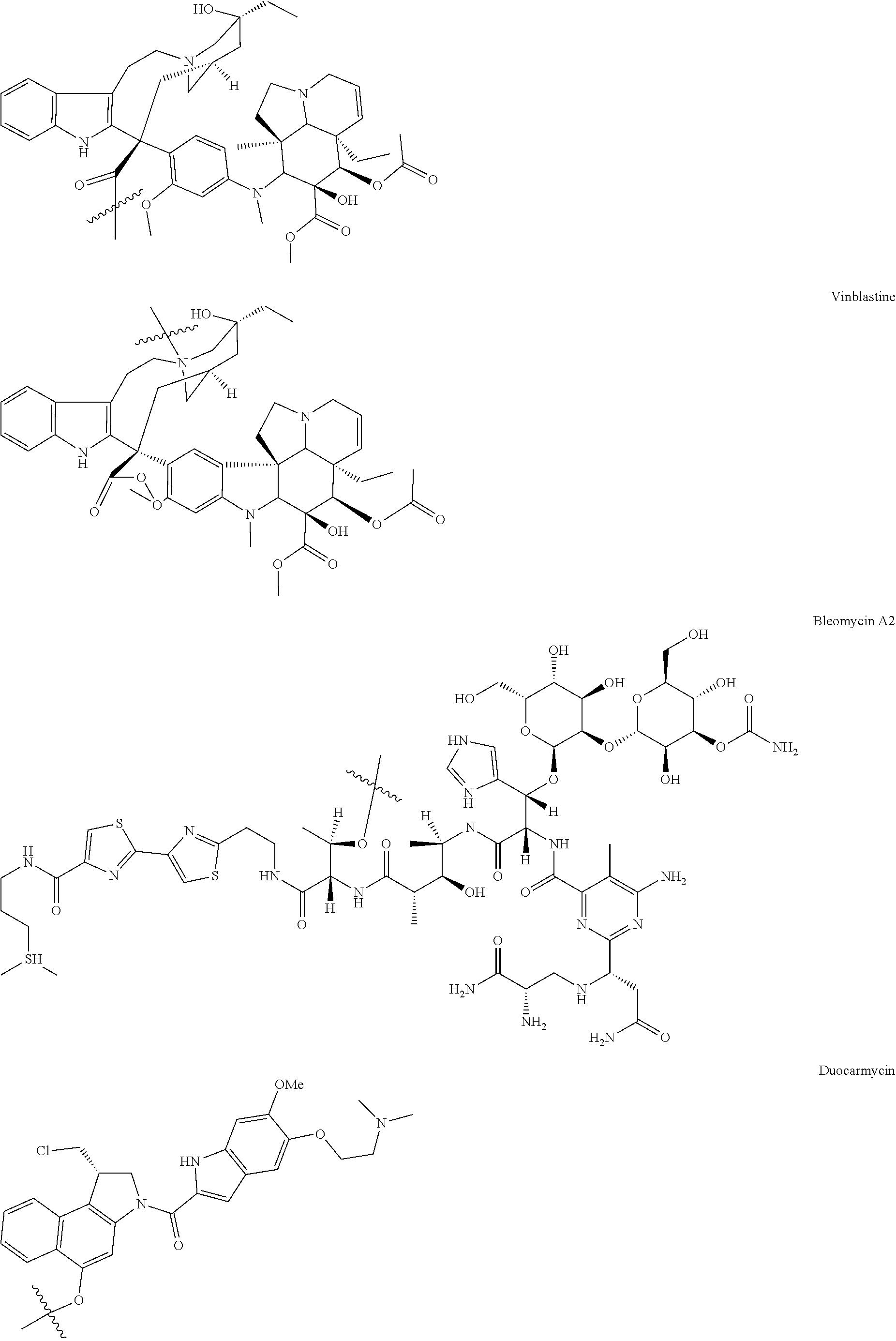

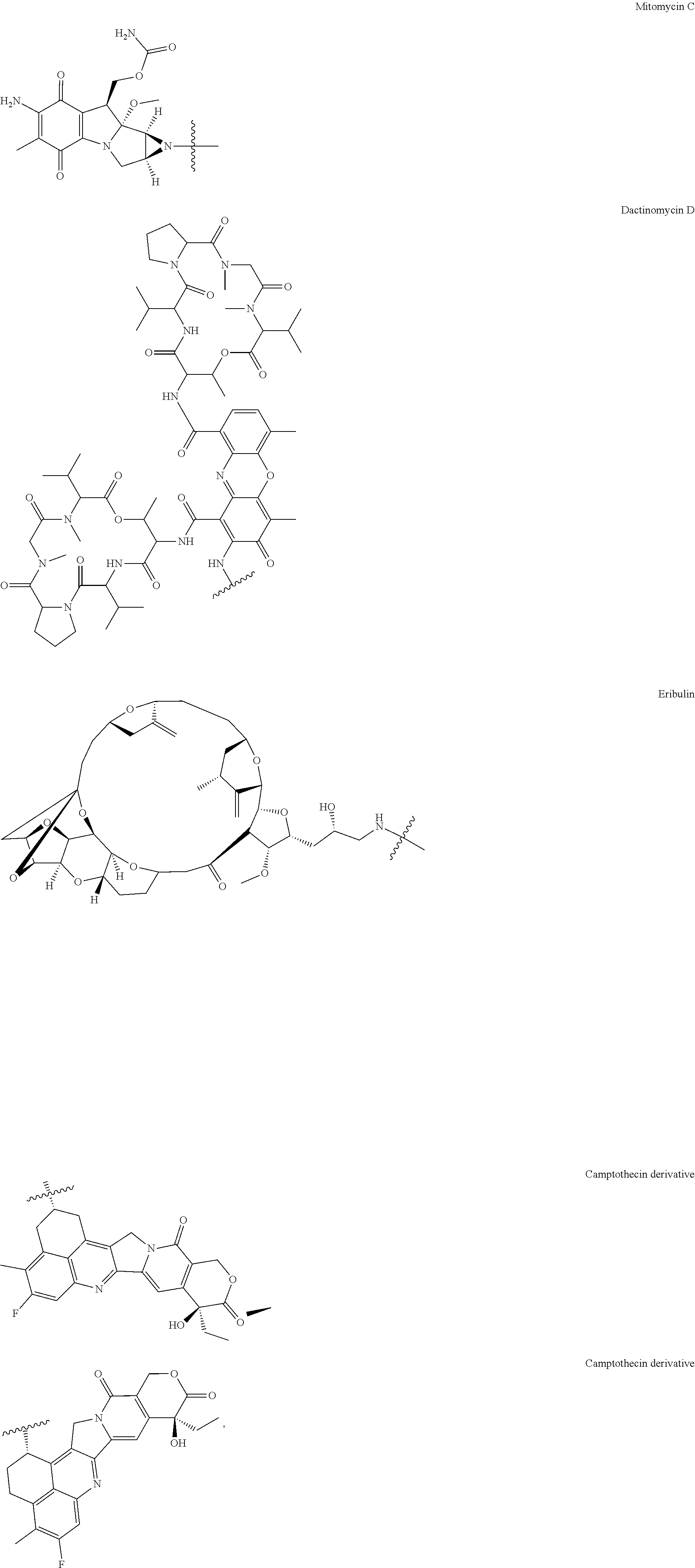

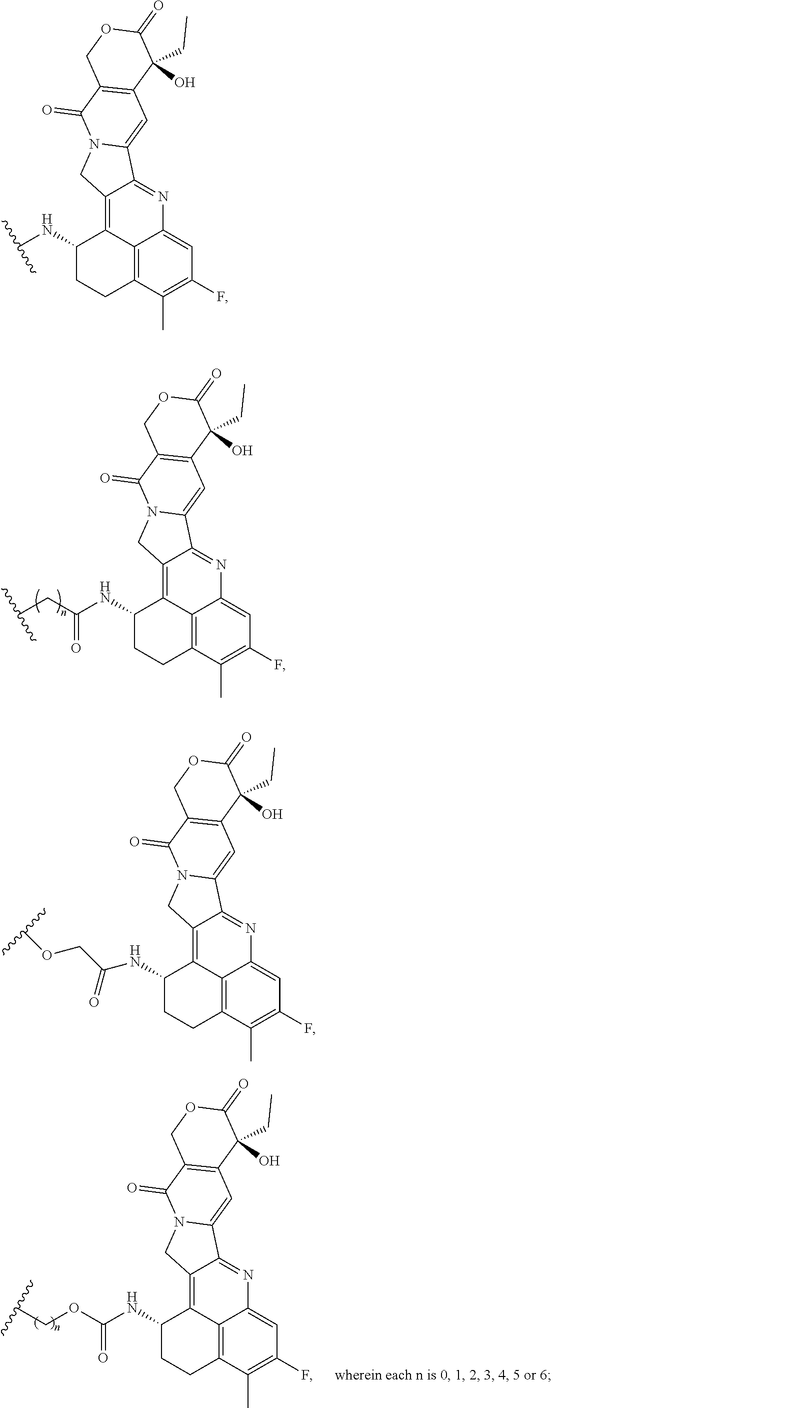

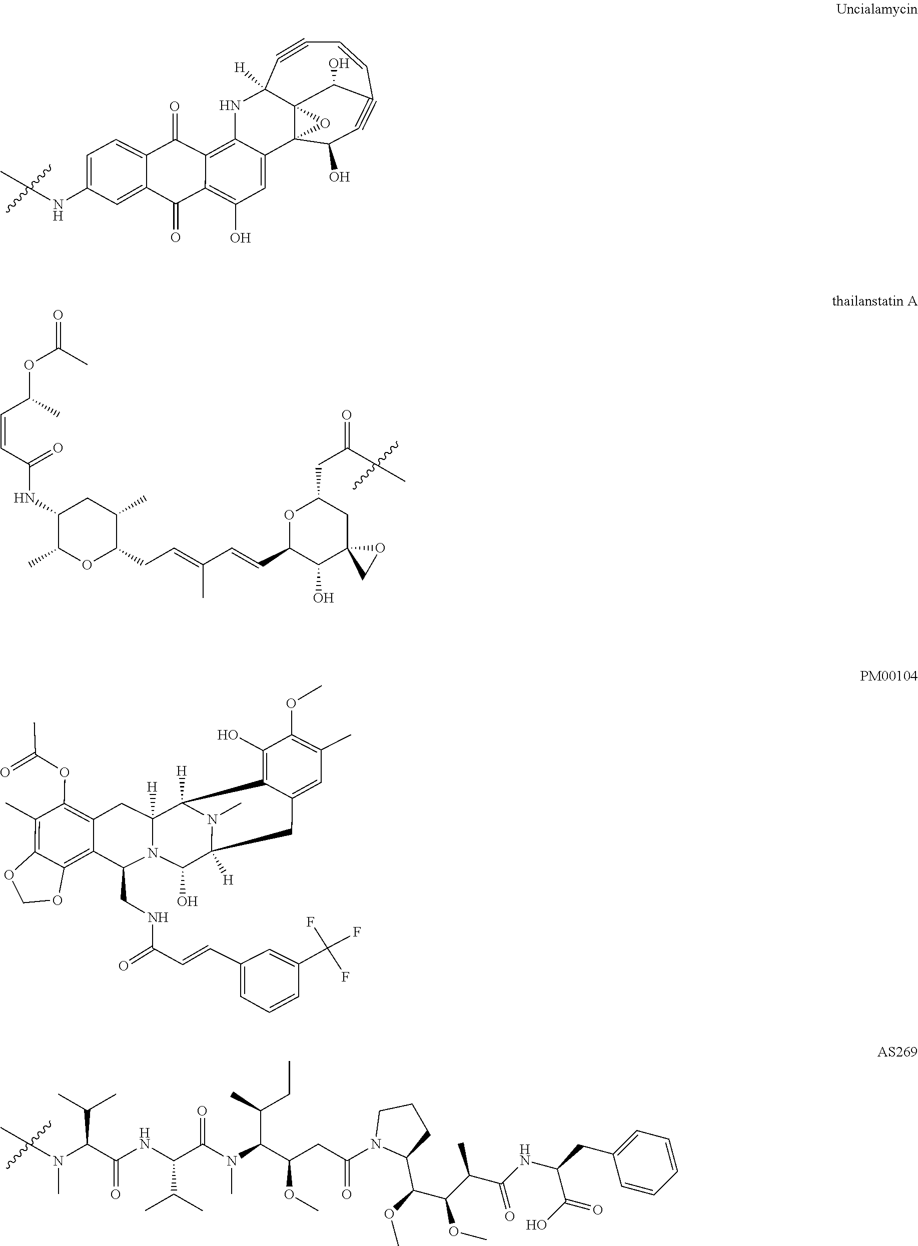

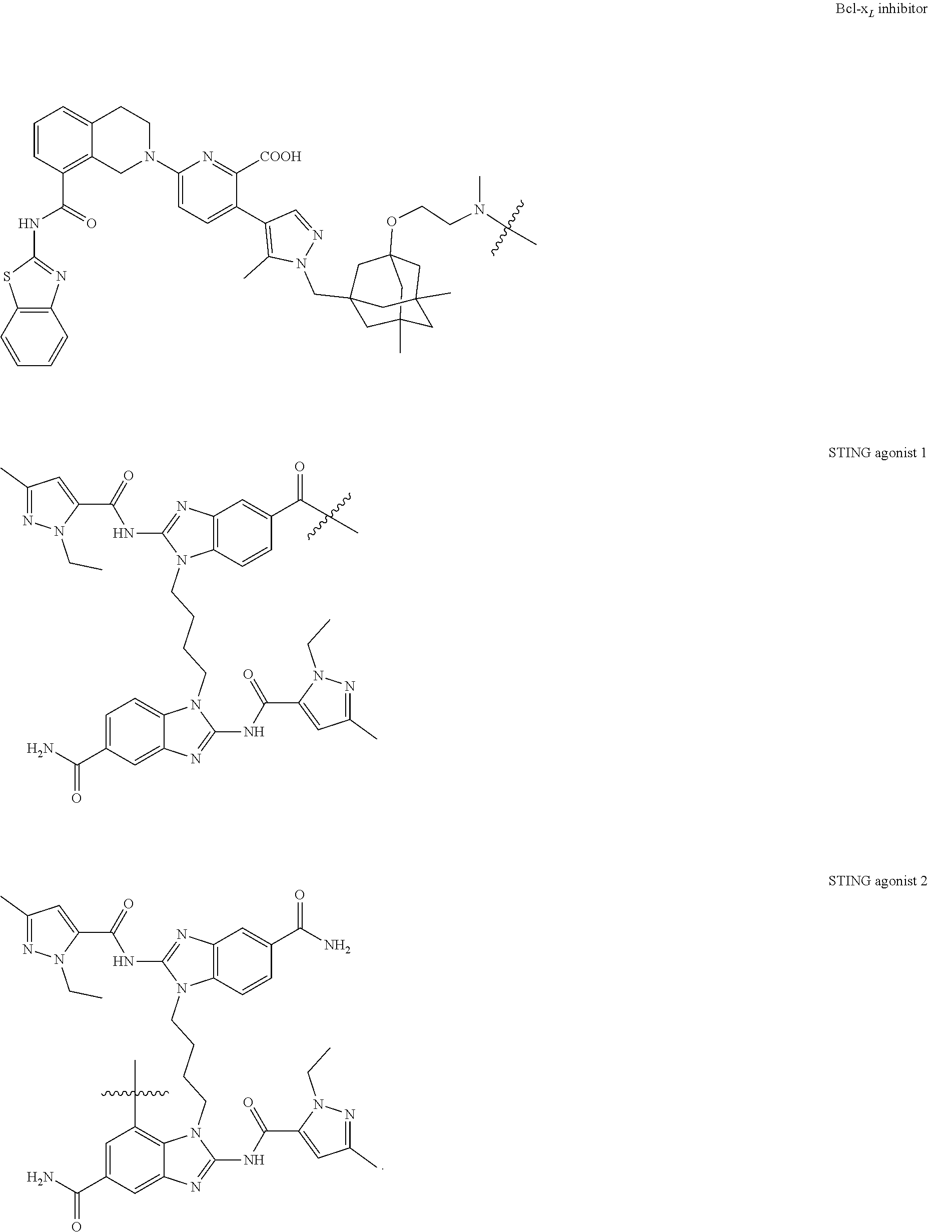

37. The compound according to any one of claims 1 to 26, wherein moiety C is an immunomodulator agent is selected from molecules known to be able to modulate the immune system, such as ligands of CD3, CD25, TLRs, STING, 4-1BBL, 4-1BB, PD-1, mTor, PDL-1, NKG-2D IMiDs, wherein ligands can be agonists and/or antagonist.

38. The compound according to any one of claim 1 to 26 or 37, wherein moiety C is a therapeutic protein selected from cytokines, such as IL2, IL10, IL12, IL15, TNF, Interferon Gamma, or is a therapeutic antibody.

39. The compound according to any one of the preceding claims, wherein the fragment ((B_{S})ₓ(B_{L})_{y})_{z}C, is represented by one of the following structures: 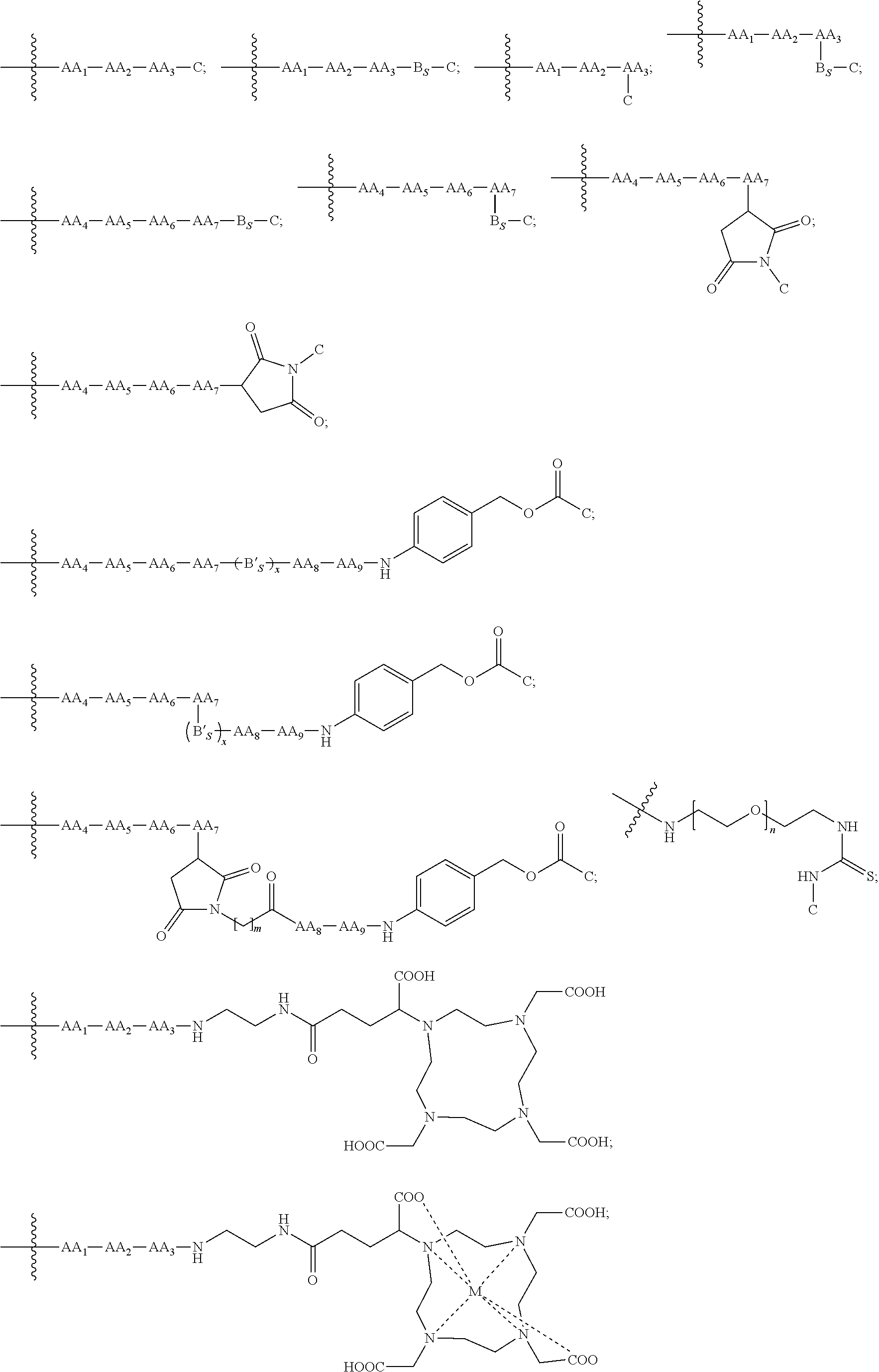 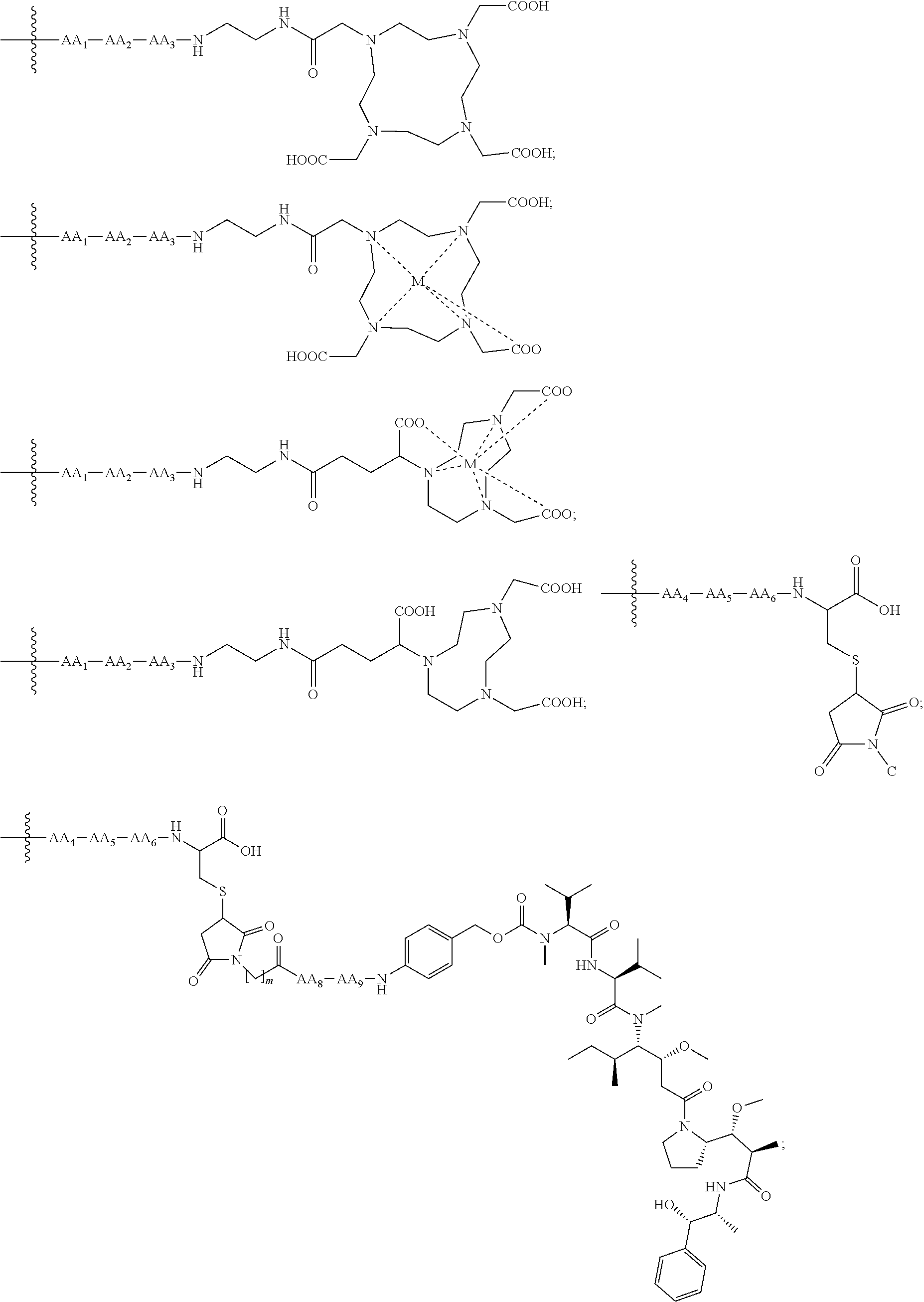 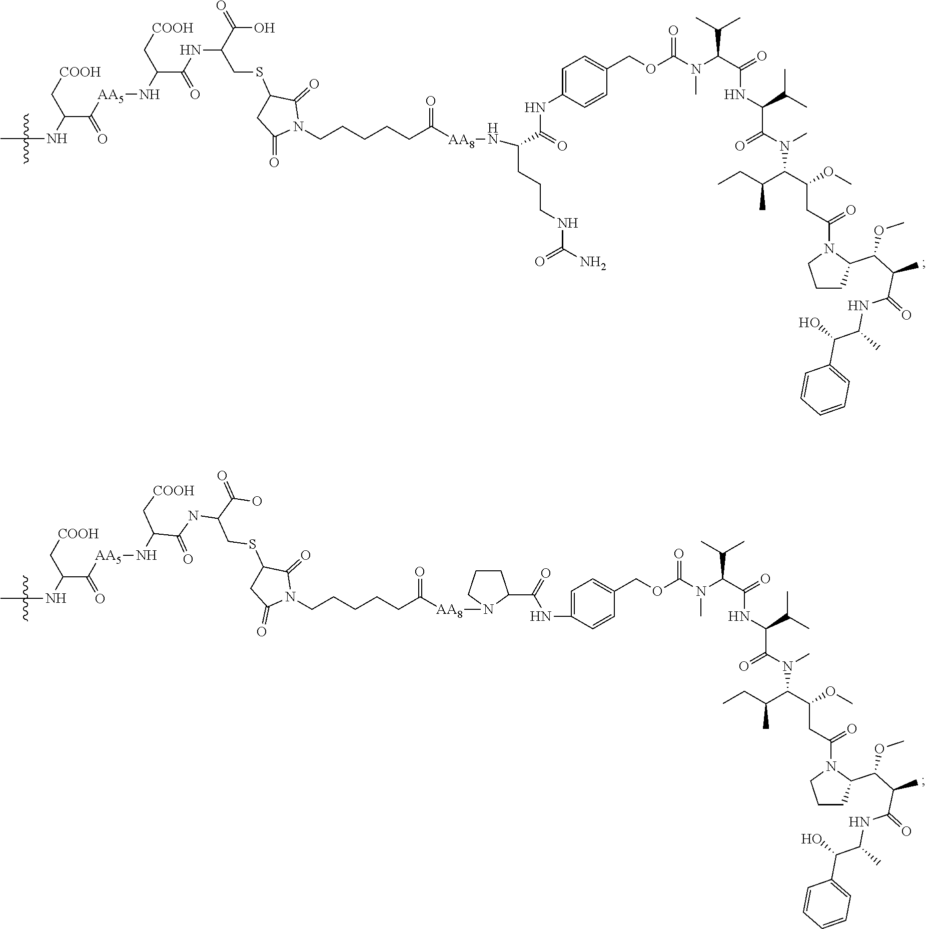
wherein each of AA₁, AA₂, AA₃, AA₄, AA₅, AA₆, AA₇, AA₈, and AA₉ represents a proteinogenic or non-proteinogenic amino acid, or is absent, and
wherein, unless otherwise specified, all groups and variables are defined as in any one of the preceding claims.

40. The compound according to claim 39, wherein AA₅ is an amino acid with a charged sidechain, and AA₈ is an amino acid with an aliphatic side chain.

41. The compound according to claim 39 or 40, wherein: AA₁ is selected from Asp and Glu, or is absent; AA₂ is selected from Asp and Glu, or is absent; AA₃ is Lys; AA₄ is selected from Asp and Glu; AA₅ is selected from Lys and Arg; AA₆ is selected from Asp and Glu; AA₇ is selected from Cys; and AA₈ is selected from Gly, Ala, and Val; and AA₉ is selected from Pro and citrulline (Cit).

42. The compound according to any one of the preceding claims represented by the following formula: 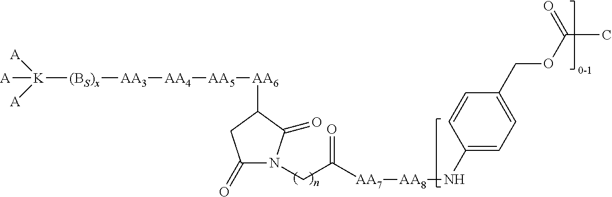
wherein K represents fragment ((L)ₐ) 3J as defined in any one of the preceding claims;
(B_{S})ₓ is independently represented by bond, —NHC(O)(CH₂)ₙC(O)—, —NH(CH₂)ₙC(O)—, —NHC(O)(CH₂CH₂O)ₘ(CH₂)ₙ—, —C(O)(CH₂CH₂O)ₘ(CH₂)ₙ—, —C(O)(CH₂CH₂O)ₘ(CH₂)ₙNH—, —(CH₂CH₂O)ₘ(CH₂)ₙ—, —(CH₂CH₂O)ₘ(CH₂)ₙNH—, —(CH₂CH₂O)ₘ(CH₂)ₙNHC(O)—, —(CH₂)ₙO(CH(CH₂CH₂O)ₘ(CH₂)ₙ—, —(CH₂)ₙO(CH₂CH₂O)ₘ(CH₂)ₙNH—, —(CH₂CH₂O)ₘ(CH₂)ₙNHC(O)—, —C(O)(CH₂)ₙO(CH₂CH₂O)ₘ(CH₂)ₙ—, —C(O)(CH₂)ₙO(CH₂CH₂O)ₘ(CH₂)ₙNH—, —(CH₂)ₙO(CH₂CH₂O)ₘ(CH₂)ₙNH—, —C(O)(CH₂)ₙC(O)—, —C(O)(CH₂)ₙ—, —C(O)(CH₂)ₙNH— or —(CH₂)ₙC(O)—, preferably —C(O)(CH₂)ₙC(O)— or —(CH₂)ₙC(O)—;
each n and m is independently an integer, preferably selected from 0, 1, 2, 3, 4, 5 and 6;
each of AA₃, AA₄, AA₅, AA₆, AA₇ and AA₈ independently represents a proteinogenic or non-proteinogenic amino acid, or is absent; and
C is a cytotoxic and/or cytostatic agent.

43. The compound according to claim 42, wherein AA₅ is an amino acid with a charged sidechain, and AA₇ is an amino acid with an aliphatic side chain;

44. The compound according to claim 42 or 43, wherein AA₃ is absent; AA₄ is selected from Asp and Glu; AA₅ is selected from Lys and Arg; AA₆ is selected from Asp and Glu; AA₇ is selected from Gly, Ala, and Val; and AA₈ is selected from Pro and citrulline (Cit).

45. The compound according to any one of claims 42 to 44, wherein C is a topoisomerase inhibitor as defined in claim 35.

46. The compound according to any one of claims 42 to 44, wherein C is an auristatin as defined in claim 34.

47. The compound according to any one of the preceding represented by one of the following formulae: 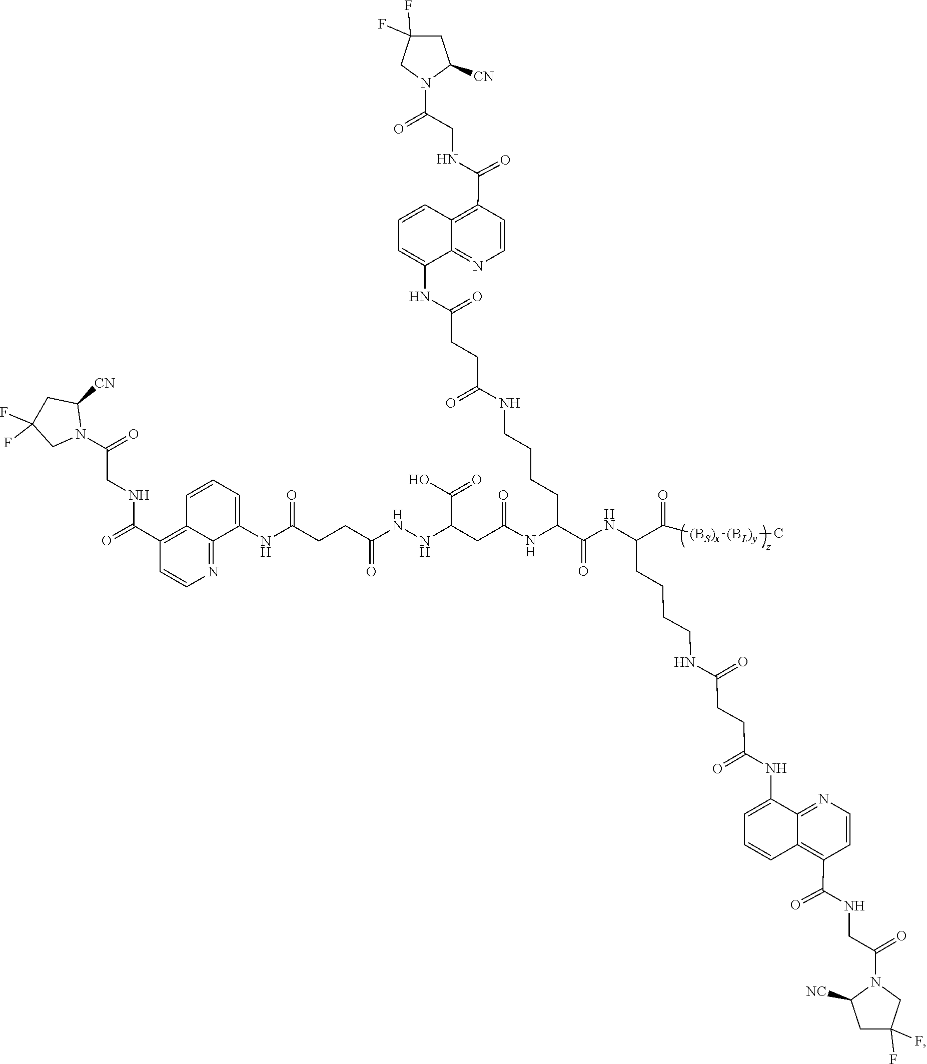 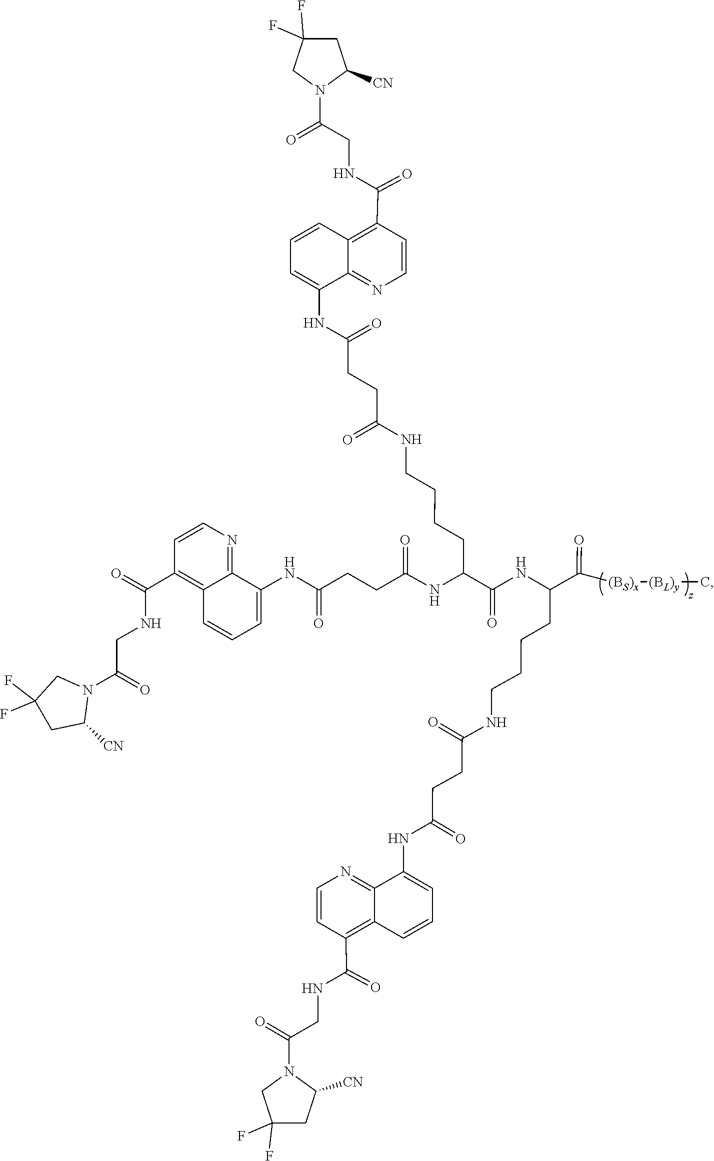 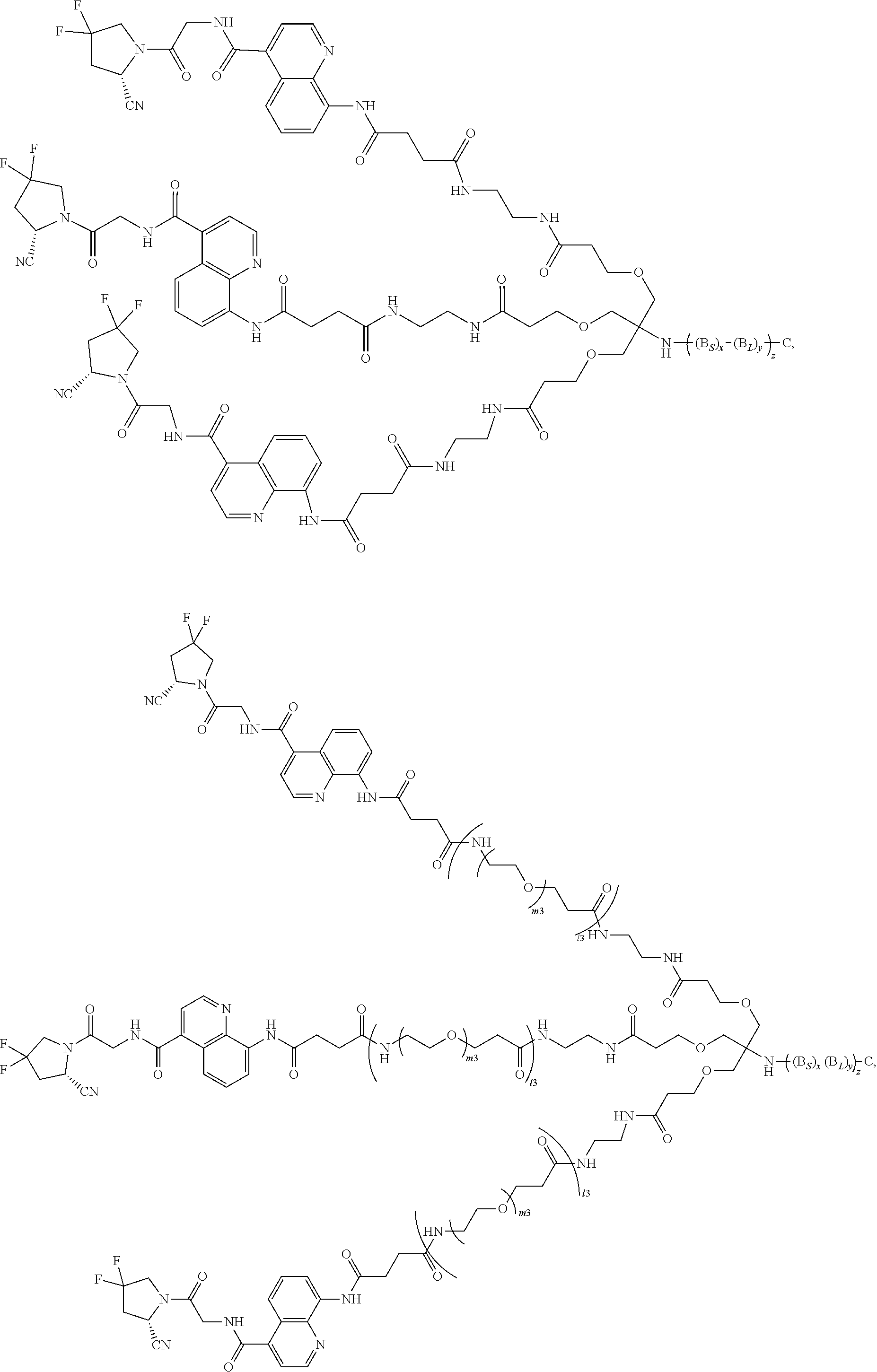
wherein each (B_{S})ₓ, (B_{L})_{y} or ((B_{S})ₓ—(B_{L})_{y}) is independently represented by bond, —NHC(O)(CH₂)ₙC(O)—, —NH(CH₂)ₙC(O)—, —NHC(O)(CH₂CH₂O)ₘ(CH₂)ₙ—, —C(O)(CH₂CH₂O)ₘ(CH₂)ₙ—, —C(O)(CH₂CH₂O)ₘ(CH₂)ₙNH—, —(CH₂CH₂O)ₘ(CH₂)ₙ—, —(CH₂CH₂O)ₘ(CH₂)ₙNH—, —(CH₂CH₂O)ₘ(CH₂)ₙNHC(O)—, —(CH₂) NO(CH(CH₂CH₂O)ₘ(CH₂)ₙ—, —(CH₂)ₙO(CH₂CH₂O)ₘ(CH₂)ₙNH—, —(CH₂CH₂O)ₘ(CH₂)ₙNHC(O)—, —C(O)(CH₂)ₙO(CH₂CH₂O)ₘ(CH₂)ₙ—, —C(O)(CH₂)ₙO(CH₂CH₂O)ₘ(CH₂)ₙNH—, —(CH₂)ₙO(CH₂CH₂O)ₘ(CH₂)ₙNH—, —C(O)(CH₂)ₙC(O)—, —C(O)(CH₂)ₙ—, —C(O)(CH₂)ₙNH— or —(CH₂)ₙC(O)—, preferably —C(O)(CH₂)ₙO(CH₂CH₂O)ₘ(CH₂)ₙNH—;
each n and m is independently an integer selected from 0, 1, 2, 3, 4, 5, 6, 7, 8, 9, 10, 11 and 12, preferably selected from 0, 1, 2, 3, 4, 5 and 6; and
each z is an integer independently selected from 0, 1, 2, 3, 4 and 5, preferably 1;
each m₃ is independently 0, 1, 2, 3, 4, 5, 6, 7, 8, 9, 10 or 11, preferably 1; and
each l₃ is independently 0, or 1, preferably 0.

48. The compound according to any one of the preceding represented by the following formula: 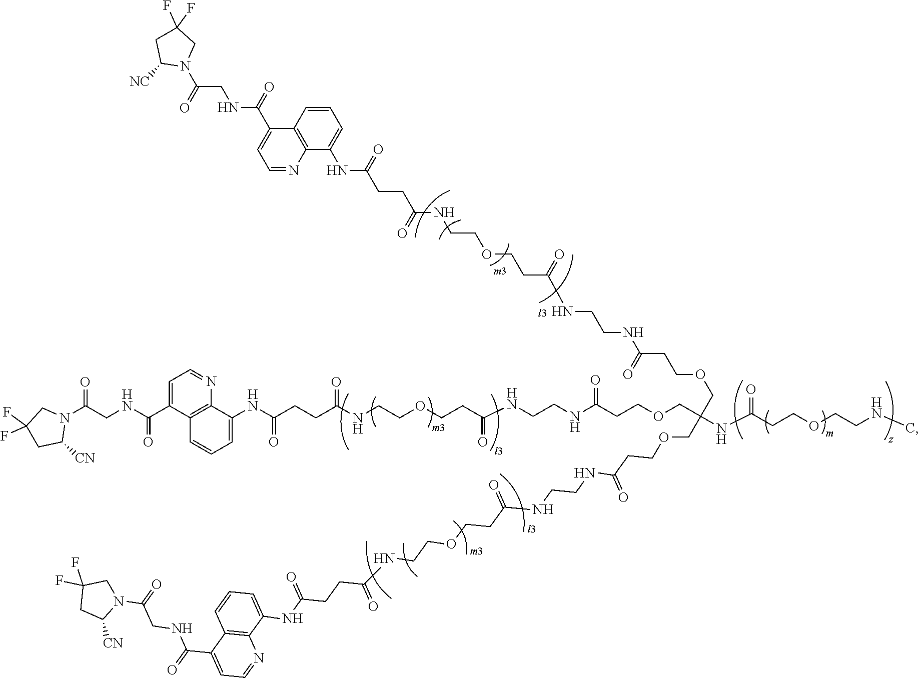
wherein each m₃ is independently 0, 1, 2, 3, 4, 5, 6, 7, 8, 9, 10 or 11, preferably 1;
each l₃ is independently 0, or 1, preferably 0;
z is an integer independently selected from 0, 1, 2, 3, 4 and 5, preferably 1; and
each m is independently an integer selected from 0, 1, 2, 3, 4, 5, 6, 7, 8, 9, 10, 11 and 12, preferably selected from 0, 1, 2, 3, 4, 5 and 6.

49. The compound according to claim 47 or 48, wherein moiety C is a chelating agent group suitable for radiolabelling with therapeutic nuclides.

50. The compound according to claim 47 or 48, wherein moiety C is as defined in any one of claims 27 to 32.

51. A compound having a structure selected from the compounds in Table 9, its individual diastereoisomers, its hydrates, its solvates, its crystal forms, its individual tautomers or a pharmaceutically acceptable salt thereof.

52. The compound according to any one of the preceding claims having one of the following structures:

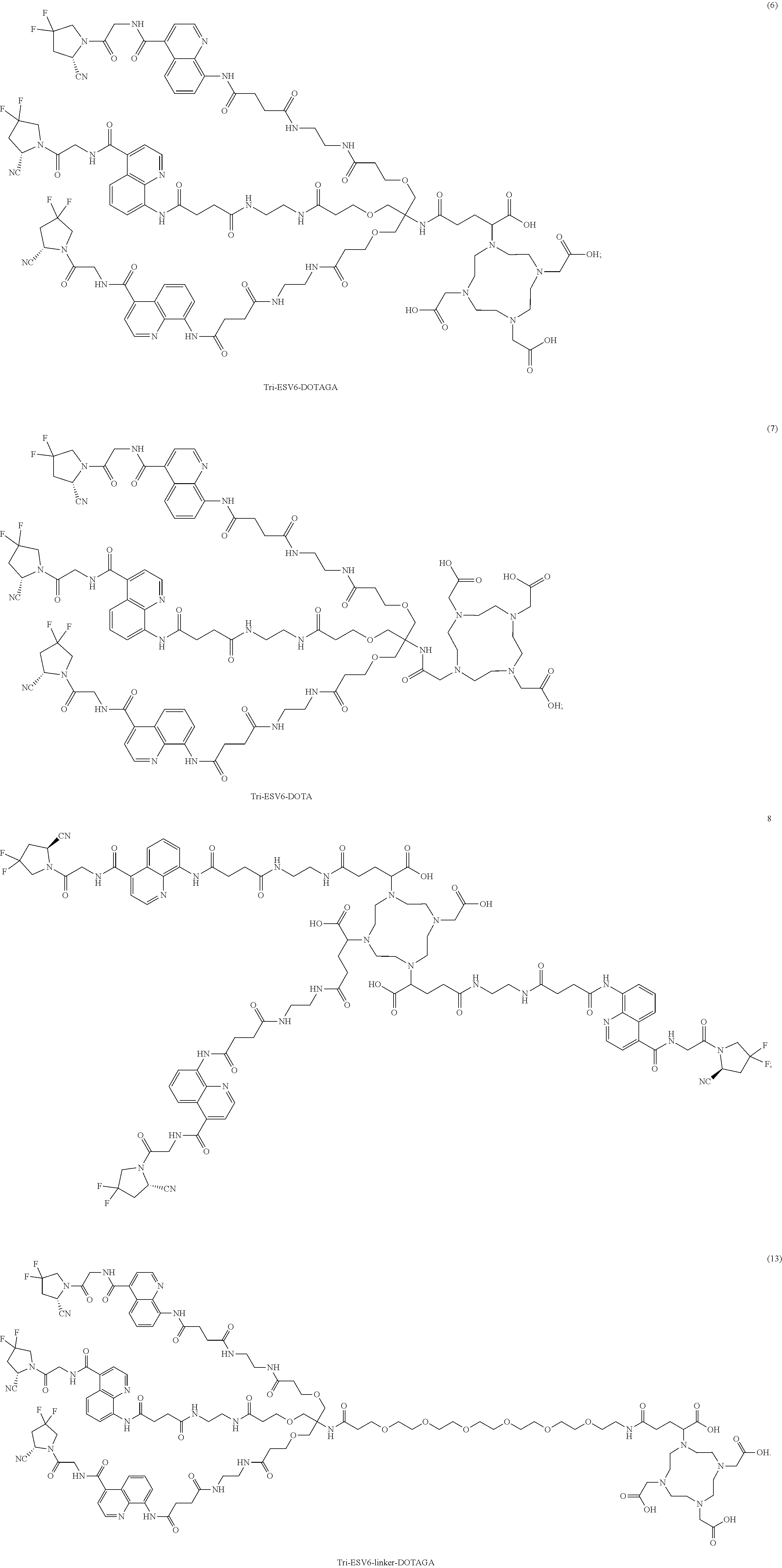

53. The compound according to any one of claims 1 to 51 having one of the following structures:

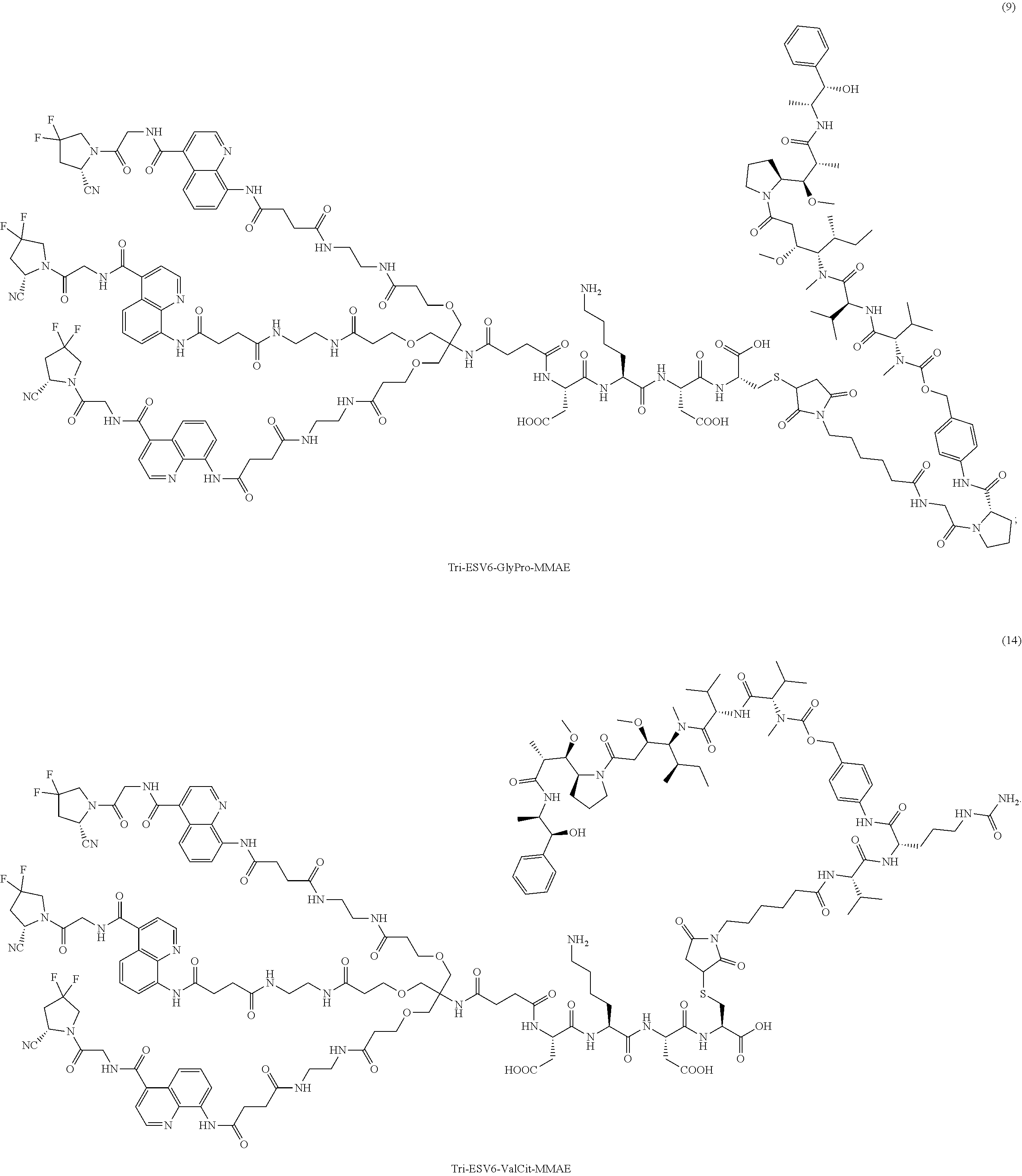

54. A pharmaceutical composition comprising the compound according to any one of the preceding claims, and a pharmaceutically acceptable excipient.

55. A pharmaceutical combination comprising (i) the compound according to any one of claims 1-53 or the pharmaceutical composition according to claim 54 and (ii) an immunocytokine.

56. The pharmaceutical combination according to claim 55, wherein the immunocytokine binds specifically to the alternatively spliced ED-B domain of fibronectin isoform B-FN, optionally to the epitope sequence represented by SEQ ID NO: 40.

57. The pharmaceutical combination according to any one of claim 55 or 56, wherein the immunocytokine comprises a sequence having IL2 activity.

58. The pharmaceutical combination according to any one of claims 55-57, wherein the immunocytokine comprises one or more of the CDRs of any one of SEQ ID NOs: 13-18, optionally all of SEQ ID NOs: 13-18, further optionally wherein the immunocytokine comprises the VH sequence of SEQ ID NO: 19 and/or the VL sequence of SEQ ID NO: 20.

59. The pharmaceutical combination according to any one of claims 55-58, wherein the immunocytokine comprises an scFv sequence, optionally a human monoclonal scFv sequence.

60. The pharmaceutical combination according to any one of claims 55-59, wherein the immunocytokine comprises the sequence of SEQ ID NO: 12.

61. The compound according to any one of claims 1-53, the pharmaceutical composition according to claim 54, or the pharmaceutical combination according to any one of claims 55-60 for use in:
(a) a method for treatment of the human or animal body by surgery or therapy; or
(b) a method for therapy or prophylaxis of a subject suffering from or having risk for a disease or disorder; or
(c) a method for targeted delivery of a therapeutic agent to a subject suffering from or having risk for a disease or disorder.

62. The compound according to any one of claims 1-53, the pharmaceutical composition according to claim 54, or the pharmaceutical combination according to any one of claims 55-60 for the use as defined in claim 61, wherein said disease or disorder is independently selected from cancer, inflammation, atherosclerosis, fibrosis, tissue remodelling and keloid disorder, preferably wherein the cancer is selected from the group consisting of breast cancer, pancreatic cancer, small intestine cancer, colon cancer, multi-drug resistant colon cancer, rectal cancer, colorectal cancer, metastatic colorectal cancer, lung cancer, non-small cell lung cancer, head and neck cancer, ovarian cancer, hepatocellular cancer, oesophageal cancer, hypopharynx cancer, nasopharynx cancer, larynx cancer, myeloma cells, bladder cancer, cholangiocarcinoma, clear cell renal carcinoma, neuroendocrine tumour, oncogenic osteomalacia, sarcoma, CUP (carcinoma of unknown primary), thymus cancer, desmoid tumours, glioma, astrocytoma, cervix cancer, skin cancer, kidney cancer and prostate cancer; and/or
wherein the compound has a prolonged residence at the disease site at a therapeutically relevant level, e.g., for at least for 5 min, 10 min, 20 min, 30 min, 45 min, 1 h, 2 h, 3 h, 4 h, 5 h 6 h, 24 h, 48 h, 72 h, or 96 h, preferably beyond 1 h, more preferably beyond 6 h, even more preferably beyond 24 h post injection.

63. The compound according to any one of claims 1-53 or the pharmaceutical composition according to claim 54 for use according to claim 61 or 62, the method further comprising administration of an immunocytokine as defined in any of claims 55-60.

64. An immunocytokine as defined in any of claims 55-60, for use as defined in claim 61 or 62, the method further comprising administration of the compound according to any one of claims 1-53 or the pharmaceutical composition according to claim 54.

65. The compound according to any one of claims 1-53 or the pharmaceutical composition according to claim 54 for use according to any one of claims 61 to 64, wherein the compound is administered to a subject at a dose of 10-500 nmol/kg, preferably 30-250 nmol/kg, more preferably 90-250 nmol/kg, even more preferably 90-160 nmol/kg, most preferably 90-125 nmol/kg, expressed as a mouse dose; or a corresponding human equivalent dose.

66. The compound according to any one of claims 1-53 or the pharmaceutical composition according to claim 54 for use according to any one of claims 61 to 64, wherein the compound is administered to a human subject at a dose of 0.8-40 nmol/kg, preferably 2-20 nmol/kg, more preferably 7-20 nmol/kg, even more preferably 7-13 nmol/kg, most preferably 7-10 nmol/kg.

67. The compound according to any one of claims 1-53 or the pharmaceutical composition according to claim 54 for use according to any one of claims 61 to 64, wherein the compound is administered to a human subject at a dose of 7 nmol/kg or more, preferably 7-20 nmol/kg.

68. The compound according to any one of claims 1-53 or the pharmaceutical composition according to claim 54 for use according to any one of claims 61 to 64, wherein the compound is administered to a human subject at a dose of 1 mg or more, preferably 1 to 3 mg.
